(19) United States
(12) Patent Application Publication
(10) Pub. No.: US 2026/0078097 A1
(43) Pub. Date: Mar. 19, 2026

(54) PIPERAZINE COMPOUNDS

(71) Applicant: Spark Therapeutics, Inc., Philadelphia, PA (US)

(72) Inventors: Rui Zhang, Winchester, MA (US); Hao Li, Drexel Hill, PA (US); Pedro Cejas, Oreland, PA (US); Ali Nahvi, Needham, MA (US)

(21) Appl. No.: 19/109,760

(22) PCT Filed: Sep. 7, 2023
(86) PCT No.: PCT/US2023/073629

§ 371 Date: Mar. 7, 2025

Related U.S. Application Data
(60) Provisional application No. 63/375,061, filed on Sep. 9, 2022.

(51) Int. Cl. C07D 241/04; A61K 31/495

(52) U.S. Cl. CPC C07D 241/04; A61K 31/495

(57) ABSTRACT

The present invention relates to novel piperazine compounds, in particular, of formula (I) or formula (II). These piperazine compounds can be used, for example, in lipid nanoparticle compositions for drug delivery and cancer treatments.

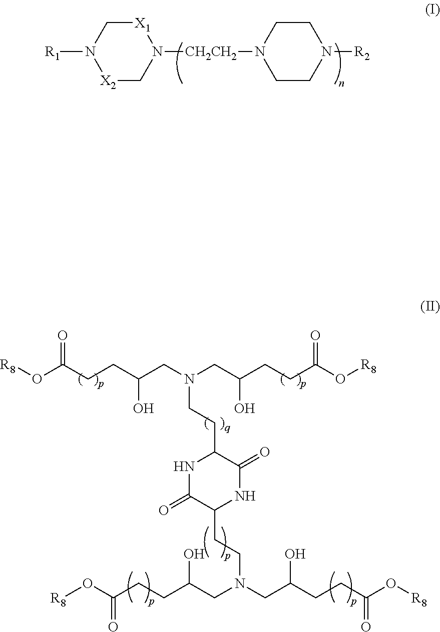

### CROSS REFERENCE TO RELATED APPLICATION

This application claims priority to U.S. Provisional Application No. 63/375,061 filed on Sep. 9, 2022, the disclosure of which is incorporated herein by reference in its entirety.

### FIELD OF THE INVENTION

This application describes novel piperazine compounds. These piperazine compounds can be used, for example, in lipid nanoparticle compositions for drug delivery and cancer treatments.

### BACKGROUND OF THE INVENTION

It is of great interest for therapeutics, diagnostics, reagents and for biological assays to be able to deliver a nucleic acid into a cell, such as to cause intracellular translation of the nucleic acid and production of the encoded protein. Delivery of nucleic acids has been explored extensively as a potential therapeutic option for certain disease states. In particular, double-stranded DNA (dsDNA) therapy has become an increasingly important option for treatment of various diseases, including cancer treatments. Intracellular delivery of dsDNA can stimulate the innate immune response, which can be helpful for treatment of various diseases.

Thus, there is a need to develop lipid compounds that can be useful to facilitate the delivery of nucleic acids to targeted cells or subjects in need thereof. The present invention satisfies this unmet need.

### BRIEF SUMMARY OF THE INVENTION

In a general aspect, the present application relates to a compound of formula (I):

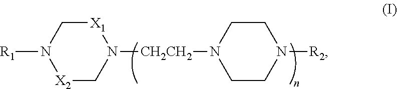

or a stereoisomer, tautomer, pharmaceutically acceptable salt or solvate thereof, wherein,
n is 0 or 1;
X₁ and X₂ are different or identical, and are each independently CHR₃;
R₁ and R₂ are different or identical, and each independently selected from the group consisting of C₁₋₈ alkyl and —C₁₋₈ alkyl-Y, wherein Y is —OR₄, —SR₄, or N(R₄)², and the C₁₋₈ alkyl is optionally substituted with one or more selected from the group consisting of halogen and C₁₋₈ alkyl;
R₃ is, independently at each occurrence, selected from the group consisting of hydrogen, alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, cycloalkyl, and heterocycle and said aryl or cycloalkyl or heterocycle is optionally substituted with one or more selected from the group consisting of halogen, hydroxyl, alkoxy, and amino;
R₄ is, independently at each occurrence, selected from the group consisting of alkyl and —C₁₋₄ alkyl-Z, wherein Z is —OR₅, —SR₅, or N(R₅)², and the alkyl is optionally substituted with one or more hydroxyl groups;
R₅ is, independently at each occurrence, selected from the group consisting of hydroxyl-substituted alkyl and (—C₁₋₄ alkyl-O)ₘ—C₁₋₄ alkyl-N(R₆)₂, wherein m is 0 or 1, R₆ is hydroxyl-substituted alkyl; and
with a proviso that at least one of R₁ and R₂ is not C₁₋₄ alkyl.

In some embodiments, X₁ and X₂ are identical, preferably are CH₂.

In some embodiments, the compound of formula (I) is a compound of formula (I-A):

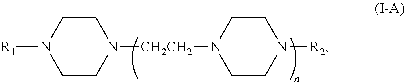

wherein R₁, R₂, and n are defined as above.

In some embodiments, n is 0.

In some embodiments, R₁ is —C₁₋₄ alkyl-Y.

In some embodiments, R₂ is —C₁₋₄ alkyl-Y.

In some embodiments, Y is —OR₄, —SR₄, or N(R₄)₂, preferably SR₄.

In some embodiments, R₄ is —C₁₋₄ alkyl-Z.

In some embodiments, Z is N(R₅)₂.

In some embodiments, R₅ is hydroxyl-substituted alkyl.

In some embodiments, R₃ is, independently at each occurrence, selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, and said aryl or cycloalkyl is optionally substituted with one or more selected from the group consisting of halogen, hydroxyl, and alkoxy.

In some embodiments, n is 0, and the compound is of formula (I-A1):

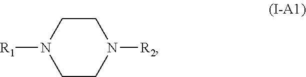

In other embodiments, n is 1, and the compound is of formula (I-A2):

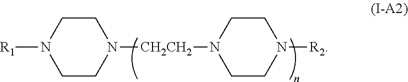

In some embodiments, the compound of formula (I) is a compound of formula (I-B):

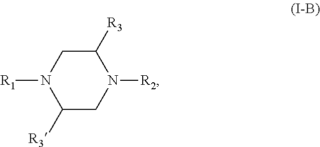

wherein R₃ and R_{3′} each is independently selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, and said aryl or cycloalkyl is optionally substituted with one or more selected from the group consisting of halogen, hydroxyl, and alkoxy, and R₁ and R₂ are defined as above.

In certain embodiments, the compound of formula (I) is a compound of formula (I-B1):

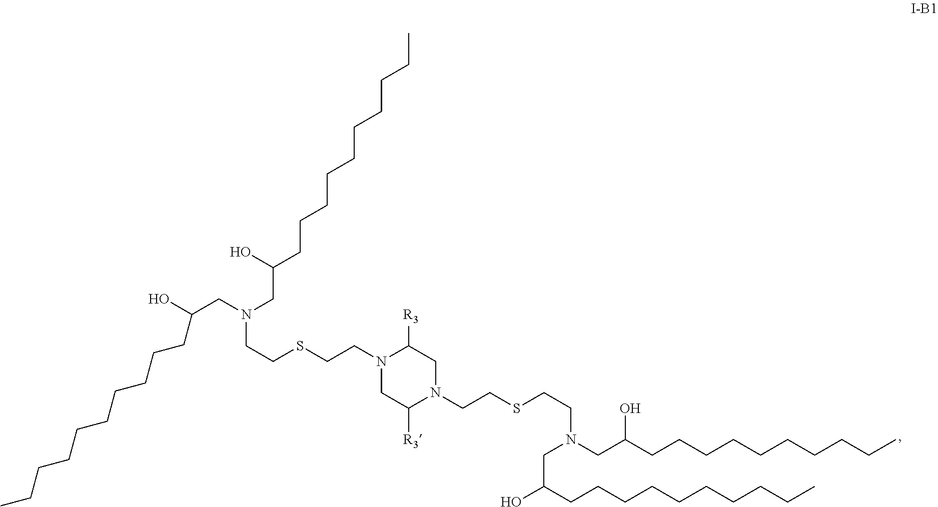

wherein R₃ and R_{3′} are different or identical, and each is independently selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, wherein the aryl or cycloalkyl is optionally substituted with one or more selected from the group consisting of halogen, hydroxyl, and alkoxy.

In further embodiments, R₃ and R_{3′} are identical.

In another general aspect, the present application relates to a compound of formula (II):

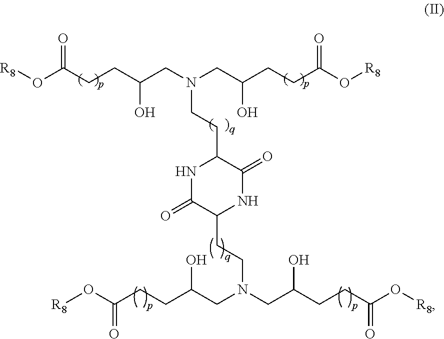

or a stereoisomer, tautomer, pharmaceutically acceptable salt or solvate thereof, wherein,
p is an integer from 1 to 10;
q is an integer from 3 to 8; and
R₈ is unbranched or branched alkyl.

In another aspect, the present application relates to a pharmaceutical composition comprising a compound as described herein or a stereoisomer, tautomer, pharmaceutically acceptable salt or solvate thereof, and a pharmaceutically acceptable carrier.

In another aspect, the present application relates to the use of a compound as described herein or a stereoisomer, tautomer, pharmaceutically acceptable salt or solvate thereof, for drug delivery or cancer treatment.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1. shows hEPO levels in the plasma evaluated at 4 hrs after administration of LNP-dsDNA formulations LNPs 1-11 using SIL1 series lipids.

FIG. 2. shows hFIX levels of HepG2 cells incubated with LNP-dsDNA formulations LNPs 915-934 at 75 ng/well using SIL2 series lipids.

FIG. 3. shows hFIX levels of HepG2 cells incubated with LNP-dsDNA formulations LNPs 915-934 at 150 ng/well using SIL2 series lipids.

FIG. 4. shows TRF levels of THP-1 incubated with LNP-dsDNA formulations LNPs 915-934 at 150 ng/well using SIL2 series lipids.

FIG. 5. shows TRF levels of THP-1 incubated with LNP-dsDNA formulations LNPs 915-934 at 75 ng/well using SIL2 series lipids.

FIG. 6. shows NFkB levels of THP-1 incubated with LNP-dsDNA formulations LNPs 915-934 at 150 ng/well using SIL2 series lipids.

FIG. 7. shows NFkB levels of THP-1 incubated with LNP-dsDNA formulations LNPs 915-934 at 75 ng/well using SIL2 series lipids.

FIG. 8. shows hEPO levels in the plasma evaluated at 4 hrs after administration of LNP-mRNA formulations LNPs 31-42 using SIL3 series lipids.

FIG. 9. shows hEPO levels in the plasma evaluated at 1 week and 2 weeks after administration of LNP-DNA formulations LNPs 43-54 using SIL3 series lipids.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible subranges, all individual numerical values within that range, including integers within such ranges and fractions of the values unless the context clearly indicates otherwise.

As used herein, “subject” means any animal, such as a mammal, to whom will be or has been treated by a method described herein. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc.

The phrase “pharmaceutically acceptable salt(s)” means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the application can form pharmaceutically acceptable salts with various amino acids. Suitable base salts include, but are not limited to, aluminum, calcium, lithium, magnesium, potassium, sodium, zinc, bismuth, and diethanolamine salts. For a review on pharmaceutically acceptable salts see Berge et al., 66 J. Pharm. Sci. 1-19 (1977), incorporated herein by reference.

As used herein, the term “alkyl” means a saturated, monovalent, unbranched or branched hydrocarbon chain. An alkyl group can be unsubstituted or substituted with one or more suitable substituents. Examples of alkyl groups include, but are not limited to, methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When numbers appear in a subscript after the symbol “C”, the subscript defines with more specificity the number of carbon atoms which that particular alkyl can contain. For example, “C₁ to C₁₀ alkyl” or “C₁₋₁₀ alkyl” is intended to include alkyl groups having 1, 2, 3, 4, 5, 6, 7, 8, 9, and 10 carbon atoms. Additionally, for example, “C₁ to C₈ alkyl” or “C₁₋₈ alkyl” denotes an alkyl having 1, 2, 3, 4, 5, 6, 7, or 8 carbon atoms.

The term “cycloalkyl” refers to any stable monocyclic or polycyclic saturated hydrocarbon ring system. A cycloalkyl group can be unsubstituted or substituted with one or more suitable substituents. A cycloalkyl group can have a specified number of carbon atoms. For example, “C₃ to C₆ cycloalkyl” or “C₃₋₆ cycloalkyl” includes cycloalkyl groups having 3, 4, 5, or 6 ring carbon atoms, i.e., cyclopropyl, cyclobutyl, cyclopentyl, and cyclohexyl. Polycyclic cycloalkyls include bridged, fused, and spiro ring structures in which all ring atoms are carbon atoms. A “spiro ring” is a polycyclic ring system in which two rings share one carbon atom, referred to as the “spiro atom,” which is typically a quaternary carbon atom. A “fused ring” is a polycyclic ring system in which two rings share two adjacent atoms, referred to as “bridgehead atoms,” i.e., the two rings share one covalent bond such that the bridgehead atoms are directly connected. A “bridged ring” is a polycyclic ring system in which two rings share three or more atoms separating the bridgehead atoms by a bridge containing at least one atom. Examples of cycloalkyl groups include, but are not limited to, cyclopropyl, cyclobutyl, cyclopentyl, cyclohexyl, cycloheptyl, cyclooctyl, etc.

As used herein, the term “alkenyl” means a linear or branched chain of hydrocarbons comprising at least one carbon to carbon double bond, optionally having the number of carbon atoms designated (i.e., C₂-C₄ alkenyl or C₂₋₄alkenyl means an alkenyl having two to four carbon atoms).

As used herein, the term “alkynyl” means a linear or branched chain of hydrocarbons comprising at least one carbon to carbon triple bond, optionally having the number of carbon atoms designated (i.e., C₂-C₄ alkenyl or C₂₋₄alkenyl means an alkenyl having two to four carbon atoms).

The term “aryl” as used herein is a group that contains any carbon-based aromatic group including, but not limited to, phenyl, naphthyl, anthracenyl, phenanthranyl, and the like. Aryl moieties are well known and described, for example, in Lewis, R. J., ed., Hawley's Condensed Chemical Dictionary, 13th Edition, John Wiley & Sons, Inc., New York (1997). An aryl group can be substituted or unsubstituted with one or more suitable substituents. An aryl group can comprise a single ring structure (i.e., monocyclic) or multiple ring structures (i.e., polycyclic, e.g., bicyclic or tricyclic). For example, an aryl group can be a monocyclic aryl group, e.g., phenyl.

The term “heterocyclyl” includes stable monocyclic and polycyclic hydrocarbons that contain at least one heteroatom ring member, such as sulfur, oxygen, or nitrogen, wherein the ring structure is saturated or partially unsaturated, provided the ring system is not fully aromatic. A heterocyclyl group can be unsubstituted, or substituted with one or more suitable substituents at any one or more of the carbon atom(s) and/or nitrogen heteroatom(s) of the heterocyclyl. A heterocyclyl can comprise a single ring structure (i.e., monocyclic) or multiple ring structures (i.e., polycyclic, e.g., bicyclic). Polycyclic heterocyclyls include bridged, fused, and spiro ring structures in which at least one ring atom of at least one of the rings of the polycyclic ring system is a heteroatom, for instance oxygen, nitrogen, or sulfur, wherein bridged, fused, and spiro rings are as defined above. A heterocyclyl ring can be attached to the parent molecule at any suitable heteroatom (typically nitrogen) or carbon atom of the ring. The term “4- to 9-membered monocyclic or bicyclic heterocyclyl” includes any four, five, six, seven, eight, or nine membered monocyclic or bicyclic ring structure containing at least one heteroatom ring member selected from oxygen, nitrogen, and sulfur, or independently selected from oxygen and nitrogen, optionally containing one to three additional heteroatoms independently selected from oxygen, nitrogen, and sulfur, or independently selected from oxygen and nitrogen, wherein the ring structure is saturated or partially unsaturated, provided the ring structure is not fully aromatic.

As used herein, the term “heteroaryl” includes stable monocyclic and polycyclic aromatic hydrocarbons that contain at least one heteroatom ring member such as sulfur, oxygen, or nitrogen. A heteroaryl group can be unsubstituted or substituted with one or more suitable substituents. A heteroaryl can comprise a single ring structure (i.e., monocyclic) or multiple ring structures (i.e., polycyclic, e.g., bicyclic or tricyclic). Each ring of a heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring, but the other fused ring or rings can be aromatic or non-aromatic. For example, for a bicyclic heteroaryl, the fused rings completing the bicyclic group can contain only carbon atoms and can be saturated, partially saturated, or unsaturated. A heteroaryl can be attached to the parent molecule at any available nitrogen or carbon atom of any ring of the heteroaryl group. In some embodiments, the term “heteroaryl” refers to 5- or 6-membered monocyclic groups and 9- or 10-membered bicyclic groups which have at least one heteroatom (O, S, or N) in at least one of the rings, wherein the heteroatom-containing ring typically has 1, 2, or 3 heteroatoms, such as 1 or 2 heteroatoms, selected from O, S, and/or N. A heteroaryl group can be unsubstituted, or substituted with one or more suitable substituents at any one or more of the carbon atom(s) and/or nitrogen heteroatom(s) of the heteroaryl. The nitrogen and sulfur heteroatom(s) of a heteroaryl can optionally be oxidized (i.e., N→O and S(O)r, wherein r is 0, 1 or 2).

The term “alkoxy” as used herein refers to an —O-alkyl group, wherein alkyl is as defined above. An alkoxy group is attached to the parent molecule through a bond to an oxygen atom. An alkoxy group can have a specified number of carbon atoms. For example, “C₁ to C₁₀ alkoxy” or “C₁₋₁₀ alkoxy” is intended to include alkoxy groups having 1, 2, 3, 4, 5, 6, 7, 8, 9, and 10 carbon atoms. Additionally, for example, “C₁ to C₄ alkoxy” or “C₁₋₄ alkoxy” denotes an alkoxy having 1, 2, 3, or 4 carbon atoms. Examples of alkoxy include, but are not limited to, methoxy, ethoxy, propoxy (e.g., n-propoxy, isopropoxy), butoxy (e.g., n-butoxy, isobutoxy, tert-butoxy), pentyloxy (e.g., n-pentyloxy, isopentyloxy, neopentyloxy), etc. An alkoxy group can be unsubstituted or substituted with one or more suitable substituents. Similarly, “alkylthio” or “thioalkoxy” represents an alkyl group as defined above attached to the parent molecule through a bond to a sulfur atom, for example, —S-methyl, —S-ethyl, etc. Representative examples of alkylthio include, but are not limited to, —SCH₃, —SCH₂CH₃, etc.

As used herein, the term “halogen” means fluorine, chlorine, bromine, or iodine. Correspondingly, the term “halo” means fluoro, chloro, bromo, and iodo.

“Haloalkyl” is intended to include both branched and straight-chain saturated aliphatic hydrocarbon radicals substituted with one or more halogen atoms. “Fluorinated alkyl” or “fluoroalkyl” in particular refers to any alkyl group as defined above substituted with at least one fluoro atom, e.g., one to three fluoro atoms, such as one, two, or three fluoroatoms. Examples of haloalkyl include, but are not limited to, fluoromethyl, difluoromethyl, trifluoromethyl, chloromethyl, dichloromethyl, trichloromethyl, pentafluoroethyl, pentachloroethyl, 2,2,2-trifluoroethyl, heptafluoropropyl, and heptachloropropyl. Suitable examples of fluoroalkyl in particular include, but are not limited to, —CF₃, —CHF₂, —CH₂F, —CH₂CF₃, —CF₂CF₃, and the like.

The terms “hydroxy” and “hydroxyl” can be used interchangeably, and refer to —OH.

The term “carboxy” refers to —COOH.

The term “ester” refers to —COOR, wherein R is alkyl as defined above.

The term “cyano” refers to —CN.

The term “oxo” refers to a double bonded oxygen group, i.e., a substituent group of the formula ═O.

The term “keto” refers to —C(O)R, wherein R is alkyl as defined above.

The term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

The term “aminoalkyl” as used herein is intended to include both branched and straight-chain saturated aliphatic hydrocarbon groups substituted with one or more amino groups. For example, “C₁₋₄ aminoalkyl” is intended to include alkyl groups having 1, 2, 3, or 4 carbon atoms substituted with one or more amino groups. Aminoalkyl groups are attached to the parent molecule through a bond to a carbon atom of the alkyl moiety of the aminoalkyl group. Representative examples of aminoalkyl groups include, but are not limited to, —CH₂NH₂, —CH₂CH₂NH₂, and —CH₂CH(NH₂)CH₃.

As used herein, “amido” refers to —C(O)N(R)₂, wherein each R is independently an alkyl group (including both branched and straight-chain alkyl groups) or a hydrogen atom. Examples of amido groups include, but are not limited to, —C(O)NH₂, —C(O)NHCH₃, and —C(O)N(CH₃)₂.

The terms “hydroxyl-substituted alkyl,” “hydroxylalkyl” and “hydroxyalkyl” are used interchangeably, and refer to a branched or straight-chain aliphatic hydrocarbon group substituted with one or more hydroxyl groups. Hydroxyalkyl groups are attached to the parent molecule through a bond to a carbon atom of the alkyl moiety of the hydroxyalkyl group. A hydroxyalkyl group can have a specified number of carbon atoms. For example, “C₁ to C₁₀ hydroxyalkyl” or “C₁₋₁₀ hydroxyalkyl” is intended to include hydroxyalkyl groups having 1, 2, 3, 4, 5, 6, 7, 8, 9, and 10 carbon atoms. Additionally, for example, “C₁ to C₄ hydroxylalkyl” or “Ct-4 hydroxyalkyl” denotes a hydroxyalkyl group having 1, 2, 3, or 4 carbon atoms. Examples of hydroxyalkyl include, but are not limited to, hydroxylmethyl (—CH₂OH), hydroxylethyl (—CH₂CH₂OH), etc.

In accordance with convention used in the art:

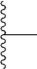

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

The term “substituted” as used herein with respect to any organic radical (e.g., alkyl, cycloalkyl, heteroaryl, aryl, heterocyclyl, etc.) means that at least one hydrogen atom is replaced with a non-hydrogen group, provided that all normal valencies are maintained and that the substitution results in a stable compound. When a particular group is “substituted,” that group can have one or more substituents, such as from one to five substituents, one to three substituents, or one to two substituents, independently selected from the list of substituents. The term “independently” when used in reference to substituents, means that when more than one of such substituents is possible, such substituents can be the same or different from each other. Examples of suitable substituents include, but are not limited to, alkyl, halo, haloalkyl, alkoxy, amido, hydroxy, hydroxyalkyl, amino, carboxyl, ester, oxo, cyano and the like.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

The terms “optional” or “optionally” mean that the event or circumstance described subsequently can, but need not, occur, and such a description includes the situation in which the event or circumstance does or does not occur. For example, “optionally substituted heterocyclyl” means that a substituent group can be, but need not be, present, and such a description includes the situation of the heterocyclyl group being substituted by a suitable substituent and the heterocyclyl group not being substituted by any substituent.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds, or otherwise indicated as having a particular configuration (e.g., R or S) around one or more atoms, contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or diastereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Certain examples contain chemical structures that comprise (R) or (S) terminology. When (R) or (S) is used in the name of a compound or in the chemical representation of the compound, it is intended to mean that the compound is a single isomer at that stereocenter, with established absolute configuration of either (R) or (S).

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diastereomeric salts with optically active acids or bases or by chiral chromatography. Pure stereoisomers can also be prepared synthetically from appropriate stereochemically pure starting materials, or by using stereoselective reactions.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the application.

Compounds described herein can exist in solvated and unsolvated forms. The term “solvate” means a physical association, e.g., by hydrogen bonding, of a compound of the application with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds of the application can form solvates with water (i.e., hydrates) or common organic solvents. Exemplary solvates include, but are not limited to, hydrates, ethanolates, methanolates, and isopropanolates. Methods of solvation are generally known in the art.

Also included within the scope of the application are all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds can generally be prepared by conventional techniques known to those skilled in the art or by processes analogous to those described herein, using an appropriate isotopically-labeled reagent in place of the non-labeled reagent otherwise employed.

As used herein, the name of a compound is intended to encompass all possible existing isomeric forms, including stereoisomers (e.g., enantiomers, diastereomers, racemate or racemic mixture, and any mixture thereof) of the compound.

### Compounds

In one general aspect, the present application relates to a compound formula (I):

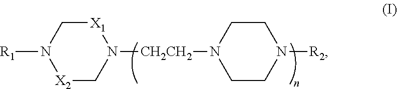

or a stereoisomer, tautomer, pharmaceutically acceptable salt or solvate thereof, wherein,
n is 0 or 1;
X₁ and X₂ are different or identical, and are each independently CHR₃;
R₁ and R₂ are different or identical, and each independently selected from the group consisting of C₁₋₄ alkyl and —C₁₋₄ alkyl-Y, wherein Y is —OR₄, —SR₄, or N(R₄)², and the C₁₋₄ alkyl is optionally substituted with one or more selected from the group consisting of halogen and C₁₋₄ alkyl;
R₃ is, independently at each occurrence, selected from the group consisting of hydrogen, alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, cycloalkyl, and heterocycle and said aryl or cycloalkyl or heterocycle is optionally substituted with one or more selected from the group consisting of halogen, hydroxyl, alkoxy, and amino;
R₄ is, independently at each occurrence, selected from the group consisting of alkyl and —C₁₋₄ alkyl-Z, wherein Z is —OR₅, —SR₅, or N(R₅)², and the alkyl is optionally substituted with one or more hydroxyl groups;
R₅ is, independently at each occurrence, selected from the group consisting of hydroxyl-substituted alkyl and (—C₁₋₄ alkyl-O)ₘ—C₁₋₄ alkyl-N(R₆)₂, wherein m is 0 or 1, R₆ is hydroxyl-substituted alkyl; and
with a proviso that at least one of R₁ and R₂ is not C₁₋₄ alkyl.

In some embodiments, X₁ and X₂ are identical. In certain embodiments, X₁ and X₂ are both CH₂.

In some embodiments, the compound of formula (I) is a compound of formula (I-A):

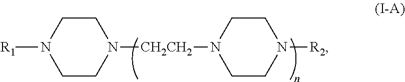

wherein R₁, R₂, and n are defined as above.

In some embodiments, n is 0, and the compound is of formula (I-A1):

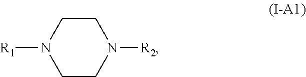

In other embodiments, n is 1, and the compound is of formula (I-A2):

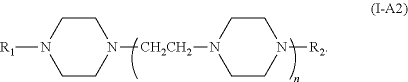

In some embodiments, R₁ is C₁₋₄ alkyl, such as methyl, ethyl, propyl (e.g., n-propyl, isopropyl), and butyl (e.g., n-butyl, isobutyl, tert-butyl).

In some embodiments, R₁ is —C₁₋₄ alkyl-Y, wherein Y is —OR₄, —SR₄, or N(R₄)², and the C₁₋₄ alkyl is optionally substituted with one or more selected from the group consisting of halogen and C₁₋₄ alkyl.

In some embodiments, R₂ is C₁₋₄ alkyl, such as methyl, ethyl, propyl (e.g., n-propyl, isopropyl), and butyl (e.g., n-butyl, isobutyl, tert-butyl).

In some embodiments, R₂ is —C₁₋₄ alkyl-Y, wherein Y is —OR₄, —SR₄, or N(R₄)², and the C₁₋₄ alkyl is optionally substituted with one or more selected from the group consisting of halogen and C₁₋₄ alkyl.

In some embodiments, Y is —OR₄, —SR₄, or N(R₄)₂, preferably SR₄.

In some embodiments, R₄ is alkyl, and the alkyl is optionally substituted with one or more hydroxyl groups.

In some embodiments, R₄ is —C₁₋₄ alkyl-Z, wherein Z is —OR₅, —SR₅, or N(R₅)².

In certain embodiments, when Y is N(R₄)₂, then R₄ is alkyl, and the alkyl is optionally substituted with one or more hydroxyl groups. In preferred embodiments, R₄ is

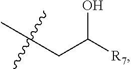

and R₇ is unbranched alkyl. In more preferred embodiments, R₄ is

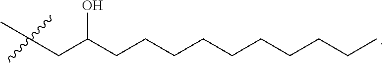 [SMILES: *CC(O)CCCCCCCCCC]

In certain embodiments, when Y is —N(R₄)₂, then R₄ is —C₁₋₄ alkyl-Z, wherein Z is —OR₅, —SR₅, or N(R₅)².

In certain embodiments, when Y is —OR₄ or —SR₄, then R₄ is —C₁₋₄ alkyl-Z, wherein Z is —OR₅, —SR₅, or N(R₅)².

In some embodiments, R₅ is hydroxyl-substituted alkyl.

In some embodiments, R₅ is (—C₁₋₄ alkyl-O)ₘ—C₁₋₄ alkyl-N(R₆)₂, wherein m is 0 or 1, R₆ is hydroxyl-substituted alkyl.

In certain embodiments, when Z is N(R₅)², then R₅ is hydroxyl-substituted alkyl, and the hydroxyl-substituted alkyl can be linear or branched. In preferred embodiments, R₅ is

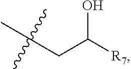

and R₇ is unbranched alkyl. In more preferred embodiments, R₅ is

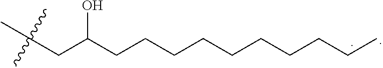 [SMILES: *CC(O)CCCCCCCCCC]

In certain embodiments, when Z is N(R₅)², then R₅ is (—C₁₋₄ alkyl-O)ₘ—C₁₋₄ alkyl N(R₆)₂, wherein m is 0 or 1, R₆ is hydroxyl-substituted alkyl.

In certain embodiments, when Z is —OR₅ or —SR₅, then R₅ is (—C₁₋₄ alkyl-O)ₘ—C₁₋₄ alkyl-N(R₆)₂, wherein m is 0 or 1, R₆ is hydroxyl-substituted alkyl.

In preferred embodiments, R₆ is

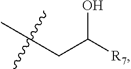

and R₇ is unbranched alkyl. In more preferred embodiments, R₆ is

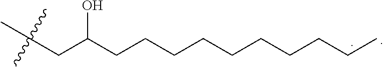 [SMILES: *CC(O)CCCCCCCCCC]

Exemplary compounds of formula (I-A) include, but are not limited to, the following compounds SIL1-1 to SIL1-17, and any tautomer, stereoisomer, pharmaceutically acceptable salt or solvate thereof:

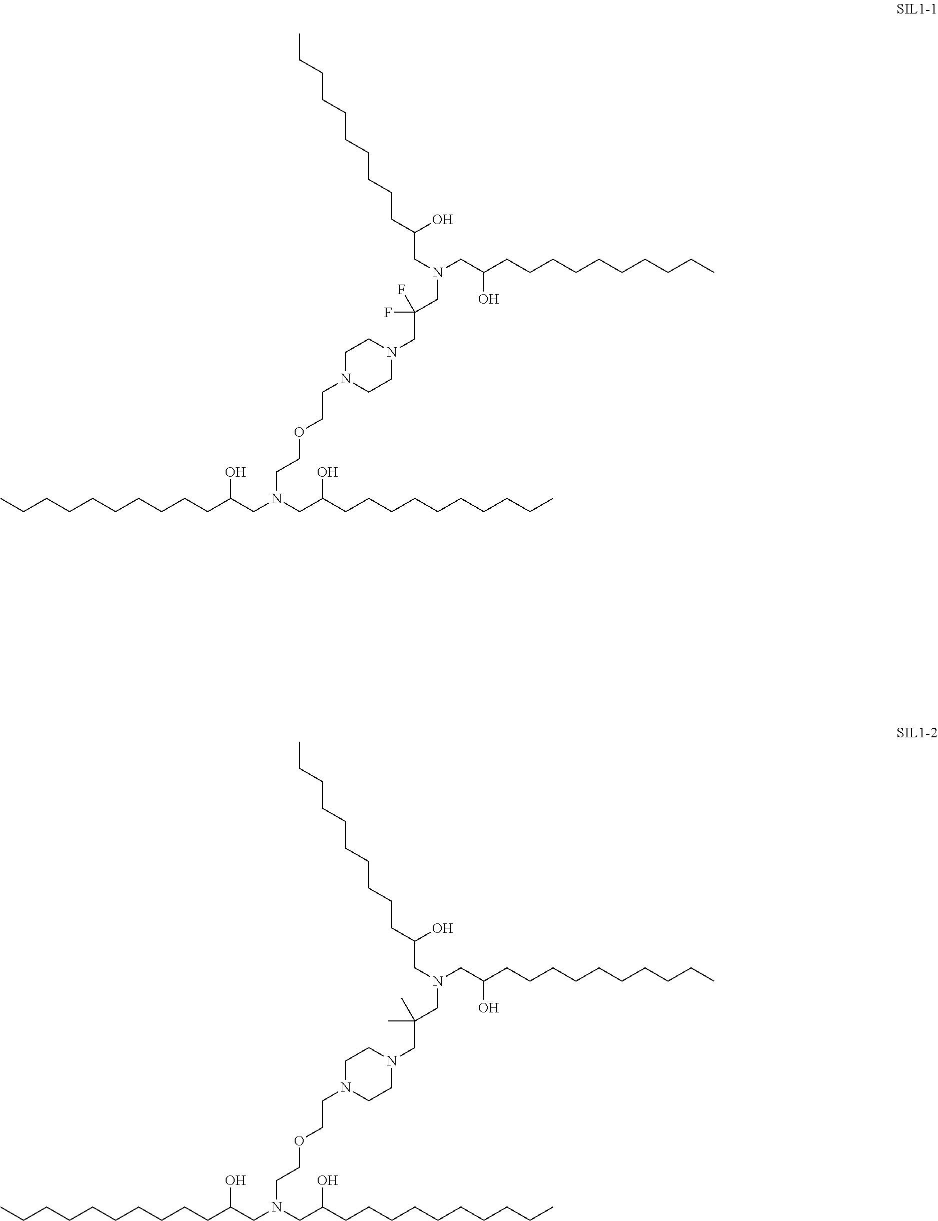

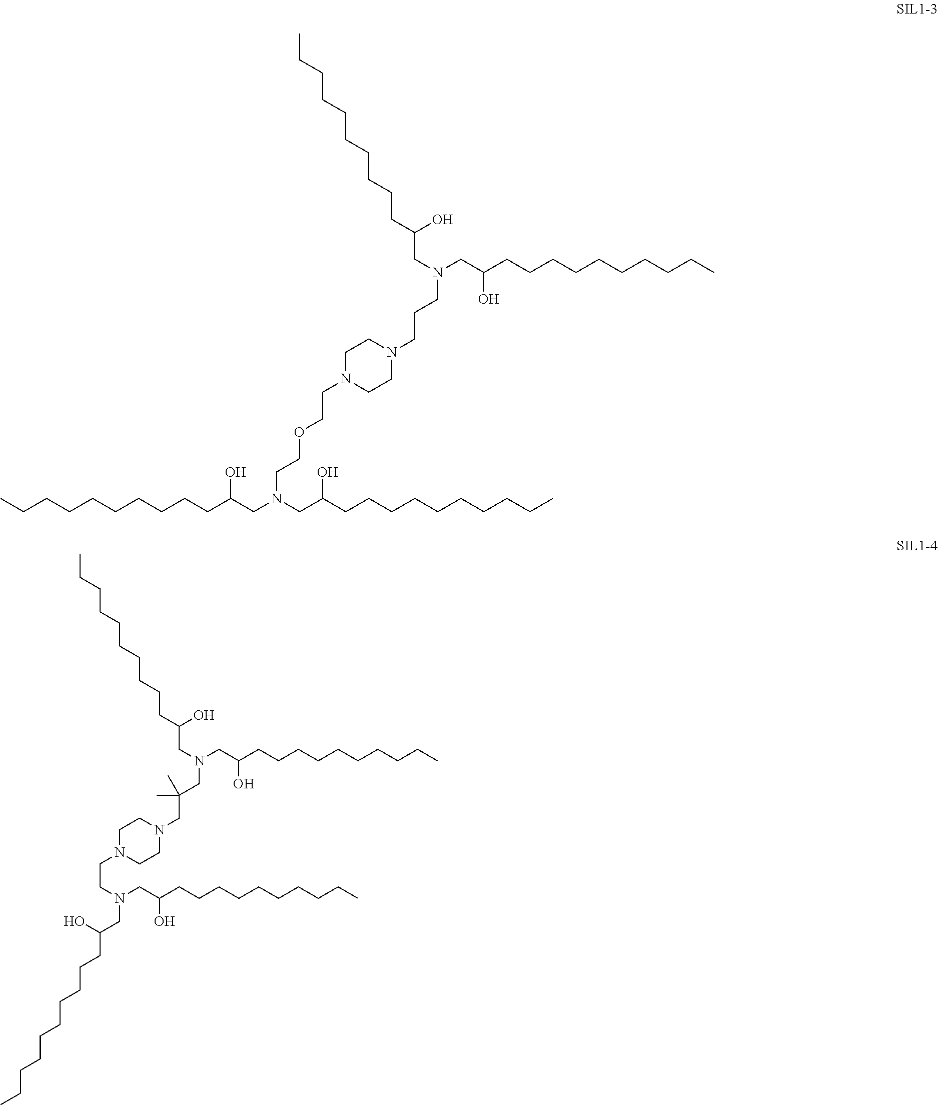

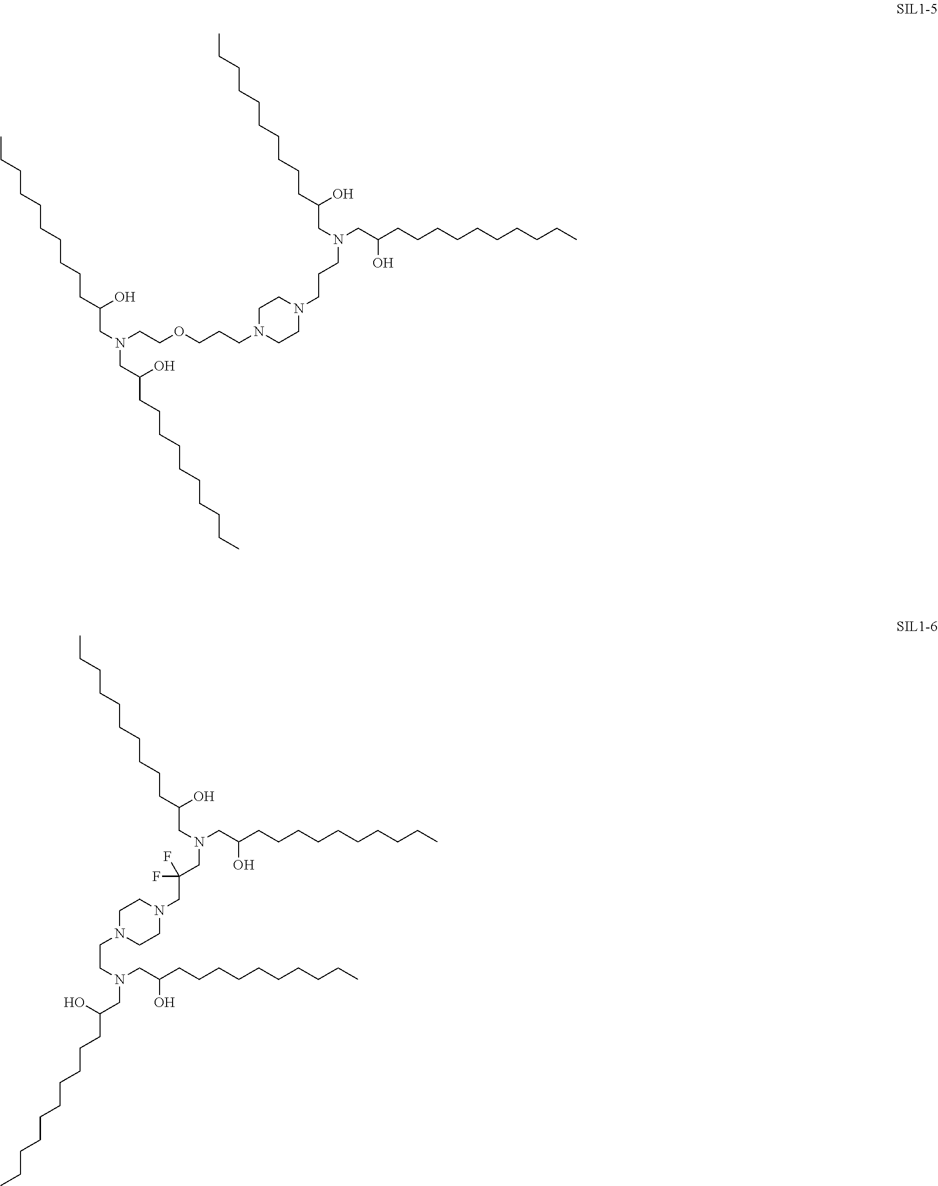

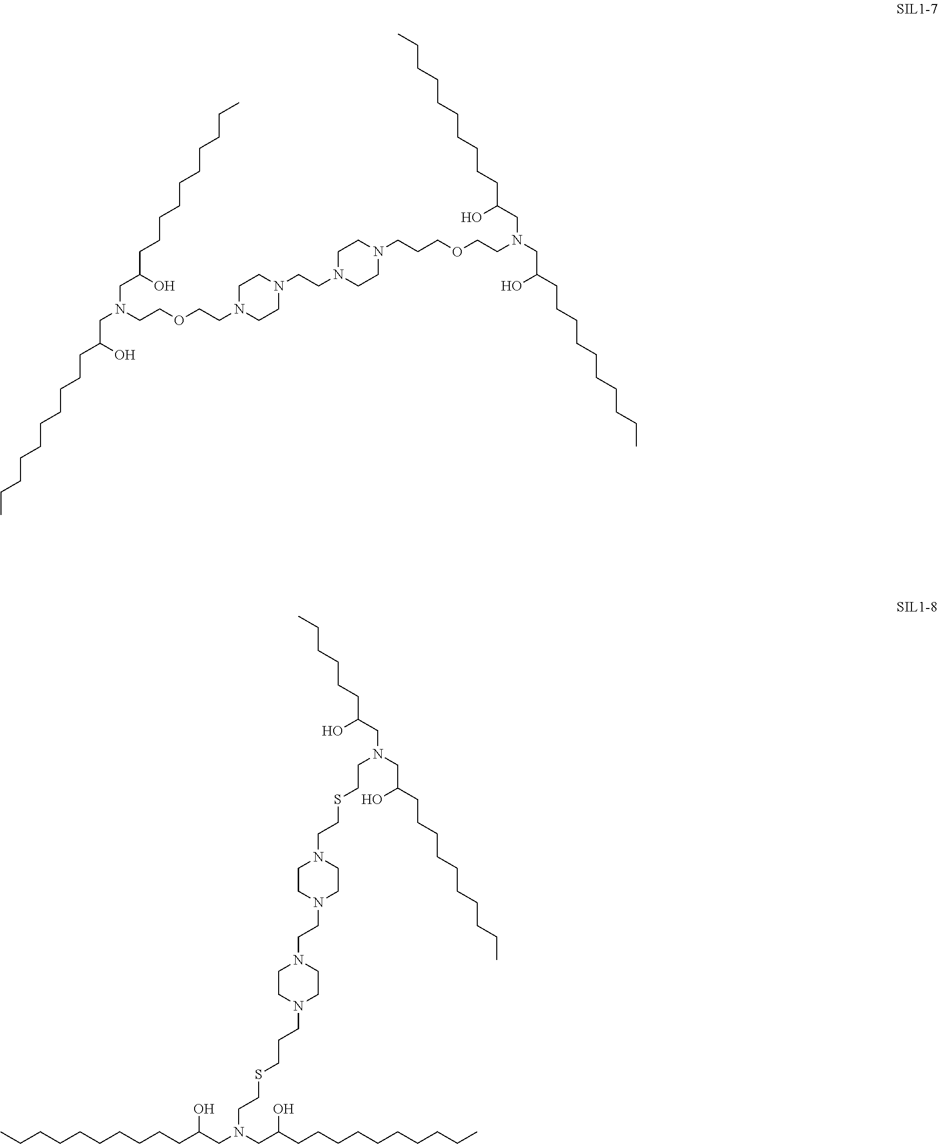

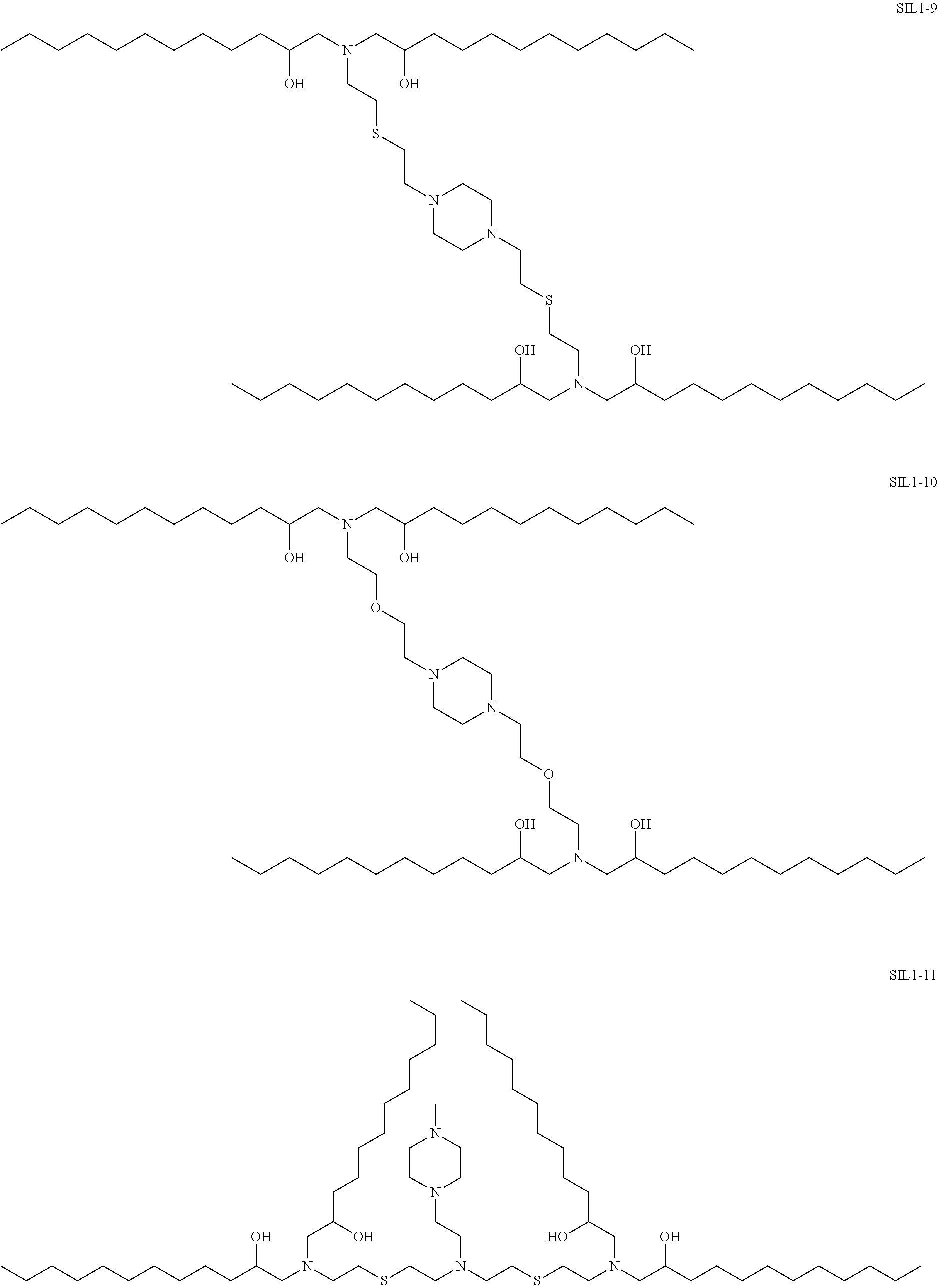

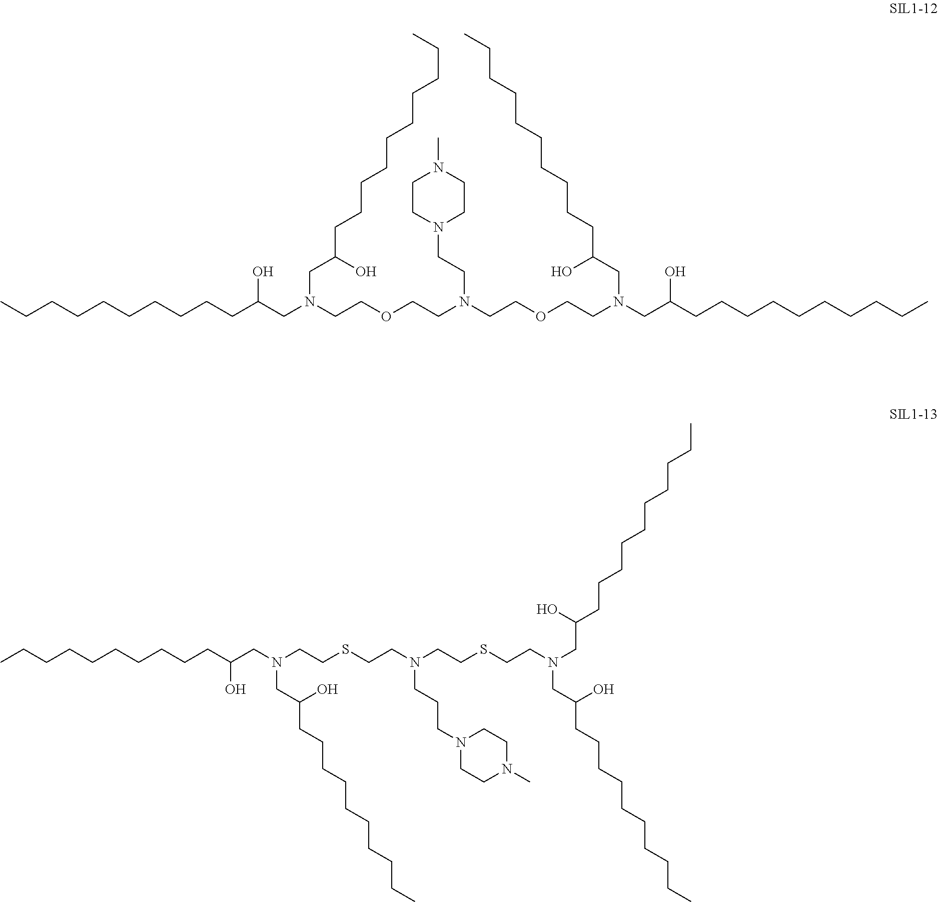

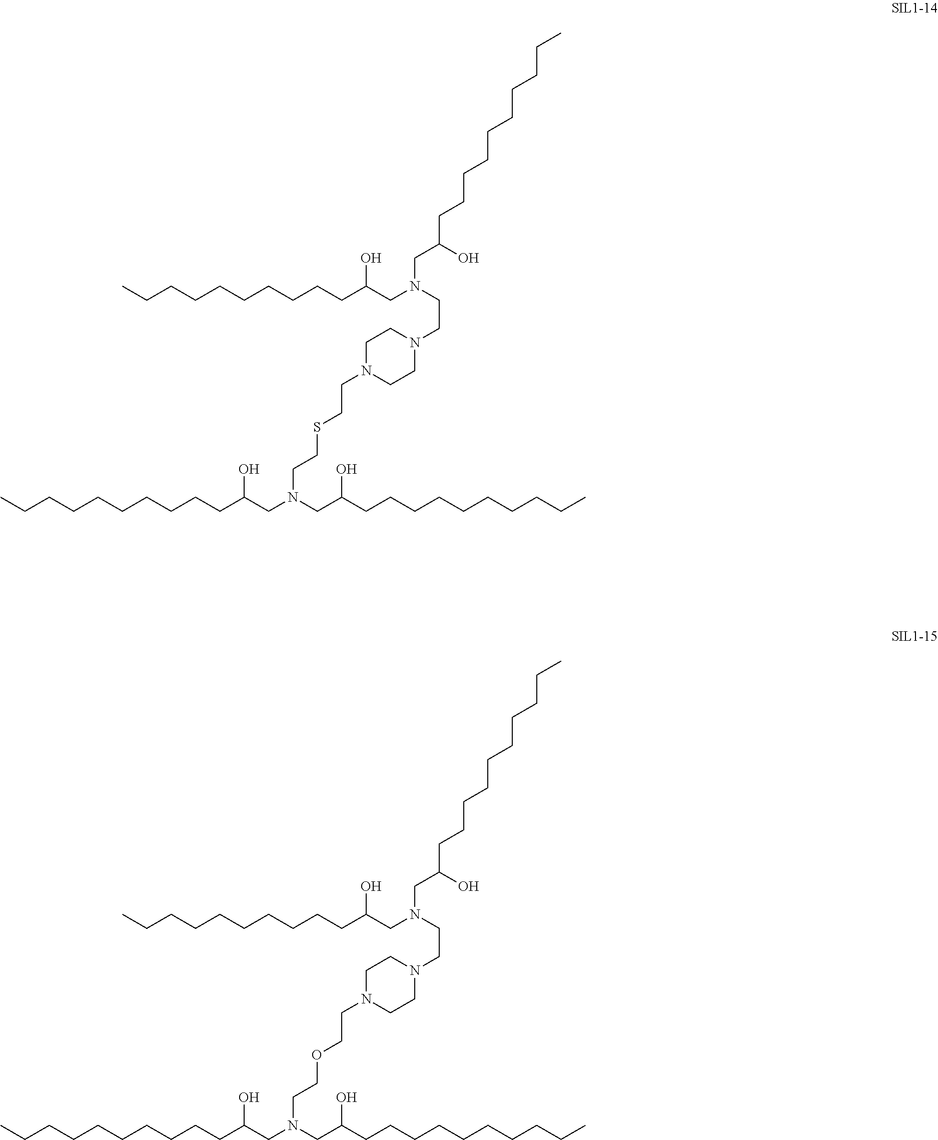

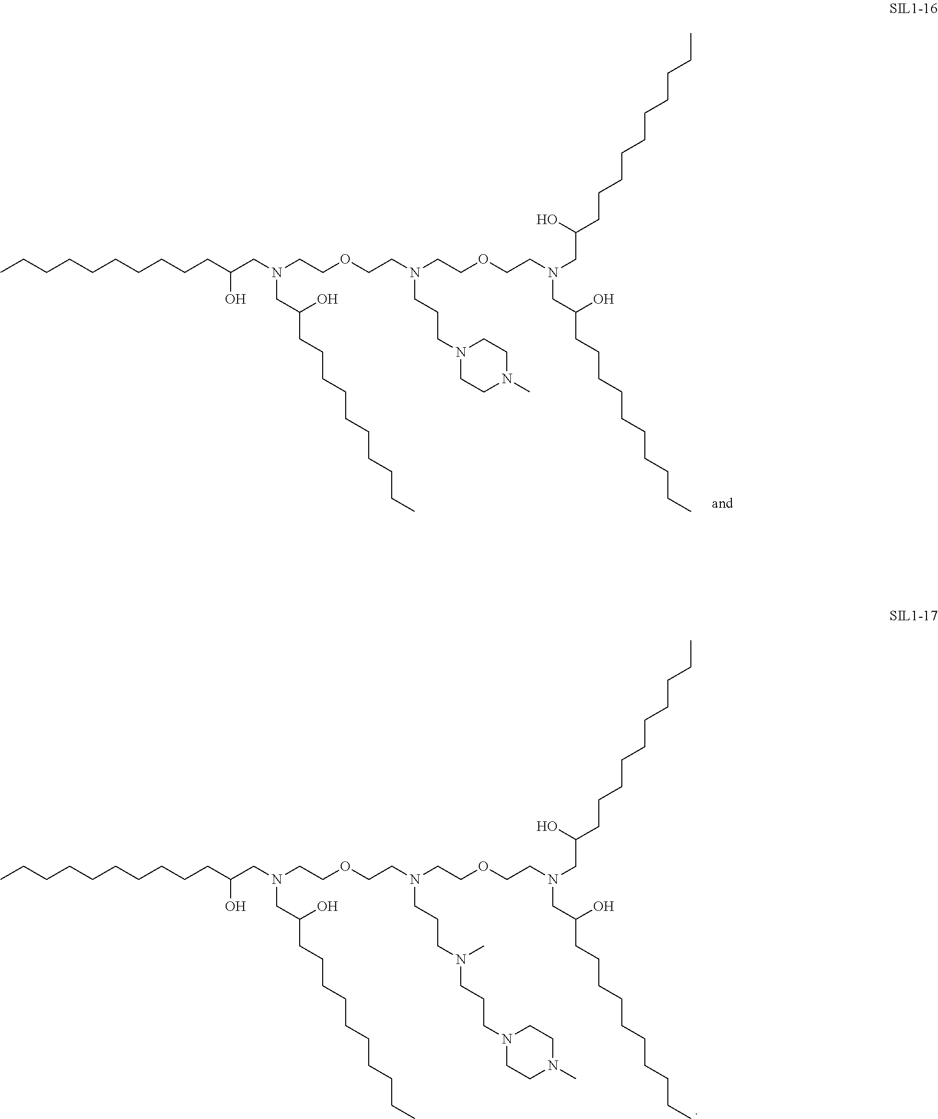

In some embodiments, X₁ and X₂ are CHR₃, wherein R₃ is, independently at each occurrence, selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, and said aryl or cycloalkyl is optionally substituted with one or more selected from the group consisting of halogen, hydroxyl, and alkoxy.

In some embodiments, the compound of formula (I) is a compound of formula (I-B):

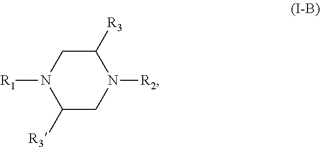

wherein R₃ and R_{3′} each is independently selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, and said aryl or cycloalkyl is optionally substituted with one or more selected from the group consisting of halogen, hydroxyl, and alkoxy, and R₁ and R₂ are defined as above.

In certain embodiments, R₃ and R_{3′} are identical.

In certain embodiments R₁ and R₂ are identical, preferably are —C₁₋₄ alkyl-Y.

In further embodiments, Y is —OR₄, —SR₄, or N(R₄)₂, preferably SR₄.

In further embodiments, R₄ is —C₁₋₄ alkyl-Z.

In further embodiments, Z is N(R₅)₂.

In further embodiments, wherein R₅ is hydroxyl-substituted alkyl. In preferred embodiments, R₅ is

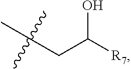

and R₇ is unbranched alkyl. In more preferred embodiments, R₅ is

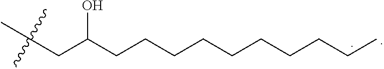 [SMILES: *CC(O)CCCCCCCCCC]

In some embodiments, the compound of formula (I-B) is a compound of formula (I-B1):

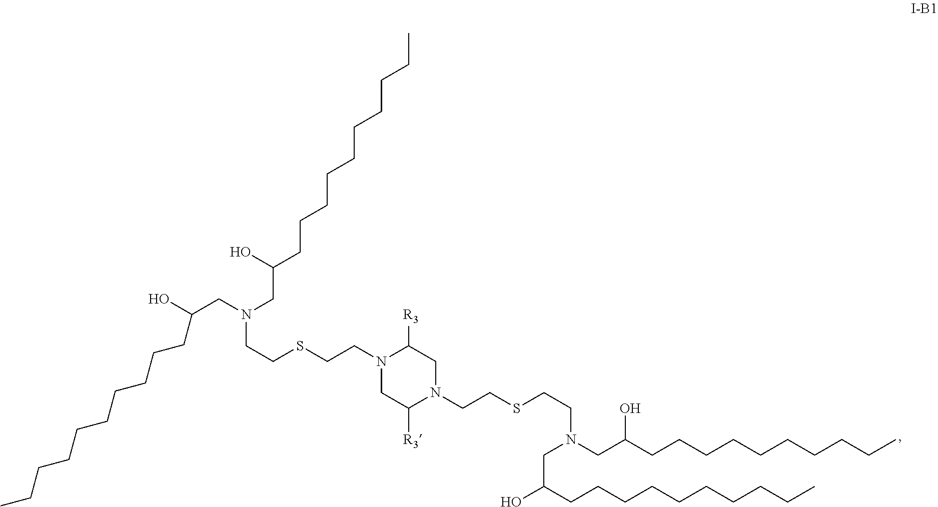

wherein R₃ and R_{3′} are different or identical, and each is independently selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, wherein the aryl or cycloalkyl is optionally substituted with one or more selected from the group consisting of halogen, hydroxyl, and alkoxy.

In some embodiments, in the compound of formula (I-B1), R₃ and R₃ are identical.

In some embodiments, the compound of formula (I-B) is a compound of formula (I-B2):

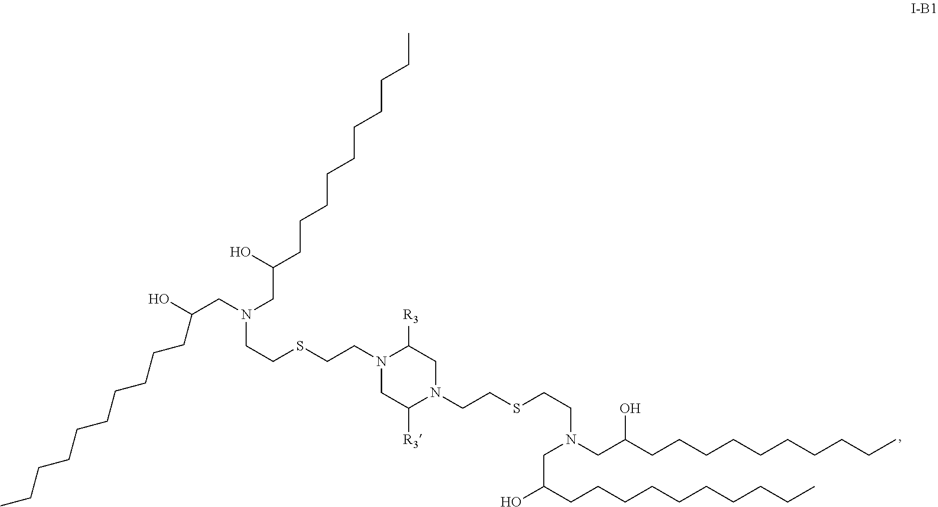

wherein R₃ is selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, wherein the aryl or cycloalkyl is optionally substituted with one or more selected from the group consisting of halogen, hydroxyl, and alkoxy.

Exemplary compounds of formula (I-B) include, but are not limited to, the following compounds SIL2-1 to SIL2-22, and any tautomer, stereoisomer, pharmaceutically acceptable salt or solvate thereof:

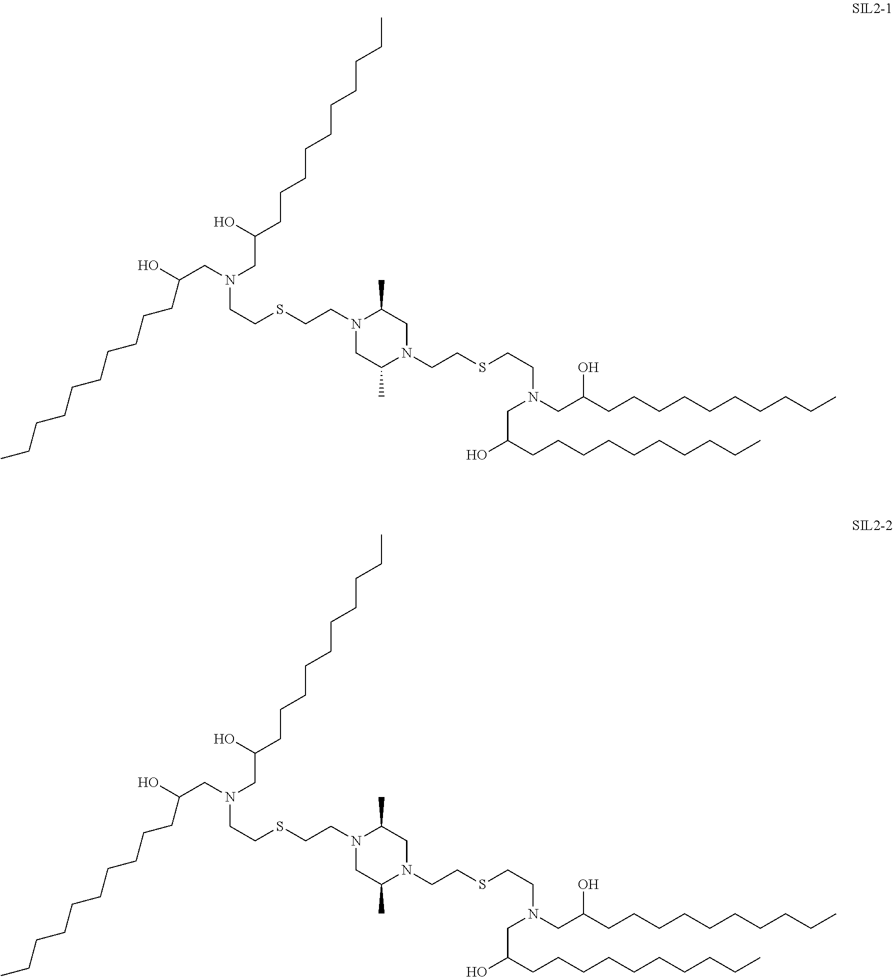

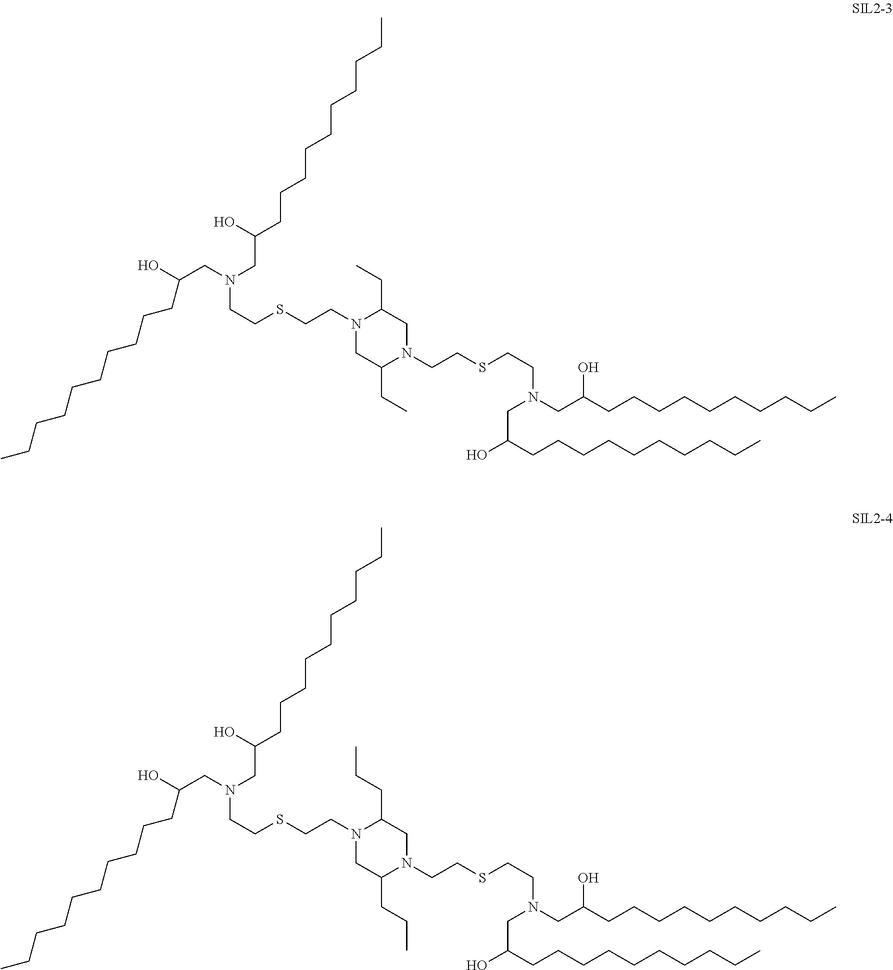

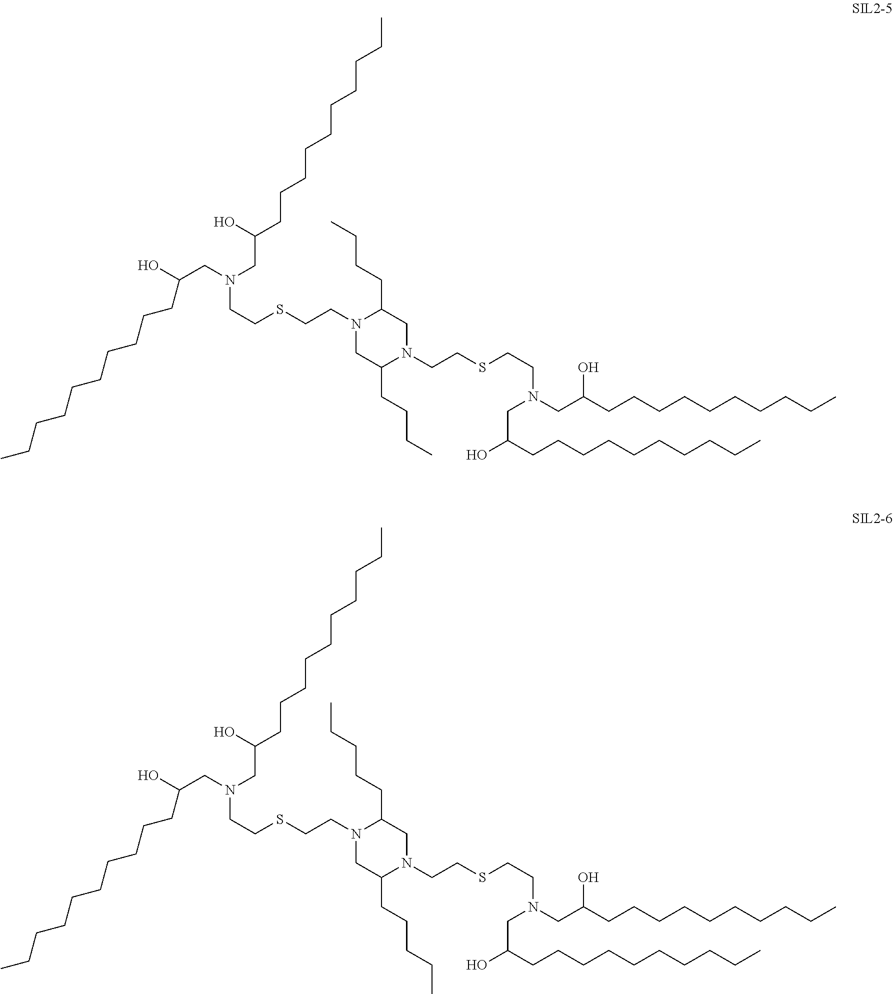

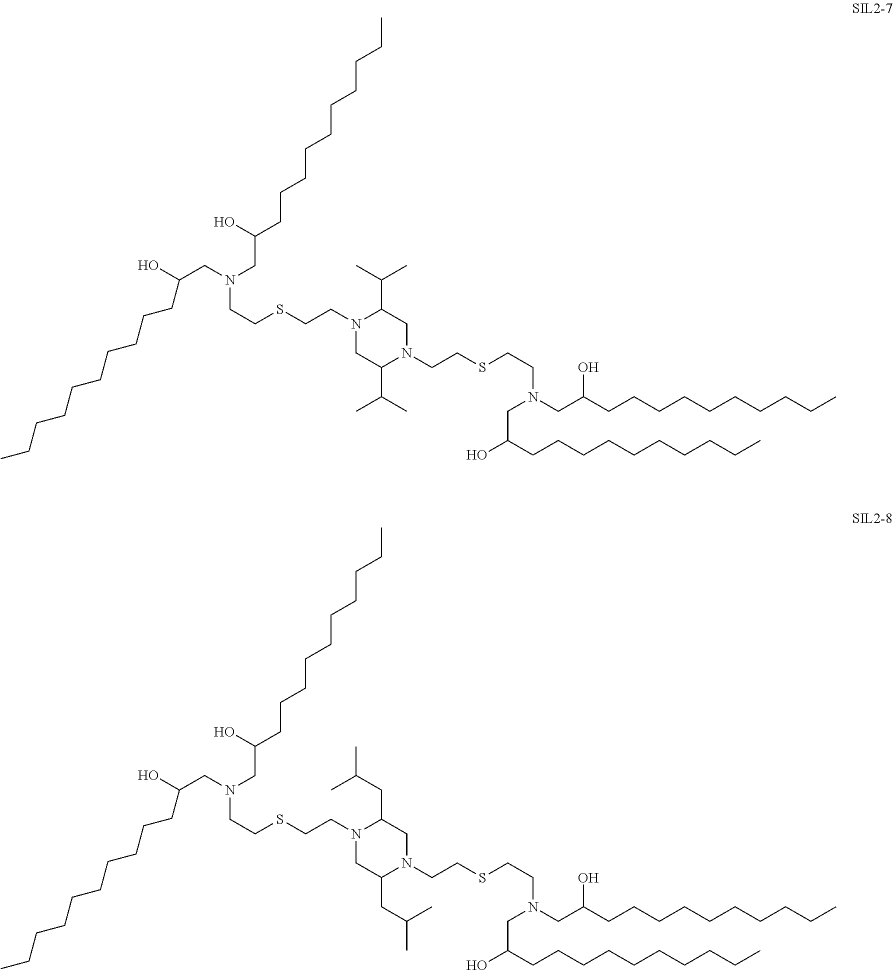

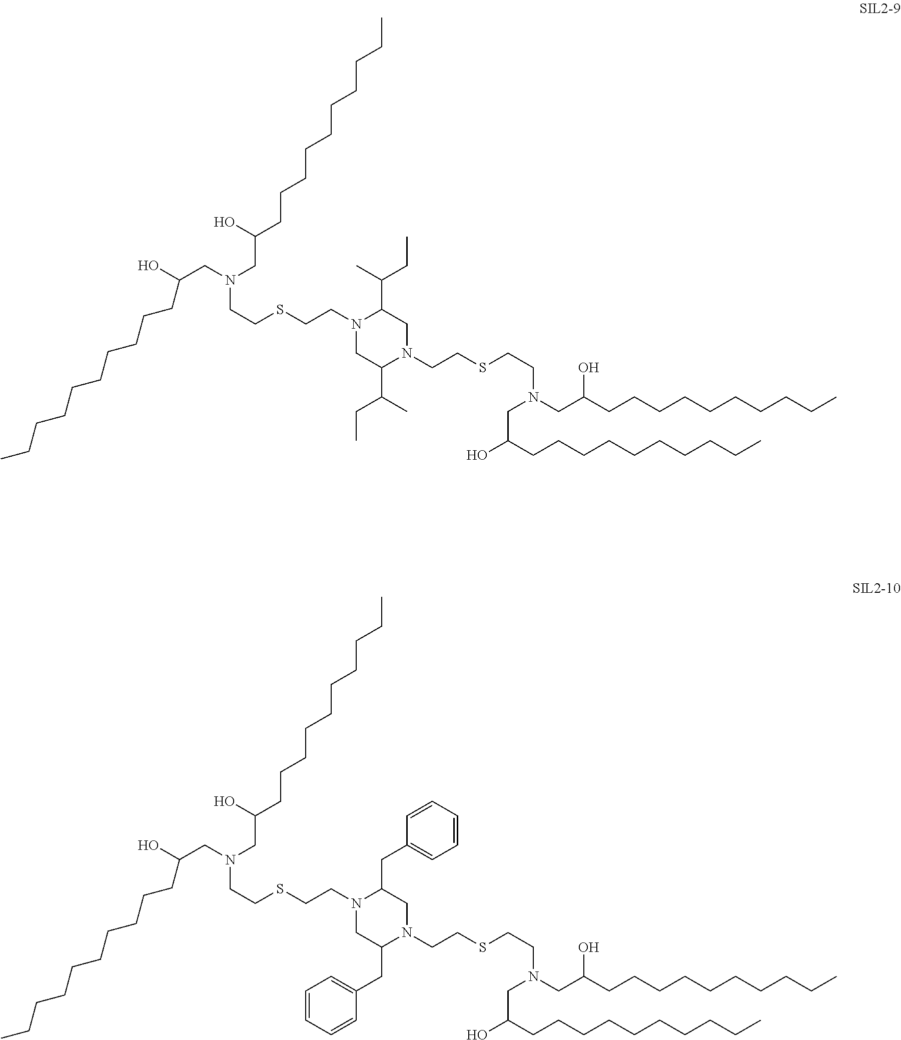

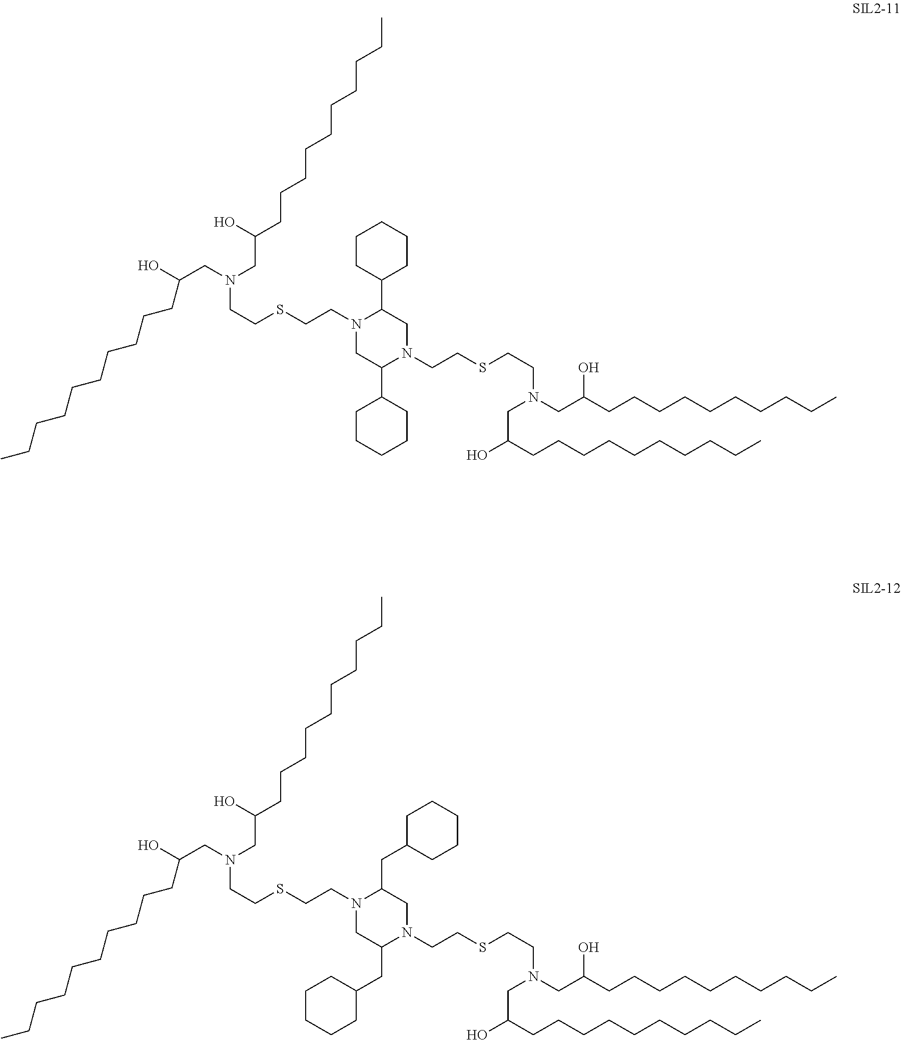

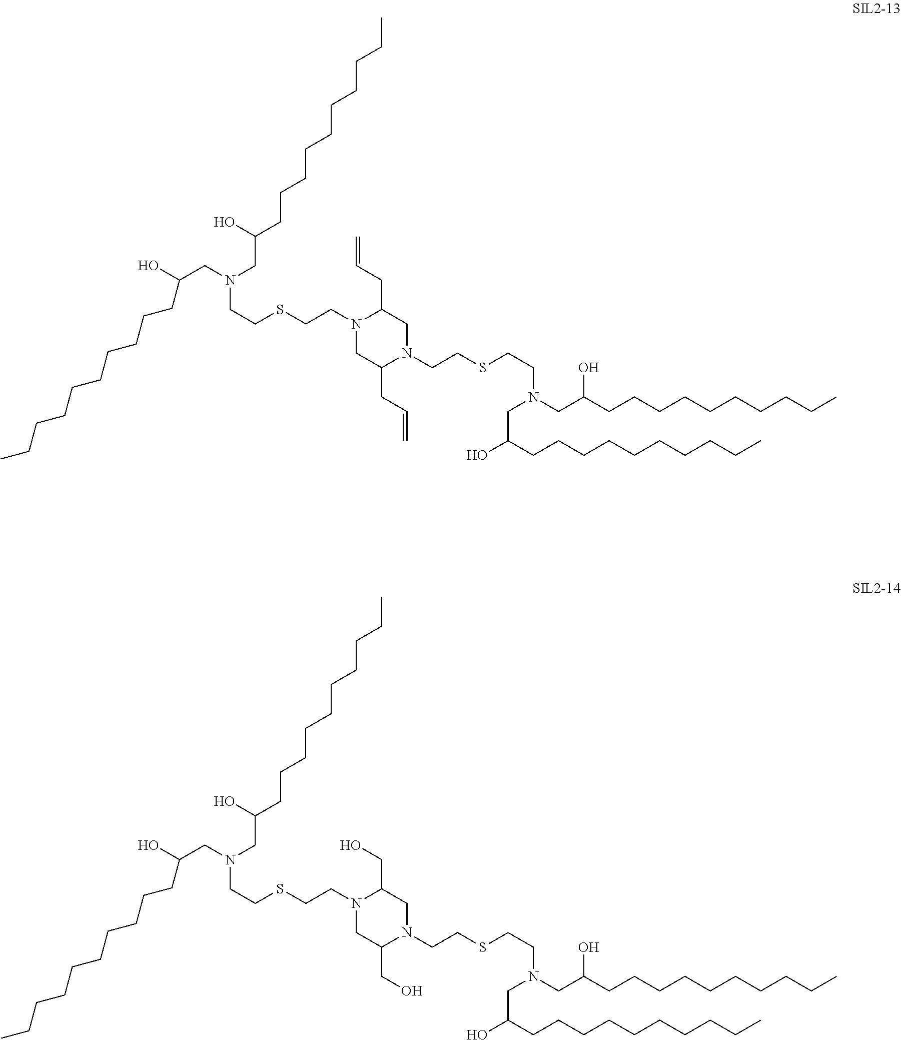

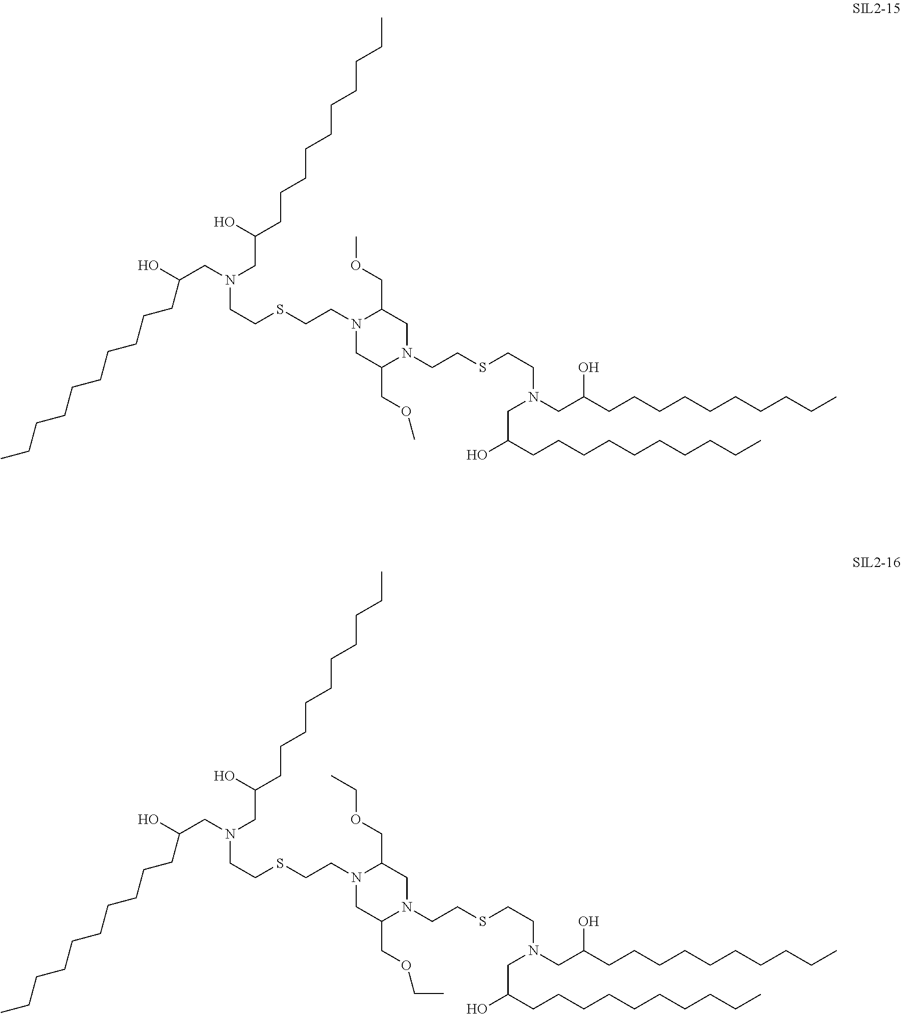

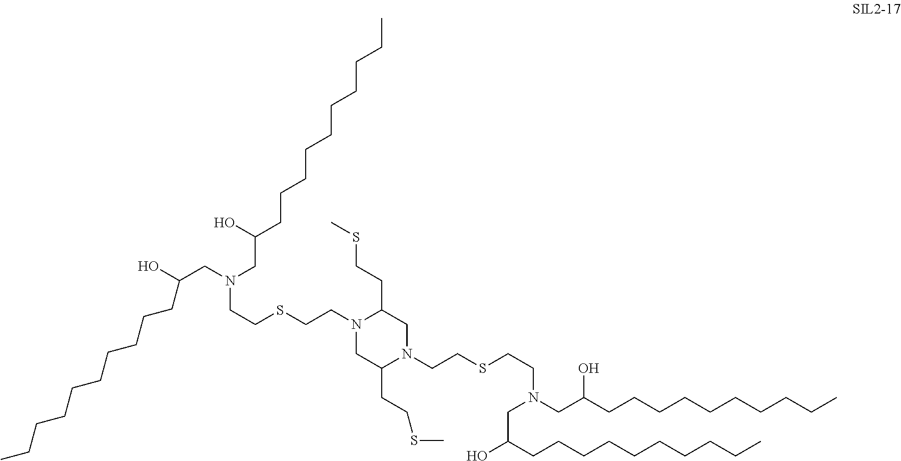 [SMILES: CCCCCCCCCCC(O)CN(CCSCCN1CC(CCSC)N(CCSCCN(CC(O)CCCCCCCCCC)CC(O)CCCCCCCCCC)CC1CCSC)CC(O)CCCCCCCCCC]

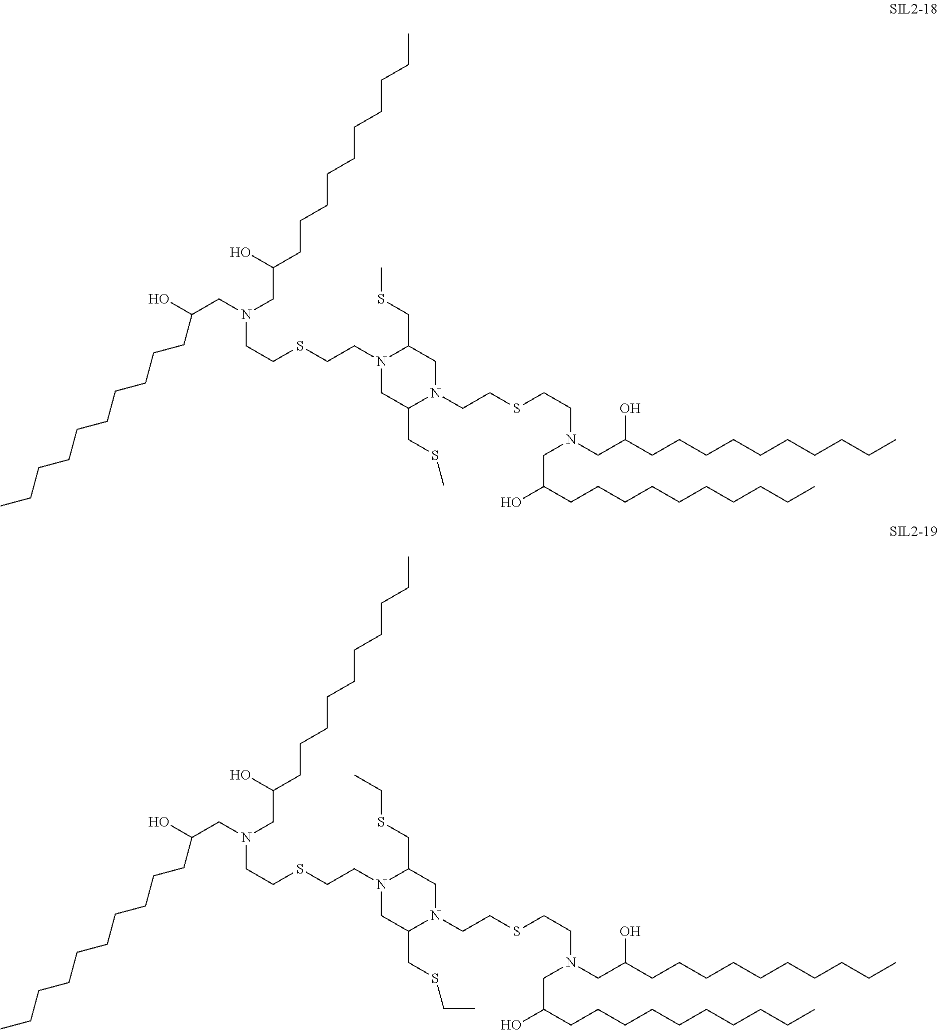

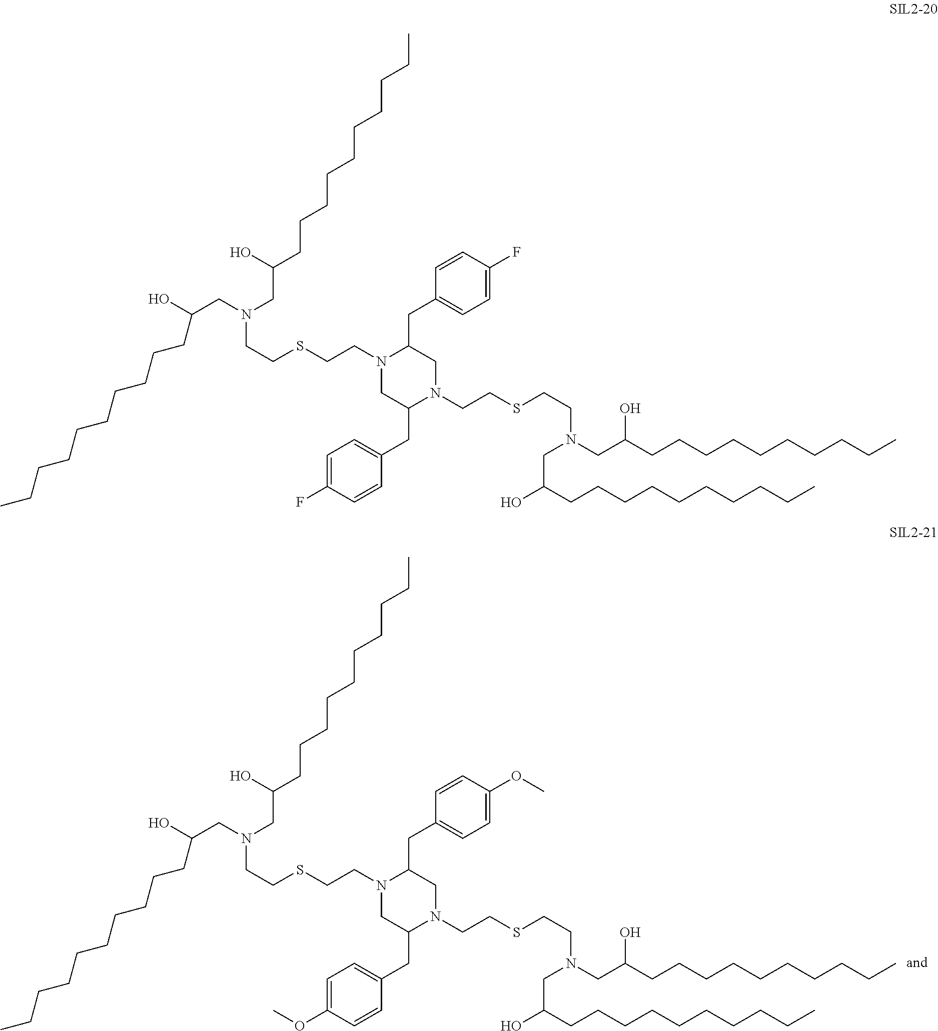

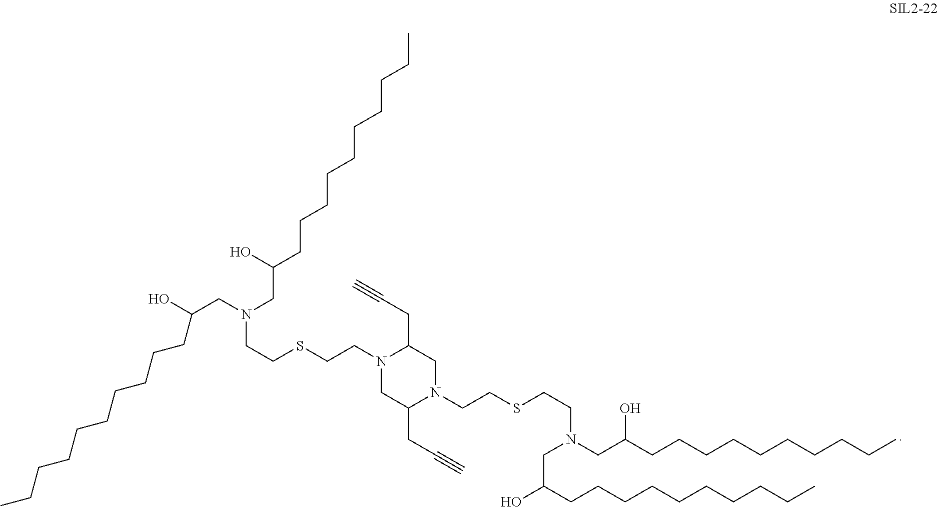 [SMILES: C#CCC1CN(CCSCCN(CC(O)CCCCCCCCCC)CC(O)CCCCCCCCCC)C(CC#C)CN1CCSCCN(CC(O)CCCCCCCCCC)CC(O)CCCCCCCCCC]

In another general aspect, the present application relates to a compound of formula (II):

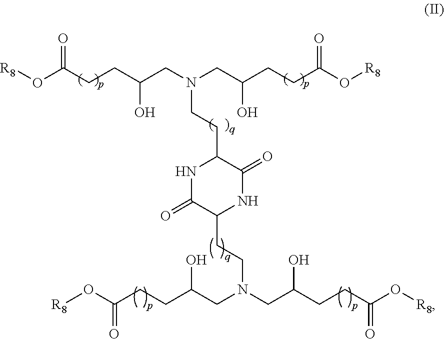

or a stereoisomer, tautomer, pharmaceutically acceptable salt or solvate thereof,
wherein,
p is an integer from 1 to 10;
q is an integer from 3 to 8; and
R₈ is unbranched or branched alkyl.

In some embodiments, q is 1-5, preferably 3.

In some embodiments, p is an integer from 1 to 3, such as 1, 2, or 3.

In some embodiments, R₈ is unbranched alkyl, preferably C₂₋₁₂ unbranched alkyl.

In some embodiments, R₈ is branched alkyl, preferably C₂₋₁₂ branched alkyl.

Exemplary compounds of formula (II) include, but are not limited to, the following compounds SIL3-1 to SIL3-11, and any tautomer, stereoisomer, pharmaceutically acceptable salt or solvate thereof:

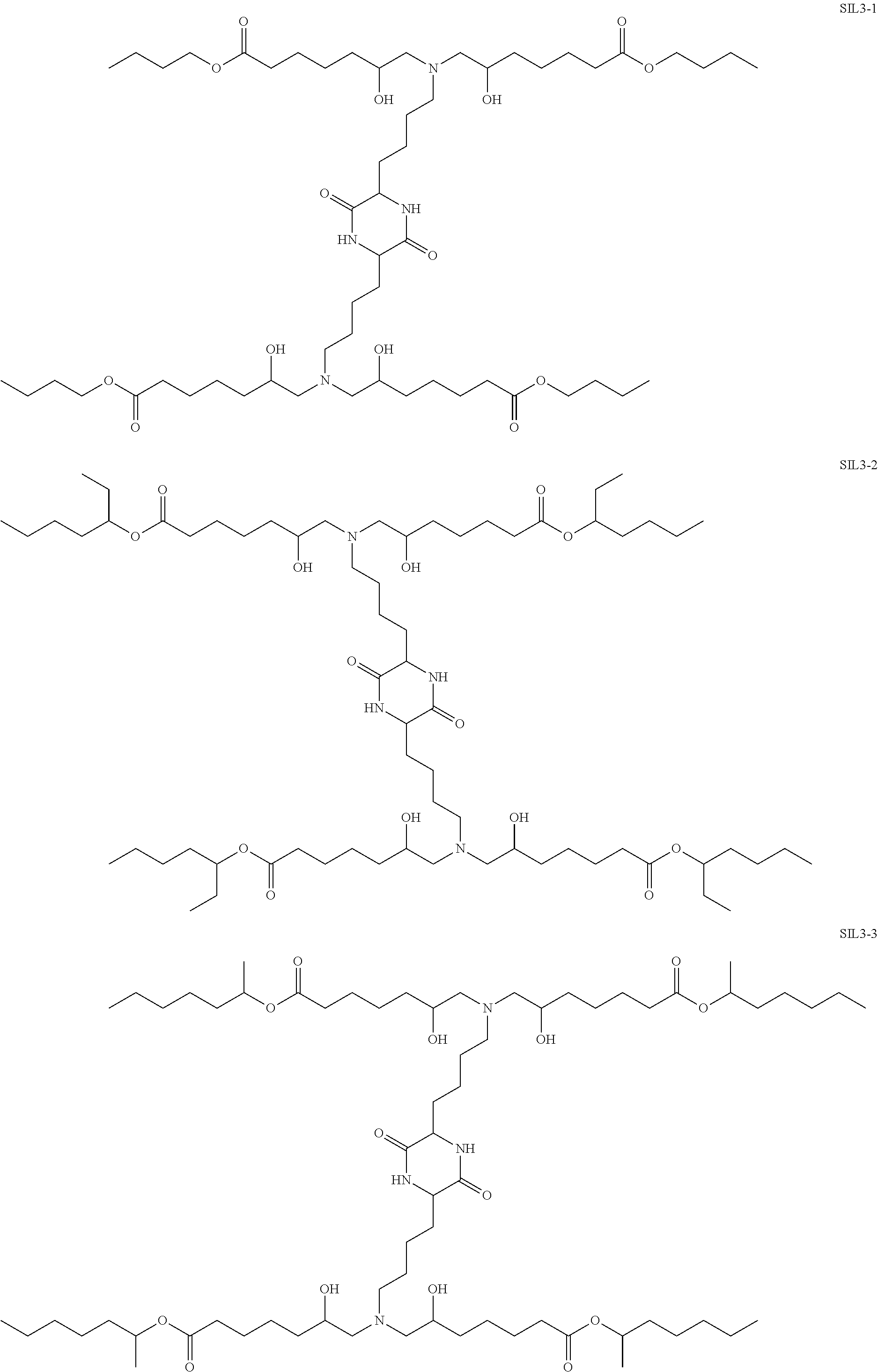

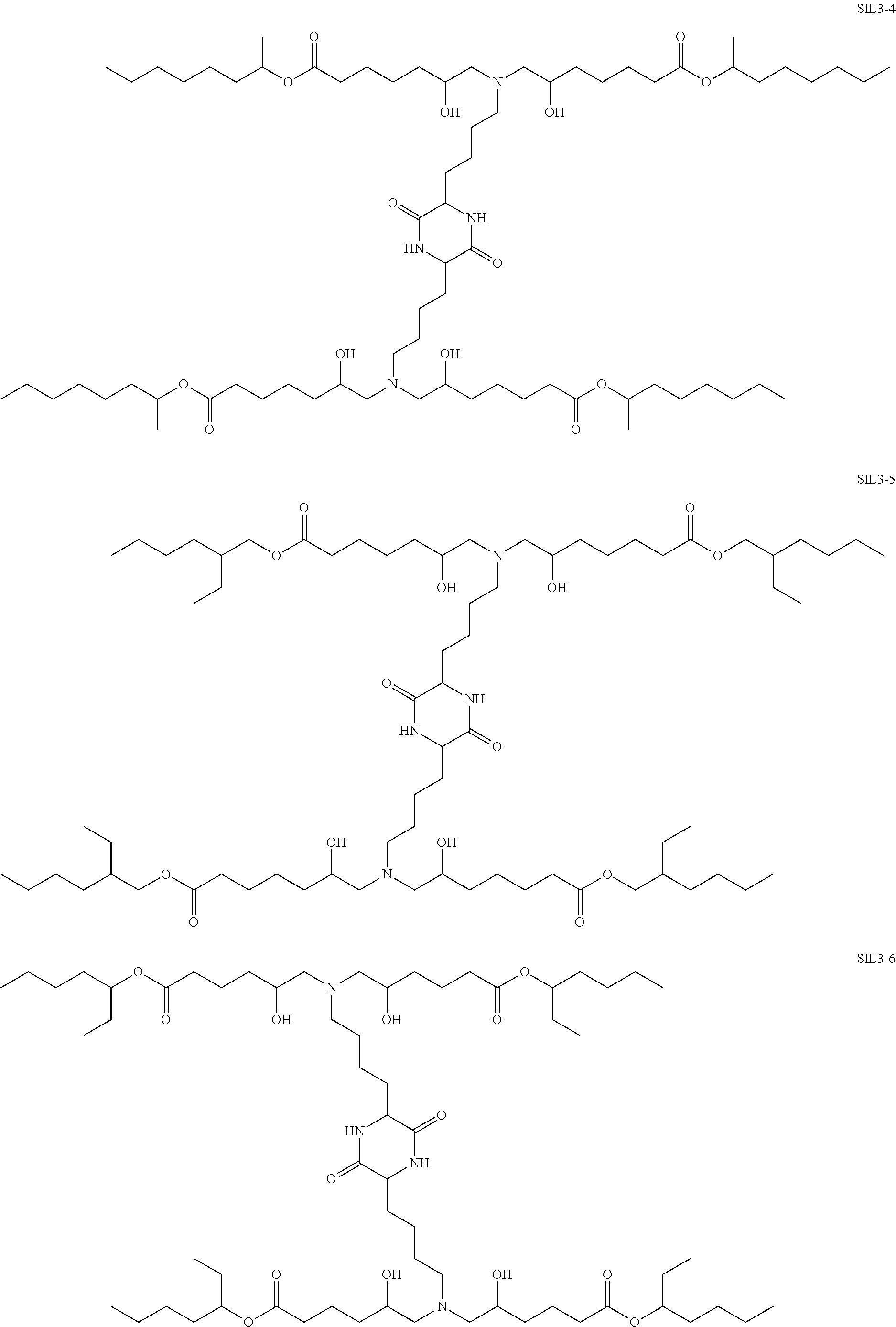

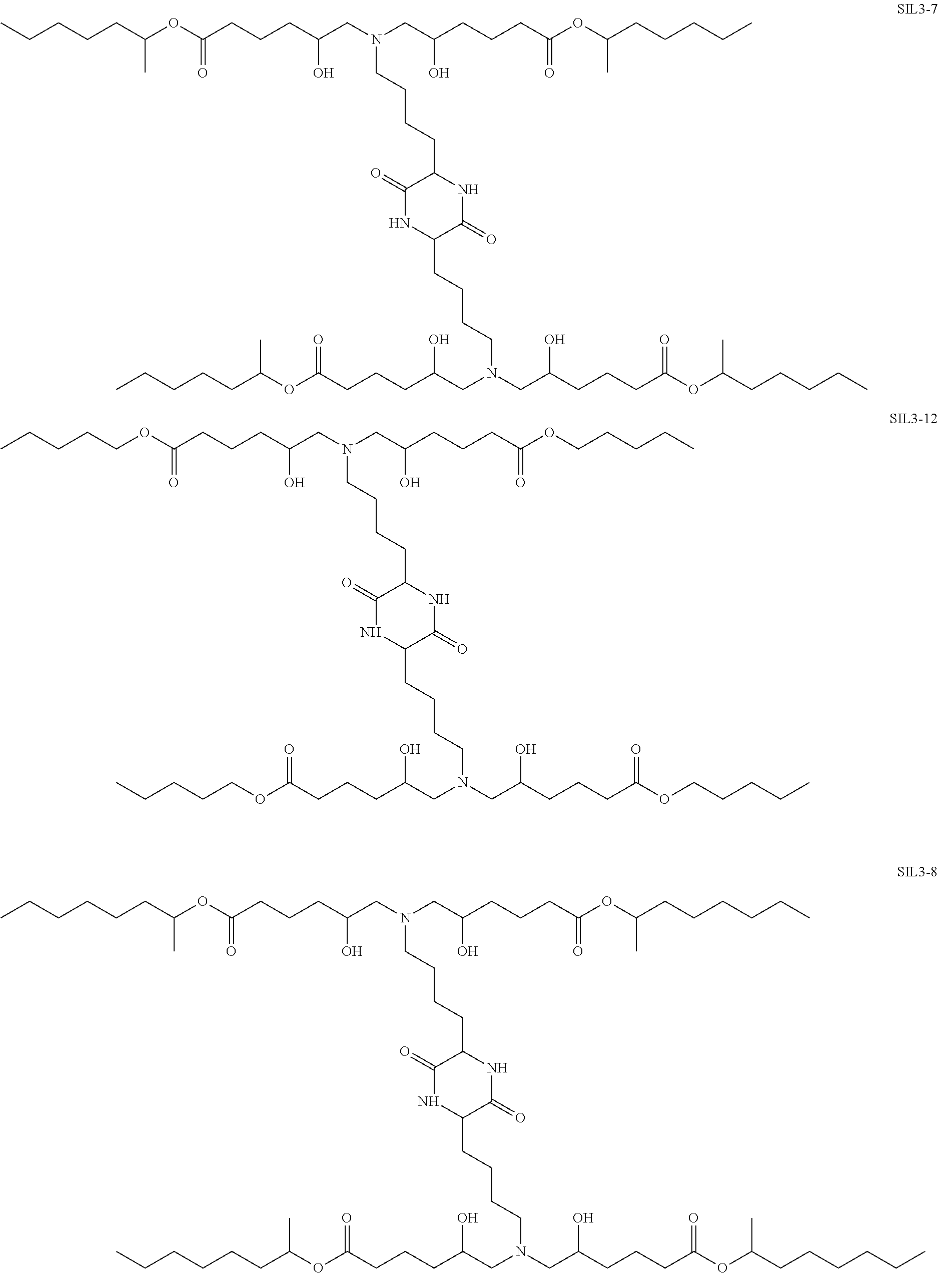

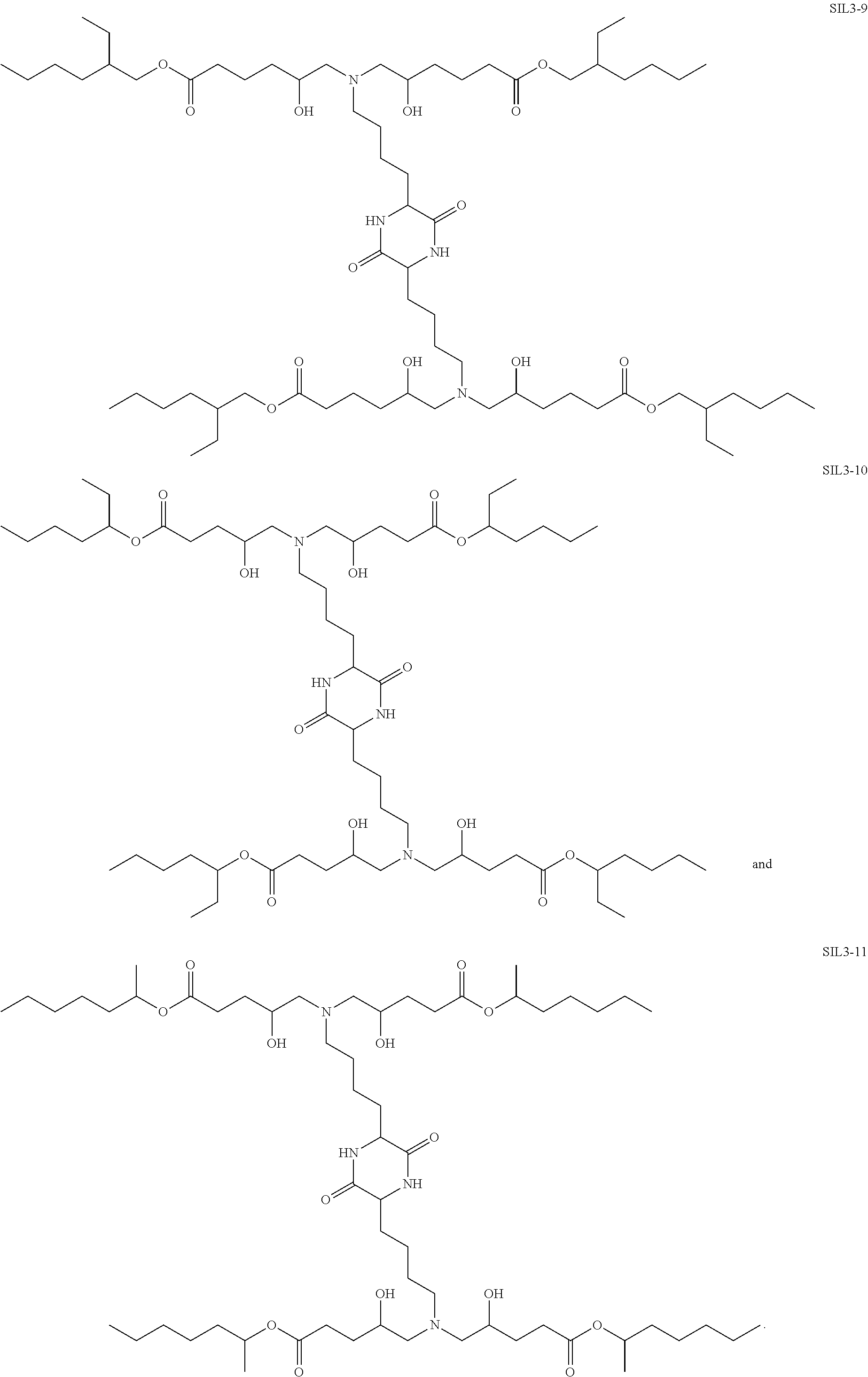

All possible combinations of the above-indicated embodiments of compounds of formula (I) or formula (II) and their tautomers, stereoisomers, pharmaceutically acceptable salts and solvates are considered to be embraced within the scope of this application.

### Methods of Preparation

Compounds described herein can be prepared by any number of processes as described generally below and more specifically illustrated by the exemplary compounds which follow in the Examples section herein. The compounds provided herein as prepared in the processes described below can be synthesized in the form of mixtures of stereoisomers (e.g., enantiomers, diastereomers), including racemic mixtures of enantiomers, which can be separated from one another using art-known resolution procedures, for instance including liquid chromatography using a chiral stationary phase. Additionally or alternatively, stereochemically pure isomeric forms of the compounds described herein can be derived from the corresponding stereochemically pure isomeric forms of the appropriate starting materials, intermediates, or reagents. For example, if a specific stereoisomer is desired, the compound can be synthesized by stereospecific methods of preparation, which typically employ stereochemically pure starting materials or intermediate compounds.

Pharmaceutically acceptable salts of compounds of the application can be synthesized from the parent compound containing an acidic or basic moiety by conventional chemical methods. Generally, such salts can be prepared by reacting the free acid or base fons of these compounds with a stoichiometric amount of the appropriate acid or base in water or in an organic solvent, or in a mixture of the two. Examples of suitable organic solvents include, but are not limited to, ether, ethyl acetate (EtOAc), ethanol, isopropanol, or acetonitrile.

By way of illustration, but not as a limitation, compounds of formula (I) or formula (II) described herein can be prepared according to the following general preparation procedures shown in Schemes 1-3 as well as the examples shown in this application. One of ordinary skill in the art will recognize that, to obtain various compounds of formula (I) or formula (II) as described herein, starting materials can be suitably selected so that the ultimately desired substituent groups will be carried through (i.e., be stable over the course of the synthesis) the reaction scheme with or without protection as appropriate to yield the desired product. Alternatively, it may be necessary or desirable to employ, in place of the ultimately desired substituent, a suitable group that may be carried through (i.e., be stable over the course of the synthesis) the reaction scheme and replaced as appropriate with the desired substituent.

Unless otherwise specified, the variables in Schemes 1-3 are as defined above in reference to the various embodiments of compounds of formula (I) or formula (II). If no temperature or temperature range is stated, it is to be understood that the reaction is to be conducted at room temperature.

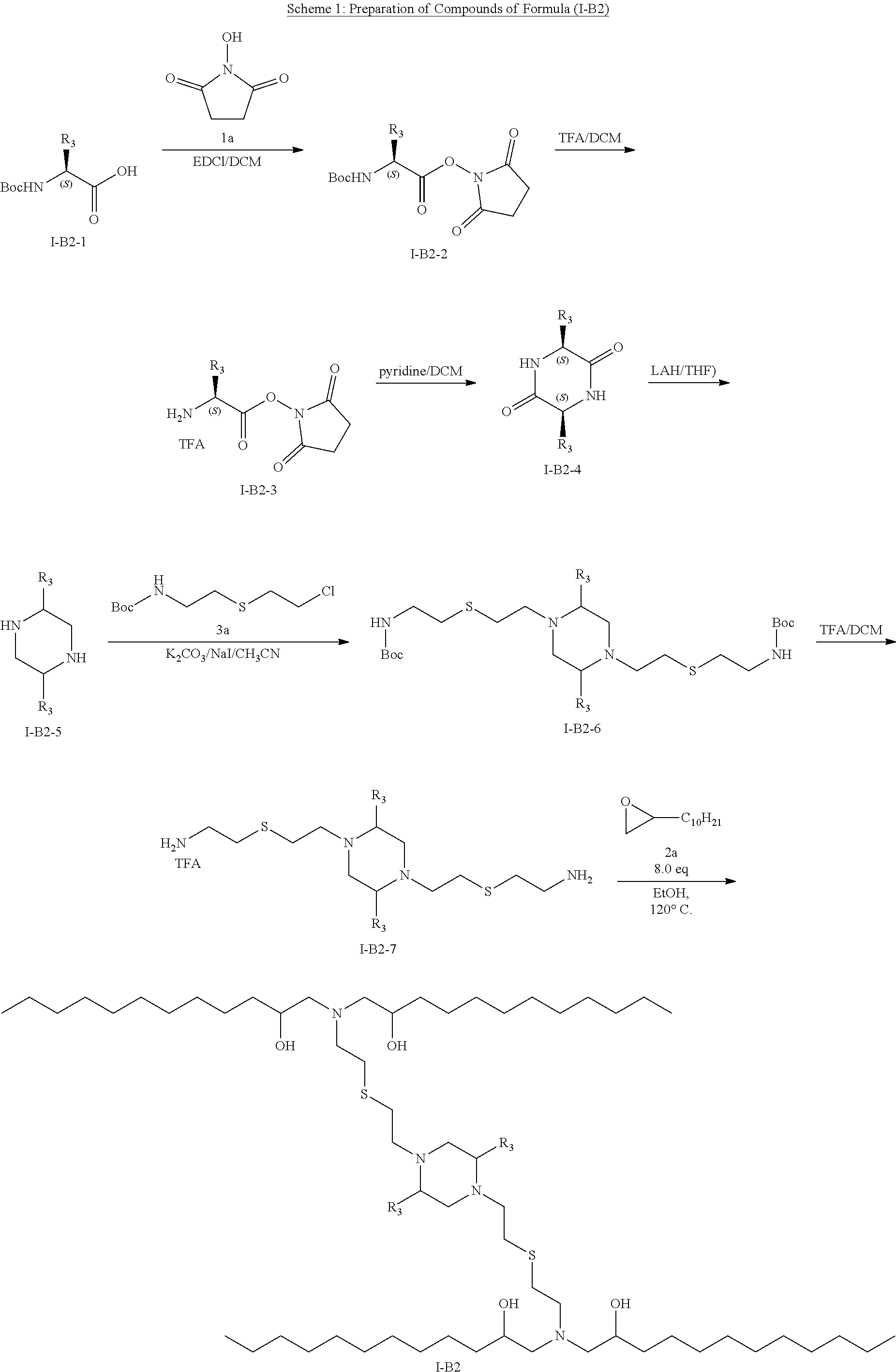

As shown in Scheme 1, a compound of formula (I-B2-2) is prepared from a compound of formula (I-B2-1) by reaction with a compound 1a, under suitable reaction conditions, wherein R₃ is defined as above. Then, the compound of formula (I-B2-2) is subjected to a deprotection under suitable conditions, such as by TFA in the solvent of dichloromethane, to obtain a compound of formula (I-B2-3). A ring forming reaction of the compound (I-B2-3) affords a cyclic compound (I-B2-4) under suitable reaction conditions, such as pyridine in dichloromethane. LAH reduction of the compound (I-B2-4), followed by substitution reaction with a compound 3a, can generate a compound of I-B2-6. Conversion of the compound of formula (I-B2-6) to a compound of formula (I-B2) is achieved by deprotection followed by another substitution with a compound of formula 2a in suitable reaction conditions.

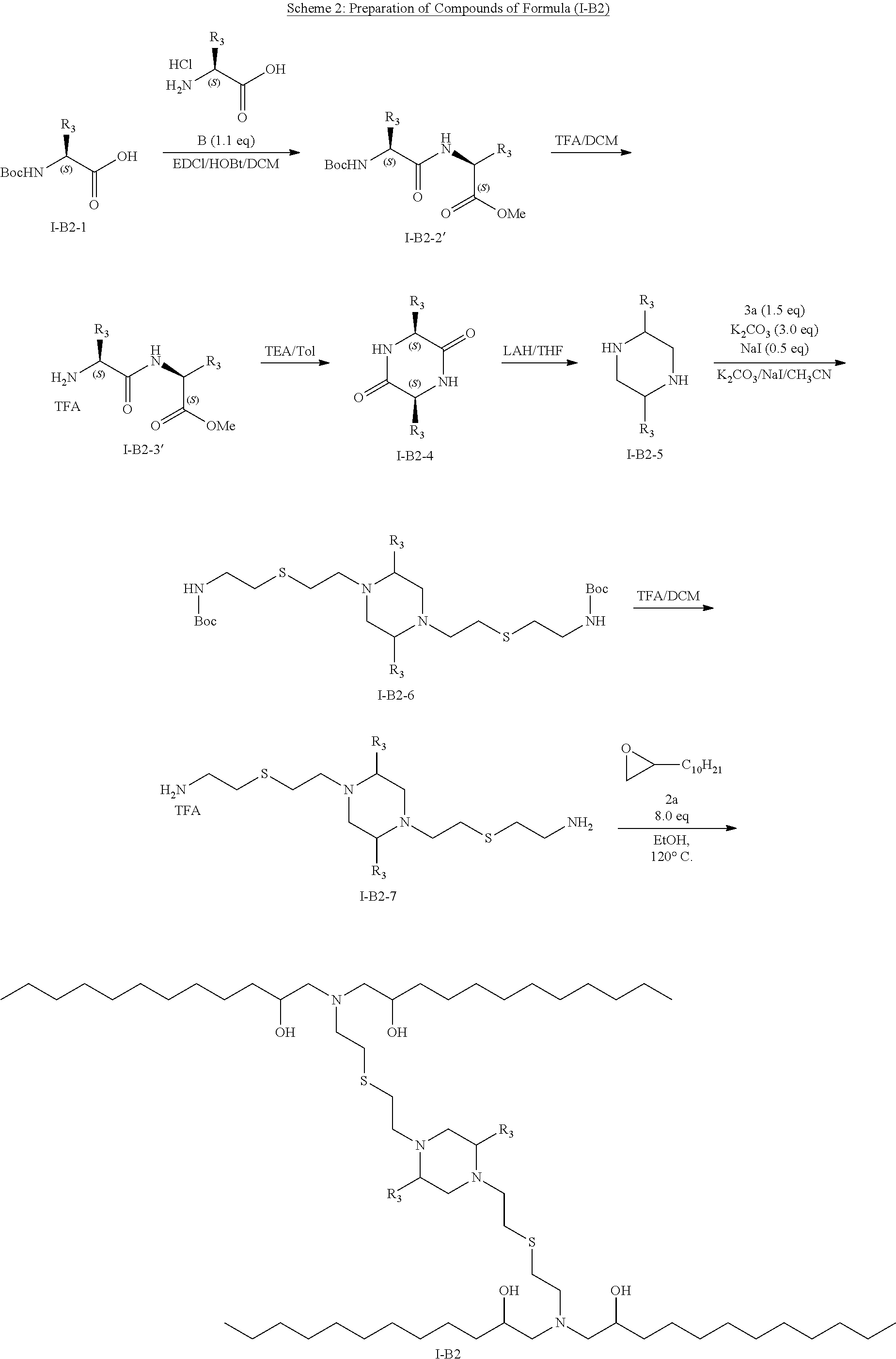

Alternatively, the compound of formula (II-B) can be prepared as shown in Scheme 2. A compound of formula (I-B2-2′) is prepared from the compound of formula (I-B2-1) by reaction with a compound B, under suitable reaction conditions, wherein R₃ is defined as above. Then, the compound of formula (I-B2-2′) is subjected to a deprotection under suitable conditions, such as by TFA in the solvent of dichloromethane, to obtain a compound of formula (I-B2-3′). A ring forming reaction of the compound (I-B2-3′) affords a cyclic compound (I-B2-4) under suitable reaction conditions, such as pyridine in dichloromethane. Then, the conversion of the compound of formula (I-B2-4) to the compound of formula (I-B2) is achieved similarly via the synthetic route as shown in Scheme 2.

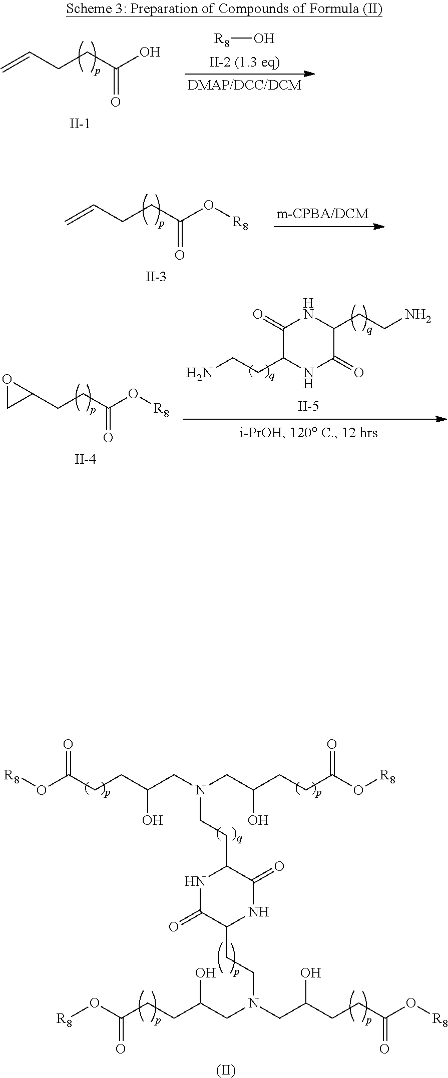

As shown in Scheme 3, a compound of formula (II-3) is prepared from a compound of formula (II-1) by reaction with an alcohol 11-2, under suitable ester bond forming conditions, wherein R₈ is defined as above. Then, the compound of formula (II-3) is subjected to epoxidation by reaction with m-CPBA in suitable solvent such as dicholoromethane to obtain a compound of formula (II-4). Conversion of the compound of formula (II-4) to a compound of formula (II) is achieved by reaction with a compound of formula (II-5) in a suitable solvent such as i-PrOH and the like, at a high temperature such as 120° C. for a period of about 12 hours.

### Compositions

In one aspect of the invention, the compounds of formula (I) or formula (II) is used in combination with a therapeutic agent (e.g., polynucleotide, small molecule, protein, peptide) to be delivered to a cell or a subject to form microparticles, nanoparticles, liposomes, or micelles. The agent to be delivered by the particles, liposomes, or micelles can be in the form of a gas, liquid, or solid, and the agent can be a polynucleotide (DNA such as dsDNA, or RNA such as mRNA or siRNA), protein, peptide, or small molecule. The compounds of formula (I) or (II) can be combined with other lipid compounds, polymers (synthetic or natural), surfactants, cholesterol, carbohydrates, proteins, lipids, etc. to form the particles. These particles can then optionally be combined with a pharmaceutically acceptable carrier to form a pharmaceutical composition.

Examples of pharmaceutically acceptable carriers include a non-toxic (in the amount used) solid, semi-solid or liquid filler, diluent, encapsulating material, or formulation. Guidance concerning formulations for small molecule, vaccines, proteins and antibodies can be found, for example, in Remington (2020) The Science and Practice of Pharmacy 23rd Edition; D'Amico et al., (2021) Drug Deliv. and Transl. Res. 11, 353-372; and Strickley and Lambert (2021) Journal of Pharmaceutical Sciences 110: 2590-2608.

The form of the pharmaceutical compositions, the route of administration, the dosage and the regimen depend upon the condition to be treated, such as the severity of the illness, the age, weight, and sex of the patient. Pharmaceutical compositions can be formulated for different modes of administration such as for topical, oral, intranasal, parenteral, intraocular, intravenous, intramuscular, or subcutaneous administration.

In an embodiment, the pharmaceutical composition contains a formulation capable of injection into a subject. Examples of injectable formulation components isotonic, sterile, saline solutions (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition, depending on the case, of sterilized water or physiological saline, permit the constitution of injectable solutions. The doses used for the administration can be adapted as a function of various parameters such as mode of administration, relevant pathology, and duration of treatment.

Other pharmaceutically acceptable forms include tablets or other solids for oral administration, including time-release capsules.

### Methods of Use

In other general aspects, provided are compounds and pharmaceutical compositions described herein for treating various diseases, in particular, for the use in the delivery of therapeutic agents or drugs (e.g., polynucleotide, small molecule, protein, peptide) to a subject or in cancer immunotherapy. For example, the compounds of formula (I) or (II) may be used in nanoparticle formulations to deliver DNA, RNA, or other polynucleotides to a subject or to a cell. For example, lipid nanoparticles (LNPs) comprising compounds of formula (I) or (II) may be used for delivery of RNA interference (RNAi) therapeutics.

As used herein, “cancer immunotherapy,” also known as immuno-oncology, is a form of cancer treatment that uses the power of the body's own immune system to prevent, control, and eliminate cancer. Cancer immunotherapy comes in a variety of forms, including targeted antibodies, cancer vaccines, adoptive cell transfer, tumor-infecting viruses, checkpoint inhibitors, cytokines, and adjuvants. mRNA is widely used in cancer immunotherapy. For example, one of the applications of mRNA is therapeutic vaccination, which can leverage the capability of mRNA to deliver immune stimulants like cytokines and chemokines.

In some embodiments, the compounds of formula (II) may be used as biodegradable lipids because these compounds contain ester groups and can undergo hydrolysis. With the biodegradable functionality, these lipids may display rapid elimination from plasma and tissues, and thus may improve their biocompatibility and facilitate their elimination once they have served their purpose to deliver therapeutic agents or drugs to the appropriate intracellular compartments in vivo.

In certain embodiments, the compounds of formula (I) or (II) can be used in nanoparticle formulations to facilitate intracellular delivery of a DNA vector to a subject. The DNA vector can deliver a variety of different transgene that can be expressed to provide a protein having a desired activity. Examples of transgenes include those providing a healthy copy of gene in a subject where the gene is defective or a new, a modified gene that can help treat a disease or disorder, or a new gene encoding for protein providing a beneficial effect.

In different embodiments, a transgene encodes GAA (acid alpha-glucosidase) for treatment of Pompe disease; TPP1 (tripeptidyl peptidase-1) for treatment of late infantile neuronal ceroid lipofuscinosis type 2 (CLN2); ATP7B (copper transporting ATPase2) for treatment of Wilson's disease; alpha galactosidase for treatment of Fabry disease; ASS1 (arginosuccinate synthase) for treatment of Citrullinemia Type 1; beta-glucocerebrosidase for treatment of Gaucher disease Type 1; beta-hexosaminidase A for treatment of Tay-Sachs disease; SERPING1 (C1 protease inhibitor or C1 esterase inhibitor) for treatment of hereditary angioedema (HAE), also known as C1 inhibitor deficiency type I and type II); or glucose-6-phosphatase for treatment of glycogen storage disease type I (GSDI).

In different embodiments, the transgene encodes insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I or II (IGF-I or IGF-II), TGFβ, activins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase.

In different embodiments, the transgene encodes thrombopoietin (TPO), an interleukin (IL-1 through IL-36), monocyte chemoattractant protein, leukemia inhibitory factor, granulocyte-macrophage colony stimulating factor, Fas ligand, tumor necrosis factors α or β, interferons α, β, or γ, stem cell factor, flk-2/flt3 ligand, IgG, IgM, IgA, IgD or IgE, chimeric immunoglobulins, an antibody, humanized antibodies, single chain antibodies, T cell receptors, chimeric T cell receptors, single chain T cell receptors, class I or class II MHC molecules. Antibodies and immunoglobulins can, for example, be provided targeting cancer cells or other disease or disorder causing cells.

In different embodiments, the transgene encodes CFTR (cystic fibrosis transmembrane regulator protein), a blood coagulation (clotting) factor (Factor XIII, Factor IX (FIX), Factor VIII (FVIII), Factor X, Factor VII, Factor VIIa, or protein C) a gain of function blood coagulation factor, erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), retinal pigment epithelium-specific 65 kDa protein (RPE65), Rab escort protein 1 (choroideremia), LCA 5 (LCA-lebercilin), ornithine ketoacid aminotransferase (gyrate atrophy), retinoschisin 1 (X-linked retinoschisis), X-linked retinitis pigmentosa GTPase (XLRP), MER proto-oncogene tyrosine kinase (MERTK) (autosomal recessive (AR) forms of retinitis pigmentosa (RP)), ABCA4 (Stargardt), ACHM 2, 3 and 4 (achromatopsia), an anti-vascular endothelial growth factor (VEGF) agent polypeptide (e.g., bevacizumab, brolucizumab, ranibizumab, aflibercept), DFNB1 (connexin 26 deafness), USH1C (Usher's syndrome 1C), PKD-1 or PKD-2 (polycystic kidney disease), TPP1 (tripeptidyl peptidase-1), a sulfatase, N-acetylglucosamine-1-phosphate transferase, cathepsin A, GM2-AP, NPC1, VPC2, a sphingolipid activator protein, or one or more donor sequences used as repair templates for genome editing.

In different embodiments, the transgene encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C—X—C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alpha1-antitrypsin for treatment of emphysema or chronic obstructive pulmonary disease (COPD); alpha-L-iduronidase for treatment of mucopolysaccharidosis I (MIPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

In a further embodiment the transgene encodes a protein for treating a disease or disorder selected from the group consisting of: hereditary angioedema, Pompe disease, hemophilia A, hemophilia B, Fabry, wet macular degeneration, Leber hereditary optic neuropathy, and Stargardt disease.

### EXAMPLES

The following examples of the application are to further illustrate the nature of the application. It should be understood that the following examples do not limit the application and the scope of the application is to be determined by the appended claims.

### Methods of Synthesis

Unless indicated otherwise, the abbreviations for chemical reagents and synthesis conditions have their ordinary meaning known in the art as follows:
“LDA” refers to lithium diisopropyl amide;
“EA” refers to ethyl acetate;
“PE” refers to petroleum ether;
“r.t.” and “rt” refer to room temperature;
“THF” refers to tetrahydrofuran;
“DEAD” refers to diethyl azodicarboxylate;
“TBAB” refers to tetrabutylammonium bromide;
“DCM” refers to dichloromethane;
“HOBT” refers to hydroxybenzotriazole;
“LAH” refers to lithium aluminum hydride;
“TLC” refers to thin layer chromatography;
“Prep-TLC” refers to preparatory thin layer chromatography;
“TMS-I” refers to trimethylsilyl iodide;
“Hex” refers to hexanes;
“DMF” refers to dimethylformamide;
“h” or “hr” refers to hours;
“min” refers to minutes;
“EDCI” refers to 1-Ethyl-3-(3-dimethylaminopropyl)carbodiimide;
“DMAP” refers to 4-Dimethylaminopyridine;
“Prep-HPLC” refers to preparative high performance liquid chromatography;
“DHP” refers to dihydropyran;
“DPPF” refers to 1,1′-Bis(diphenylphosphino)ferrocene;
“DIEA” refers to diisopropylethylamine;
“TEA” refers to triethyl amine;
“m-CPBA” refers to meta-chloroperoxybenzoic acid;
“MeOH” refers to methanol.
“EtOH” refers to ethanol.
“i-PrOH” refers to isopropanol; and
“Tol” refers to toluene.

### Synthesis of Compounds of Formula (I-A)

### Example 1. Synthesis of Compound SIL1-17

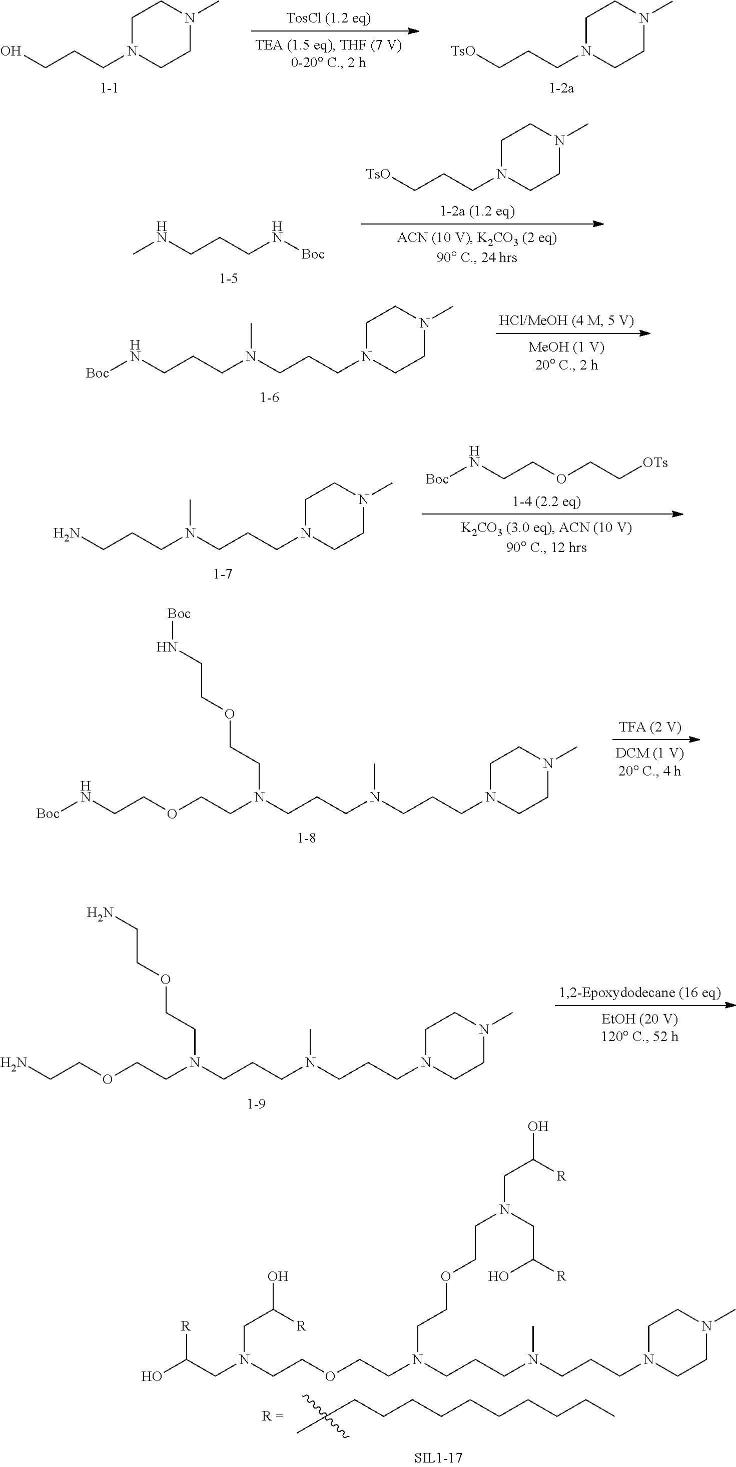

### Step 1. Preparation of Compound 1-2a

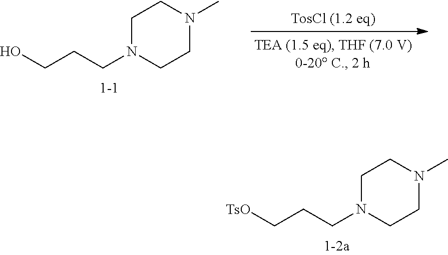 [SMILES: CN1CCN(CCCO)CC1 ; Cc1ccc(S(=O)(=O)OCCCN2CCN(C)CC2)cc1]

To the solution of Compound 1-1 (2.00 g, 12.6 mmol, 1.00 eq) in DCM (14.0 mL) was added TEA (1.92 g, 18.9 mmol, 2.64 mL, 1.50 eq). Then TsCl (2.89 g, 15.1 mmol, 1.20 eq) was added dropwise at 0° C. The reaction was stirred at 20° C. for 2 hrs. LC-MS showed Compound 1-1 was consumed completely and desired mass was detected. The reaction mixture was filtered and the filter cake was washed with THF (20.0 mL) and the filtrate was concentrated under reduced pressure. The crude product was used into the next step without further purification. Compound 1-2a (5.00 g, crude) was obtained as a yellow oil.

### Step 2. Preparation of Compound 1-6

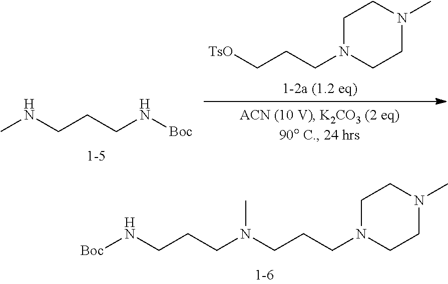 [SMILES: Cc1ccc(S(=O)(=O)OCCCN2CCN(C)CC2)cc1 ; CNCCCNC(=O)OC(C)(C)C ; CN(CCCNC(=O)OC(C)(C)C)CCCN1CCN(C)CC1]

To the solution of Compound 1-5 (2.5 g, 13.2 mmol, 1.00 eq) in ACN (25.0 mL) was added K₂CO₃ (3.67 g, 26.5 mmol, 2.00 eq) and Compound 1-2a (4.98 g, 15.9 mmol, 1.20 eq). The reaction was stirred at 90° C. for 24 hrs. LC-MS showed Compound 1-5 was consumed completely and desired mass was detected. The reaction mixture was filtered and the filter cake was washed with ACN (40.0 mL) and the filtrate was concentrated under reduced pressure. The crude product was used into the next step without further purification. Compound 1-6 (3.3 g, crude) was obtained as a white solid.

### Step 3. Preparation of Compound 1-7

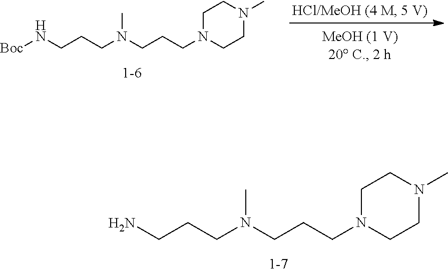 [SMILES: CN(CCCNC(=O)OC(C)(C)C)CCCN1CCN(C)CC1 ; CN(CCCN)CCCN1CCN(C)CC1]

To the solution of Compound 1-6 (3.30 g, 10.1 mmol, 1.00 eq) in MeOH (3.00 mL) was added HCl/MeOH (15.0 mL). The reaction was stirred at 20° C. for 2 hrs. TLC (Dichloromethane:Methanol=3:1) indicated Compound 1-6 was consumed completely. The reaction mixture was concentrated under reduced pressure to remove solvent and adjust pH to neutral with macroporous ion basic exchange resin. The crude product was used into the next step without further purification. Compound 1-7 (2.5 g, crude, 4HCl) was obtained as a white solid.

¹H NMR: 400 MHz MeOD

δ 2.88-2.85 (m, 2H), 2.50-2.45 (m, 4H), 2.43-2.40 (m, 6H), 2.38-2.36 (m, 3H), 1.29-2.26 (m, 3H), 1.76-1.70 (m, 4H).

### Step 4. Preparation of Compound 1-8

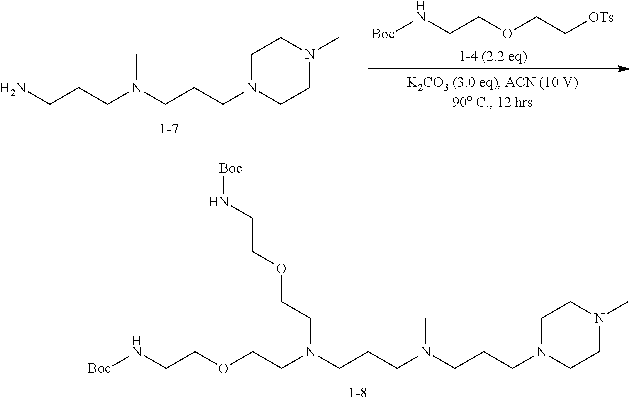 [SMILES: Cc1ccc(S(=O)(=O)OCCOCCNC(=O)OC(C)(C)C)cc1 ; CN(CCCN)CCCN1CCN(C)CC1 ; CN(CCCN(CCOCCNC(=O)OC(C)(C)C)CCOCCNC(=O)OC(C)(C)C)CCCN1CCN(C)CC1]

To the solution of Compound 1-7 (0.600 g, 1.31 mmol, 50% purity, 1.00 eq) in ACN (6.00 mL) was added K₂CO₃ (544 mg, 3.94 mmol, 3.00 eq), NaI (98.45 mg, 656 μμmol, 0.500 eq) and Compound 1-4 (1.04 g, 2.89 mmol, 2.20 eq). The reaction was stirred at 90° C. for 12 hrs. LC-MS showed Compound 1-7 was consumed completely and desired mass was detected. The reaction mixture was filtered and the filter cake was washed with ACN (10.0 mL), the filtrate was concentrated under reduced pressure. The residue was purified by silica gel chromatography (Dichloromethane:Methanol=10/1 to 3/1). Compound 1-8 (0.300 g, crude) was obtained as a white solid.

### Step 5. Preparation of Compound 1-9

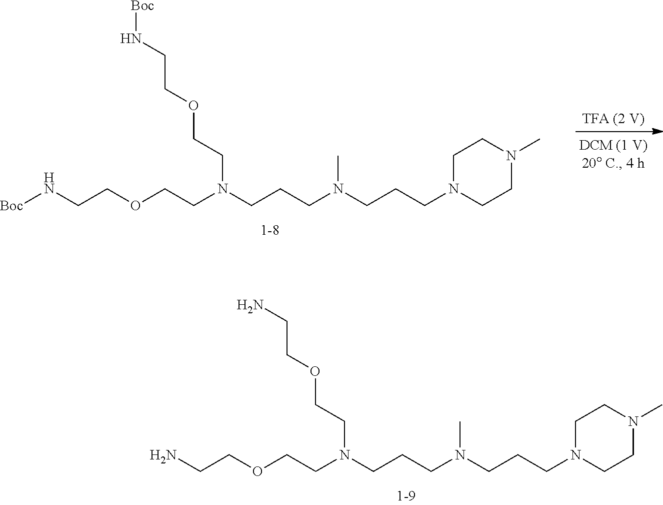 [SMILES: CN(CCCN(CCOCCNC(=O)OC(C)(C)C)CCOCCNC(=O)OC(C)(C)C)CCCN1CCN(C)CC1 ; CN(CCCN(CCOCCN)CCOCCN)CCCN1CCN(C)CC1]

To the solution of Compound 1-8 (0.300 g, 365 μμmol, 1.00 eq) in DCM (0.300 mL) was added TFA (924 mg, 8.10 mmol, 0.600 mL, 22.19 eq). The reaction was stirred in 20° C. for 4 hrs. TLC (Dichloromethane:Methanol=1:1) indicated Compound 1-8 was consumed completely. The reaction mixture was concentrated under reduced pressure to remove solvent and adjust pH to neutral with macroporous ion basic exchange resin. The crude product was used into the next step without further purification. Compound 1-9 (0.130 g, crude) was obtained as a yellow oil.

¹H NMR: 400 MHz MeOD

δ 3.54-3.51 (m, 5H), 3.47-3.45 (m, 4H), 2.85-2.82 (m, 5H), 2.75-2.72 (m, 5H), 2.34-2.33 (m, 4H), 2.33-2.32 (m, 5H), 2.32-2.29 (m, 7H), 2.29-2.27 (m, 4H), 2.20 (s, 3H), 1.68-1.60 (m, 5H), 1.29-1.26 (m, 3H).

### Step 6. Preparation of Compound SIL1-17

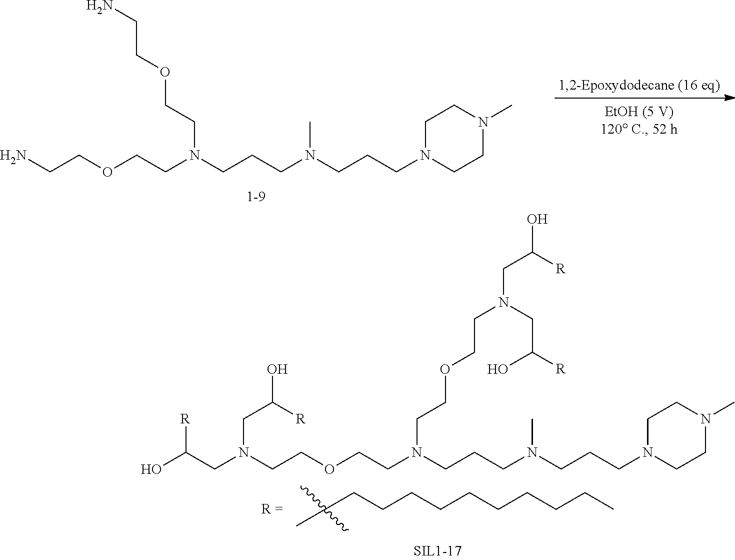

To the solution of Compound 1-9 (0.0900 g, 223 μmol, 1.00 eq) in EtOH (0.5 mL) was added 1,2-Epoxydodecane (659 mg, 3.58 mmol, 16.0 eq). The reaction was stirred for 120° C. at 2 hrs. LC-MS showed Compound 1-9 was consumed completely and desired mass was detected. The reaction was concentrated under vacuum to get the desired product. The residue was purified by silica gel chromatography (Dichloromethane:Methanol=80/1-1/8). Compound SIL1-17 (50 mg, 39.0 μμmol, 17.4% yield, 89.1% purity) was obtained as a white solid.

¹H NMR: 400 MHz CDCl₃

δ 3.54-3.44 (m, 14H), 2.82-2.32 (m, 32H), 1.45-1.72 (m, 5H), 1.43-1.26 (m, 69H), 0.90-0.842 (m, 12H).

### LCMS: (M/2+1): 570.7

### Example 2. Synthesis of Compound SIL1-7

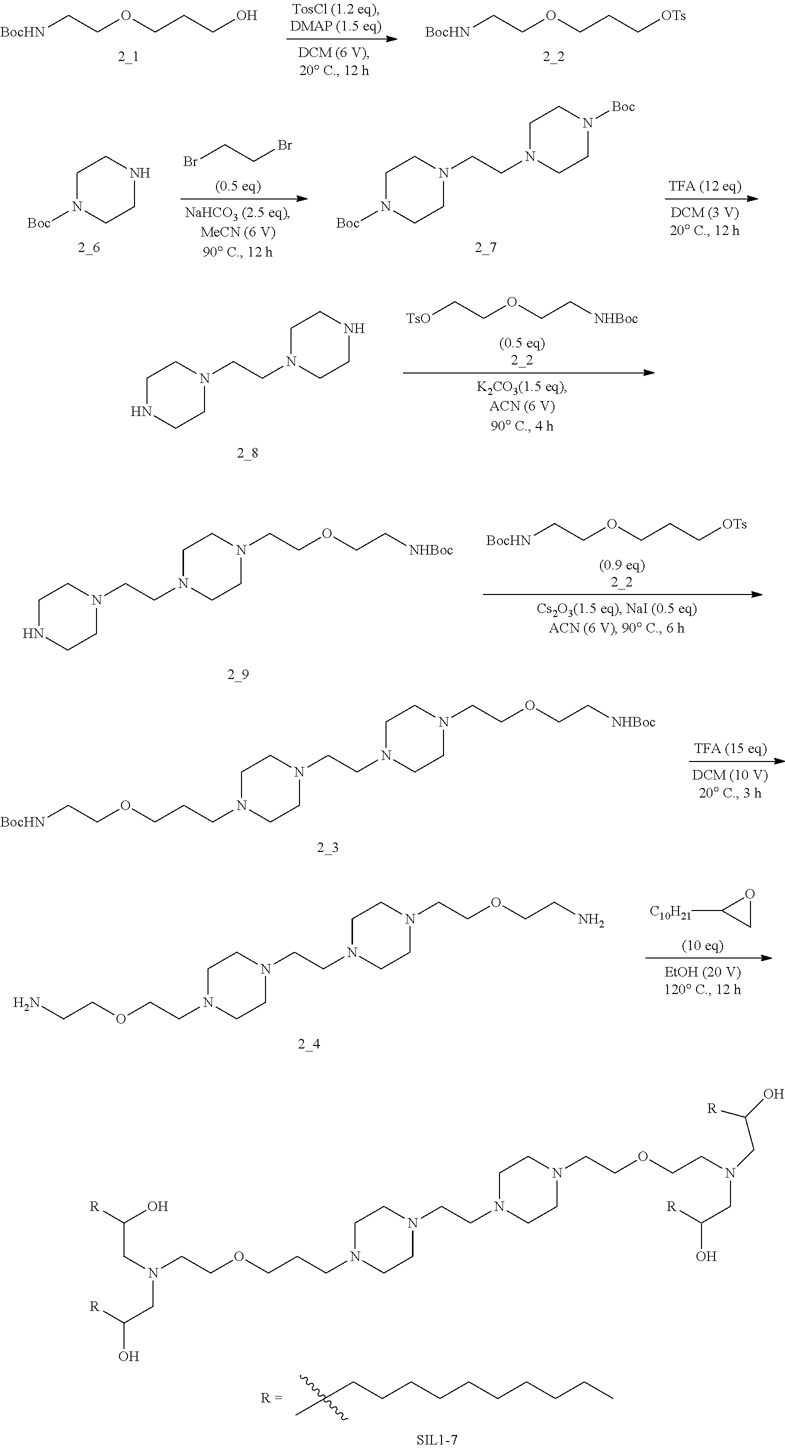

### Step 1. Preparation of Compound 2_2

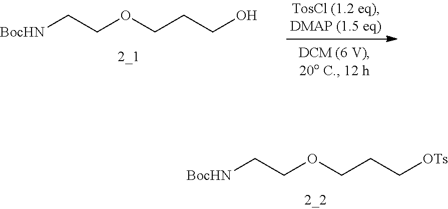 [SMILES: CC(C)(C)OC(=O)NCCOCCCO ; Cc1ccc(S(=O)(=O)OCCCOCCNC(=O)OC(C)(C)C)cc1]

To a solution of Compound 2_1 (2.00 g, 9.12 mmol, 1.0 eq) in DCM (12.0 mL) was added DMAP (1.67 g, 13.7 mmol, 1.5 eq) and TsCl (2.09 g, 11.0 mmol, 1.2 eq). The mixture was stirred at 20° C. for 12 h. TLC (Petroleum ether:Ethyl acetate=2:1) showed Compound 2_1 (2.00 g, 9.12 mmol, 1.0 eq) was used up. The reaction mixture was diluted with brine 30 mL and extracted with Ethyl acetate (50 mL). The combined organic layer was dried over Na₂SO4. Filtered and concentrated under reduced pressure to give a residue. The residue was purified by column chromatography (SiO₂, Petroleum ether/Ethyl acetate=2/1). Compound 2_2 (1.60 g, 4.28 mmol, 47.0% yield) was obtained as light yellow oil, checked by HNMR.

¹H NMR: (400 MHz, CDCl₃)

δ ppm 7.78-7.84 (m, 2H), 7.36 (d, J=8.00 Hz, 2H), 4.84 (s, 1H), 4.14 (t, J=6.13 Hz, 2H), 3.46 (t, J=5.94 Hz, 2H), 3.37-3.41 (m, 2H), 3.17-3.28 (m, 2H), 2.46 (s, 3H), 1.90 (m, J=6.03 Hz, 2H), 1.46 (s, 9H)

### Step 2. Preparation of Compound 2_7

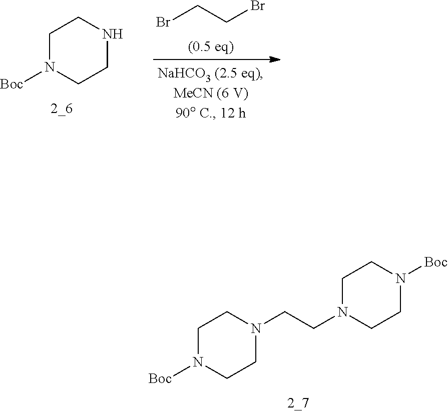

To a solution of Compound 2_6 (55.0 g, 295 mmol, 1.0 eq) in MeCN (330 mL) was added 1,2-dibromoethane (27.7 g, 148 mmol, 11.1 mL, 0.50 eq) and NaHCO₃ (62.0 g, 738 mmol, 28.7 mL, 2.5 eq). The mixture was stirred at 90° C. for 12 h. TLC (Dichloromethane:Methanol=10:1, R_{f}=0.42) indicated Compound 2_6 was consumed completely and three new spots formed. Until the reaction mixture was completely reactive, cooling to RT, the solvent was removed and the residue was dissolved in Ethyl acetate (500 ml). The organic solution was washed with water (300 ml) and brine (200 ml), dried over by Na₂SO4 and concentrated under the reduced pressure. The crude product was purified by re-crystallization from MeCN (150 mL) at 25° C. Compound 27 (51.0 g, 128 mmol, 43.3% yield) was obtained as a white solid.

¹H NMR: (400 MHz, MeOD)

δ ppm 3.43 (s, 8H), 2.56 (s, 4H), 2.45-2.48 (m, 8H), 1.46 (s, 18H).

### Step 3. Preparation of Compound 4

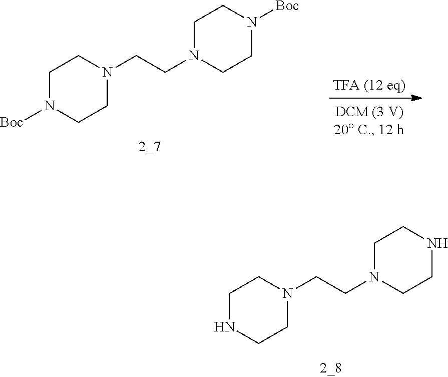 [SMILES: CC(C)(C)OC(=O)N1CCN(CCN2CCN(C(=O)OC(C)(C)C)CC2)CC1 ; C1CN(CCN2CCNCC2)CCN1]

To a solution of Compound 2_7 (10.0 g, 25.1 mmol, 1.0 eq) in DCM (30 mL) was added TFA (34.3 g, 301 mmol, 22.3 mL, 12 eq). The mixture was stirred at 20° C. for 12 h. HNMR (ET42897-1-P1A2) showed Compound 2_7 (10.0 g, 25.1 mmol, 1.0 eq) was consumed. The reaction mixture was concentrated under reduced pressure to give a residue. Compound 2_8 was dissociated with alkaline resin to a pH of about 7-8 for filtration and rotary evaporation. Compound 2_8 (6.00 g, crude) was obtained as a light yellow solid, checked by 1 HNMR.

¹H NMR: (400 MHz, D₂O)

δ ppm 3.36-3.46 (m, 8H), 3.14-3.30 (m, 12H).

### Step 4. Preparation of Compound 2_9

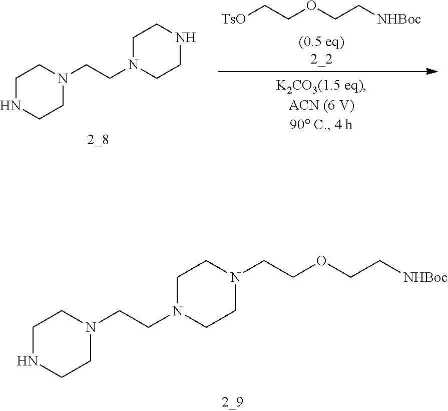 [SMILES: Cc1ccc(S(=O)(=O)OCCOCCNC(=O)OC(C)(C)C)cc1 ; C1CN(CCN2CCNCC2)CCN1 ; CC(C)(C)OC(=O)NCCOCCN1CCN(CCN2CCNCC2)CC1]

To a solution of Compound 2_8 (2.43 g, 12.2 mmol, 2.2 eq) in ACN (12 mL) was added K₂CO₃ (1.15 g, 8.35 mmol, 1.5 eq), 2-[2-(tert-butoxycarbonylamino)ethoxy]ethyl 4-methylbenzenesulfonate (2 g, 5.56 mmol, 1.0 eq). The mixture was stirred at 90° C. for 12 h. TLC (Dichloromethane:Methanol=10:1) showed Compound 2_8 (2.43 g, 12.2 mmol, 2.2 eq) and 2-[2-(tert-butoxycarbonylamino)ethoxy]ethyl 4-methylbenzenesulfonate (2.00 g, 5.56 mmol, 1.0 eq) was used up. The reaction mixture was filtered and concentrated under reduced pressure to give a residue. Combine with ET42897-14 purification. The residue was purified by column chromatography (SiO₂, DCM:MeOH=20/1 to 5/1). Compound 2_9 (1.2 g) was obtained as white oil. Checked by HNMR.

¹H NMR: (400 MHz, MeOD)

δ ppm 3.61 (s, 2H), 3.47 (s, 2H), 3.34 (d, J=3.63 Hz, 2H), 3.18-3.25 (m, 1H), 3.21 (s, 1H), 3.12 (s, 4H), 2.45-2.83 (m, 15H), 1.44 (s, 9H).

### Step 5. Preparation of Compound 2_3

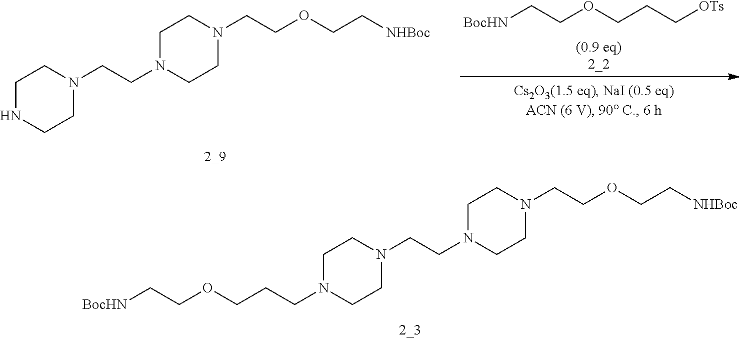 [SMILES: CC(C)(C)OC(=O)NCCOCCN1CCN(CCN2CCNCC2)CC1 ; Cc1ccc(S(=O)(=O)OCCCOCCNC(=O)OC(C)(C)C)cc1 ; CC(C)(C)OC(=O)NCCOCCCN1CCN(CCN2CCN(CCOCCNC(=O)OC(C)(C)C)CC2)CC1]

To a solution of Compound 2_2 (959 mg, 2.57 mmol, 1.1 eq) in ACN (6.0 mL) was added NaI (175 mg, 1.17 mmol, 0.50 eq), Cs₂CO₃ (1.14 g, 3.50 mmol, 1.5 eq) and Compound 29 (900 mg, 2.33 mmol, 1.0 eq). The mixture was stirred at 90° C. for 6 h. TLC (Petroleum ether:Ethyl acetate=1:1) showed Compound 2_9 (900 mg, 2.33 mmol, 1.0 eq) was used up. The reaction mixture was filtered and concentrated under reduced pressure to give a residue. Combined with ET42897-18, 21, 24 purification. The residue was purified by prep-HPLC (column: Phenomenex Gemini-NX C18 75×30 mm×3 um; mobile phase: [water(0.1% TFA)-ACN]; B %: 5%-35%, 12 min). Compound 2_3 (TFA) (500 mg, yield 23.0%) was obtained as white oil, checked by LCMS.

### Step 6. Preparation of Compound 2_4

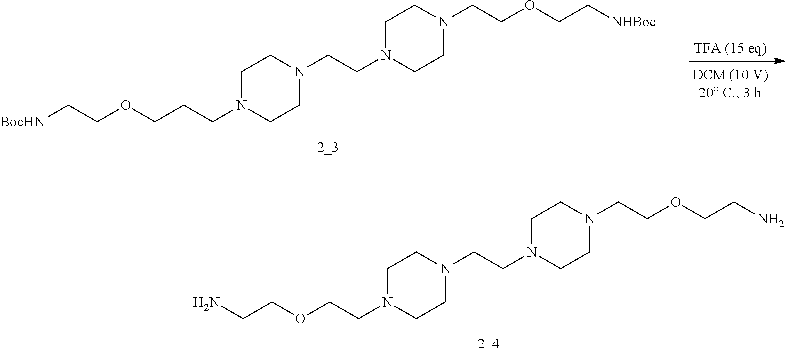 [SMILES: CC(C)(C)OC(=O)NCCOCCCN1CCN(CCN2CCN(CCOCCNC(=O)OC(C)(C)C)CC2)CC1 ; NCCOCCN1CCN(CCN2CCN(CCOCCN)CC2)CC1]

To a solution of Compound 2_3 (500 mg, 713 μμmol, 1.0 eq, TFA) in DCM (5.0 mL) was added TFA (1.22 g, 10.7 mmol, 792 μL, 15 eq). The mixture was stirred at 20° C. for 3 h. HNMR (ET42897-27-P1A2) showed Compound 2_3 (500 mg, 713 μμmol, 1.0 eq, TFA) was used up. The reaction mixture was filtered and concentrated under reduced pressure to give a residue. The crude was dissociated with alkaline resin to a pH of about 7-8 for filtration and rotary evaporation. Compound 2_4 (0.28 g, crude) was obtained as yellow oil, checked by ¹H NMR.

¹H NMR: (400 MHz, MeOD)

δ ppm 3.61 (t, J=5.57 Hz, 2H), 3.43-3.53 (m, 6H), 2.77 (td, J=5.28, 3.19 Hz, 4H), 2.42-2.65 (m, 20H), 1.73-1.84 (m, 2H).

### Step 7. Preparation of Compound SIL1-7

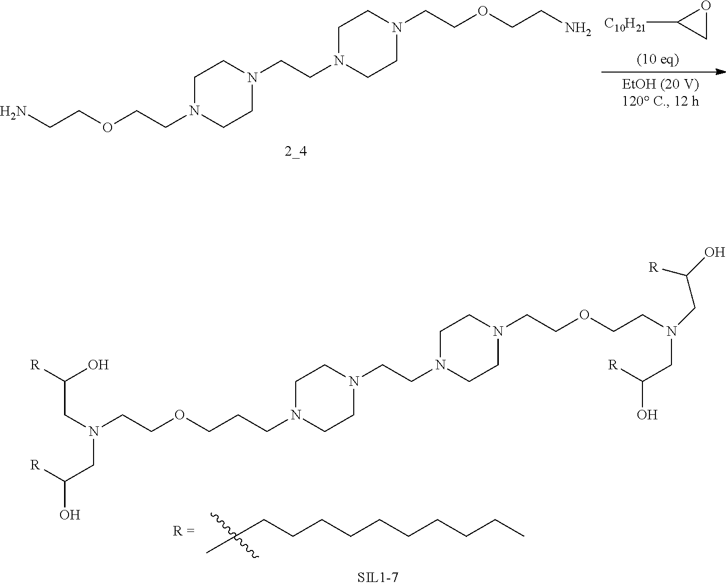

To a solution of Compound 2_4 (0.25 g, 671.06 μmol, 1.0 eq) in EtOH (5.0 mL) was added 2-decyloxirane (1.24 g, 6.71 mmol, 10 eq). The mixture was stirred at 120° C. for 12 h. LCMS (ET42897-29-P1A1) showed Compound 2_4 (0.25 g, 671.06 μmol, 1.0 eq) was used up, and the product RT=0.704 min. The reaction mixture was filtered and concentrated under reduced pressure to give a residue. Combined with ET42897-28 purification, the residue was purified by column chromatography (SiO₂, Dichloromethane:Methanol=20/1 to 10/1). Compound SIL1-7 (60 mg, yield 7%) was obtained as yellow oil, checked by ¹H NMR.

¹H NMR: (400 MHz, CDCl₃)

δ ppm 3.39-3.68 (m, 11H), 2.27-2.91 (m, 34H), 1.82 (s, 2H), 1.15-1.55 (m, 77H), 0.89 (t, J=6.48 Hz, 13H)

### Example 3. Synthesis of Compound SIL1-8

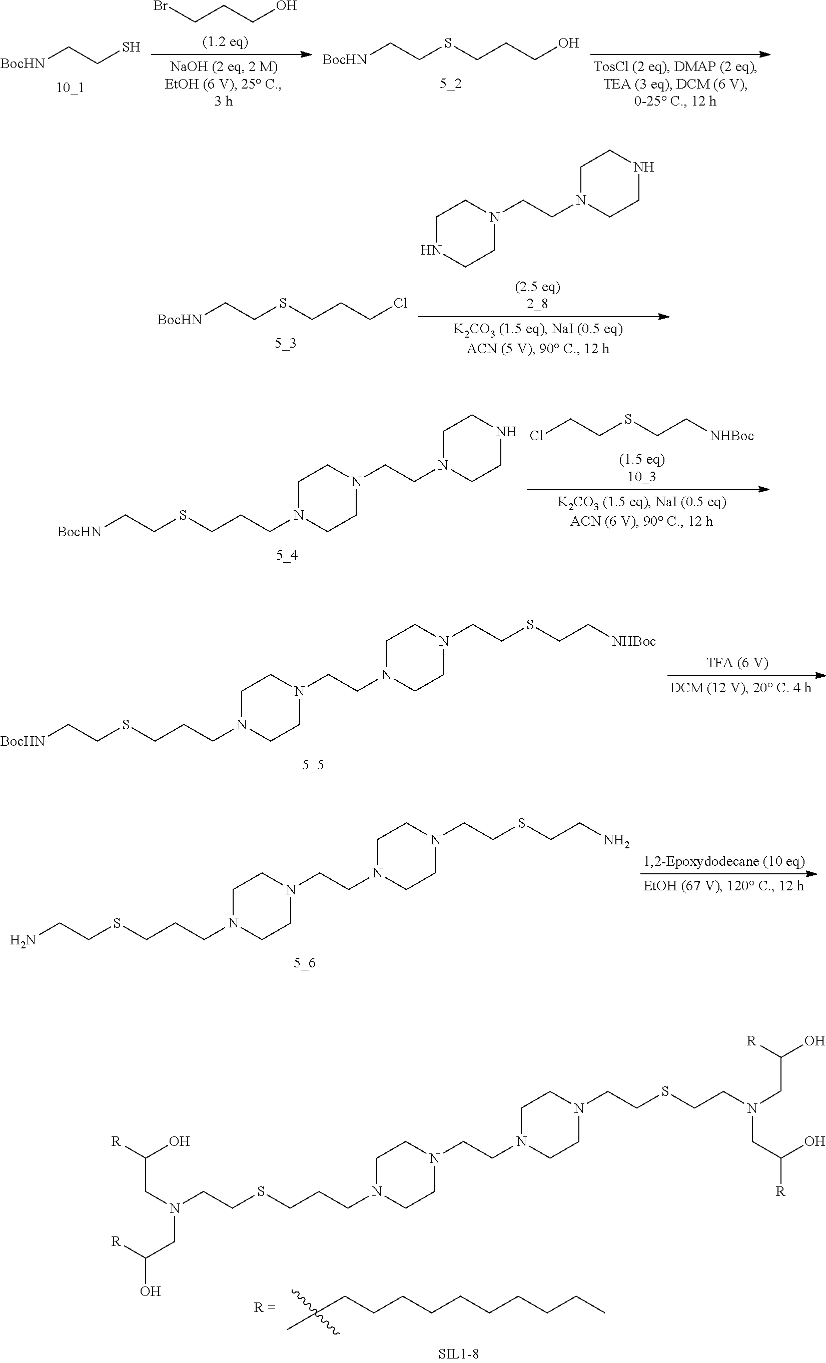

### Step 1. Preparation of Compound 5_2

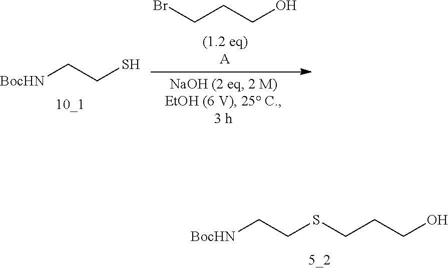 [SMILES: OCCCBr ; CC(C)(C)OC(=O)NCCS ; CC(C)(C)OC(=O)NCCSCCCO]

To a solution of Compound 10_1 (25 g, 141 mmol, 1.0 eq) and Compound A (23.52 g, 169 mmol, 15.27 mL, 1.2 eq) in EtOH (150 mL) was added NaOH (2 M, 141.03 mL, 2.0 eq). The mixture was stirred at 25° C. for 3 h. TLC (Petroleum ether:Ethyl acetate=1:1, product R_{f}=0.43) showed the raw material response to complete. The mixture was added H₂O (100 mL). The aqueous phase was extracted with MTBE (500 mL, 200 mL). The combined organic phase was washed with brine (100 mL), dried with anhydrous Na₂SO4, filtered and concentrated in vacuum. The residue was purified by column chromatography (SiO₂, Petroleum ether:Ethyl acetate=100:1 to 2:1). Compound 5_2 (24 g, 101 mmol, 72.3% yield) was obtained as yellow oil.

¹H NMR: 400 MHz CDCl₃

δ=4.89 (br s, 1H), 3.76 (t, J=5.9 Hz, 2H), 3.28-3.39 (m, 2H), 2.67 (q, J=6.7 Hz, 4H), 1.82-1.90 (m, 2H), 1.46 (s, 9H).

### Step 2. Preparation of Compound 5_3

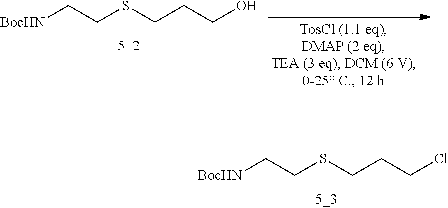 [SMILES: CC(C)(C)OC(=O)NCCSCCCO ; CC(C)(C)OC(=O)NCCSCCCCl]

To a solution of Compound 5_2 (15.4 g, 65.4 mmol, 1.0 eq), DMAP (15.9 g, 130 mmol, 2.0 eq) and DIEA (16.9 g, 130 mmol, 22.8 mL, 2.0 eq) in DCM (92 mL) was added TsCl (13.7 g, 71.9 mmol, 1.1 eq) at 0° C. The mixture was stirred at 25° C. for 12 h. TLC (Petroleum ether:Ethyl acetate=2:1, product R_{f}=0.59) showed the raw material response to complete. The mixture was evaporated to dryness. The residue was purified by column chromatography (SiO₂, Petroleum ether/Ethyl acetate=50:1 to 5:1). Compound 5_3 (8.54 g, 33.6 mmol, 51.4% yield) was obtained as yellow oil.

¹H NMR: 400 MHz CDCl₃

δ=4.91 (br s, 1H), 3.66 (t, J=6.3 Hz, 2H), 3.33 (br d, J=5.9 Hz, 2H), 2.68 (td, J=6.8, 15.7 Hz, 4H), 1.98-2.09 (m, 2H), 1.45 (s, 9H)

### Step 3. Preparation of Compound 5_4

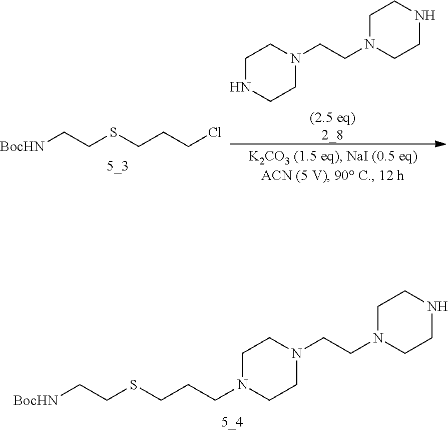 [SMILES: C1CN(CCN2CCNCC2)CCN1 ; CC(C)(C)OC(=O)NCCSCCCCl ; CC(C)(C)OC(=O)NCCSCCCN1CCN(CCN2CCNCC2)CC1]

To a solution of Compound 2_8 (11.72 g, 59.10 mmol, 2.5 eq) and Compound 5_3 (6 g, 23.64 mmol, 1 eq) in ACN (30 mL) was added NaI (1.77 g, 11.8 mmol, 0.5 eq) and K₂CO₃ (4.90 g, 35.46 mmol, 1.5 eq). The mixture was stirred at 90° C. for 12 h. TLC (Dichloromethane:Methanol=10:1, product R_{f}=0.38) showed the raw material response to complete. The mixture was filtered and the filtrate was evaporated to dryness. The residue was purified by column chromatography (SiO₂, Dichloromethane:Methanol=100:1 to 40:1). Compound 54 (5 g, 12.03 mmol, 50.88% yield) was obtained as a white solid.

¹H NMR: 400 MHz CDCl₃

δ=4.95 (br dd, J=3.0, 6.1 Hz, 1H), 3.26-3.35 (m, 2H), 2.89 (t, J=4.9 Hz, 4H), 2.37-2.67 (m, 22H), 1.76 (m, J=7.3 Hz, 2H), 1.44 (s, 9H)

### Step 4. Preparation of Compound 5_5

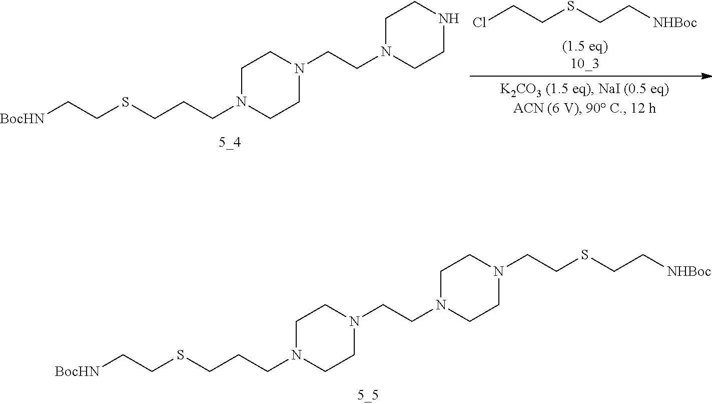

To a solution of Compound 5_4 (4.1 g, 9.86 mmol, 1 eq) and Compound 10_3 (3.55 g, 14.80 mmol, 1.5 eq) in ACN (24 mL) was added K₂CO₃ (2.05 g, 14.80 mmol, 1.5 eq) and NaI (739 mg, 4.93 mmol, 0.5 eq). The mixture was stirred at 90° C. for 12 h. TLC (Dichloromethane:Methanol=10:1, product R_{f}=0.43) showed the raw material response to complete. The mixture was evaporated to dryness. The residue was purified by column chromatography (SiO₂, Ethyl acetate:Methanol=100:1 to 20:1). Compound 5_5 (4.2 g, crude) was obtained as yellow oil

### Step 5. Preparation of Compound 5_6

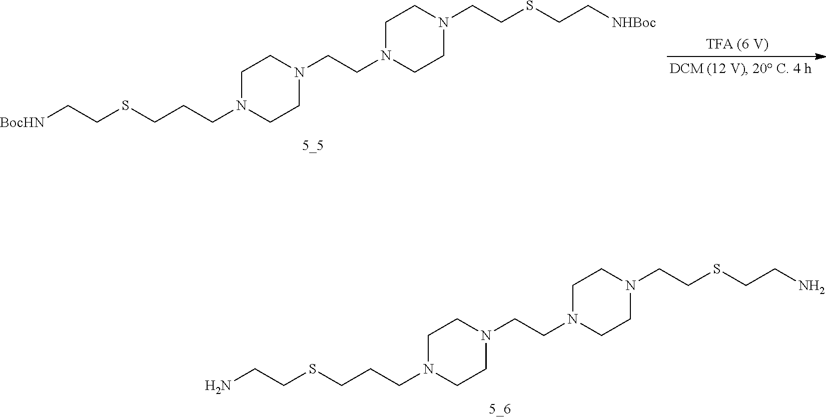 [SMILES: CC(C)(C)OC(=O)NCCSCCCN1CCN(CCN2CCN(CCSCCNC(=O)OC(C)(C)C)CC2)CC1 ; NCCSCCCN1CCN(CCN2CCN(CCSCCN)CC2)CC1]

To a solution of Compound 5_5 (800 mg, 1.29 mmol, 1 eq) in DCM (10 mL) was added TFA (7.70 g, 67.5 mmol, 5 mL, 52.3 eq). The mixture was stirred at 20° C. for 4 h. LCMS showed the starting material was consumed completely. The mixture was evaporated to dryness. Compound 5_6 (450 mg, crude) was obtained as yellow oil.

### Step 6. Preparation of Compound SIL8

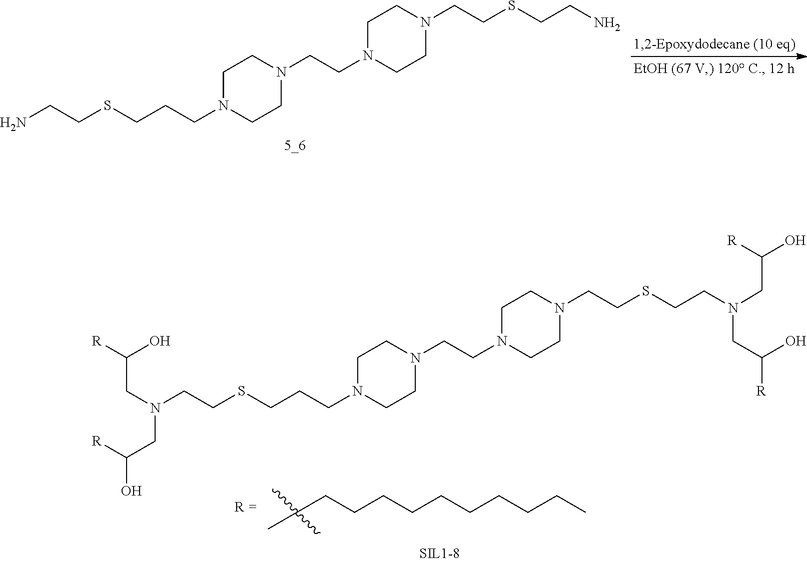

Two batches. To a solution of Compound 5_6 (225 mg, 537 μmol, 1 eq) in EtOH (15 mL) was added 2-decyloxirane (990 mg, 5.37 mmol, 10 eq). The mixture was stirred at 120° C. for 12 h. LCMS (ET42415-63-P1A1, product: RT=0.703 min). The mixture was evaporated to dryness. The residue was purified by column chromatography (SiO₂, Ethyl acetate:Methanol=100:1 to 40:1). Compound SIL1-8 (70 mg, 60.55 μmol, 5.63% yield) was obtained as yellow oil.

¹H NMR: 400 MHz CDCl₃

δ=3.58-3.67 (m, 4H), 2.26-2.85 (m, 45H), 1.72-1.82 (m, 2H), 1.21-1.51 (m, 77H), 0.86-0.91 (m, 12H).

### Example 4. Synthesis of Compound SIL1-9

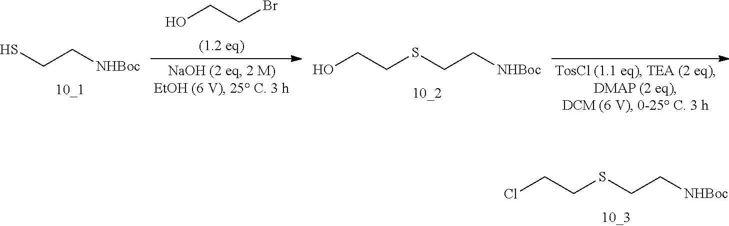

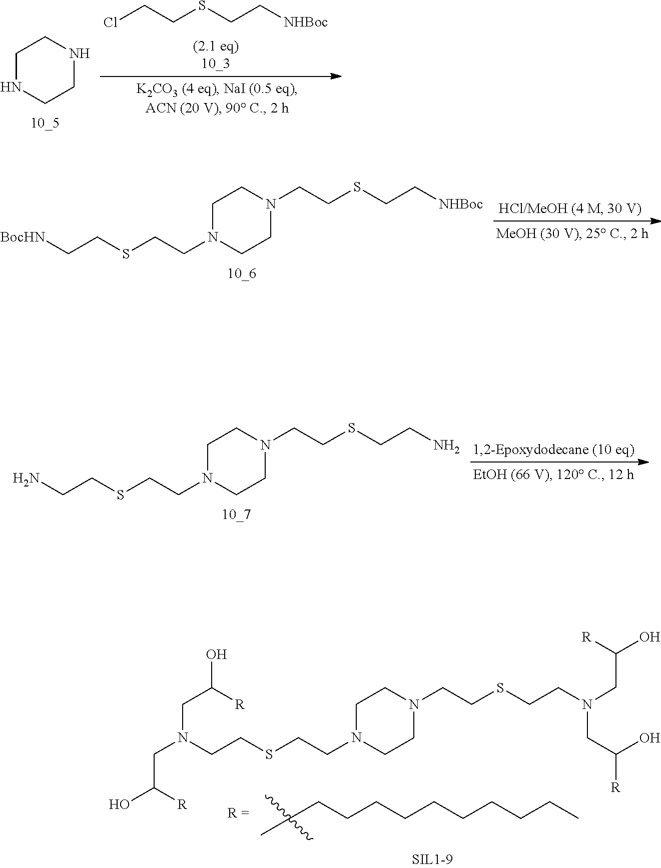

### Step 1. Preparation of Compound 10_2

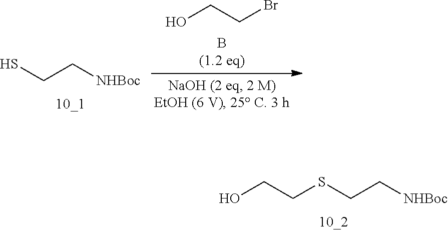

To a solution of Compound 10_1 (25 g, 141.03 mmol, 1 eq) and Compound B (21.2 g, 169 mmol, 12.0 mL, 1.2 eq) in EtOH (150 mL) was added NaOH (2 M, 141.03 mL, 2 eq). The mixture was stirred at 25° C. for 3 h. TLC (Petroleum ether:Ethyl acetate=1:1, product R_{f}=0.43) showed the raw material response to complete. The mixture was added H₂O (100 mL). The aqueous phase was extracted with MTBE (500 mL, 200 mL). The combined organic phase was washed with brine (100 mL), dried with anhydrous Na₂SO4, filtered and concentrated under reduced pressure. The residue was purified by column chromatography (SiO₂, Petroleum ether:Ethyl acetate=100:1 to 2:1). Compound 10_2 (19 g, 85.8 mmol, 60.8% yield) was obtained as yellow oil.

¹H NMR: 400 MHz CDCl₃

δ=4.80-4.99 (m, 1H), 3.76 (t, J=5.9 Hz, 2H), 3.34 (br t, J=6.4 Hz, 2H), 2.76 (t, J=5.9 Hz, 2H), 2.67 (t, J=6.6 Hz, 2H), 2.23 (br d, J=4.1 Hz, 1H), 1.46 (s, 9H).

### Step 2. Preparation of Compound 10_3

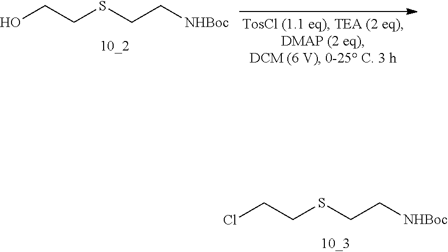 [SMILES: CC(C)(C)OC(=O)NCCSCCO ; CC(C)(C)OC(=O)NCCSCCCl]

To a solution of Compound 10_2 (5 g, 22.5 mmol, 1 eq), DMAP (5.52 g, 45.1 mmol, 2 eq) and TEA (4.57 g, 45.2 mmol, 6.29 mL, 2 eq) in DCM (30 mL) was added TosCl (4.74 g, 24.8 mmol, 1.1 eq) at 0° C. The mixture was stirred at 0-25° C. for 3 h. TLC (Petroleum ether:Ethyl acetate=2:1, product R_{f}=0.53) showed the raw material response to complete. The mixture was evaporated to dryness. The residue was purified by column chromatography (SiO₂, Petroleum ether:Ethyl acetate=50:1 to 5:1). Compound 10_3 (2.5 g, 10.4 mmol, 46.1% yield) was obtained as yellow oil.

¹H NMR: 400 MHz CDCl₃

δ=4.91 (br s, 1H), 3.61-3.68 (m, 2H), 3.28-3.37 (m, 2H), 2.85-2.92 (m, 2H), 2.70 (t, J=6.5 Hz, 2H), 1.45 (s, 9H).

### Step 3. Preparation of Compound 10_6

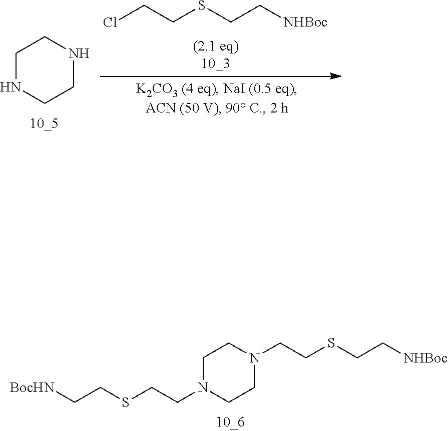 [SMILES: CC(C)(C)OC(=O)NCCSCCCl ; C1CNCCN1 ; CC(C)(C)OC(=O)NCCSCCN1CCN(CCSCCNC(=O)OC(C)(C)C)CC1]

To a solution of Compound 10_5 (400 mg, 4.64 mmol, 1 eq) and Compound 10_3 (2.34 g, 9.75 mmol, 2.1 eq) in ACN (20 mL) was added NaI (348 mg, 2.32 mmol, 0.5 eq) and K₂CO₃ (2.57 g, 18.5 mmol, 4 eq). The mixture was stirred at 90° C. for 2 h. TLC indicated ˜10% of Compound 10_3 was remained, and one major new spot with larger polarity was detected. The mixture was filtered and the filtrate was evaporated in vacuum. The residue was purified by column chromatography (SiO₂, Dichloromethane:Methanol=100:1 to 40:1). Compound 10_6 (900 mg, 1.83 mmol, 39.3% yield) was obtained as a white solid.

¹H NMR: 400 MHz CDCl₃

δ=5.03 (br s, 2H), 3.32 (q, J=5.8 Hz, 4H), 2.47-2.73 (m, 20H), 1.45 (s, 18H)

### Step 4. Preparation of Compound 10_7

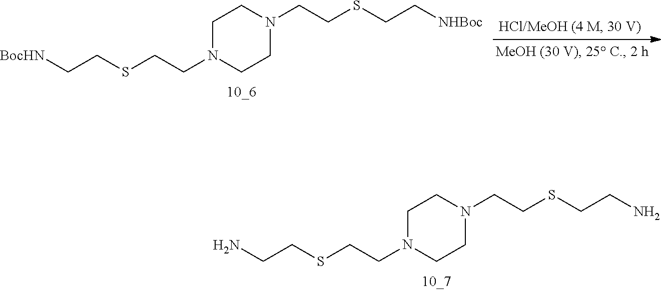 [SMILES: CC(C)(C)OC(=O)NCCSCCN1CCN(CCSCCNC(=O)OC(C)(C)C)CC1 ; NCCSCCN1CCN(CCSCCN)CC1]

To a solution of Compound 10_6 (900 mg, 1.83 mmol, 1 eq) in MeOH (27 mL) was added HCl/MeOH (4 M, 27.00 mL, 59.1 eq). The mixture was stirred at 25° C. for 2 h. TLC (Dichloromethane:Methanol=10:1, product R_{f}=0.04) showed the raw material response to complete. The mixture was filtered with MTBE (20 mL) and the filter cake was evaporated in vacuum. Compound 10_7 (600 mg, crude) was obtained as a white solid.

¹H NMR: 400 MHz DMSO-d₆

δ=2.97 (t, J=7.2 Hz, 2H), 2.59-2.66 (m, 2H), 2.53-2.58 (m, 4H), 2.31-2.48 (m, 13H).

### Step 5. Preparation of Compound SIL1-9

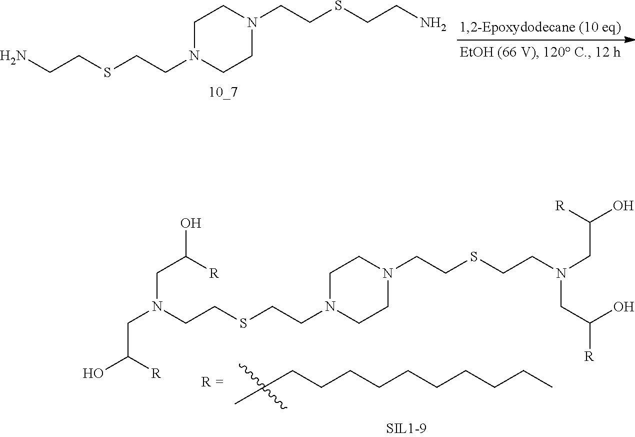

To a solution of Compound 10_7 (300 mg, 1.03 mmol, 1 eq) and 2-decyloxirane (1.13 g, 6.15 mmol, 6 eq) in EtOH (20 mL). The mixture was stirred at 120° C. for 12 hrs. LCMS (ET42415-26-P1A1, product: RT=0.737 min). The mixture was evaporated to dryness. The residue was purified by column chromatography (SiO₂, Dichloromethane:Methanol=100:1 to 40:1). Compound SIL1-9 (70 mg, 60.5 μmol, 5.90% yield, 89% purity) was obtained as yellow oil.

¹H NMR: 400 MHz CDCl₃

δ=3.62 (br d, J=3.6 Hz, 4H), 3.17-3.28 (m, 3H), 2.31-2.85 (m, 30H), 1.25-1.51 (m, 73H), 0.89 (t, J=6.7 Hz, 12H)

### Example 5. Synthesis of Compound SIL1-10

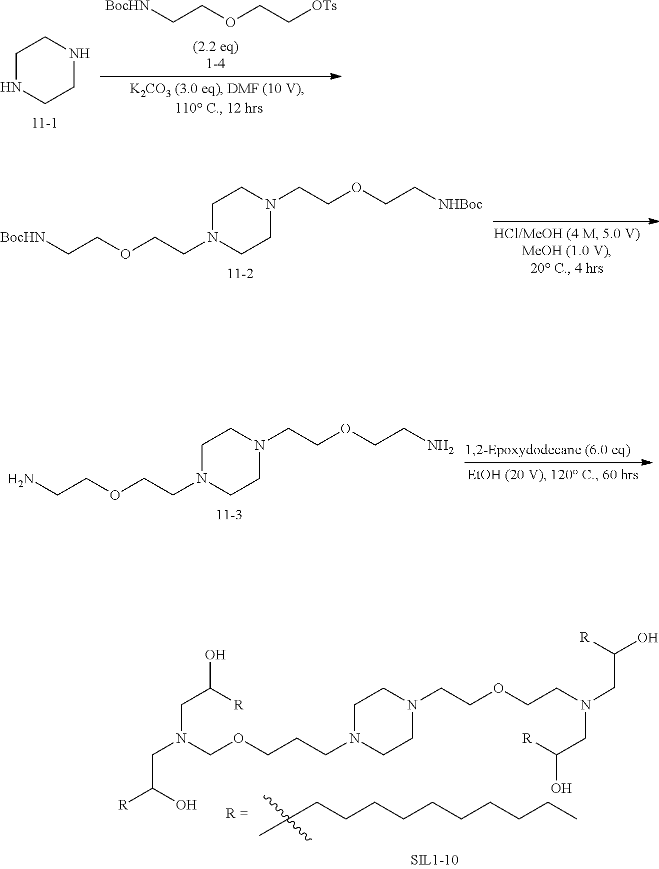

### Step 1. Preparation of Compound 11-2

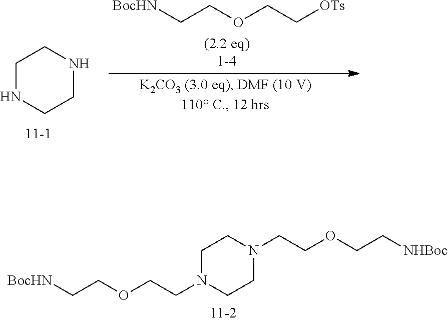 [SMILES: Cc1ccc(S(=O)(=O)OCCOCCNC(=O)OC(C)(C)C)cc1 ; C1CNCCN1 ; CC(C)(C)OC(=O)NCCOCCN1CCN(CCOCCNC(=O)OC(C)(C)C)CC1]

To the solution of Compound 11-1 (0.10 g, 1.16 mmol, 1.00 eq) in DMF (0.60 mL) was added K₂CO₃ (401 mg, 2.90 mmol, 2.50 eq) and Compound 1-4 (918 mg, 2.55 mmol, 2.20 eq). The reaction was stirred at 110° C. for 12 hrs. LC-MS showed compound 11-1 was consumed completely and desired mass was detected. The residue was poured into water (2.00 mL). The aqueous phase was extracted with DCM (5.00 mL). The combined organic phase was dried with anhydrous Na₂SO4, filtered and concentrated in vacuum. The residue was purified by silica gel chromatography (Dichloromethane:Methanol=30/1). Compound 11-2 (0.26 g, 564.47 μmol, 48.62% yield) was obtained as a white solid.

¹H NMR: 400 MHz CDCl₃

δ 5.17 (s, 2H), 3.60-3.56 (m, 4H), 3.54-3.49 (m, 4H), 3.31-3.29 (m, 4H), 2.59-2.41 (m, 11H), 1.45 (s, 18H)

### Step 2. Preparation of Compound 11-3

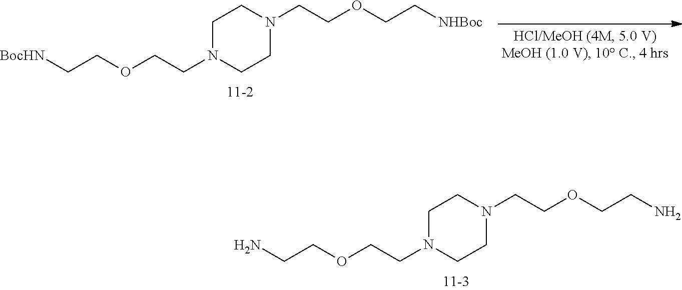 [SMILES: CC(C)(C)OC(=O)NCCOCCN1CCN(CCOCCNC(=O)OC(C)(C)C)CC1 ; NCCOCCN1CCN(CCOCCN)CC1]

To the solution of Compound 11-2 (0.260 g, 564 μmol, 1.00 eq) in MeOH (0.300 mL) was added HCl/MeOH (4M, 1.50 mL). The reaction was stirred at 10° C. for 4 hrs. TLC (Dichloromethane:Methanol=10:1) indicated Compound 11-2 was consumed completely. The reaction was concentrated under reduced pressure and adjusted pH to alkalinity by 6M K₂CO₃ (aq.). The crude product was used into the next step without further purification. Adjust the Ph of the solution to alkalinity. Compound 11-3 (0.158 g, crude) was obtained as a white solid.

¹H NMR: 400 MHz DMSO

δ 2.78-2.66 (m, 10H), 2.63-2.65 (m, 5H), 2.14-2.05 (m, 4H), 2.04-2.00 (m, 4H).

### Step 3. Preparation of Compound SIL1-10

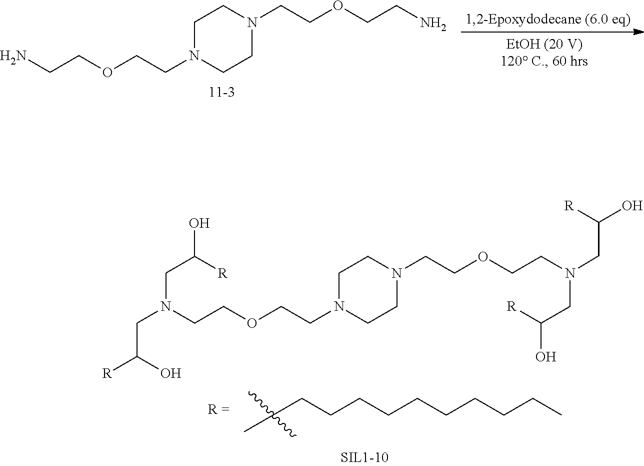

To the solution of Compound 11-3 (158 mg, 606 μmol, 1.00 eq) in EtOH (4.00 mL) was added 2-decyloxirane (671 mg, 3.64 mmol, 6.00 eq). The reaction was stirred at 120° C. for 60 hr. The mixture was stirred at 15° C. for 16 hrs. TLC (Dichloromethane:Methanol=10/1) indicated Compound 11-3 was consumed completely. The residue was purified by silica gel chromatography (Dichloromethane:Methanol=80/1, 20/1). Compound SIL1-10 (50.0 mg, 47.9 μmol, 7.90% yield, 95.7% purity) was obtained as a gray oil.

¹H NMR: 400 MHz CDCl₃

δ 3.50-3.80 (m, 16H), 2.75-2.84 (m, 2H), 2.53-2.64 (m, 10H), 2.18-2.51 (m, 7H), 1.26-1.44 (m, 105H), 0.89 (t, J=6.4 Hz, 12H).

LCMS: (M/2+1): 499.6

### Example 6. Synthesis of Compound SIL1-11

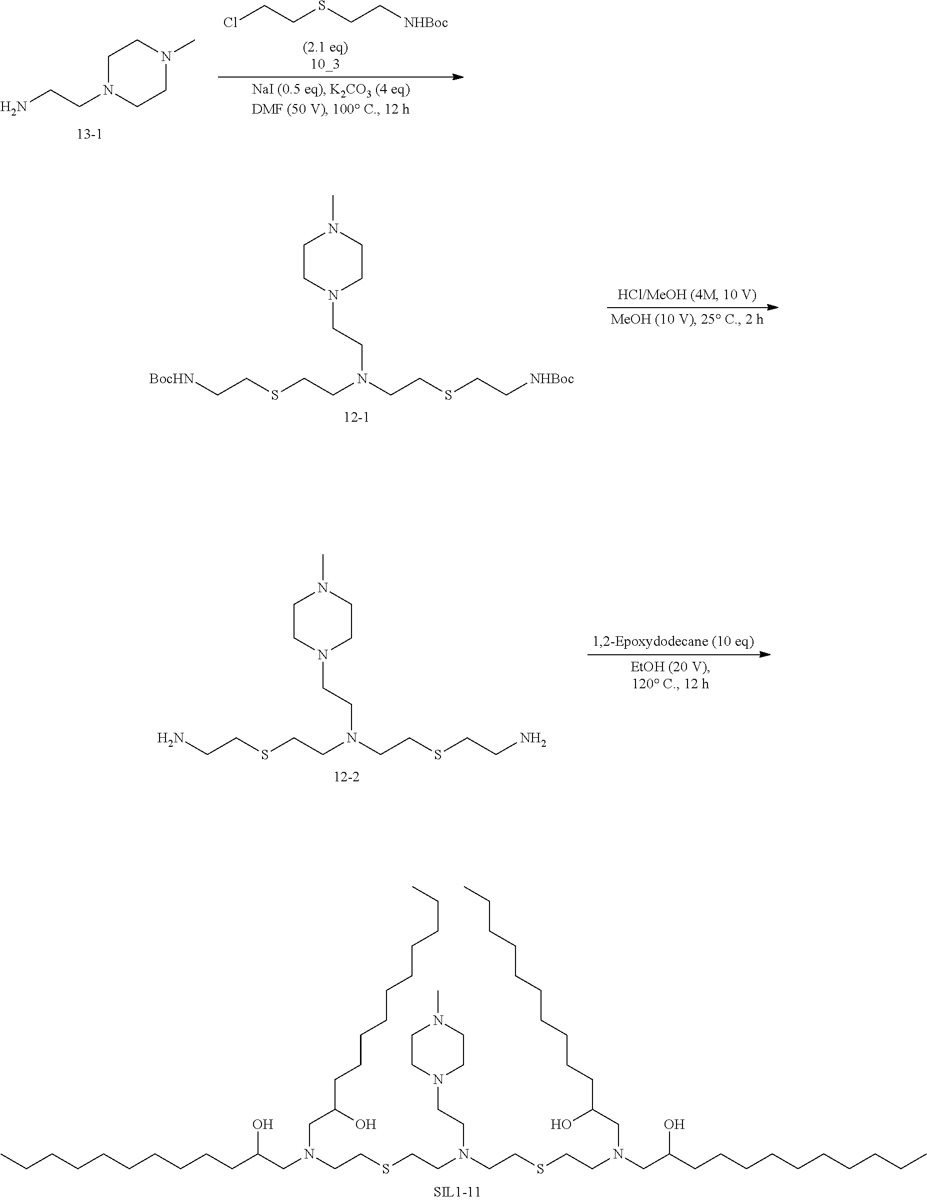

### Step 1. Preparation of Compound 12_1

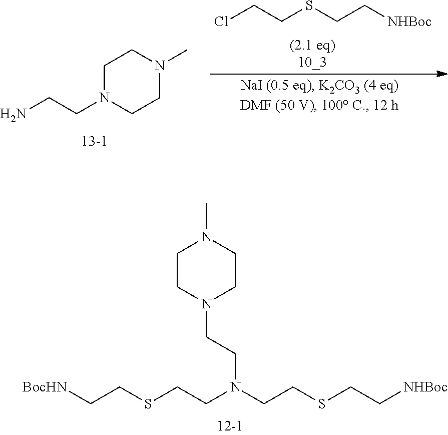

To a solution of Compound 13_1 (1 g, 6.98 mmol, 1 eq) and Compound 10_3 (3.52 g, 14.6 mmol, 2.1 eq) in DMF (50 mL) was added NaI (523 mg, 3.49 mmol, 0.5 eq) and K₂CO₃ (3.86 g, 27.9 mmol, 4 eq). The mixture was stirred at 100° C. for 12 h. TLC (Dichloromethane:Methanol=8:1, product R_{f}=0.28) showed the raw material response to complete. The mixture was evaporated to dryness. The residue was purified by column chromatography (SiO₂, Dichloromethane:Methanol=100:1 to 20:1). Compound 12_1 (400 mg, 688 μmol, 9.86% yield, 94.6% purity) was obtained as yellow oil.

### Step 2. Preparation of Compound 12_2

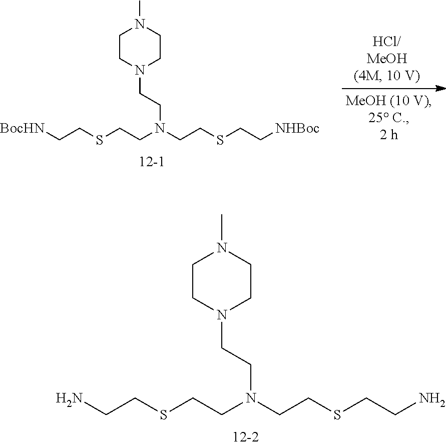 [SMILES: CN1CCN(CCN(CCSCCNC(=O)OC(C)(C)C)CCSCCNC(=O)OC(C)(C)C)CC1 ; CN1CCN(CCN(CCSCCN)CCSCCN)CC1]

To a solution of Compound 12_1 (400 mg, 727 μmol, 1 eq) in MeOH (4 mL) was added HCl/MeOH (4 M, 4 mL, 21.9 eq). The mixture was stirred at 25° C. for 2 h. TLC (Dichloromethane:Methanol=10:1, product R_{f}=0.02) showed the raw material response to complete. The mixture was evaporated to dryness. Compound 12_2 (230 mg, crude) was obtained as yellow oil.

### Step 3. Preparation of Compound SIL1-11

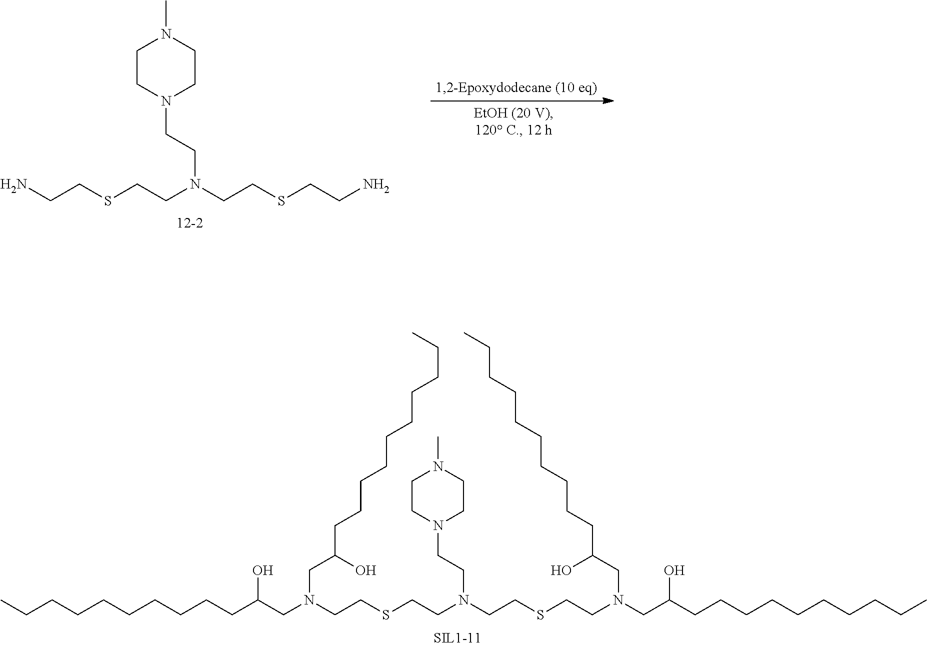 [SMILES: CN1CCN(CCN(CCSCCN)CCSCCN)CC1 ; CCCCCCCCCCC(O)CN(CCSCCN(CCSCCN(CC(O)CCCCCCCCCC)CC(O)CCCCCCCCCC)CCN1CCN(C)CC1)CC(O)CCCCCCCCCC]

To a solution of Compound 12_2 (100 mg, 286 μmol, 1 eq) in EtOH (2 mL) was added 2-decyloxirane (527 mg, 2.86 mmol, 10 eq). The mixture was stirred at 120° C. for 12 h. LCMS (ET42415-42-P1A1, product: RT=0.713 min). The mixture was evaporated to dryness. The residue was purified by column chromatography (SiO₂, Ethyl acetate:Methanol=100:1 to 40:1). Compound SIL1-11 (70 mg, 62.9 μmol, 22.00% yield, 97.7% purity) was obtained as yellow oil

¹H NMR: 400 MHz CDCl₃

δ=3.58-3.74 (m, 4H), 2.28-2.90 (m, 37H), 1.51-1.52 (m, 1H), 1.25-1.51 (m, 73H), 0.92 (t, J=6.8 Hz, 12H).

### Example 7. Synthesis of Compound SIL1-12

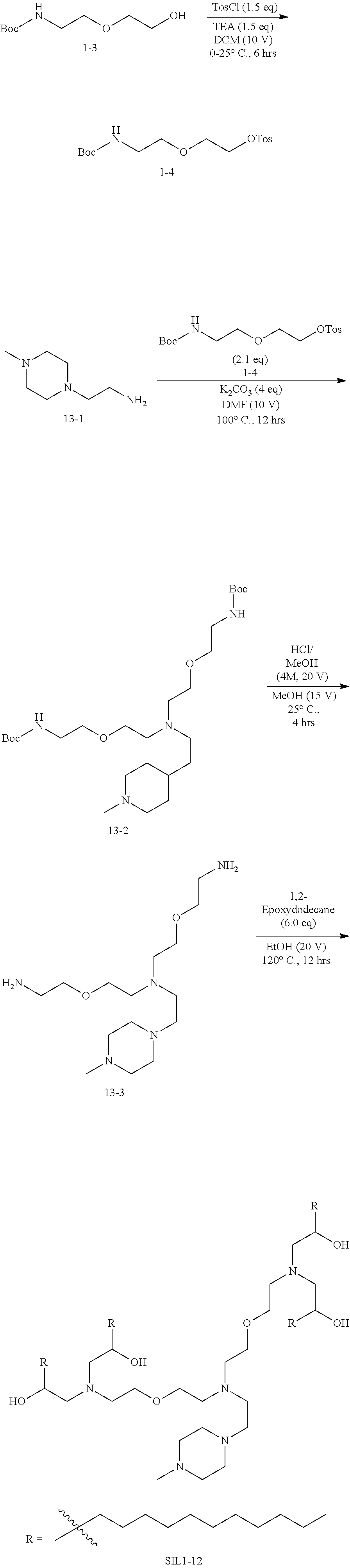

### Step 1. Preparation of Compound 12_1

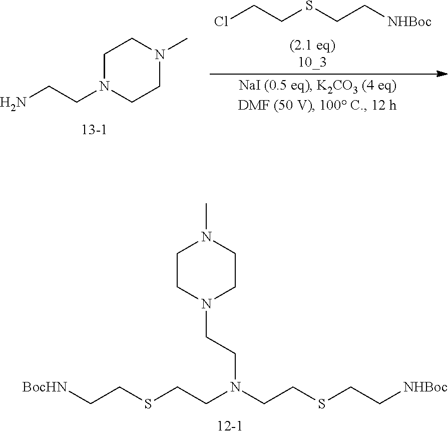

To a solution of Compound 13_1 (1 g, 6.98 mmol, 1 eq) and Compound 10_3 (3.52 g, 14.6 mmol, 2.1 eq) in DMF (50 mL) was added NaI (523 mg, 3.49 mmol, 0.5 eq) and K₂CO₃ (3.86 g, 27.9 mmol, 4 eq). The mixture was stirred at 100° C. for 12 h. TLC (Dichloromethane:Methanol=8:1, product R_{f}=0.28) showed the raw material response to complete. The mixture was evaporated to dryness. The residue was purified by column chromatography (SiO₂, Dichloromethane:Methanol=100:1 to 20:1). Compound 12_1 (400 mg, 688 μmol, 9.86% yield, 94.6% purity) was obtained as yellow oil.

### Step 2. Preparation of Compound 12_2

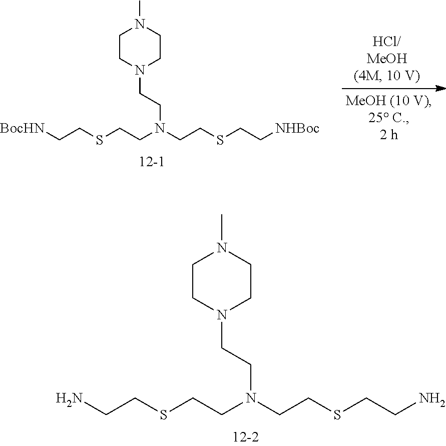 [SMILES: CN1CCN(CCN(CCSCCNC(=O)OC(C)(C)C)CCSCCNC(=O)OC(C)(C)C)CC1 ; CN1CCN(CCN(CCSCCN)CCSCCN)CC1]

To a solution of Compound 12_1 (400 mg, 727 μmol, 1 eq) in MeOH (4 mL) was added HCl/MeOH (4 M, 4 mL, 21.9 eq). The mixture was stirred at 25° C. for 2 h. TLC (Dichloromethane:Methanol=10:1, product R_{f}=0.02) showed the raw material response to complete. The mixture was evaporated to dryness. Compound 12_2 (230 mg, crude) was obtained as yellow oil.

### Step 3. Preparation of Compound SIL1-12

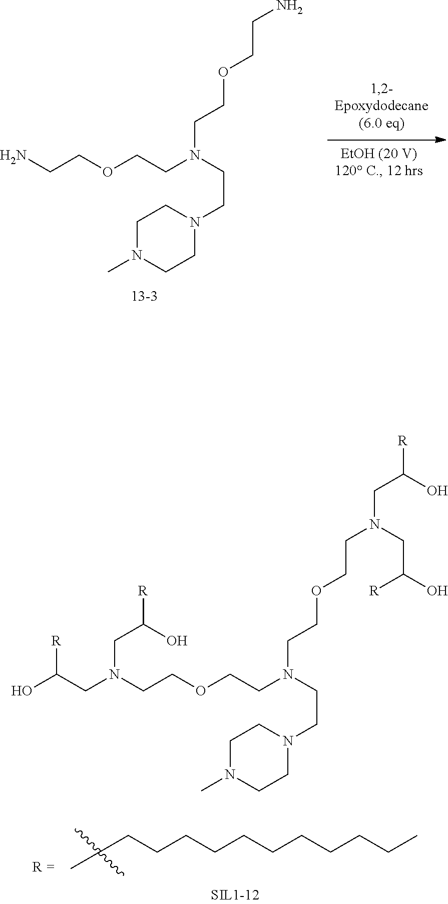

To a solution of Compound 13-3 (560 mg, 1.76 mmol, 1 eq) in EtOH (11 mL) was added 2-decyloxirane (1.95 g, 10.6 mmol, 6 eq). The mixture was stirred at 120° C. for 12 hrs. LCMS (ET42513-15-P1A1, product RT=0.690 min) showed the reaction was completed. The reaction was filtered and concentrated under reduced pressure to give a residue. The residue was purified by column chromatography (SiO₂, Dichloromethane:Methanol=100/1 to 0/1) to give Compound SIL1-12 (200 mg, 190 μmol, 10.8% yield) as a yellow oil.

LCMS: [M+H]⁺: m/z=1054.0

¹H NMR: (400 MHz CD₃OD)

δ=2.62-2.82 (m, 7H), 2.54-2.58 (m, 16H), 2.30-2.50 (m, 3H), 2.16 (s, 1H), 1.30 (m, 75H), 0.89-0.92 (m, 12H)

### Example 8. Synthesis of Compound SIL1-13

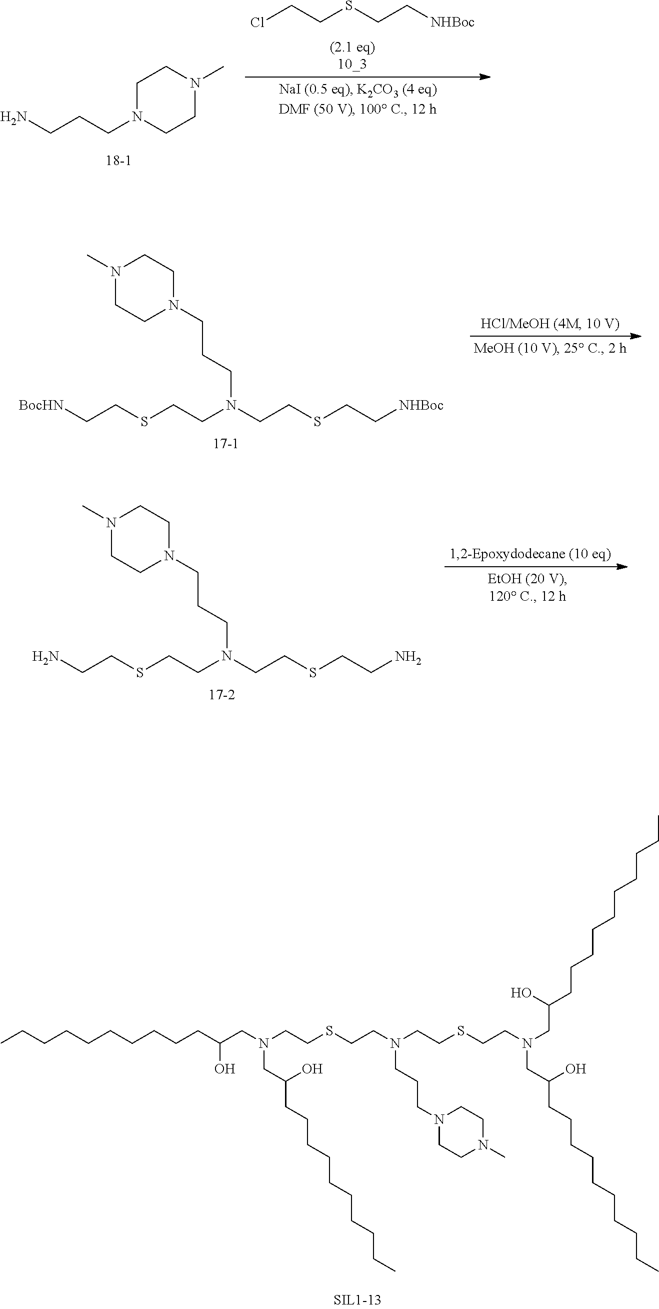 [SMILES: CC(C)(C)OC(=O)NCCSCCCl ; CN1CCN(CCCN)CC1 ; CN1CCN(CCCN(CCSCCNC(=O)OC(C)(C)C)CCSCCNC(=O)OC(C)(C)C)CC1 ; CN1CCN(CCCN(CCSCCN)CCSCCN)CC1 ; CCCCCCCCCCC(O)CN(CCSCCN(CCCN1CCN(C)CC1)CCSCCN(CC(O)CCCCCCCCCC)CC(O)CCCCCCCCCC)CC(O)CCCCCCCCCC]

### Step 1. Preparation of Compound 17_1

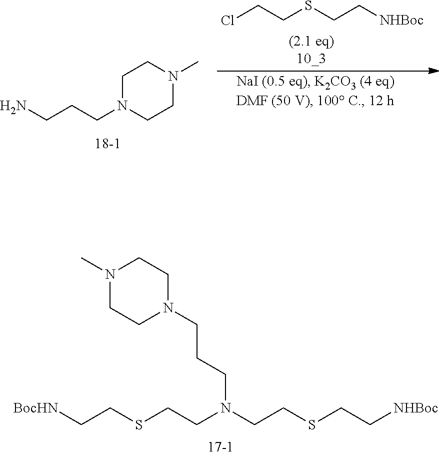 [SMILES: CC(C)(C)OC(=O)NCCSCCCl ; CN1CCN(CCCN)CC1 ; CN1CCN(CCCN(CCSCCNC(=O)OC(C)(C)C)CCSCCNC(=O)OC(C)(C)C)CC1]

To a solution of Compound 18_1 (1 g, 6.36 mmol, 1 eq) and Compound 17_1 (3.20 g, 13.3 mmol, 2.1 eq) in DMF (50 mL) was added NaI (476 mg, 3.18 mmol, 0.5 eq) and K₂CO₃ (3.52 g, 25.4 mmol, 4 eq). The mixture was stirred at 100° C. for 12 h. TLC (Dichloromethane:Methanol=10:1, product R_{f}=0.36) showed the raw material response to complete. The mixture was evaporated to dryness. The residue was purified by column chromatography (SiO₂, Dichloromethane:Methanol=100:1 to 40:1). Compound 17_1 (400 mg, 695 μmol, 10.9% yield, 98% purity) was obtained as yellow oil.

### Step 2. Preparation of Compound 17_2

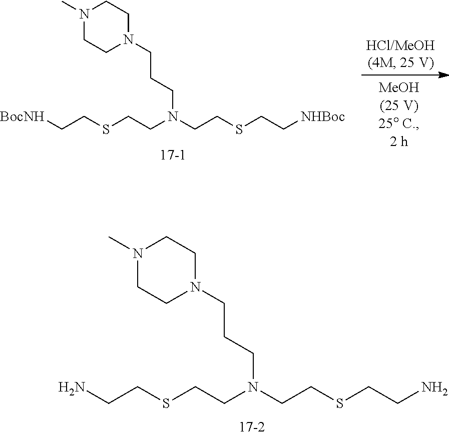 [SMILES: CN1CCN(CCCN(CCSCCNC(=O)OC(C)(C)C)CCSCCNC(=O)OC(C)(C)C)CC1 ; CN1CCN(CCCN(CCSCCN)CCSCCN)CC1]

To a solution of Compound 17_1 (400 mg, 709 μmol, 1 eq) in MeOH (10 mL) was added HCl/MeOH (4 M, 10 mL, 56.3 eq). The mixture was stirred at 25° C. for 2 h. LCMS showed the starting material was consumed completely. The mixture was evaporated to dryness. Compound 17_2 (290 mg, crude) was obtained as yellow oil

### Step 3. Preparation of Compound SIL1-13

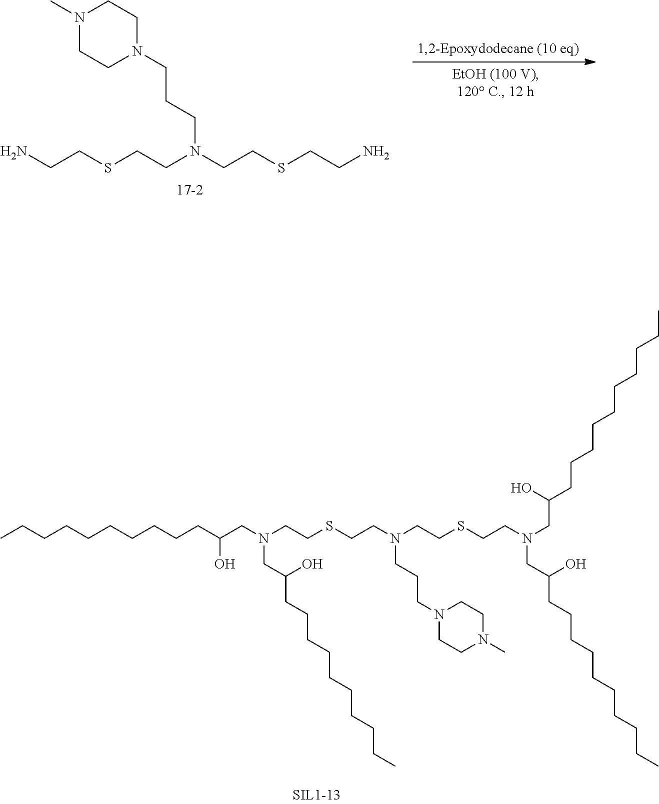 [SMILES: CN1CCN(CCCN(CCSCCN)CCSCCN)CC1 ; CCCCCCCCCCC(O)CN(CCSCCN(CCCN1CCN(C)CC1)CCSCCN(CC(O)CCCCCCCCCC)CC(O)CCCCCCCCCC)CC(O)CCCCCCCCCC]

To a solution of Compound 17_2 (130 mg, 357 μmol, 1 eq) in EtOH (13 mL) was added 2-decyloxirane (658 mg, 3.58 mmol, 10 eq). The mixture was stirred at 120° C. for 12 h. LCMS (ET42415-43-P1A1, product: RT=0.712 min). The mixture was evaporated to dryness. The residue was purified by column chromatography (SiO₂, Ethyl acetate:Methanol=100:1 to 40:1). Compound SIL1-13 (70 mg, 60.3 μmol, 8.44% yield, 94.9% purity) was obtained as yellow oil.

¹H NMR: 400 MHz CDCl₃

δ=3.57-3.69 (m, 4H), 2.28-2.86 (m, 35H), 1.17-1.53 (m, 78H), 0.89 (t, J=6.7 Hz, 12H)

LCMS: ET42415-43-P1A1

### Example 9. Synthesis of Compound SIL1-16

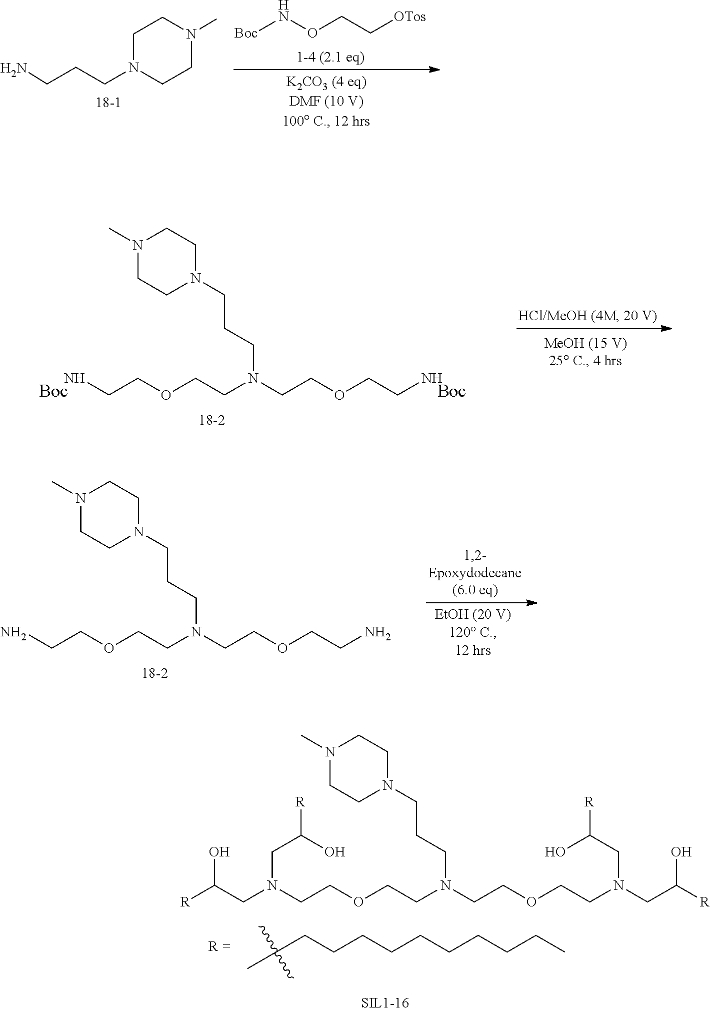

### Step 1. Preparation of Compound 18-2

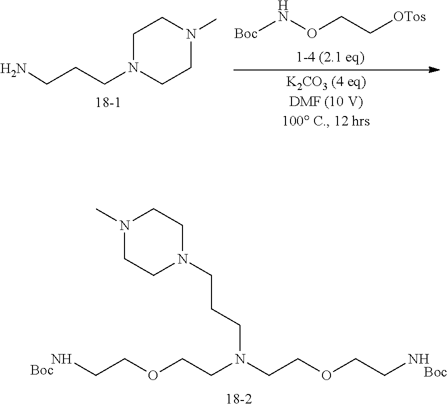

To a solution of Compound 18-1 (450 mg, 2.86 mmol, 1 eq), Compound 1-4 (2.16 g, 6.01 mmol, 2.1 eq) and K₂CO₃ (1.58 g, 11.5 mmol, 4 eq) in DMF (4.5 mL) was degassed and purged with N₂ for 3 times, and then the mixture was stirred at 90° C. for 12 hrs under N₂ atmosphere. LCMS (ET42513-10-P1A2, product RT=0.051 min) showed the reaction was completed. The reaction was filtered and concentrated under reduced pressure to give a residue. The residue was purified by column chromatography (SiO₂, Dichloromethane:Methanol=100/1 to 0/1) to give Compound 18-2 (760 mg, 1.43 mmol, 50.0% yield) as a yellow oil.

LCMS: [M+H]⁺: m/z=532.0

¹H NMR: (400 MHz CDCl₃)

δ=3.49-3.59 (m, 13H), 3.29-3.35 (m, 6H), 2.36 (s, 3H), 1.44 (m, 22H)

### Step 2. Preparation of Compound 18-3

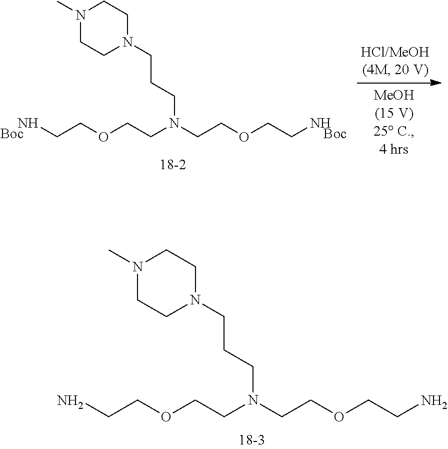 [SMILES: CN1CCN(CCCN(CCOCCN)CCOCCN)CC1 ; CN1CCN(CCCN(CCOCCN)CCOCCN)CC1]

To a solution of Compound 18-2 (760 mg, 1.43 mmol, 1 eq) in MeOH (11 mL) was added HCl/MeOH (4 M, 357 μL, 1 eq). The mixture was stirred at 25° C. for 4 hrs. LCMS (ET42513-12-P1A1, product RT=0.056 min) showed the reaction was completed. The pH value of the reaction solution was adjusted to 7-8 with aqueous saturated K₂CO₃. The crude was used for next step directly without further purification. Compound 18-3 (810 mg, crude) was obtained as a yellow oil.

LCMS: [M+H]+: m/z=332.0

### Step 3. Preparation of Compound SIL1-16

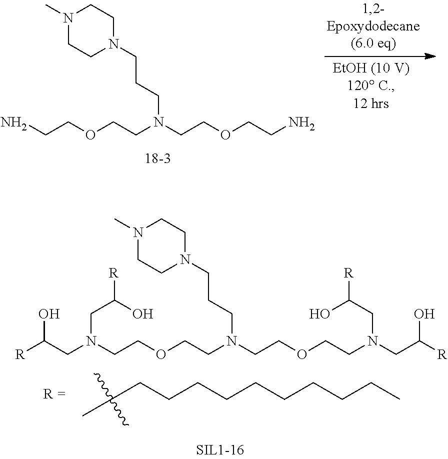

To a solution of Compound 18-3 (810 mg, 2.44 mmol, 1 eq) in EtOH (8 mL) was added 2-decyloxirane (2.70 g, 14.7 mmol, 6 eq). The mixture was stirred at 120° C. for 12 hrs. LCMS (ET42513-13-P1A1, product RT=0.699 min) showed the reaction was completed. The reaction was filtered and concentrated under reduced pressure to give a residue. The residue was purified by column chromatography (SiO₂, Dichloromethane:Methanol=100/1 to 0/1). The crude product was purified by reversed-phase HPLC (column: Phenomenex Luna C18 100*30 mm*5 um; mobile phase: [water(0.1% TFA)-ACN]; B %: 35%-70%, 10 min) to give Compound SIL1-16 (40 mg, 37.4 μmol, 1.53% yield) as a yellow oil.

LCMS: [M+H]+: m/z=1068.0

¹H NMR: (400 MHz CD₃OD)

δ=2.31 (s, 3H), 2.16 (s, 1H), 1.30-1.48 (m, 76H), 0.89-0.92 (m, 12H)

Other compounds, such as SIL1-1, SIL1-2, SIL1-3, SIL1-4, SIL1-5, SIL1-6, SIL1-14, SIL1-15, and SIL1-16, were prepared similarly with the methods described above.

### Synthesis of Compounds of Formula (I-B)

### Example 1. Synthesis of Compound SIL2-1

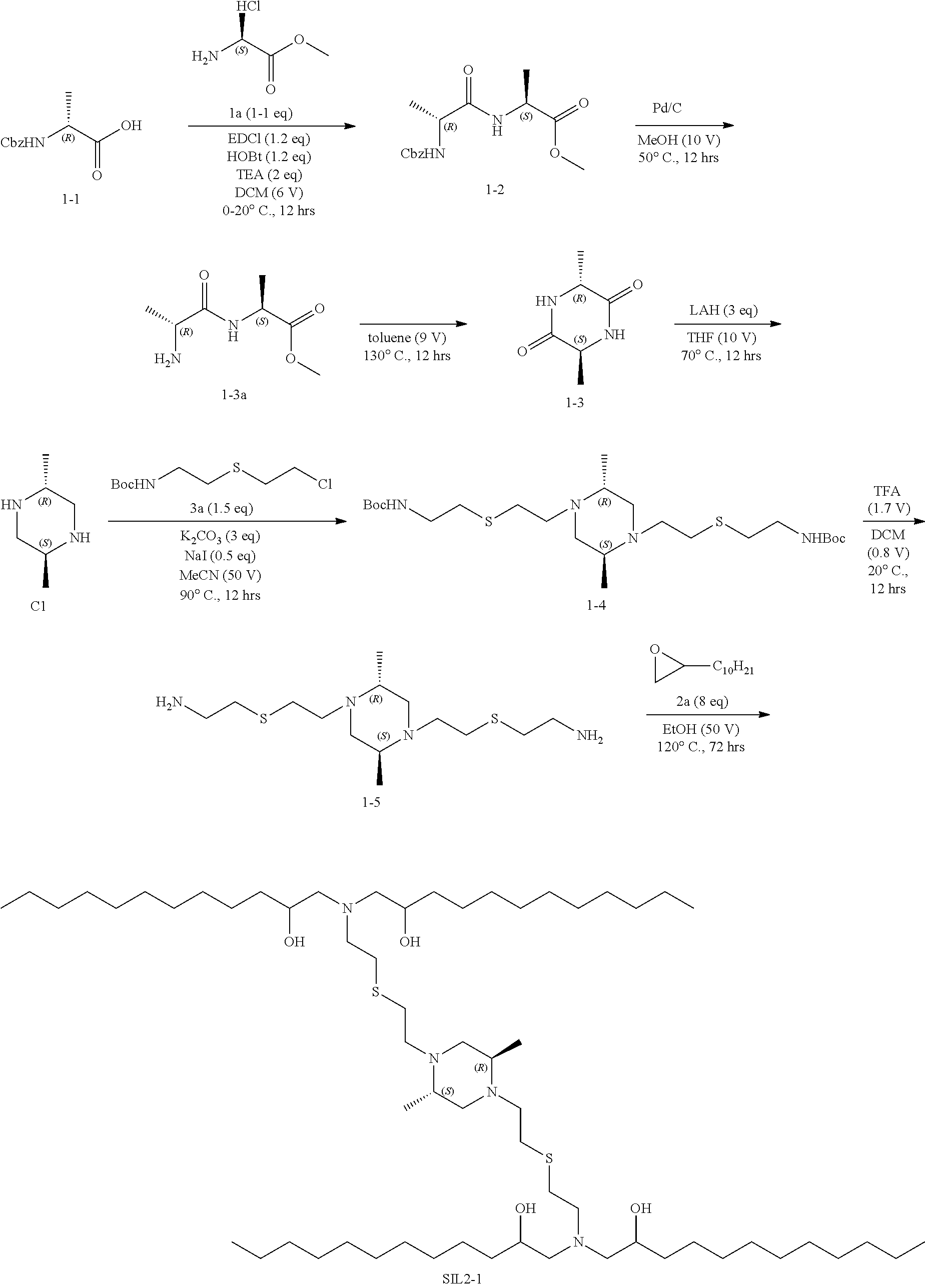

### Step 1. Preparation of Compound 1-2

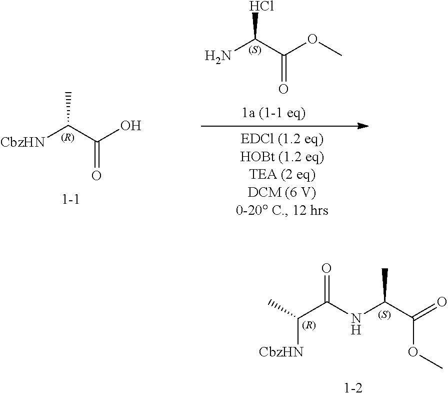

Compound 1a (3.44 g, 24.64 mmol, 1.1 eq, HCl), EDCI (5.15 g, 26.88 mmol, 1.2 eq), HOBt (3.63 g, 26.88 mmol, 1.2 eq), and TEA (4.53 g, 44.80 mmol, 6.24 mL, 2 eq) to a solution of Compound 1-1 (5 g, 22.40 mmol, 1 eq) in DCM (30 mL) at 0° C. Stir the mixture at 20° C. for 12 hrs. TLC (Petroleum ether:Ethyl acetate=2:1, R_{f}=0.1) indicated Compound 1-1 was consumed completely and many new spots formed. The reaction mixture was washed with water (30 mL) and NaHCO₃ (50 mL). The organic phase was separated, washed with brine (30 mL), dried over Na₂SO₄, filtered and concentrated under reduced pressure to give a residue. The crude product was used in the next step without further purification. Compound 1-2 (7.5 g, crude) was obtained as a white solid.

¹H NMR: 400 MHz CDCl₃

δ 7.27-7.40 (m, 5H) 6.70 (br s, 1H) 5.39 (br s, 1H) 5.06-5.17 (m, 2H) 4.49-4.61 (m, 1H) 4.22-4.35 (m, 1H) 3.72 (s, 3H) 1.38 (d, J=7.00 Hz, 6H).

Step 2. preparation of compound 1-3a

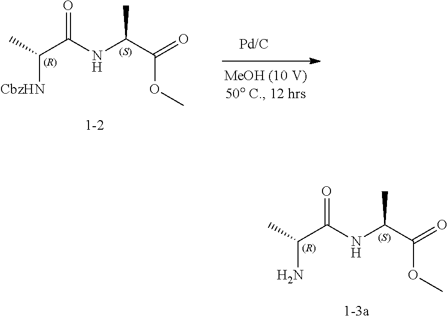

To a solution of Compound 1-2 (7 g, 22.70 mmol, 1 eq) in MeOH (70 mL) was added Pd/C (2 g, 1.69 mmol, 10% purity) under N₂. The suspension was degassed under vacuum and purged with H₂ several times. The mixture was stirred under H₂ (30 psi) at 50° C. for 12 hrs. TLC (Petroleum ether:Ethyl acetate=2:1, R_{f}=0.00) indicated Compound 1-2 was consumed completely and many new spots formed. After filtration with Celite, the filter cake washed with MeOH, and the filtrate was concentrated. The crude product was used into the next step without further purification. Compound 1-3a (4.2 g, crude) was obtained as a gray solid.

### Step 3. Preparation of Compound 1-3

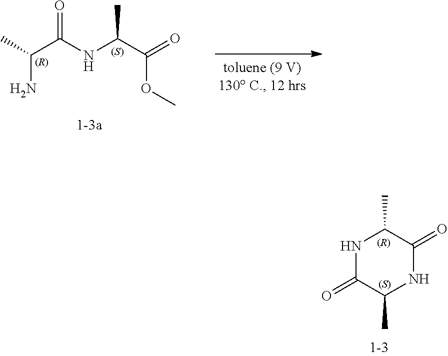

The Compound 1-3a (4.2 g, 24.11 mmol, 1 eq) was dissolved in toluene (39 mL) and then stirred at 130° C. for 12 hrs. ¹HNMR showed the starting material was consumed completely. The suspension mixture was cooled to 0° C. The solid was filtered and washed with petroleum ether (20 mL*2). The crude product was used into the next step without further purification. Compound 1-3 (2.7 g, 18.99 mmol, 78.78% yield) was obtained as a gray solid.

¹H NMR: 400 MHz DMSO-d₆

δ 8.09 (br s, 2H) 3.78-3.94 (m, 2H) 1.26 (d, J=7.00 Hz, 6H).

### Step 4. Preparation of Compound C1

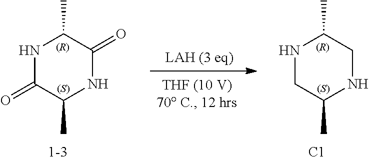

Compound 1-3 (2.2 g, 15.48 mmol, 1 eq) was added in portions to a solution of LiAlH₄ (1.76 g, 46.43 mmol, 3 eq), THE (22 mL), the reaction mixture was stirred at 70° C. for 12 hrs. I HNMR showed the starting material was consumed completely. The reaction mixture was quenched by addition water 1.8 mL at 0° C., and then diluted with a solution of 15% NaOH 1.8 mL and water 5.4 mL, filtered, filter cake washed with THF and filtrate concentrated under reduced pressure to give a residue. Compound C1 (2 g, crude) was obtained as a yellow solid.

¹H NMR: 400 MHz MeOD

δ 2.90 (dd, J=12.26, 2.75 Hz, 2H) 2.63-2.74 (m, 2H) 2.37 (dd, J=12.19, 10.69 Hz, 2H) 1.03 (d, J=6.38 Hz, 6H).

### Step 5. Preparation of Compound 1-4

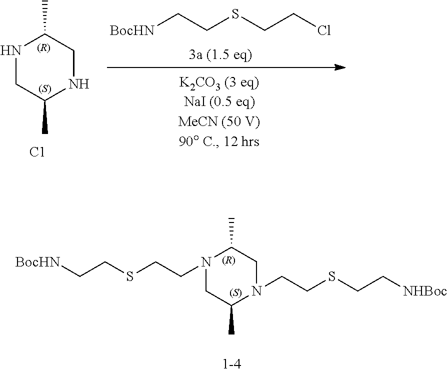

To a solution of Compound 3a (1.89 g, 7.88 mmol, 1.5 eq) in MeCN (30 mL) was added K₂CO₃ (2.18 g, 15.76 mmol, 3 eq) and NaI (393.80 mg, 2.63 mmol, 0.5 eq), Compound C1 (600 mg, 5.25 mmol, 1 eq). The mixture was stirred at 90° C. for 12 hrs. LCMS (ET54476-78-P1 A1, product: RT=0.503 min) showed the starting material was consumed completely. TLC (Dichloromethane:Methanol=10:1, R_{f}=0.30) indicated Compound C1 was consumed completely and many new spots formed. The reaction mixture was filtered, the filter cake was washed with ACN. The residue was purified by column chromatography (SiO₂, Petroleum ether/Ethyl acetate=20/1 to 0/1). Compound 1-4 (1.2 g, 2.30 mmol, 43.85% yield) was obtained as a yellow oil.

¹H NMR: 400 MHz CDCl₃

δ 4.97 (br s, 2H) 3.32 (br d, J=5.88 Hz, 4H) 3.02-3.08 (m, 2H) 2.78 (br d, J=8.88 Hz, 2H) 2.64-2.69 (m, 8H) 2.39-2.55 (m, 4H) 2.06-2.17 (m, 2H) 1.45 (s, 18H) 1.07 (br d, J=4.25 Hz, 6H)

### Step 6. Preparation of Compound 1-5

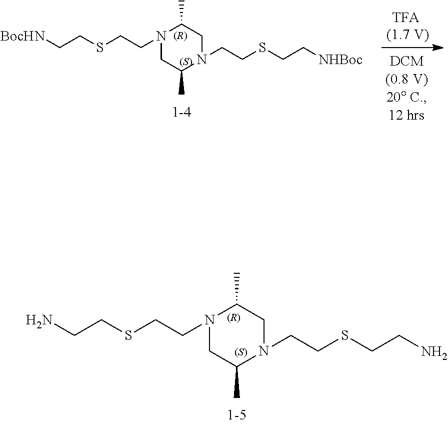

To a solution of Compound 1-4 (1.2 g, 2.30 mmol, 1 eq) in DCM (1 mL) was added TFA (3.08 g, 27.01 mmol, 2.00 mL, 11.72 eq). The mixture was stirred at 20° C. for 12 hrs. TLC (Dichloromethane:Methanol=10:1, R_{f}=0.00) indicated Compound 1-4 was consumed completely and one new spot formed. The reaction mixture was concentrated. The crude product was used into the next step without further purification. Compound 1-5 (1.9 g, crude, TFA) was obtained as a brown oil.

### Step 7. Preparation of Compound SIL2-1

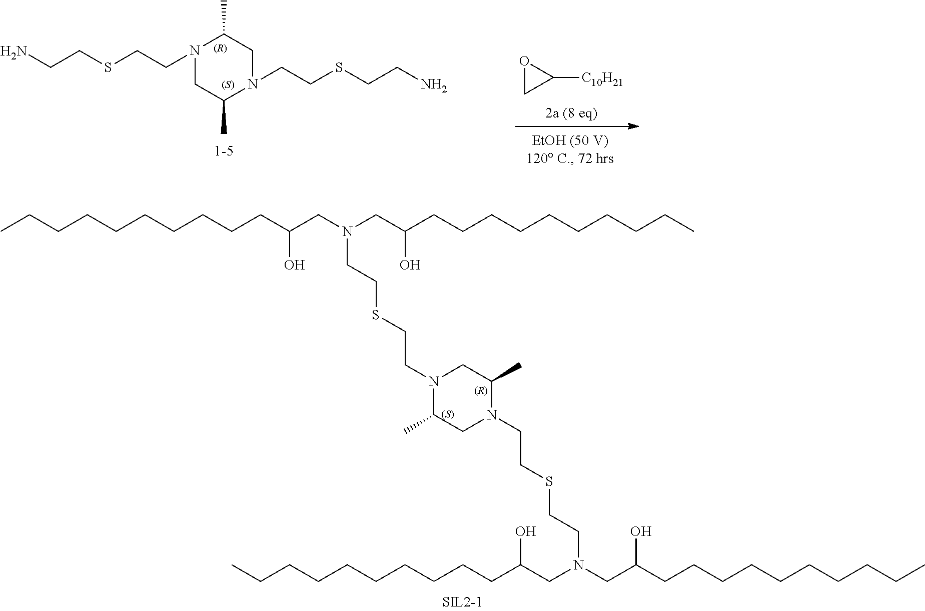

The Compound 1-5 (0.5 g, 1.15 mmol, 1 eq, TFA) was dissolved in methanol and free with alkaline resin to pH=8-9, filter and concentrated to give 200 mg residue. To a solution of the residue in EtOH (10 mL) was added 2-decyloxirane (1.70 g, 9.20 mmol, 8 eq). The mixture was stirred at 120° C. for 72 hrs. TLC (Dichloromethane:Methanol=10:1, R_{f}=0.42) indicated Compound 1-5 was consumed completely and many new spots formed. The reaction mixture was concentrated. The residue was purified by column chromatography (SiO₂, Dichloromethane:Methanol=100/1 to 10/1). Compound SIL2-1 (70 mg, 66.17 μmol, 5.75% yield) was obtained as a yellow oil.

¹H NMR: 400 MHz CDCl₃

δ 3.58-3.74 (m, 4H) 2.97-3.17 (m, 4H) 2.16-2.96 (m, 30H) 1.25-1.48 (m, 72H) 1.14 (br s, 6H) 0.89 (t, J=6.82 Hz, 12H)

### Example 2. Synthesis of Compound SIL2-2

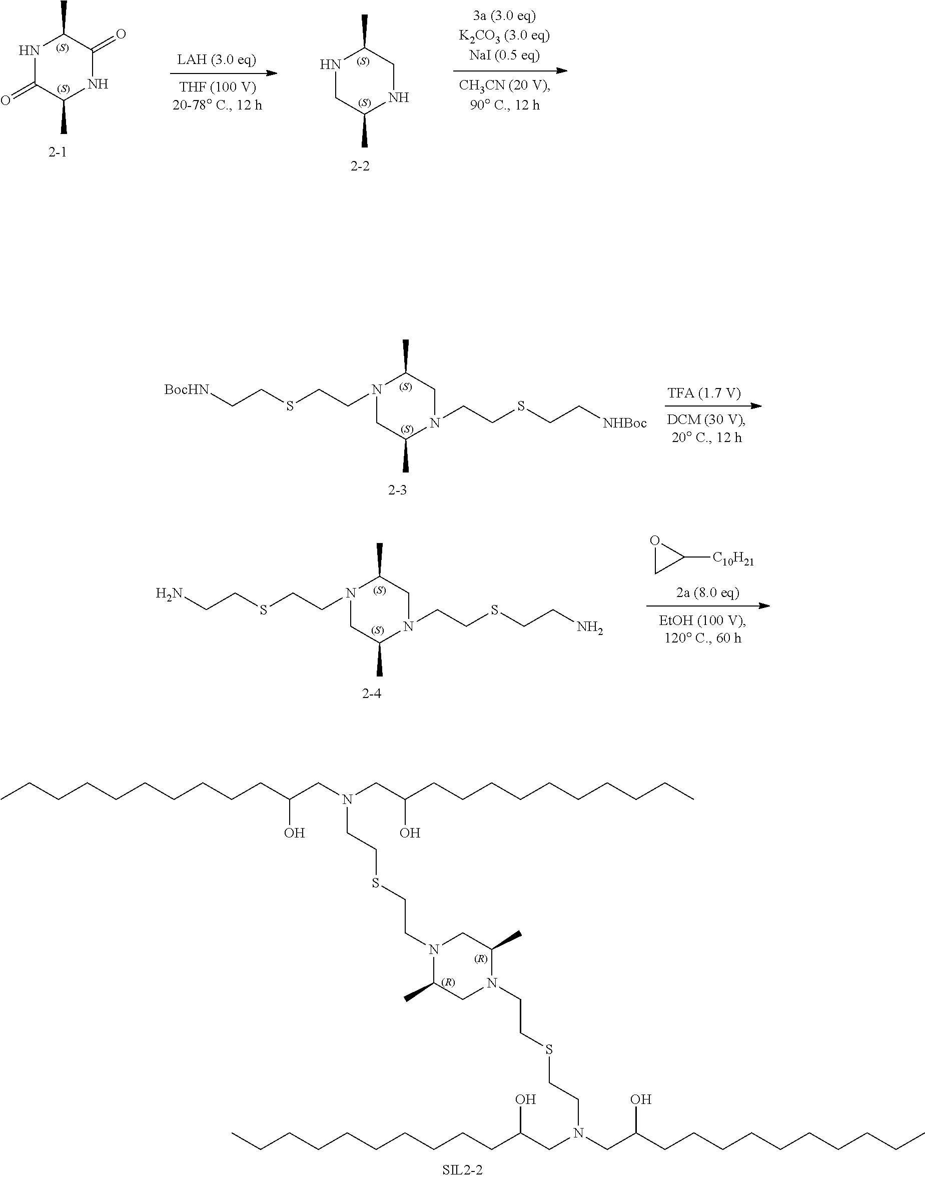

### Step 1. Preparation of Compound 2-2

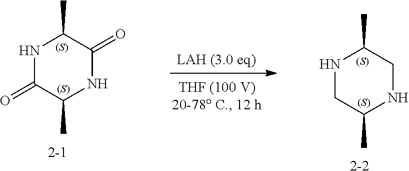

To a solution of Compound 2-1 (0.9 g, 6.33 mmol, 1.0 eq) in THF (90 mL) was added LAH (721 mg, 19.0 mmol, 3.0 eq) at 20° C. The mixture was stirred at 78° C. for 12 hrs. TLC (Dichloromethane:Methanol=10:1) showed reactant was used up, and the product R_{f}=0.0. The reaction mixture was quenched by addition H₂O 0.72 mL at 0° C., NaOH 0.72 mL (15%), H₂O 2.16 mL, Na₂SO₄ was added, filtered and concentrated under reduced pressure to give a residue. Without purification. Compound 2-2 (0.7 g, crude) was obtained as a white solid, checked by HNMR.

¹H NMR: (400 MHz MeOD)

δ ppm 2.75-2.93 (m, 4H), 2.61-2.70 (m, 2H), 1.15 (d, J=6.75 Hz, 6H).

### Step 2. Preparation of Compound 2-3

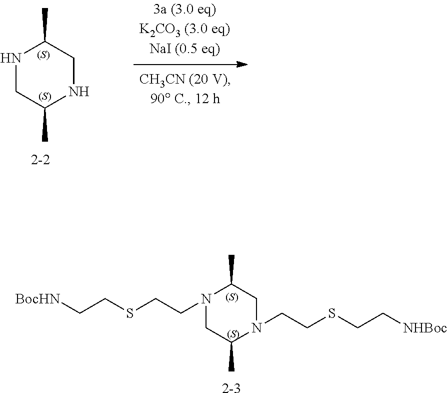

To a solution of Compound 3a (4.17 g, 13.9 mmol, 3.0 eq) in ACN (10 mL) was added K₂CO₃ (1.92 g, 13.9 mmol, 3.0 eq) and NaI (348 mg, 2.32 mmol, 0.5 eq), Compound 2-2 (0.53 g, 4.64 mmol, 1.0 eq). The mixture was stirred at 90° C. for 12 hrs. LCMS showed reactant was used up, the product RT=0.658 min. The reaction mixture was filtered, the filter cake was washed with ACN. The residue was purified by column chromatography (SiO₂, Petroleum ether/Ethyl acetate=100/1 to 3/1). Compound 2-3 (0.3 g, 576 μmol, 12.4% yield) was obtained as a light yellow solid, checked by HNMR.

¹H NMR: ET54385-43-P1 (400 MHz, CDCl₃)

δ ppm 4.89-5.06 (m, 2H), 3.25-3.39 (m, 4H), 1.46 (s, 18H), 2.39-2.74 (m, 16H), 1.04 (d, 1=6.13 Hz, 5H).

### Step 3. Preparation of Compound 2-4

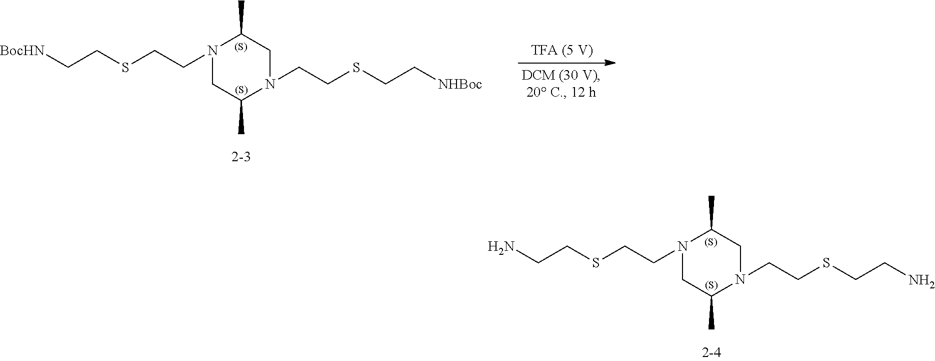

To a solution of Compound 2-3 (0.3 g, 576 μmol, 1.0 eq) in DCM (10 mL) was added TFA (2.31 g, 20.3 mmol, 1.5 mL, 35 eq). The mixture was stirred at 20° C. for 12 hrs. LCMS showed reactant was used up, the product RT=0.044 min. The reaction mixture was concentrated under reduced pressure to remove DCM (10 mL). The crude product was dissolved in MeOH (50 mL), alkaline resin (NH₄HCO₃) was added to adjust pH=7. Without purification. Compound 2-4 was obtained as brown oil, checked by HNMR.

¹H NMR: 400 MHz, CDCl₃)

δ ppm 2.89-2.97 (m, 4H), 2.44-2.81 (m, 19H), 2.01-2.28 (m, 9H), 1.06 (d, J=6.38 Hz, 6H).

### Step 4. Preparation of Compound SIL2-2

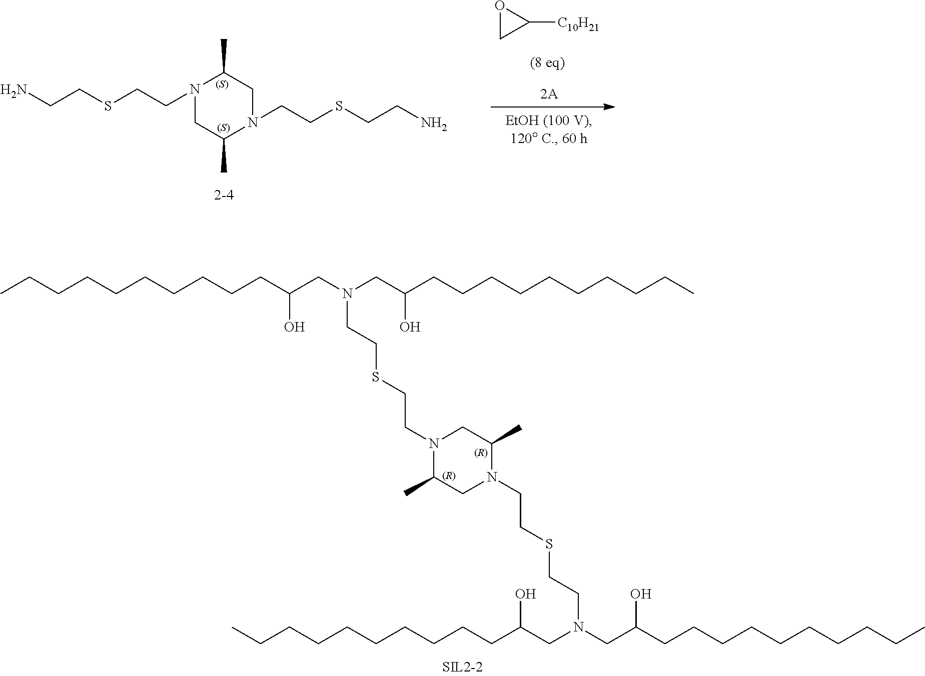

To a solution of Compound 2-4 (0.16 g, 499 μmol, 1.0 eq) in EtOH (16 mL) was added Compound 2A (736 mg, 3.99 mmol, 8.0 eq). The mixture was stirred at 120° C. for 60 hrs. LCMS showed reactant was used up, the product RT=0.349 min. The reaction mixture was concentrated under reduced pressure to remove EtOH (16 mL). The residue was purified by column chromatography (SiO₂, Petroleum ether/Ethyl acetate=5/1 to DCM:MeOH=30/1).
Compound SIL2-2 (52.5 mg, 47.3 μmol, 9.47% yield) was obtained as light yellow oil, checked by HNMR.

¹H NMR: (400 MHz, CDCl₃)

δ ppm 3.76 (s, 1H), 3.63 (s, 4H), 3.63 (s, 4H), 3.21 (s, 3H), 2.30-2.89 (m, 25H), 1.86 (t, J=6.54, 3.30 Hz, I H), 1.19-1.54 (m, 68H), 1.04 (s, 6H), 0.89 (t, J=6.75 Hz, 12H).

### Example 3. Synthesis of Compound SIL2-3

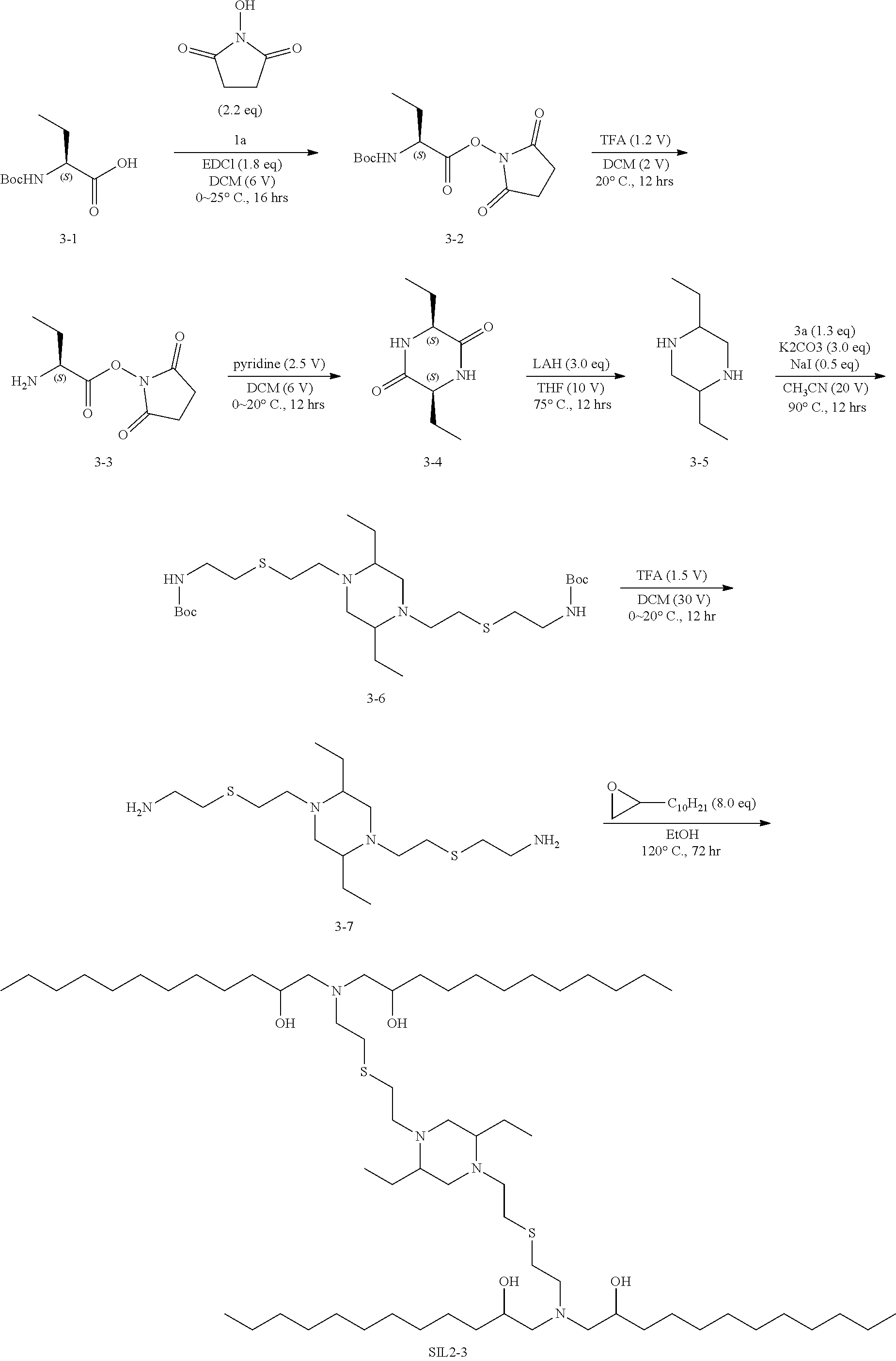

### Step 1. Preparation of Compound 3-2

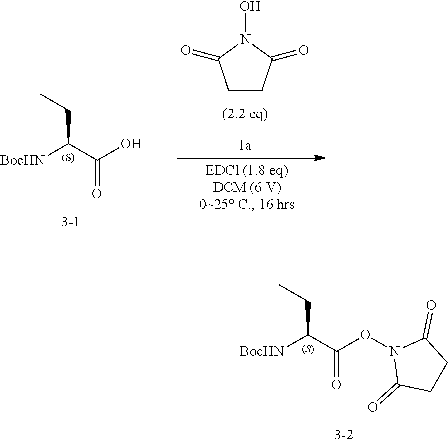

To a solution of Compound 3-1 (8.50 g, 41.8 mmol, 1.0 eq) and Compound 1 a (10.6 g, 92.0 mmol, 2.2 eq) in DCM (60.0 mL) was added EDCI (14.4 g, 75.3 mmol, 1.8 eq) at 0° C. The mixture was stirred at 25° C. for 12 hr. TLC (Dichloromethane:Methanol=10:1) showed reactant was consumed completely and many new spots formed. The residue was poured into saturated sodium bicarbonate (10.0 mL) and stirred for 5 min. The aqueous phase was extracted with DCM (15.0 mL*2). The combined organic phase was washed with brine (10.0 mL), dried with anhydrous Na₂SO₄, filtered and concentrated in vacuum. Without purification. Compound 3-2 (17.5 g, crude) was obtained as a yellow oil.

### Step 2. Preparation of Compound 3-3

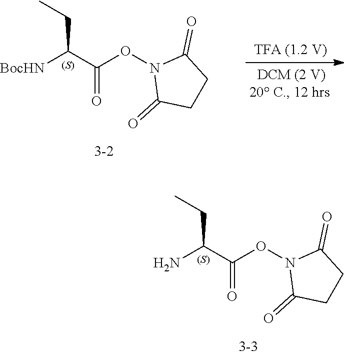

To a solution of Compound 3-2 (17.5 g, 58.4 mmol, 1.0 eq) in DCM (30.0 mL) was added TFA (21.3 mL, 288 mmol, 4.93 eq) at 0° C. The mixture was stirred at 25° C. for 12 hr. TLC indicated Reactant 1 was consumed completely and many new spots formed. Filtered and concentrated in vacuum. Without purification. Compound 3-3 (26.2 g, crude) was obtained as a yellow oil.

### Step 3. Preparation of Compound 3-4

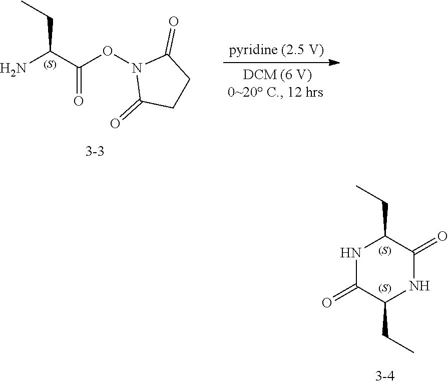

To a solution of Compound 3-3 (26.2 g, 131 mmol, 1.0 eq) in DCM (160 mL) was added pyridine (64.1 g, 810 mmol, 65.4 mL, 6.20 eq) at 0° C. The mixture was stirred at 20° C. for 12 hrs. The IN-MIR showed the reaction was complete and the product was the desired product. Filtered and concentrated in vacuum. The crude product was stirred with ethyl acetate at 20° C. for 1 hr. Compound 3-4 (1.50 g, crude) was obtained as white solid, checked by HNMR.

¹H NMR: (400 MHz, DMSO-d₆)

δ 8.08 (s, 2H), 3.81 (m, 2H), 1.68 (m, 4H), 0.84 (m, 6H).

### Step 4. Preparation of Compound 3-5

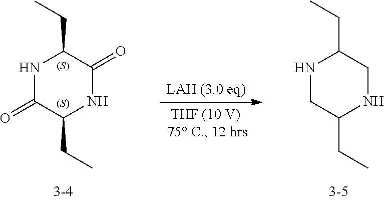

To a solution of Compound 3-4 (1.50 g, 8.81 mmol, 1.0 eq) in THE (15.0 mL) was added LAH (1.00 g, 26.4 mmol, 3.0 eq). The mixture was stirred at 75° C. for 12 hr. LCMS showed reactant 1 was consumed completely and one main peak with desired mass was detected. The mixture was cooled to 5° C. Then was added water (1.00 mL), NaOH-water (1.00 mL), then the mixture was added water (3.00 mL) and stirred for 5 min. Filtered and concentrated in vacuum. Without purification. Compound 3-5 (1.00 g, crude) was obtained as a yellow oil.

### Step 5. Preparation of Compound 3-6

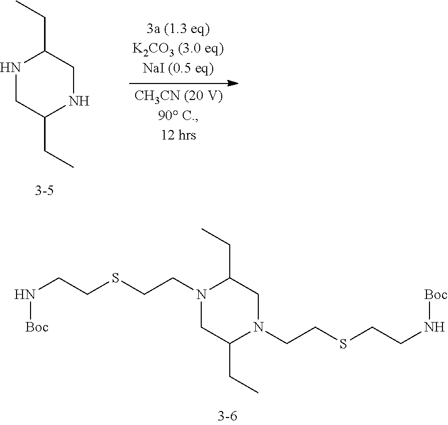

To a solution of tert-butyl N-[2-(2-chloroethylsulfanyl) ethyl]carbamate (1.75 g, 7.31 mmol, 1.3 eq) in ACN (15.0 mL) was added K₂CO₃ (2.33 g, 16.9 mmol, 3 eq) and NaI (422 mg, 2.81 mmol, 0.5 eq), Compound 3-5 (800 mg, 5.62 mmol, 1.0 eq). The mixture was stirred at 90° C. for 12 hr. LCMS showed reactant was used up, and the product RT=0.550 min. The reaction mixture was filtered, the filter cake was washed with ACN. The residue was purified by silica gel chromatography (column height: 250 mm, diameter: 100 mm, 100-200 mesh silica gel, Petroleum ether/Ethyl acetate=100/1, 0/1). Compound 3-6 (900 mg, 1.64 mmol, 29.2% yield) was obtained as yellow oil.

### Step 6. Preparation of Compound 3-7

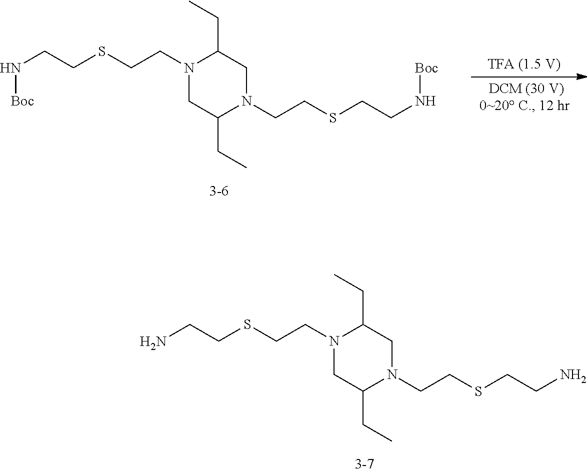 [SMILES: CCC1CN(CCSCCNC(=O)OC(C)(C)C)C(CC)CN1CCSCCNC(=O)OC(C)(C)C ; CCC1CN(CCSCCN)C(CC)CN1CCSCCN]

To a solution of Compound 3-6 (300 mg, 547 μmol, 1.0 eq) in DCM (10.0 mL) was added TFA (693 mg, 6.08 mmol, 450 μL, 11.1 eq) at 0° C. The mixture was stirred at 20° C. for 12 hr. LCMS showed reactant was used up. Filtered and concentrated in vacuum. Without purification, Compound 3-7 (180 mg, crude) was obtained as a yellow oil.

### Step 7. Preparation of Compound SIL2-3

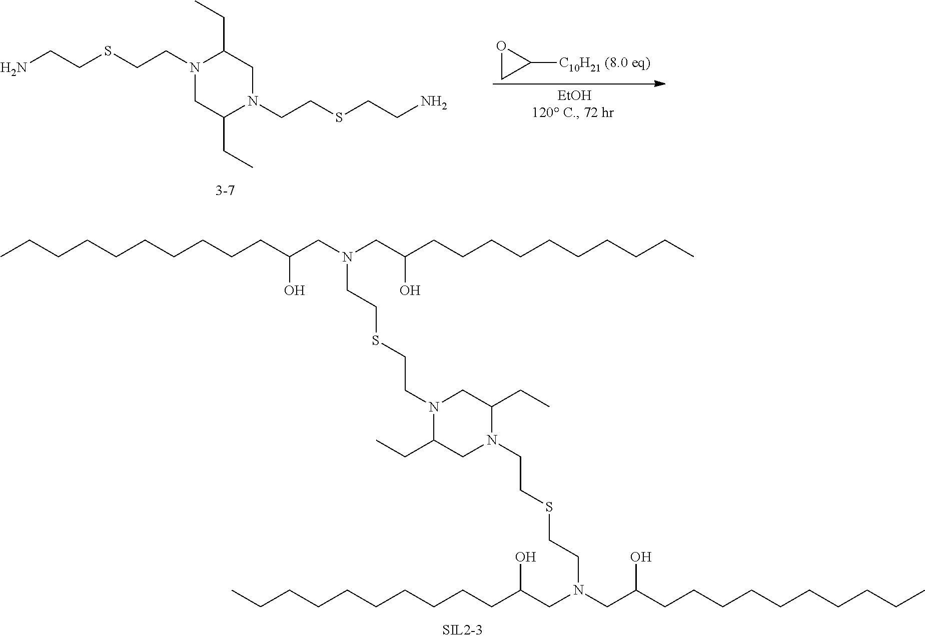

To a solution of Compound 3-7 (180 mg, 516 μmol, 1.0 eq) in EtOH (20.0 mL) was added 2-decyloxirane (761 mg, 4.13 mmol, 8.0 eq). The mixture was stirred at 120° C. for 72 hr. LC-MS showed Compound 3-7 was consumed completely. Filtered and concentrated in vacuum. The residue was purified by silica gel chromatography (column height: 250 mm, diameter: 100 mm, 100-200 mesh silica gel, DCM/MeOH=100/1, 0/1). Compound SIL2-3 (160 mg, 147 μmol, 28.5% yield) was obtained as a yellow oil, checked by ¹HNMR.

¹H NMR: (400 MHz, Chloroform-d)

δ 3.65 (s, 4H), 3.25 (s, 3H), 2.75 (m, 6H), 2.60 (m, 12H), 2.35 (m, 9H), 1.45 (m, 80H), 0.89 (m, 18H).

Compounds SIL2-4 to SIL2-8 were prepared similarly with the methods described above. Their structures and 1H-NMR data are summarized in Table 1. In addition, Compound SIL2-9 can be prepared similarly with the methods described above.

TABLE 1

| | ¹H NMR (δ ppm, |
|---|---|
| Compound | 400 MHz) |
| 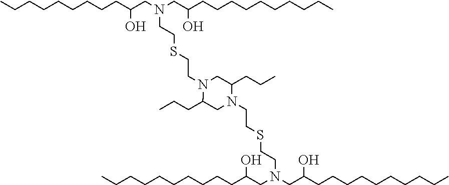 [SMILES: CCCCCCCCCCC(O)CN(CCSCCN1CC(CCC)N(CCSCCN(CC(O)CCCCCCCCCC)CC(O)CCCCCCCCCC)CC1CCC)CC(O)CCCCCCCCC] | 3.64 (br s, 4 H) 2.30 − 2.95 (m, 30 H) 1.24 − 1.51 (m, 80 H) 0.85 − 0.97 (m, 18 H) (CDCl₃) |
| SIL2-4 | |
| 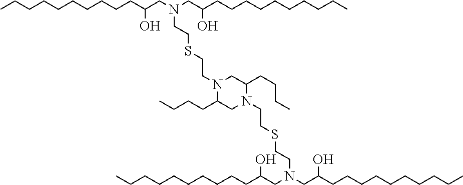 [SMILES: CCCCCCCCCCC(O)CN(CCSCCN1CC(CCCC)N(CCSCCN(CC(O)CCCCCCCCCC)CC(O)CCCCCCCCCC)CC1CCCC)CC(O)CCCCCCCCCC] | 3.64 (s, 4 H), 2.25 − 3.25 (m, 32 H), 1.13 − 1.58 (m, 86 H), 0.87 − 0.93 (m, 18 H) (CDCl₃) |
| SIL2-5 | |
| 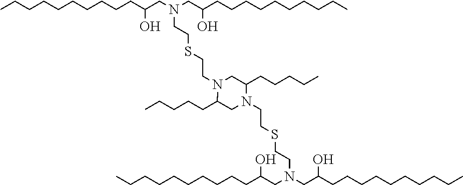 [SMILES: CCCCCCCCCCC(O)CN(CCSCCN1CC(CCCCC)N(CCSCCN(CC(O)CCCCCCCCCC)CC(O)CCCCCCCCCC)CC1CCCCC)CC(O)CCCCCCCCCC] | 3.65 (m, 6H), 3.45 (m, 2H), 3.25 (m, 2H), 2.55 (m, 22H), 1.95 (m, 2H), 1.35 (m, 92H), 0.89 (m,18H) (CDCl₃) |
| SIL-2-6 | |
| 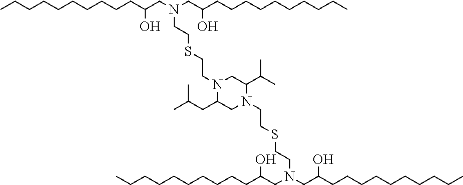 [SMILES: CCCCCCCCCCC(O)CN(CCSCCN1CC(C(C)C)N(CCSCCN(CC(O)CCCCCCCCCC)CC(O)CCCCCCCCCC)CC1CC(C)C)CC(O)CCCCCCCCCC] | 3.64 (s, 4H), 2.70 (m, 18H), 2.40 (m, 7H), 2.10 (m, 3H), 1.25 (m, 79H), 0.89 (m, 24H (CDCl₃) |
| SIL2-7 | |
| 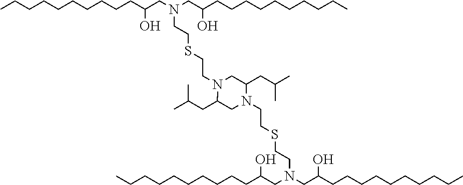 [SMILES: CCCCCCCCCCC(O)CN(CCSCCN1CC(CC(C)C)N(CCSCCN(CC(O)CCCCCCCCCC)CC(O)CCCCCCCCCC)CC1CC(C)C)CC(O)CCCCCCCCCC] | 3.65 − 3.69 (m, 5 H), 3.42-3.47 (m, 2 H), 3.21 (br s, 3 H), 2.43- 2.76 (m, 21 H), 1.27 − 1.45 (m, 85 H), 0.87 − 0.91(m, 24 H) (CDCl₃) |
| SIL2-8 | |

### Example 4. Synthesis of Compound SIL2-10

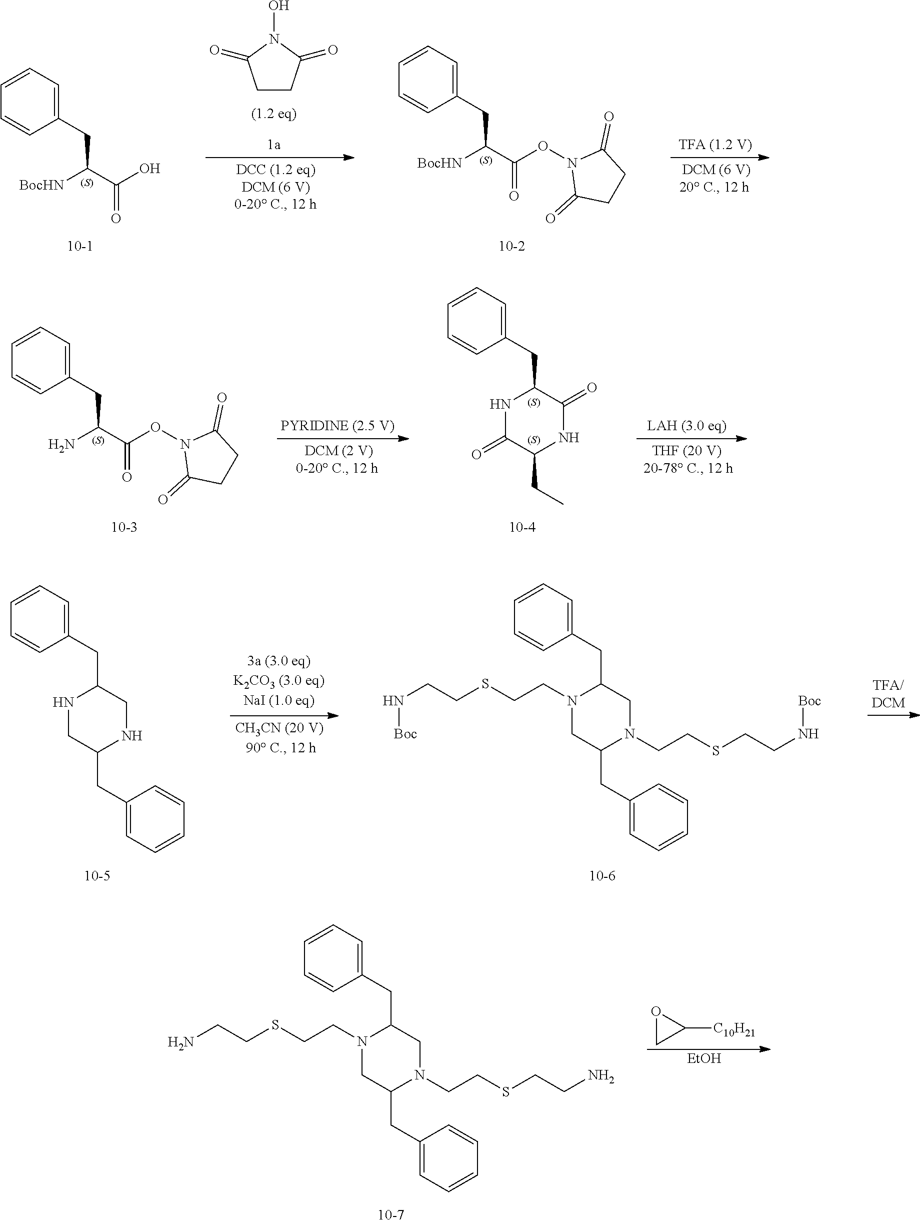

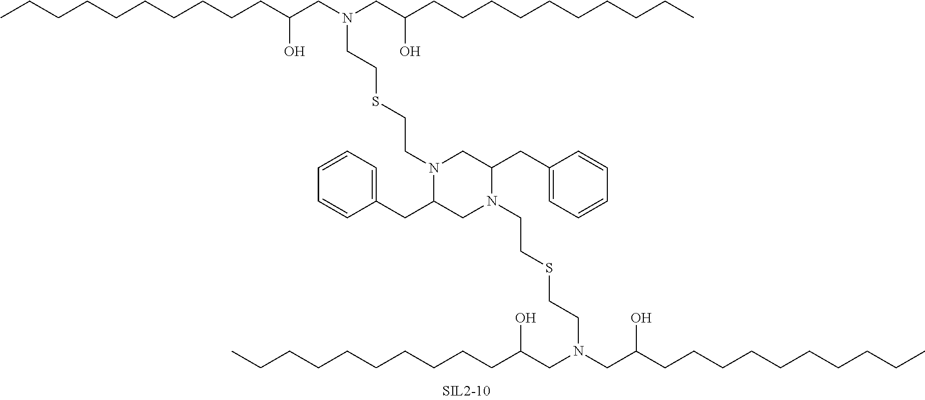 [SMILES: CCCCCCCCCCC(O)CN(CCSCCN1CC(Cc2ccccc2)N(CCSCCN(CC(O)CCCCCCCCCC)CC(O)CCCCCCCCCC)CC1Cc1ccccc1)CC(O)CCCCCCCCCC]

### Step 1. Preparation of Compound 10-2

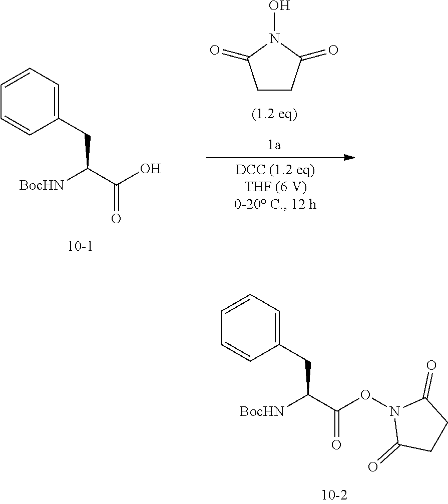 [SMILES: CC(C)(C)OC(=O)N[C@@H](Cc1ccccc1)C(=O)O ; O=C1CCC(=O)N1O ; CC(C)(C)OC(=O)N[C@@H](Cc1ccccc1)C(=O)ON1C(=O)CCC1=O]

To a solution of Compound 10-1 (10.0 g, 37.7 mmol, 1.0 eq) in THE (60 mL) was added DCC (9.33 g, 45.2 mmol, 9.15 mL, 1.2 eq) and Compound 1a (5.21 g, 45.2 mmol, 1.2 eq) at 0° C. The mixture was stirred at 20° C. for 12 hrs. TLC (Dichloromethane:Methanol=10:1, product R_{f}=0.60) showed reactant was used up. The reaction mixture was filtered, the filtered cake was washed with THF, the combined organic layers were concentrated under reduced pressure to give a residue. Without purification. Compound 2 (14.0 g, crude) was obtained as a white solid.

### Step 2. Preparation of Compound 10-3

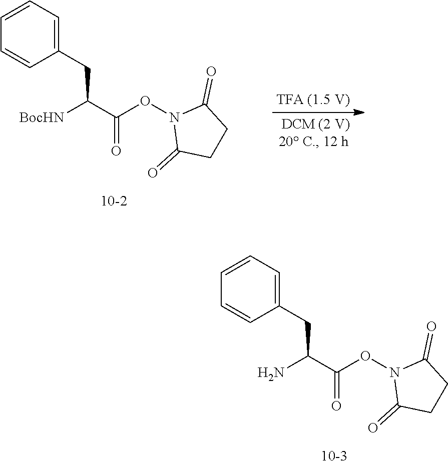 [SMILES: CC(C)(C)OC(=O)N[C@@H](Cc1ccccc1)C(=O)ON1C(=O)CCC1=O ; N[C@@H](Cc1ccccc1)C(=O)ON1C(=O)CCC1=O]

To a solution of Compound 10-2 (14.0 g, 38.6 mmol, 1.0 eq) in DCM (20 mL) was added TFA (32.3 g, 284 mmol, 21.0 mL, 7.34 eq). The mixture was stirred at 20° C. for 12 hrs. TLC (Dichloromethane:Methanol=10:1, product R_{f}=0.43) showed reactant was used up. The reaction mixture was concentrated under reduced pressure to remove DCM (20 mL). The crude was dissolved in Tol. (100 mL) to remove TFA for 2 times. Without purification. Compound 10-3 (18.0 g, crude) was obtained as yellow oil

### Step 3. Preparation of Compound 10-4

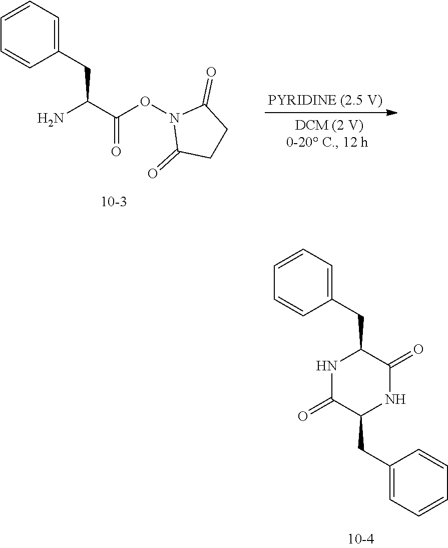 [SMILES: N[C@@H](Cc1ccccc1)C(=O)ON1C(=O)CCC1=O ; O=C1N[C@@H](Cc2ccccc2)C(=O)N[C@H]1Cc1ccccc1]

To a solution of Compound 10-3 (10.1 g, 38.6 mmol, 1.0 eq) in DCM (30 mL) was added PYRIDINE (24.5 g, 310 mmol, 25.0 mL, 8.0 eq). The mixture was stirred at 20° C. for 12 hrs. LCMS showed reactant was used up, and the product RT=0.592 min. The reaction mixture was concentrated under reduced pressure to remove DCM (30 mL). The crude product was triturated with MeOH (50 mL) at 25° C. for 60 min. Compound 10-4 (0.82 g, 2.79 mmol, 14.4% yield) was obtained as a white solid, checked by HNMR.

¹H NMR: (400 MHz, DMSO-d₆)

δ ppm 7.92 (s, 2H), 7.11-7.36 (m, 7H), 7.03 (d, J=7.25 Hz, 4H), 3.97 (s, 2H), 2.57 (dd, J=13.51, 4.63 Hz, 2H), 2.23 (dd, J=13.51, 6.13 Hz, 2H).

### Step 4. Preparation of Compound 10-5

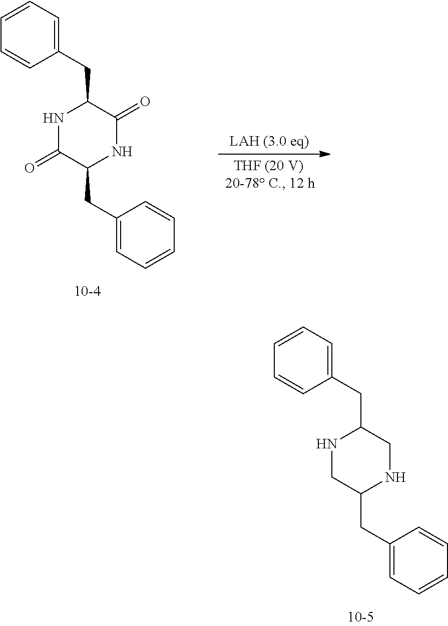 [SMILES: O=C1N[C@@H](Cc2ccccc2)C(=O)N[C@H]1Cc1ccccc1 ; c1ccc(CC2CNC(Cc3ccccc3)CN2)cc1]

To a solution of Compound 10-4 (1.60 g, 5.44 mmol, 1.0 eq) in THF (80 mL) was added LAH (619 mg, 16.3 mmol, 3.0 eq) at 20° C. The mixture was stirred at 78° C. for 12 hrs. LCMS showed reactant was used up and the product RT=0.612 min. The reaction mixture was quenched by addition H₂O 0.62 mL at 0° C., NaOH 0.62 mL (15%), H₂O 1.86 mL, Na₂SO4 was added, filtered and concentrated under reduced pressure to give a residue. The residue was purified by column chromatography (SiO₂, Petroleum ether/Ethyl acetate=100/1 to 3/1). Compound 10-5 (0.93 g, 3.49 mmol, 64.2% yield) was obtained as a light yellow solid, checked by 1 HNMR.

¹H NMR: (400 MHz, DMSO-d₆)

δ ppm 7.11-7.35 (m, 8H), 3.33 (s, 2H), 3.17 (s, 1H), 2.72-2.86 (m, 4H), 2.53-2.71 (m, 4H).

### Step 5. Preparation of Compound 10-6

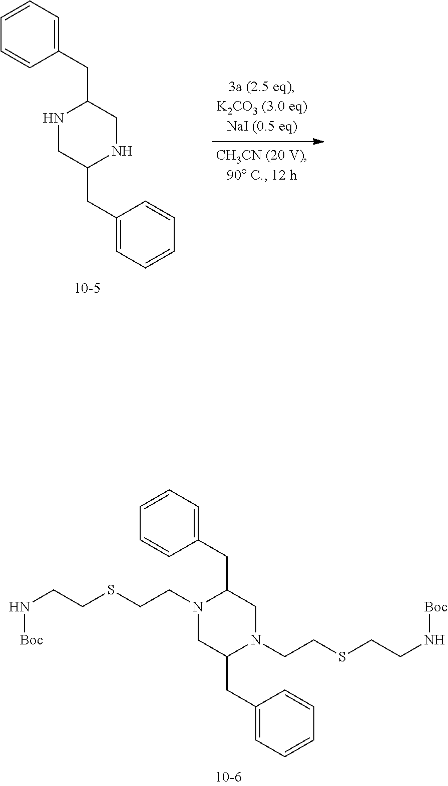 [SMILES: c1ccc(CC2CNC(Cc3ccccc3)CN2)cc1 ; CC(C)(C)OC(=O)NCCSCCN1CC(Cc2ccccc2)N(CCSCCNC(=O)OC(C)(C)C)CC1Cc1ccccc1]

To a solution of Compound 3a (2.09 g, 8.73 mmol, 2.5 eq) in ACN (30 mL) was added K₂CO₃ (1.45 g, 10.5 mmol, 3.0 eq) and NaI (262 mg, 1.75 mmol, 0.5 eq), Compound 10-5 (0.93 g, 3.49 mmol, 1.0 eq). The mixture was stirred at 90° C. for 12 hrs. LCMS showed reactant was used up, and the product RT=1.001 min. The reaction mixture was filtered, the filter cake was washed with ACN. The residue was purified by column chromatography (SiO₂, Petroleum ether/Ethyl acetate=100/1 to 2/1). Compound 10-6 (1.60 g, 2.38 mmol, 68.1% yield) was obtained as yellow oil, checked by HNMR.

¹H NMR: (400 MHz, CDCl₃)

δ ppm 7.16-7.40 (m, 9H), 4.95 (s, 2H), 3.18-3.46 (m, 4H), 2.51-3.06 (m, 18H), 2.35 (d, J=11.25 Hz, 2H), 1.48 (s, 18H).

### Step 6. Preparation of Compound 10-7

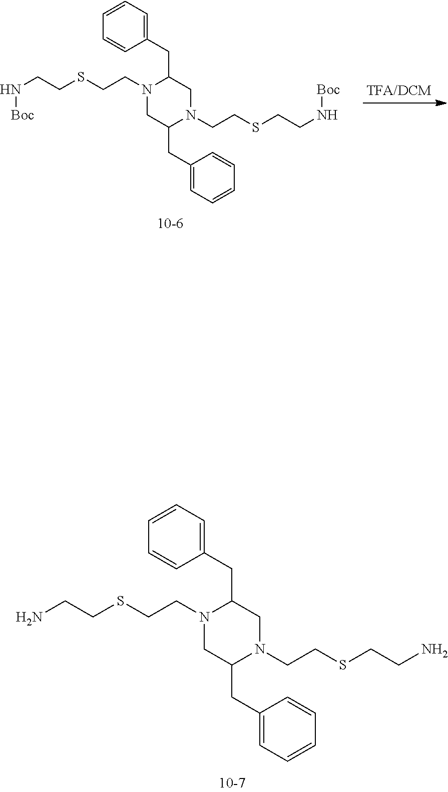 [SMILES: CC(C)(C)OC(=O)NCCSCCN1CC(Cc2ccccc2)N(CCSCCNC(=O)OC(C)(C)C)CC1Cc1ccccc1 ; NCCSCCN1CC(Cc2ccccc2)N(CCSCCN)CC1Cc1ccccc1]

To a solution of Compound 10-6 (1.60 g, 2.38 mmol, 1.0 eq) in DCM (10 mL) was added TFA (7.70 g, 67.5 mmol, 5 mL, 28.4 eq). The mixture was stirred at 20° C. for 12 hrs. TLC (Dichloromethane:Methanol=10:1) showed reactant was used up. The reaction mixture was concentrated under reduced pressure to remove DCM (10 mL). The crude product was dissolved in MeOH (50 mL), alkaline resin (NH₄HCO₃) was added to adjust pH=7. Without purification. Compound 10-7 (0.5 g, 1.06 mmol, 44.5% yield) was obtained as brown oil, checked by HNMR.

¹H NMR: (400 MHz, CDCl₃)

δ ppm 7.29 (br d, J=7.00 Hz, 3H), 7.17-7.25 (m, 5H), 5.78-6.24 (m, 2H), 2.46-3.02 (m, 24H).

### Step 7. Preparation of Compound SIL2-10

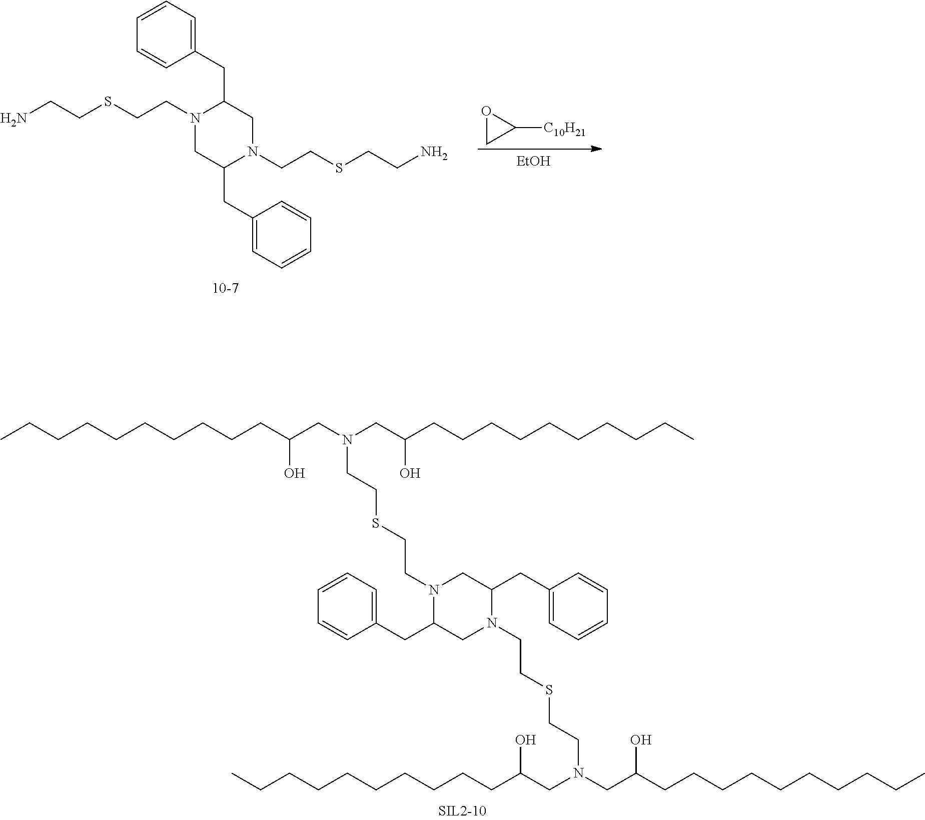

To a solution of Compound 10-7 (0.5 g, 1.06 mmol, 1.0 eq) in EtOH (50 mL) was added 2-decyloxirane (1.56 g, 8.46 mmol, 8.0 eq). The mixture was stirred at 120° C. for 72 hrs. LCMS showed reactant was used up, the product RT=0.918 min. The reaction mixture was concentrated under reduced pressure to remove EtOH (50 mL). The residue was purified by column chromatography (SiO₂, Petroleum ether/Ethyl acetate=5/1 to DCM:MeOH=30/1). Compound SIL2-10 (52.25 mg, 43.2 μmol, 4.08% yield) was obtained as yellow oil, checked by ¹H NMR.

¹H NMR: (400 MHz, CDCl₃)

δ ppm 7.27-7.32 (m, 3H), 7.17-7.24 (m, 4H), 3.54-3.84 (m, 5H), 2.24-3.02 (m, 26H), 1.17-1.57 (m, 72H), 0.89 (t, J=6.82 Hz, 12H).

### Example 5. Synthesis of Compound SIL2-11

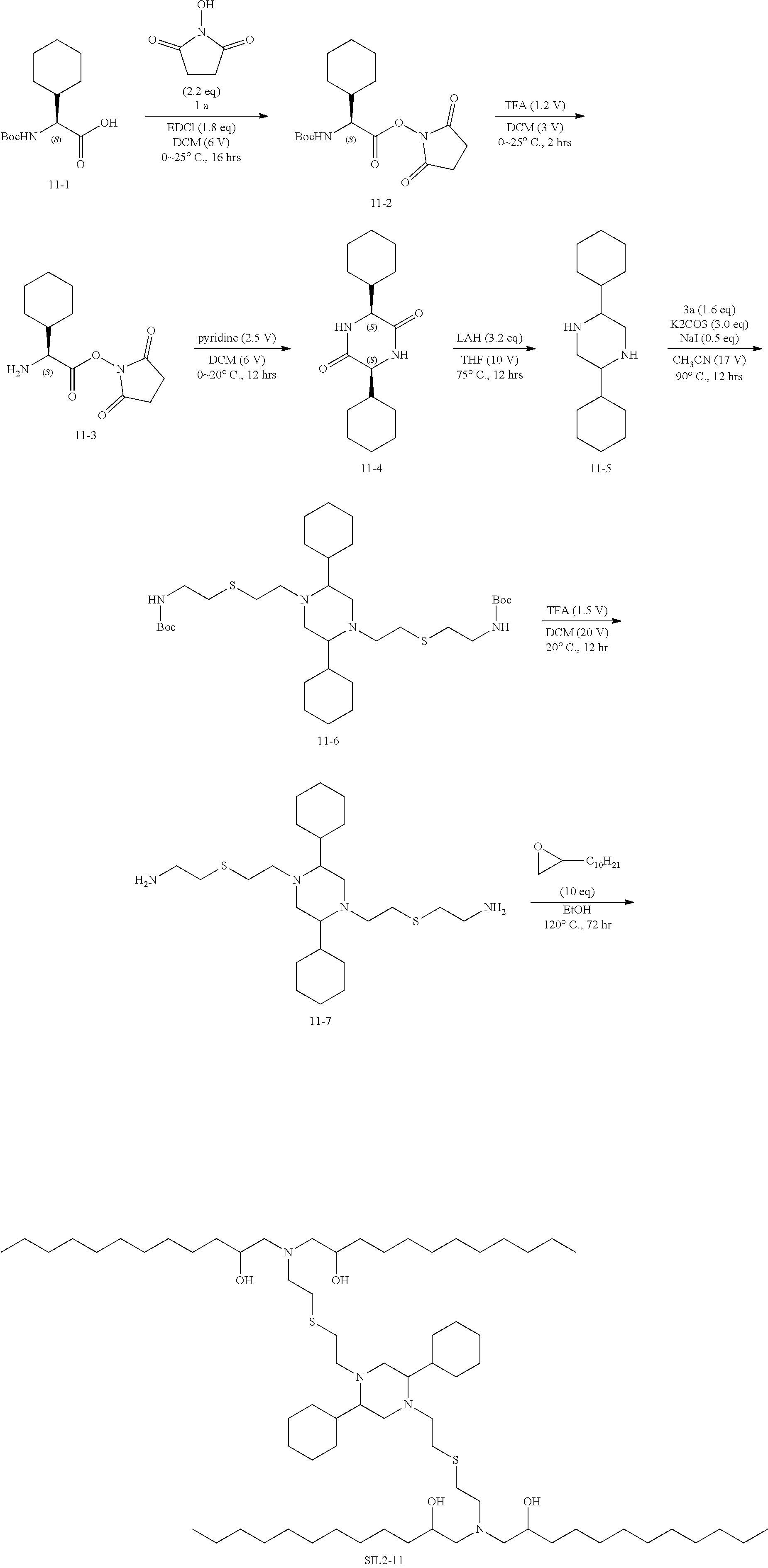

### Step 1. Preparation of Compound 11-2

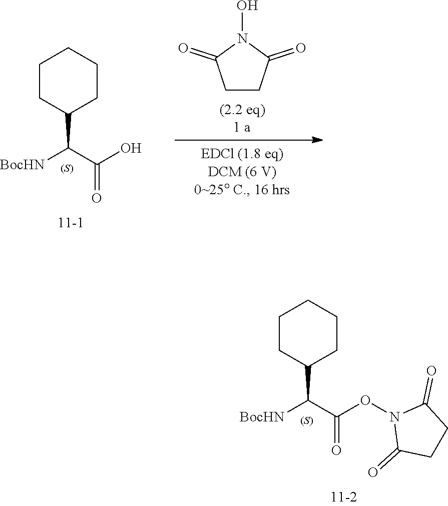

To a solution of Compound 11-1 (5.00 g, 19.4 mmol, 1.0 eq) and Compound 1 a (4.92 g, 42.8 mmol, 2.2 eq) in DCM (30.0 mL) was added EDCI (6.70 g, 35.0 mmol, 1.8 eq) at 0° C. The mixture was stirred at 25° C. for 16 hr. TLC (Dichloromethane:Methanol=10:1) showed reactant was consumed completely and many new spots formed. The residue was poured saturated sodium bicarbonate (5.00 mL) and stirred for 5 min. The aqueous phase was extracted with DCM (10.0 mL*2). The combined organic phase was washed with brine (5.00 mL), dried with anhydrous Na₂SO₄, filtered and concentrated in vacuum. Without purification. Compound 11-2 (7.95 g, crude) was obtained as a white solid.

### Step 2. Preparation of Compound 11-3

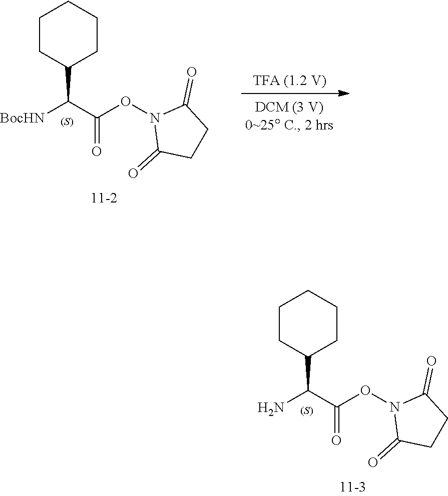

To a solution of Compound 11-2 (7.95 g, 22.4 mmol, 1.0 eq) in DCM (20.0 mL) was added TFA (9.54 mL, 129 mmol, 5.74 eq) at 0° C. The mixture was stirred at 25° C. for 2 hr. TLC indicated Reactant 1 was consumed completely and many new spots formed. Filtered and concentrated in vacuum. Without purification. Compound 11-3 (12.7 g, crude) was obtained as a yellow oil.

### Step 3. Preparation of Compound 11-4

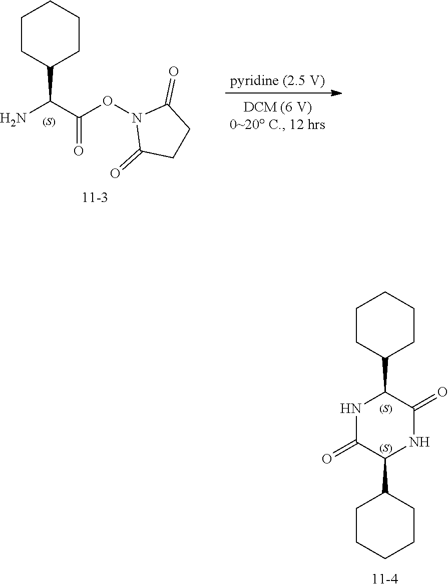

To a solution of Compound 11-3 (12.7 g, 50.1 mmol, 1.0 eq) in DCM (80.0 mL) was added pyridine (31.2 g, 394 mmol, 31.8 mL, 7.88 eq) at 0° C. The mixture was stirred at 20° C. for 12 hr. The HNMR showed the reaction was complete and the product was the desired product. Filtered and concentrated in vacuum. The crude product was stirred with ethyl acetate at 20° C. for 1 hr. Compound 11-4 (1.70 g, crude) was obtained as white solid, checked by ¹H NMR.

¹H NMR: (400 MHz, DMSO-d₆)

δ 7.95 (s, 1H), 3.62 (s, 1H), 3.25 (s, 2H), 2.76 (m, 2H), 1.55 (m, 10H), 1.05 (m, 10H).

### Step 4. Preparation of Compound 11-5

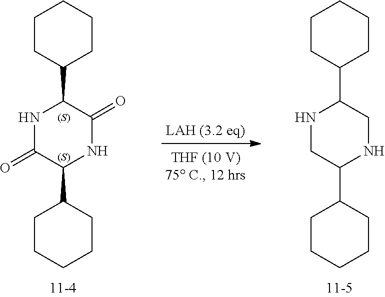

To a solution of Compound 11-4 (2.00 g, 7.18 mmol, 1.0 eq) in THF (20.0 mL) was added LAH (872 mg, 23.0 mmol, 3.2 eq). The mixture was stirred at 75° C. for 12 hr. LCMS showed reactant 1 was consumed completely and one main peak with desired mass was detected. The mixture was cooled to 5° C. Then was added water (0.90 mL), NaOH-water (0.90 mL), then the mixture was added water (2.70 mL) and stirred for 5 min. Filtered and concentrated in vacuum. Without purification. Compound 11-5 (1.40 g, crude) was obtained as a white oil.

### Step 5. Preparation of Compound 11-6

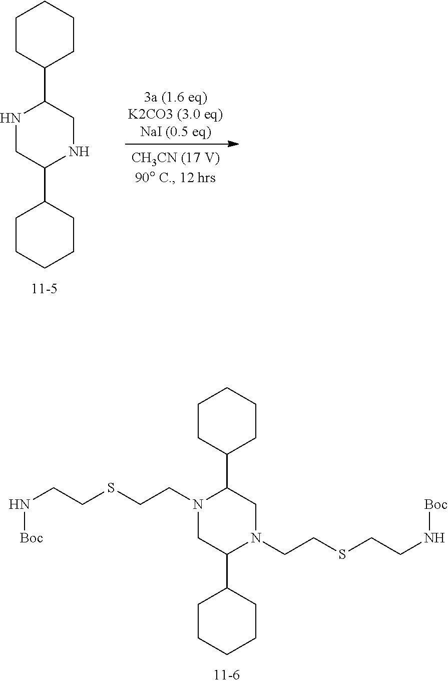 [SMILES: C1CCC(C2CNC(C3CCCCC3)CN2)CC1 ; CC(C)(C)OC(=O)NCCSCCN1CC(C2CCCCC2)N(CCSCCNC(=O)OC(C)(C)C)CC1C1CCCCC1]

To a solution of tert-butyl N-[2-(2-chloroethylsulfanyl) ethyl]carbamate (2.14 g, 8.94 mmol, 1.6 eq) in ACN (20.0 mL) was added K₂CO₃ (2.32 g, 16.8 mmol, 3 eq) and NaI (419 mg, 2.80 mmol, 0.5 eq), Compound 11-5 (1.40 g, 5.59 mmol, 1.0 eq). The mixture was stirred at 90° C. for 12 hr. LCMS showed reactant was used up, and the product RT=0.649 min. The reaction mixture was filtered, the filter cake was washed with ACN.
The residue was purified by silica gel chromatography (column height: 250 mm, diameter: 100 mm, 100-200 mesh silica gel, Petroleum ether/Ethyl acetate=100/1, 0/1). Compound 11-6 (400 mg, 609 μmol, 10.9% yield) was obtained as yellow oil.

### Step 6. Preparation of Compound 11-7

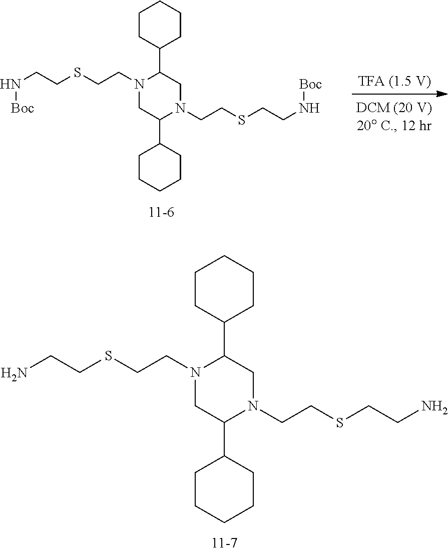 [SMILES: CC(C)(C)OC(=O)NCCSCCN1CC(C2CCCCC2)N(CCSCCNC(=O)OC(C)(C)C)CC1C1CCCCC1 ; NCCSCCN1CC(C2CCCCC2)N(CCSCCN)CC1C1CCCCC1]

To a solution of Compound 11-6 (370 mg, 563 μmol, 1.0 eq) in DCM (6.00 mL) was added TFA (855 mg, 7.50 mmol, 555 μL, 13.3 eq). The mixture was stirred at 20° C. for 12 hrs. LCMS showed reactant was used up. Filtered and concentrated in vacuum. Without purification. Compound 11-7 (250 mg, crude) was obtained as a yellow oil.

### Step 7. Preparation of Compound SIL2-11

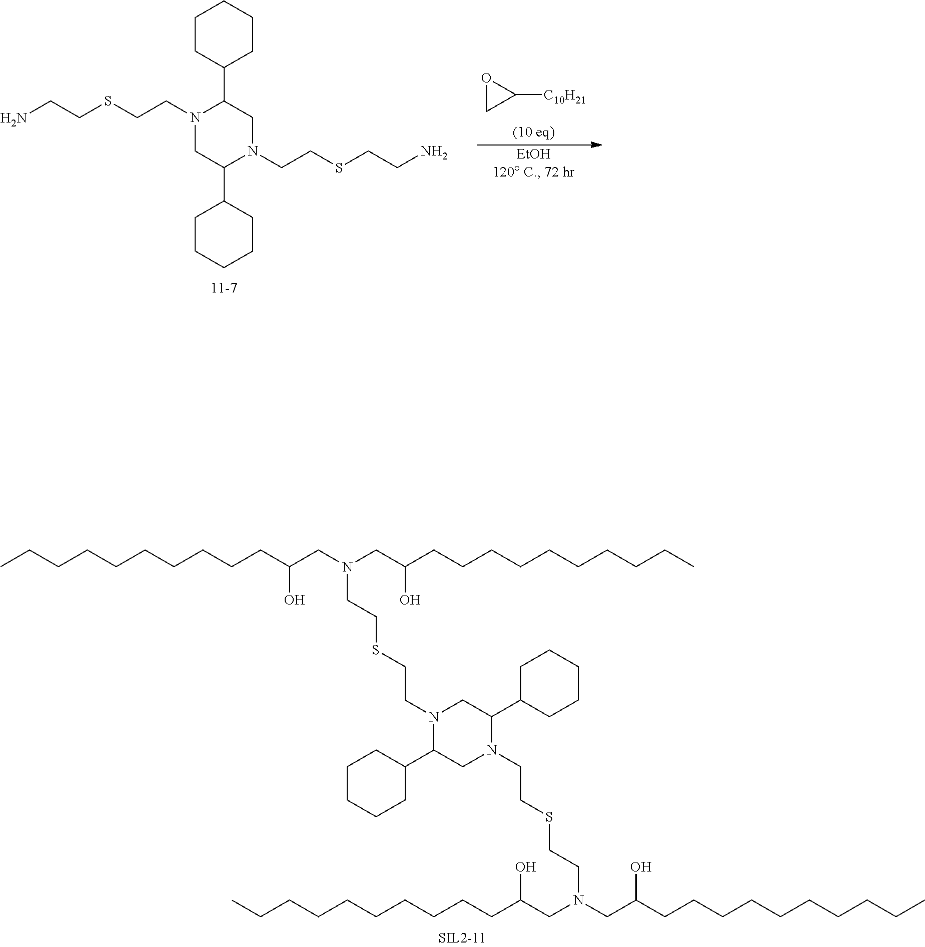

To a solution of Compound 11-7 (150 mg, 328 μmol, 1.0 eq) in EtOH (40.0 mL) was added 2-decyloxirane (605 mg, 3.28 mmol, 10 eq). The mixture was stirred at 120° C. for 72 hr. LC-MS showed Compound 11-7 was consumed completely. Filtered and concentrated in vacuum. The residue was purified by silica gel chromatography (column height: 250 mm, diameter: 100 mm, 100-200 mesh silica gel, DCM/MeOH=100/1, 0/1). Compound SIL2-11 (120 mg, 101 μmol, 30.6% yield) was obtained as a yellow oil, checked by ¹H NMR.

¹H NMR: (400 MHz, CDCl₃)

δ 3.65 (m, 4H), 3.45 (m, 1H), 3.25 (m, 2H), 2.55 (m, 16H), 2.45 (m, 5H), 2.10 (s, 1H), 1.95 (s, 1H), 1.55 (m, 10H), 1.35 (m, 71H), 0.89 (m, 12H).

### Example 6. Synthesis of Compound SIL2-12

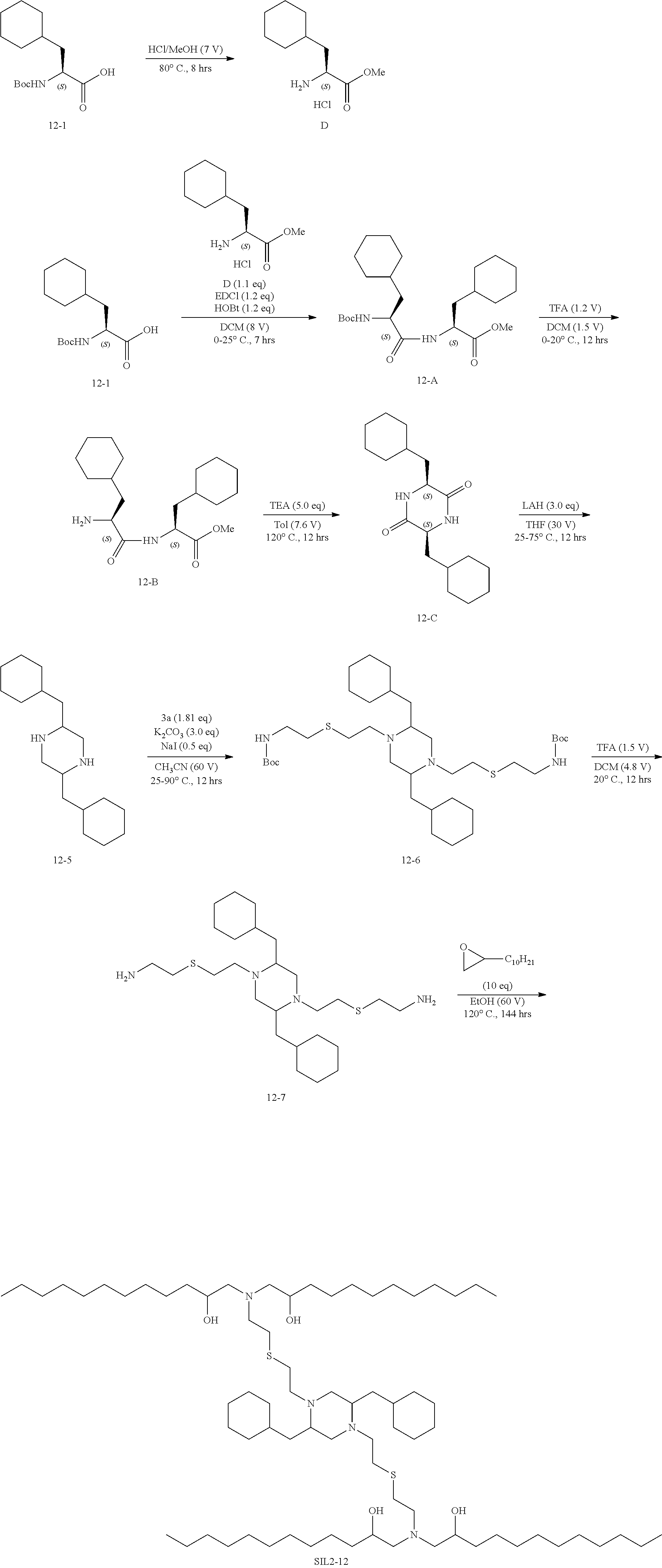

### Step 1. Preparation of Compound D

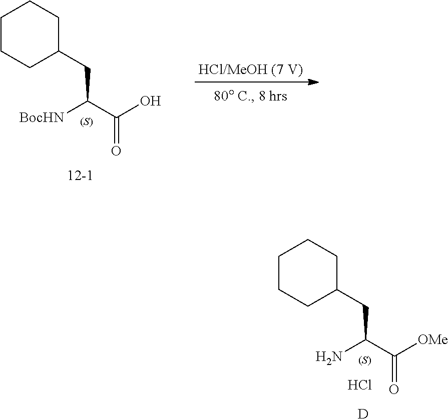

To a solution of Compound 12-1 (3.00 g, 11. mmol, 1.00 eq) in HCl/MeOH (4.00 M, 21.0 mL, 1.00 eq) at 0° C. The mixture was stirred at 80° C. for 8 hrs. LCMS (ET54505-120-P1A1, RT=0.357 min) indicated desired product. Concentrate under reduced pressure to give a residue. Without further purification. Compound D (1.84 g, 9.93 mmol, 89.8% yield) was obtained as an off-white solid.

¹H NMR: (400 MHz, CDCl₃)

δ ppm 8.88 (s, 2H), 4.10 (s, 1H), 3.83 (s, 3H), 3.50 (s, 1H), 1.55-2.12 (m, 10H), 0.85-1.44 (m, 5H).

### Step 2. Preparation of Compound 12-A

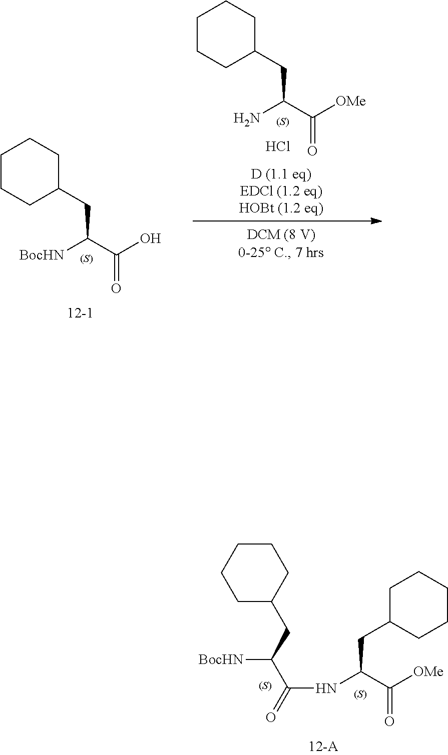

At 0° C., to a solution of Compound 12-1 (2.50 g, 9.21 mmol, 1.00 eq) in DCM (20.0 mL) were added HOBt (1.49 g, 11.1 mmol, 1.20 eq) and EDCI (2.12 g, 11.1 mmol, 1.20 eq). After 30 min, Compound D (1.88 g, 10.1 mmol, 1.10 eq) and DIEA (1.49 g, 11.5 mmol, 2.01 mL, 1.25 eq) were added. Stir reaction at 25° C. for 7 hrs. TLC (Dichloromethane:Methanol=10:1) indicated the material was consumed. The DCM solution washed with 1 N HCl, saturated NaHCO₃ and brine, dried over MgSO₄, and evaporated under vacuum, without further purification. Compound 12-A (3.30 g, 7.52 mmol, 81.7% yield) was obtained as a brown oil.

¹H NMR: (400 MHz, CDCl₃)

δ ppm 6.25-6.50 (m, 1H), 4.78-4.92 (m, 1H), 4.64 (m, 1H), 4.13 (m, 1H), 3.73 (s, 3H), 1.67 (m, 11H), 1.49 (s, 9H), 1.19 (m, 7H), 0.80-1.04 (m, 4H).

### Step 3. Preparation of Compound 12-B

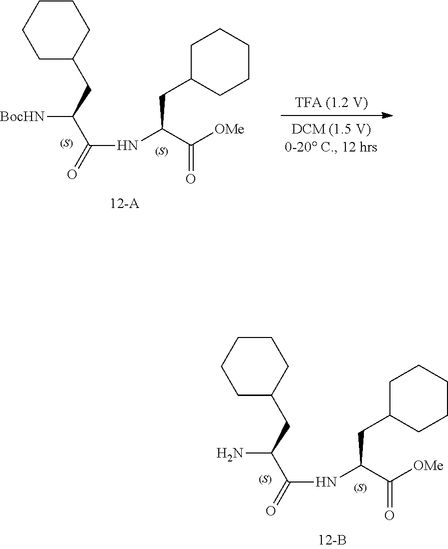

Dissolve Compound 12-A (3.30 g, 7.52 mmol, 1.00 eq) with DCM (5.00 mL). Add TFA (6.16 g, 54.0 mmol, 4.00 mL, 7.18 eq) into the mixture at 20° C. Stir the mixture for 12 hrs. TLC (Petroleum ether:Ethyl acetate=0:1, the material R_{f}=0.42, the product R_{f}=0.00) indicated the material was consumed. Concentrate under reduced pressure to give a residue. Without further purification. Compound 12-B (3.96 g, crude) was obtained as a brown oil.

### Step 3. Preparation of Compound 12-C

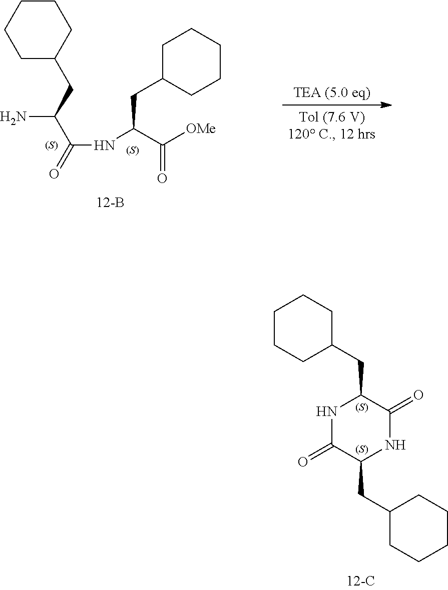

Dissolved Compound 12-B (3.96 g, 11.7 mmol, 1.00 eq) in Tol. (30.0 mL), and degassed and purged the mixture with N₂ for 3 times. Add TEA (5.92 g, 58.5 mmol, 8.14 mL, 5.00 eq) to the mixture. Stir the mixture at 120° C. for 12 hrs. TLC (Dichloromethane:Methanol=5:1, the material rf=0.31, Dichloromethane:Methanol=20:1 the product rf=0.33) indicated the material was consumed. Concentrate under reduced pressure to give a residue. The crude product was triturated with H₂O at 20° C. for 30 min. Without further purification. Compound 12-C (1.45 g, 4.73 mmol, 40.4% yield) was obtained as a white solid.

¹H NMR: (400 MHz, DMSO)

δ ppm 8.17 (s, 2H), 3.75 (s, 2H), 1.45-1.65 (m, 16H), 1.15-1.24 (m, 6H), 0.81-0.91 (m, 4H).

### Step 5. Preparation of Compound 12-5

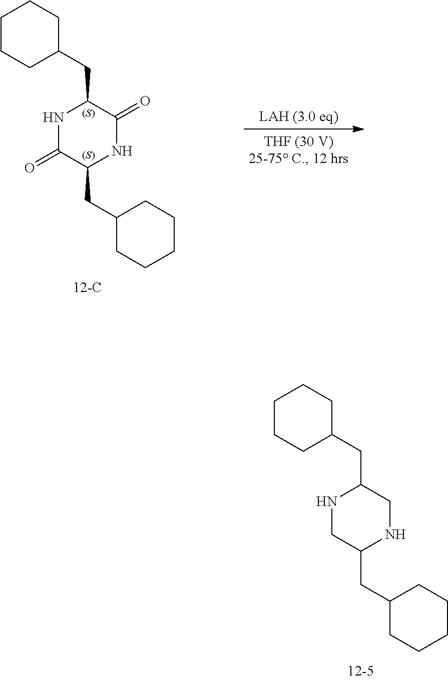

Dissolve Compound 12-C (1.00 g, 3.26 mmol, 1.00 eq) in THF (30.0 mL) at 25° C. Add LAH (619 mg, 16.3 mmol, 5.00 eq) into the reaction at 25° C. Stir the reaction at 75° C. for 12 hrs. TLC (Dichloromethane:Methanol=5:1, the material rf=0.63) indicated the material was remained. Add 0.6 mL of H₂O into the reaction at 0° C. and pour 0.6 mL of 15% NaOH aq, 1.80 mL of H₂O into the mixture. Add anhydrous sodium sulfate into the reaction. Filter and concentrated under reduced pressure to give a residue. Without further purification. Compound 12-5 (1.20 g, crude) was obtained as an off-white solid.

### Step 6. Preparation of Compound 12-6

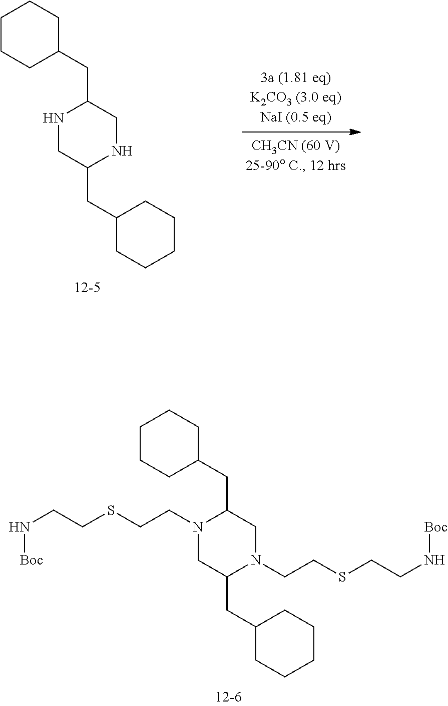 [SMILES: C1CCC(CC2CNC(CC3CCCCC3)CN2)CC1 ; CC(C)(C)OC(=O)NCCSCCN1CC(CC2CCCCC2)N(CCSCCNC(=O)OC(C)(C)C)CC1CC1CCCCC1]

Dissolve Compound 3a (781 mg, 3.26 mmol, 1.81 eq) in MeCN (30.0 mL) and add K₂CO₃ (744 mg, 5.39 mmol, 3.00 eq), NaI (135 mg, 898 μmol, 0.5 eq) into the mixture at 20° C. Add Compound 12-5 (0.5 g, 1.80 mmol, 1.00 eq) into the mixture at 20° C. Stir the reaction at 90° C. for 12 hrs. TLC (Dichloromethane:Methanol=5:1, Petroleum ether:Ethyl acetate=0:1, the material rf=0.03, the product rf=0.52) and LCMS (ET54505-129-P1A1, RT=0.669 min) indicated desired product. Concentrate under reduced pressure to give a residue. The residue was purified by column chromatography (SiO₂, Petroleum ether/Ethyl acetate=1/0 to 0/1). Compound 12-6 (0.629 g, 584 μmol, 32.5% yield) was obtained as a brown solid.

### Step 7. Preparation of Compound 12-7

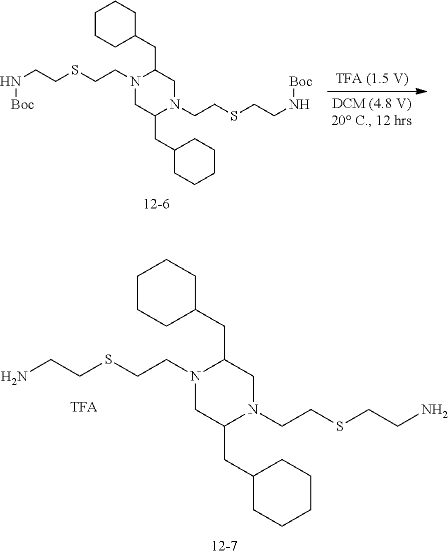 [SMILES: CC(C)(C)OC(=O)NCCSCCN1CC(CC2CCCCC2)N(CCSCCNC(=O)OC(C)(C)C)CC1CC1CCCCC1 ; NCCSCCN1CC(CC2CCCCC2)N(CCSCCN)CC1CC1CCCCC1]

Dissolve Compound 12-6 (0.629 g, 918 μmol, 1.00 eq) with DCM (3.00 mL). Add TFA (1.45 g, 12.7 mmol, 944 μL, 14.0 eq) into the mixture at 20° C. Stir the mixture for 12 hrs. TLC (Petroleum ether:Ethyl acetate=0:1, the material rf=0.42, the product rf=0.00) indicated the material was consumed. Concentrate under reduced pressure to give a residue. Add resin (5.00 g) to adjust pH=7-9. Without further purification. Compound 12-7 (0.40 g, 825 μmol, 89.8% yield) was obtained as a brown oil.

### Step 8. Preparation of Compound SIL2-12

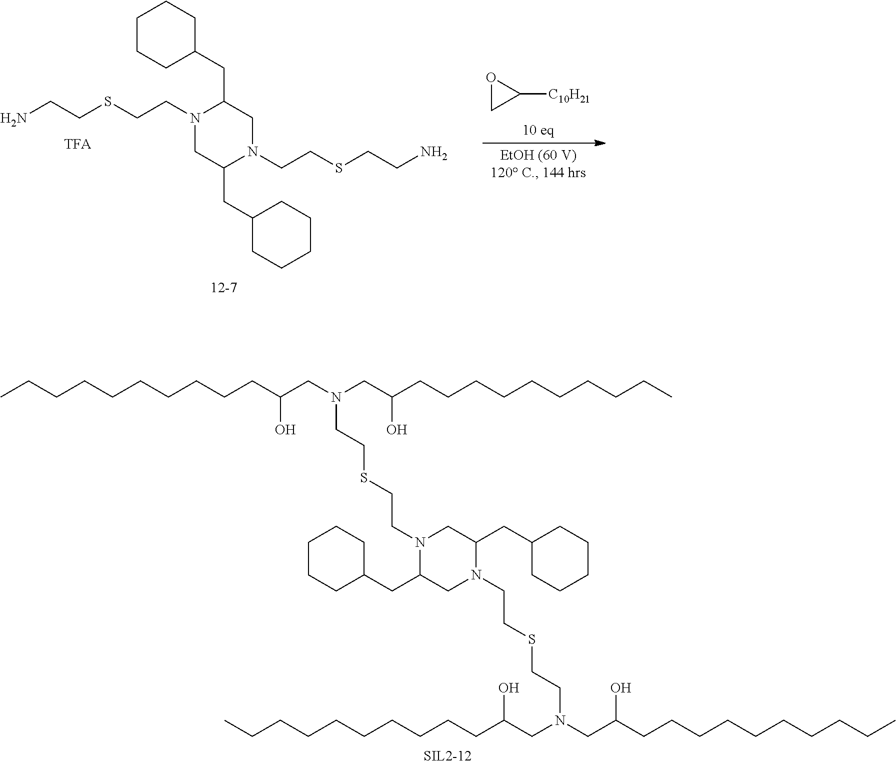

Dissolve Compound 12-7 (0.124 g, 256 μmol, 1.00 eq) with EtOH (40.0 mL). Add 2-decyloxirane (471 mg, 2.56 mmol, 10.0 eq) into the mixture. Stir the mixture at 120° C. for 144 hrs. TLC (Dichloromethane:Methanol=10:1, the product rf=0.49) indicated the material was consumed. Concentrate under reduced pressure to give a residue. The residue was purified by column chromatography (SiO₂, Petroleum ether/Ethyl acetate=10/1 to 0/1). Compound SIL2-12 (0.05 g, 40.9 μmol, 16.0% yield) was obtained as a brown oil.

¹H NMR: (400 MHz, CDCl₃)

δ ppm 3.20-3.85 (m, 13H), 2.23-2.92 (m, 23H), 1.26-1.76 (m, 100H), 0.85-0.95 (m, 12H).

LCMS: (MS/2+H=611, RT=3.432 min)

### Example 7. Synthesis of Compound SIL2-13

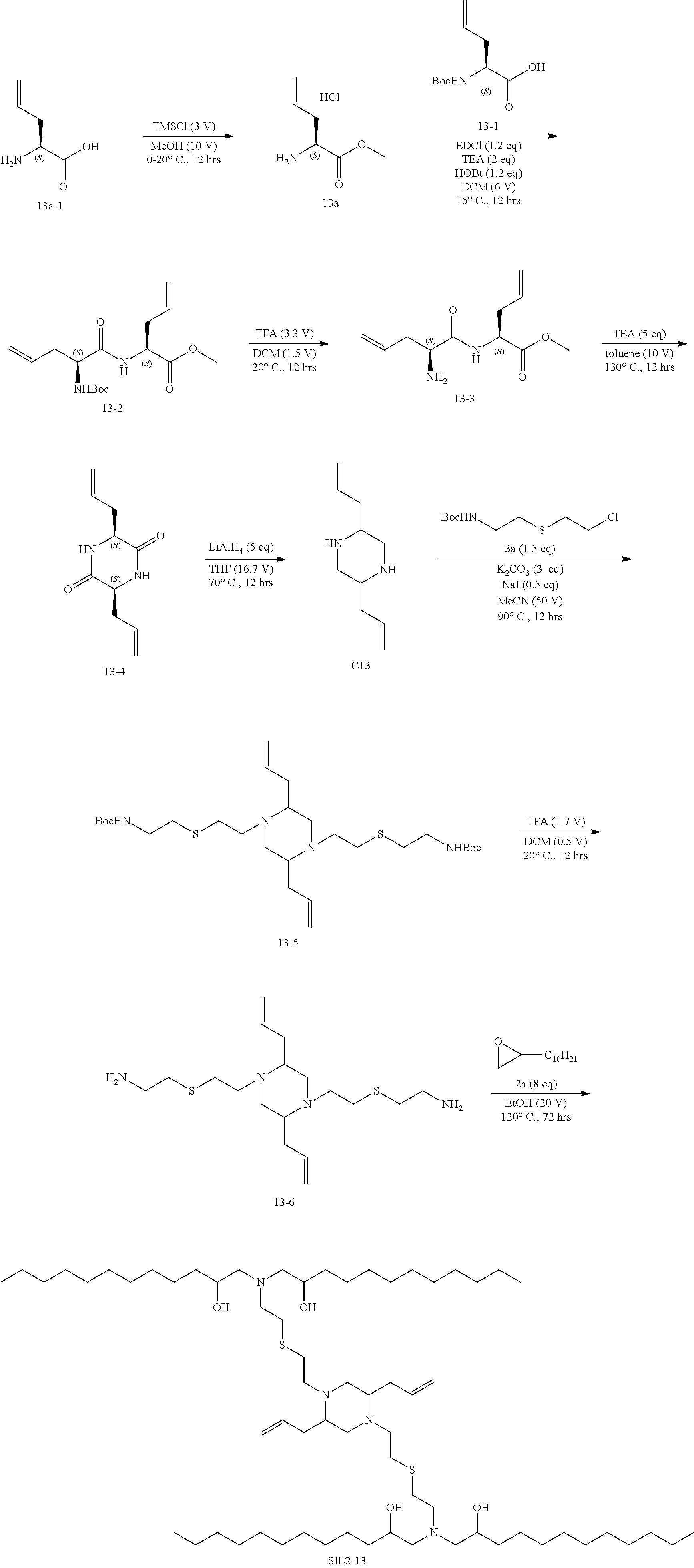

### Step 1 Preparation of Compound 13a

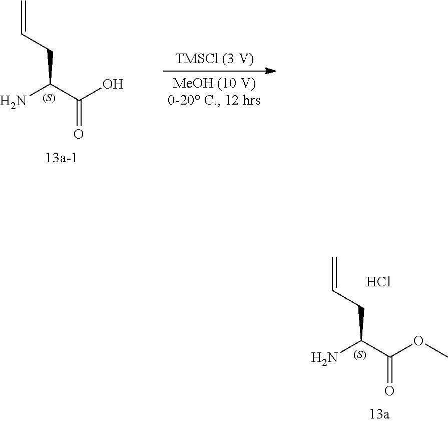

Compound 13α-1 (1 g, 8.69 mmol, 1 eq) was dissolved in MeOH (10 mL), cooled to 0° C., and then TMSCl (2.57 g, 23.64 mmol, 3 mL, 2.72 eq) was added. The mixture was allowed to warm to 20° C. and stirred for 12 hrs. LCMS (ET54476-35-P1A1, product: RT=0.048 min) showed the starting material was consumed completely. The solvent was removed in vacuo. And the resulting yellow oil was dissolved in hot EtOAc and precipitated with cyclohexane. Compound 13a (1.2 g, 7.25 mmol, 83.42% yield, HCl) was obtained as a white solid.

¹H NMR: 400 MHz CDCl₃

δ 8.71 (br s, 3H) 5.86 (ddt, J=17.01, 9.91, 7.24, 7.24 Hz, 1H) 5.20-5.38 (m, 2H) 4.23-4.33 (m, 1H) 3.80 (s, 3H) 2.79-2.91 (m, 2H)

### Step 2. Preparation of Compound 13-2

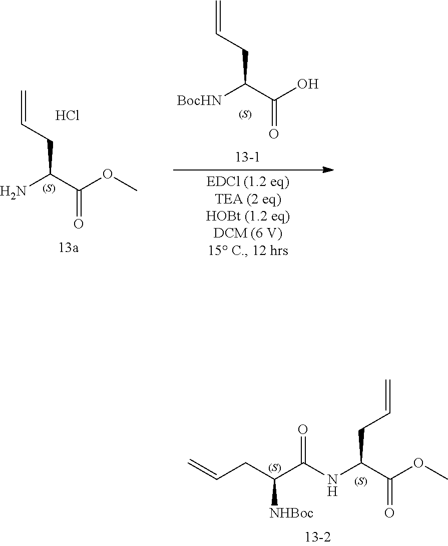

To a solution of Compound 13-1 (1 g, 4.65 mmol, 1 eq) in DCM (6 mL) was added HOBt (753.30 mg, 5.58 mmol, 1.2 eq), TEA (940.23 mg, 9.29 mmol, 1.29 mL, 2 eq), EDCI (1.07 g, 5.58 mmol, 1.2 eq) and Compound 13a (846.38 mg, 5.11 mmol, 1.1 eq, HCl). The mixture was stirred at 15° C. for 12 hrs. TLC (Petroleum ether:Ethyl acetate=2:1, R_{f}=0.24) indicated Compound 13-1 was consumed completely and two new spots formed. The reaction mixture was washed with water (10 mL) and the organic phase was separated, water phase was extracted with EtOAc (20 mL), washed with NaHCO₃ (30 mL), brine (30 mL), dried over Na₂SO4, filtered and concentrated under reduced pressure to give a residue. Compound 13-2 (1.3 g, crude) was obtained as a yellow oil.

¹H NMR: 400 MHz CDCl₃

δ 6.64 (br d, J=7.25 Hz, 1H) 5.59-5.83 (m, 2H) 5.07-5.21 (m, 4H) 4.99 (br s, 1H) 4.62-4.71 (m, 1H) 4.09-4.23 (m, 1H) 3.74 (s, 3H) 2.43-2.66 (m, 4H) 1.45 (s, 9H)

### Step 3. Preparation of Compound 13-3

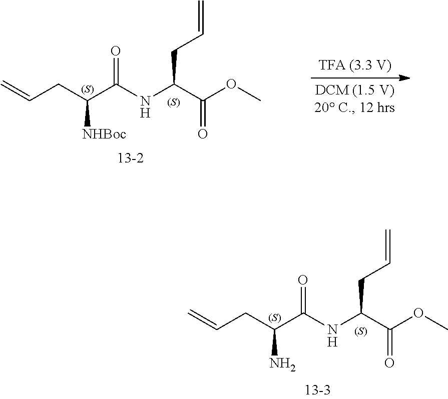

To a solution of Compound 13-2 (9.7 g, 29.72 mmol, 1 eq) in DCM (15 mL) was added TFA (49.30 g, 432.34 mmol, 32.01 mL, 14.55 eq) and. The mixture was stirred at 20° C. for 12 hrs. TLC (Petroleum ether:Ethyl acetate=2:1, R_{f}=0.00) indicated Compound 13-2 was consumed completely and one new spot formed. The reaction mixture was concentrated under reduced pressure. The crude product was used into the next step without further purification. Compound 13-3 (14.7 g, crude, TFA) was obtained as a yellow oil.

### Step 4. Preparation of Compound 13-4

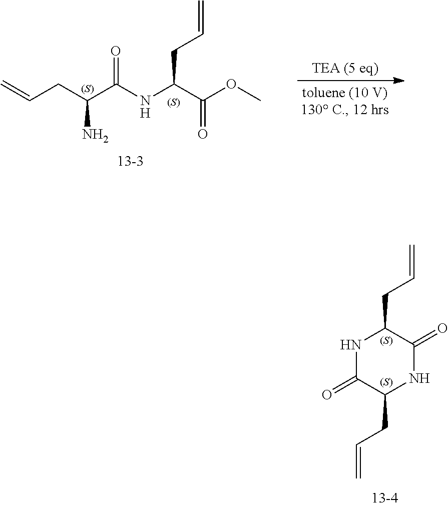

To a solution of Compound 13-3 (14.7 g, 64.97 mmol, 1 eq) in toluene (147 mL) was added TEA (32.87 g, 324.83 mmol, 45.21 mL, 5 eq). The mixture was stirred at 130° C. for 12 hrs. i HNMR showed the starting material was consumed completely. The reaction mixture was diluted with EtOAc (10 mL) and filtered. The filter cake was concentrated. The crude product was used into the next step without further purification. Compound 13-4 (3.6 g, 18.53 mmol, 28.53% yield) was obtained as a white solid.

¹H NMR: 400 MHz DMSO-d₆

δ 8.02-8.15 (m, 2H) 5.71 (ddt, J=16.96, 10.27, 7.08, 7.08 Hz, 2H) 5.06-5.15 (m, 4H) 3.83-3.97 (m, 2H) 2.34-2.48 (m, 4H)

### Step 5. Preparation of Compound C13

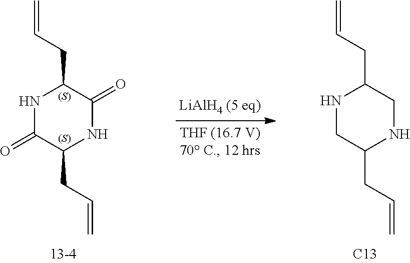

Compound 13-4 (3.6 g, 18.53 mmol, 1 eq) was added in portions to a solution of LiAlH₄ (3.52 g, 92.67 mmol, 5 eq) in THF (60 mL) at 20° C., the reaction mixture was stirred at 70° C. for 12 hrs. LCMS (ET54476-64-P1A1, product: RT=0.044 min) showed the starting material was consumed completely. The reaction mixture was cooled to 0° C. and quenched by dropwise added water 3.5 mL at 0° C., and then diluted with a solution of 15% NaOH 3.5 mL and water 10.5 mL, filtered and concentrated under reduced pressure to give a residue. Compound C13 (3.6 g, crude) was obtained as a white solid.

¹H NMR: 400 MHz CDCl₃

δ 5.70-5.84 (m, 2H) 5.04-5.15 (m, 5H) 2.81-2.88 (m, 2H) 2.70-2.81 (m, 4H) 2.29-2.40 (m, 2H) 2.18-2.26 (m, 2H) 1.68 (dt, J=5.94, 2.78 Hz, 2H)

### Step 6. Preparation of Compound 13-5

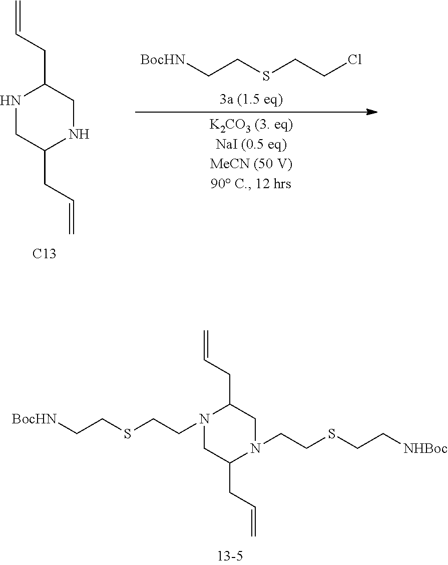 [SMILES: C=CCC1CNC(CC=C)CN1 ; CC(C)(C)OC(=O)NCCSCCCl ; "C13" ; C=CCC1CN(CCSCCNC(=O)OC(C)(C)C)C(CC=C)CN1CCSCCNC(=O)OC(C)(C)C]

To a solution of Compound 3a (1.30 g, 5.41 mmol, 1.5 eq) in MeCN (30 mL) was added K₂CO₃ (1.50 g, 10.83 mmol, 3 eq) and NaI (270.46 mg, 1.80 mmol, 0.5 eq), Compound C13 (600 mg, 3.61 mmol, 1 eq). The mixture was stirred at 90° C. for 12 hrs. LCMS (ET54476-69-P1A1, product: RT=0.572 min) showed the starting material was consumed completely. TLC (Dichloromethane:Methanol=10:1, R_{f}=0.50) indicated Compound C13 was consumed completely and many new spots formed. The reaction mixture was filtered, the filter cake was washed with ACN. The residue was purified by column chromatography (SiO₂, Petroleum ether/Ethyl acetate=20/1 to 0/1). Compound 13-5 (880 mg, 1.54 mmol, 42.57% yield) was obtained as a yellow oil.

¹H NMR: 400 MHz CDCl₃

δ 5.78 (ddt, J=16.93, 9.80, 7.33, 7.33 Hz, 2H) 5.01-5.13 (m, 4H) 4.95 (br d, J=0.88 Hz, 2H) 3.25-3.37 (m, 4H) 2.73-2.87 (m, 2H) 2.51-2.70 (m, 13H) 2.11-2.47 (m, 7H) 1.45 (s, 18H)

### Step 7. Preparation of Compound 13-6

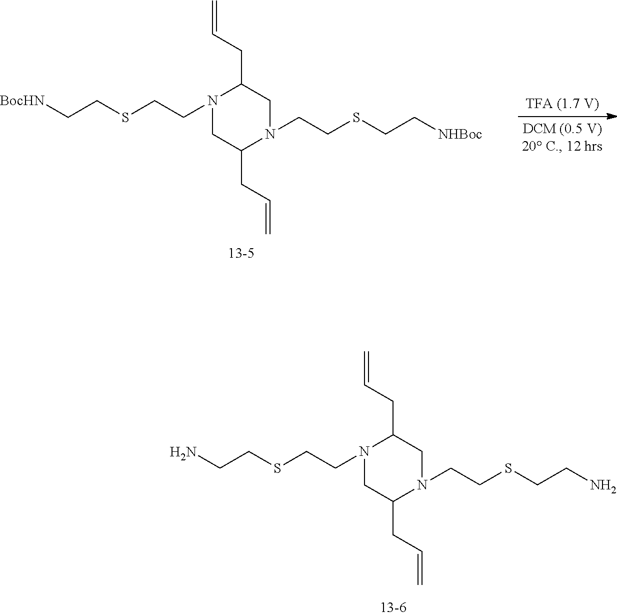 [SMILES: C=CCC1CN(CCSCCNC(=O)OC(C)(C)C)C(CC=C)CN1CCSCCNC(=O)OC(C)(C)C ; C=CCC1CN(CCSCCN)C(CC=C)CN1CCSCCN]

To a solution of Compound 13-5 (1.1 g, 1.92 mmol, 1 eq) in DCM (0.5 mL) was added TFA (2.93 g, 25.66 mmol, 1.9 mL, 13.36 eq). The mixture was stirred at 20° C. for 12 hrs. TLC (Dichloromethane:Methanol=10:1, R_{f}=0.00) indicated Compound 13-5 was consumed completely and one new spot formed. The reaction mixture was concentrated. The crude product was used into the next step without further purification. Compound 13-6 (1.9 g, crude, TFA) was obtained as a brown oil.

### Step 7. Preparation of Compound SIL2-13

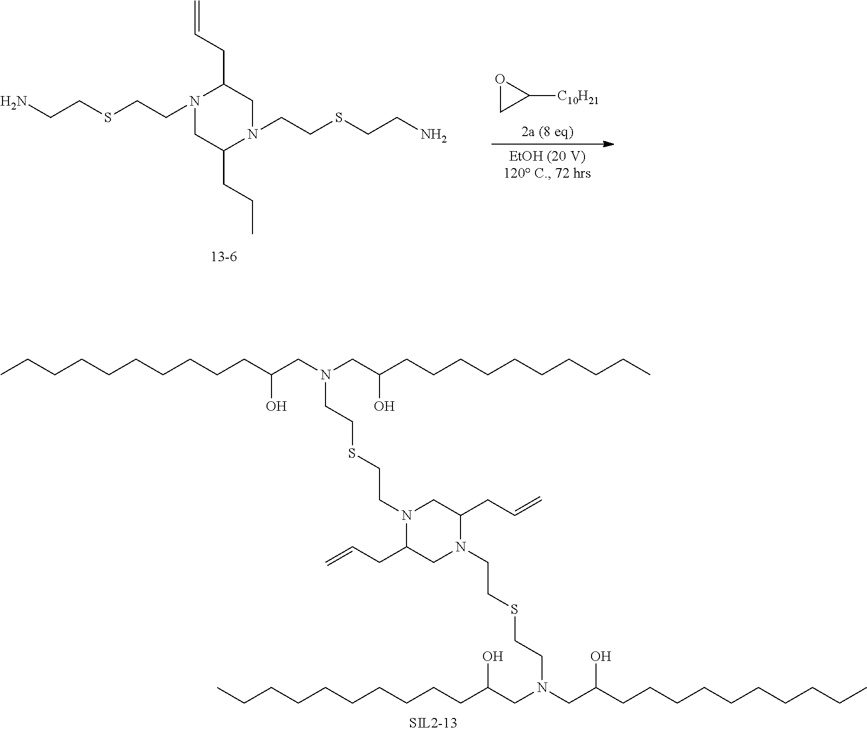

The Compound 13-6 (500 mg, 1.03 mmol, 1 eq, TFA) was dissolved in methanol and free with alkaline resin to pH=8-9, filter and concentrated to give 200 mg residue. To a solution of the residue in EtOH (4 mL) was added Compound 2a (1.51 g, 8.22 mmol, 8 eq). The mixture was stirred at 120° C. for 72 hrs. LCMS (ET54476-92-P1A1, product: RT=0.953 min) showed the starting material was consumed completely. TLC (Dichloromethane:Methanol=10:1, R_{f}=0.44) indicated many new spots formed. The reaction mixture was concentrated. The residue was purified by column chromatography (SiO₂, Dichloromethane:Methanol=100/1 to 10/1). Compound SIL2-13 (100 mg, 90.10 μmol, 8.77% yield) was obtained as a yellow oil.

¹H NMR: 400 MHz CDCl₃

δ 5.69-5.86 (m, 2H) 4.98-5.16 (m, 4H) 3.63 (br s, 4H) 3.01-3.39 (m, 4H) 2.52-2.98 (m, 22H) 2.28-2.51 (m, 10H) 2.11-2.27 (m, 2H) 1.25-1.48 (m, 72H) 0.89 (t, J=6.75 Hz, 12H)

### Example 8. Synthesis of Compound SIL2-15

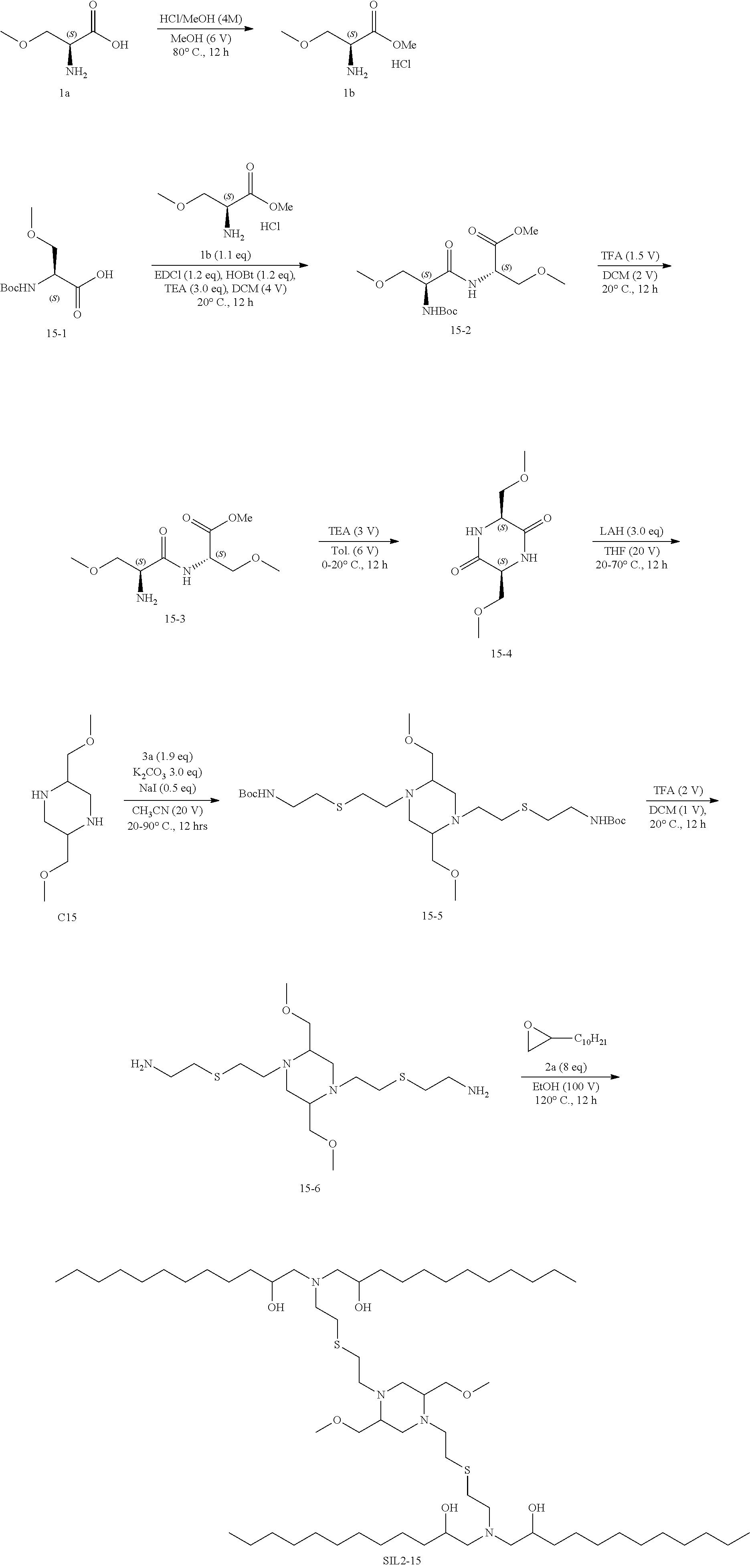

Step 1, preparation of Compound 1b

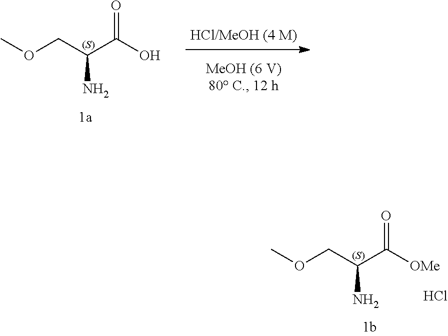

To a solution of Compound 1a (4.50 g, 28.9 mmol, 1.0 eq, HCl) in MeOH (30 mL) was added HCl/MeOH (4 M, 45 mL, 6.2 eq). The mixture was stirred at 80° C. for 12 hrs. LCMS showed reactant was used up, the product RT=0.044 min. The reaction mixture was concentrated under reduced pressure to remove MeOH (30 mL). Without purification. Compound 1b (6.00 g, crude, 2HCl) was obtained as a white solid, checked by ¹H NMR.

¹H NMR: (400 MHz, MeOD)

δ ppm 4.25-4.33 (m, 1H), 3.83-3.86 (m, 3H), 3.73-3.82 (m, 1H), 3.38-3.41 (m, 3H), 3.33-3.37 (m, 1H).

### Step 2. Preparation of Compound 15-2

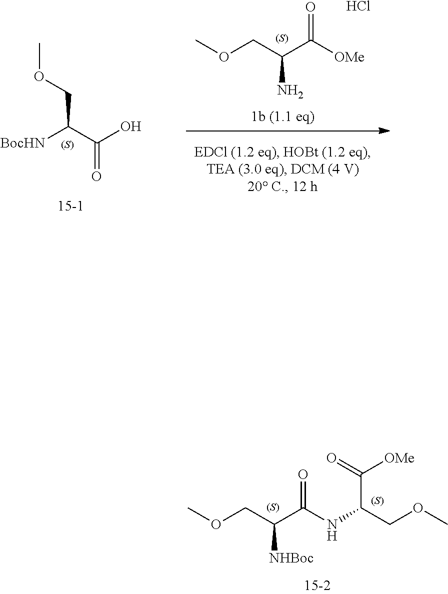

To a solution of Compound 15-1 (5.50 g, 25.1 mmol, 1.0 eq) in DCM (24 mL) was added EDCI (5.77 g, 30.1 mmol, 1.2 eq) and HOBt (4.07 g, 30.1 mmol, 1.2 eq), Compound 1b (4.08 g, 27.6 mmol, 90% purity, 1.1 eq), TEA (7.62 g, 75.3 mmol, 10.5 mL, 3.0 eq). The mixture was stirred at 20° C. for 12 hrs. TLC (Dichloromethane:Methanol=10:1) showed reactant was used up. The reaction mixture was quenched by addition H₂0 50 mL, and extracted with solvent DCM 100 mL. The organic layer was washed with brine 50 mL, dried over Na₂SO₄, filtered and concentrated under reduced pressure to give a residue. Without purification. Compound 15-2 (6.00 g, crude) was obtained as light yellow oil, checked by HNMR.

¹H NMR: (400 MHz, CDCl₃)

δ ppm 5.36-5.47 (m, 1H), 4.65-4.75 (m, 1H), 4.20-4.37 (m, 1H), 3.79-3.88 (m, 2H), 3.72-3.79 (m, 3H), 3.57-3.64 (m, 1H), 3.46-3.54 (m, 1H), 3.38-3.43 (m, 3H), 3.31-3.36 (m, 3H), 1.38-1.52 (m, 9H).

### Step 3. Preparation of Compound 15-3

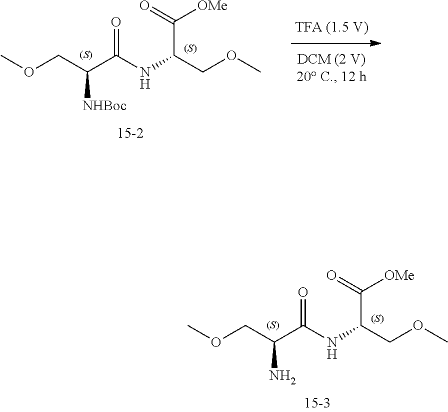

To a solution of Compound 15-3 (6.00 g, 17.9 mmol, 1.0 eq) in DCM (12 mL) was added TFA (13.9 g, 122 mmol, 9.00 mL, 6.8 eq). The mixture was stirred at 20° C. for 12 hrs. TLC (Dichloromethane:Methanol=10:1, product R_{f}=0.0) showed reactant was used up. The reaction mixture was concentrated under reduced pressure to remove DCM (12 mL). Without purification. Compound 15-3 (14.5 g, crude, TFA) was obtained as light yellow oil.

### Step 4. Preparation of Compound 15-4

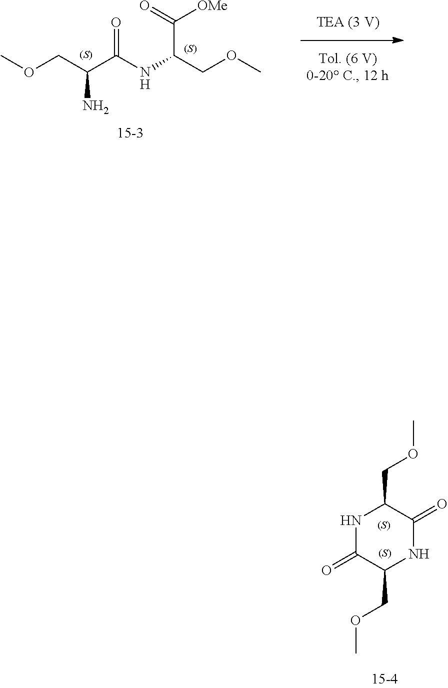

To a solution of methyl Compound 15-3 (14.5 g, 61.9 mmol, 1.0 eq) in Tol. (87 mL) was added TEA (31.3 g, 310 mmol, 43 mL, 5.0 eq). The mixture was stirred at 130° C. for 12 hrs. ¹H NMR showed reactant was used up. The reaction mixture was concentrated under reduced pressure to remove Tol. (87 mL). The crude product was triturated with ethyl acetate (50 mL) at 25° C. for 60 min. Compound 15-4 (2.6 g, 12.9 mmol, 41.5% yield) was obtained as a brown solid, checked by H NMR.

¹H NMR: (400 MHz, DMSO)

δ ppm 8.03-8.15 (m, 2H), 3.86 (d, J=1.63 Hz, 2H), 3.64-3.71 (m, 2H), 3.44 (dd, J=9.63, 2.75 Hz, 2H), 3.20-3.25 (m, 6H)

### Step 5. Preparation of Compound C15

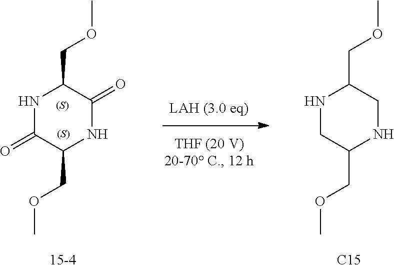

A suspension of Compound 15-4 (2.60 g, 12.9 mmol, 1.0 eq) in THF (52 mL) at 20° C., LAH (1.46 g, 38.6 mmol, 3.0 eq) was added to the mixture at 20° C. for 2 min. Then the reaction solution was heated at 70° C. and stirred for 12 hrs. LCMS showed reactant was used up, the product RT=0.044 min. The reaction was cooled to 0° C., water (1.46 mL) was dropped in the reaction solution under N₂ and created a lot of bubbles, then 15% aq. NaOH (1.46 mL) was added dropwise slowly at 0° C.; after 5 min, water (4.38 mL) was dropped the reaction solution at 0° C., then Na₂SO4 added the mixture and warmed to 25° C. stirred 15 min. The mixture was filtered and the filter cake concentrated under reduced pressure to give a residue. The residue was purified by column chromatography (SiO₂, petroleum ether/ethyl acetate=100/1 to 1/1). Compound C15 (1.10 g, 6.31 mmol, 49.1% yield) was obtained as a white solid, checked by ¹H NMR.

¹H NMR: (400 MHz, DMSO-d₆)

δ ppm 3.20-3.25 (m, 6H), 3.10-3.20 (m, 4H), 2.77-2.84 (m, 2H), 2.60-2.70 (m, 2H), 2.22 (t, J=10.69 Hz, 2H)

### Step 6. Preparation of Compound 15-5

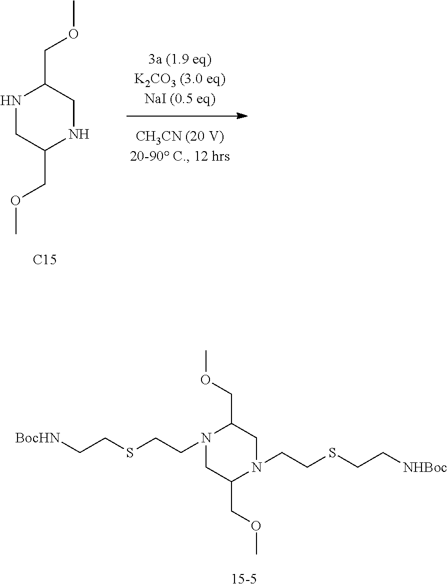 [SMILES: COCC1CNC(COC)CN1 ; "C15" ; COCC1CN(CCSCCNC(=O)OC(C)(C)C)C(COC)CN1CCSCCNC(=O)OC(C)(C)C]

To a solution of Compound 3a (2.61 g, 10.9 mmol, 1.9 eq) in ACN (20 mL) was added K₂CO₃ (2.38 g, 17.2 mmol, 3.0 eq) and NaI (430 mg, 2.87 mmol, 0.5 eq), Compound C15 (1.0 g, 5.74 mmol, 1.0 eq). The mixture was stirred at 90° C. for 12 hrs. LCMS showed reactant was used up, product RT=0.515 min. The reaction mixture was filtered, the filter cake was washed with ACN. Combined with ET54385-77 purification. The residue was purified by column chromatography (SiO₂, Petroleum ether/Ethyl acetate=100/1 to 2/1). Compound 15-5 (0.8 g, yield 22%) was obtained as a yellow solid, checked by ¹H NMR.

¹H NMR: (400 MHz, CDCl₃)

δ ppm 4.99 (s, 2H), 3.38-3.51 (m, 4H), 3.23-3.37 (m, 10H), 2.80-3.05 (m, 4H), 2.51-2.76 (m, 12H), 2.31 t, J=10.15 Hz, 2H), 1.39-1.54 (m, 18H).

Step 7. General procedure for preparation of Compound 15-6

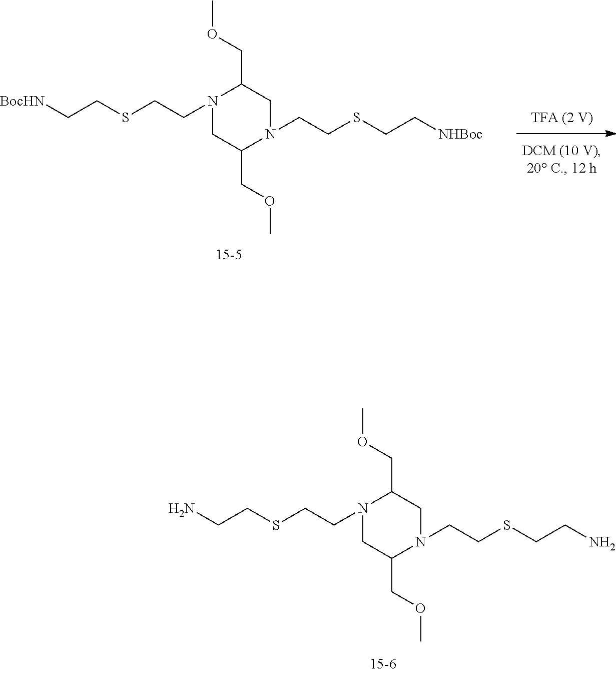 [SMILES: COCC1CN(CCSCCNC(=O)OC(C)(C)C)C(COC)CN1CCSCCNC(=O)OC(C)(C)C ; COCC1CN(CCSCCN)C(COC)CN1CCSCCN]

To a solution of Compound 15-5 (0.5 g, 861 μmol, 1.0 eq) in DCM (5.0 mL) was added TFA (1.54 g, 13.5 mmol, 1.0 mL, 15.7 eq). The mixture was stirred at 20° C. for 12 hrs. TLC (Dichloromethane:Methanol=10:1) showed reactant was used up. The reaction mixture was concentrated under reduced pressure to remove DCM (5.0 mL). The crude product was dissolved in MeOH (50 mL), alkaline resin (NH₄HCO₃) was added to adjust pH=7. Without purification. Compound 15-6 (0.386 g, crude) was obtained as yellow oil.

### Step 8. Preparation of Compound SIL2-15

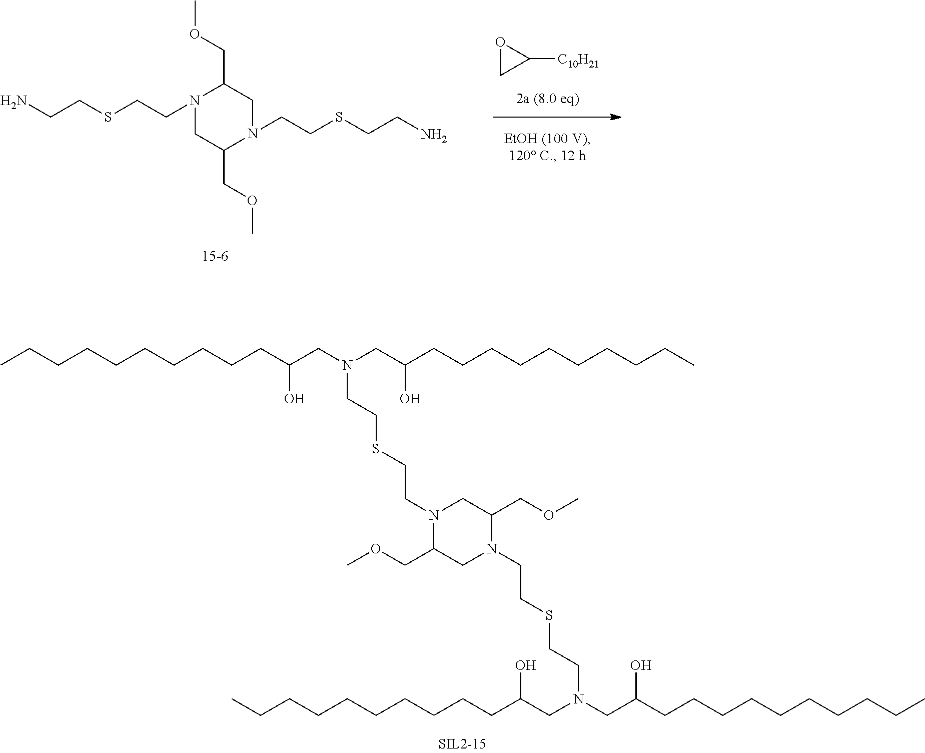

To a solution of Compound 15-6 (0.386 g, 935 μmol, 1.0 eq) in EtOH (40 mL) was added 2-decyloxirane (1.38 g, 7.48 mmol, 8.0 eq). The mixture was stirred at 120° C. for 72 hrs. LCMS showed reactant was used up, product RT=0.729 min. The reaction mixture was concentrated under reduced pressure to remove EtOH (40 mL). The residue was purified by column chromatography (SiO₂, Petroleum ether/Ethyl acetate=5/1 to DCM:MeOH=30/1). Compound SIL2-15 (50.35 mg, 45.0 μmol, 4.82% yield) was obtained as yellow oil, checked by ¹H NMR.

¹H NMR: (400 MHz, CDCl₃)

δ ppm 3.58-3.70 (m, 4H), 3.35-3.49 (m, 5H), 3.31-3.34 (m, 6H), 2.24-3.07 (m, 32H), 1.16-1.56 (m, 75H), 0.83-0.94 (m, 12H).

### Example 9. Synthesis of Compound SIL2-17

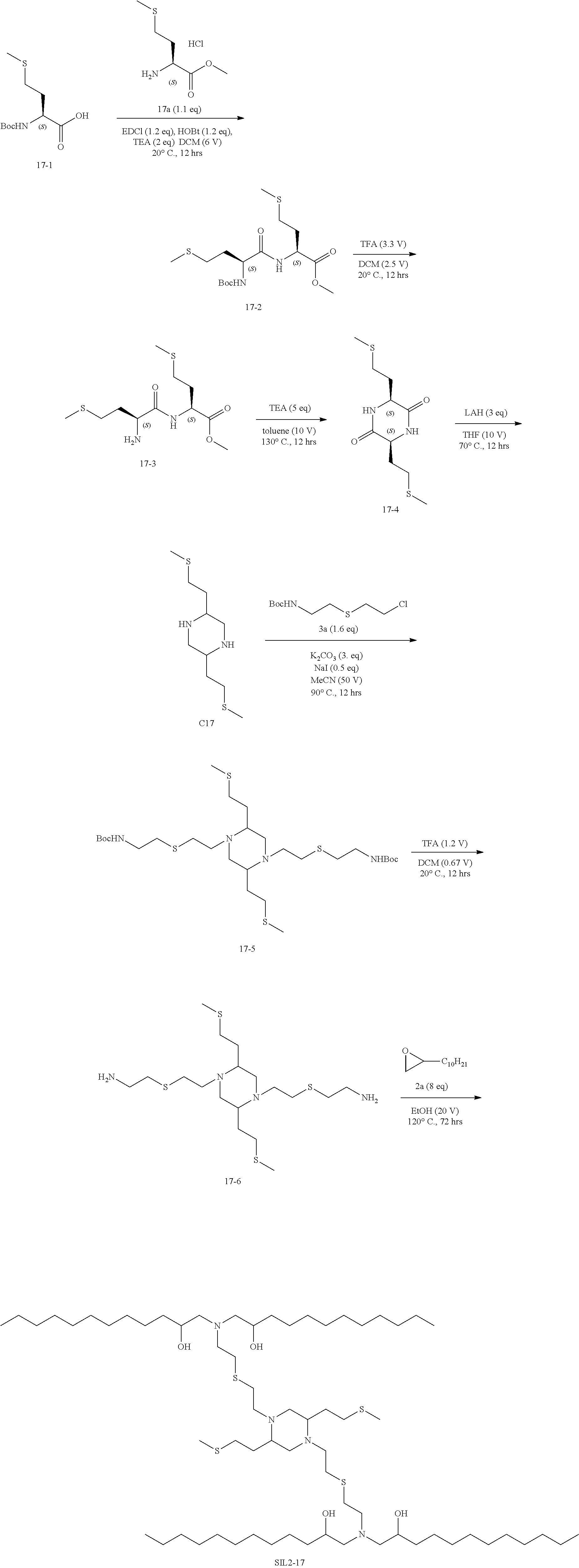

### Step 1. Preparation of Compound 17-2

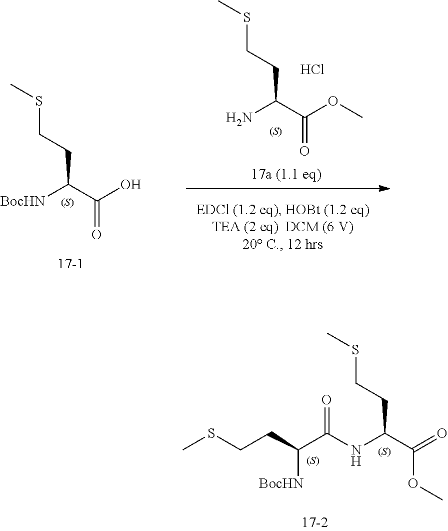

To a solution of Compound 17-1 (2 g, 8.02 mmol, 1 eq) in DCM (12 mL) was added EDCI (1.85 g, 9.63 mmol, 1.2 eq), TEA (1.62 g, 16.04 mmol, 2.23 mL, 2 eq), HOBt (1.30 g, 9.63 mmol, 1.2 eq) and Compound 17a (1.76 g, 8.82 mmol, 1.1 eq, HCl). The mixture was stirred at 20° C. for 12 hrs. TLC (Petroleum ether:Ethyl acetate=2:1, R_{f}=0.14) indicated Compound 17-1 was consumed completely and many new spots formed. The reaction mixture was washed with water (10 mL) and the organic phase was separated, water phase was extracted with DCM (20 mL*2), washed with NaHCO₃ (30 mL), brine (30 mL), dried over Na₂SO4, filtered and concentrated under reduced pressure to give a residue. Compound 17-2 (3.2 g, crude) was obtained as a yellow mixture of solid and oil.

¹H NMR: 400 MHz CDCl₃

δ 6.86 (br d, J=7.88 Hz, 1H) 5.22 (br d, J=8.13 Hz, 1H) 4.70 (td, J=7.79, 5.07 Hz, 1H) 4.29 (br d, J=6.88 Hz, 1H) 3.74 (s, 3H) 2.58 (t, J=7.13 Hz, 2H) 2.50 (t, J=7.38 Hz, 2H) 2.12-2.21 (m, 1H) 2.09 (d, J=10.13 Hz, 6H) 1.88-2.06 (m, 3H) 1.43 (s, 9H)

Step 2. preparation of compound 17-3

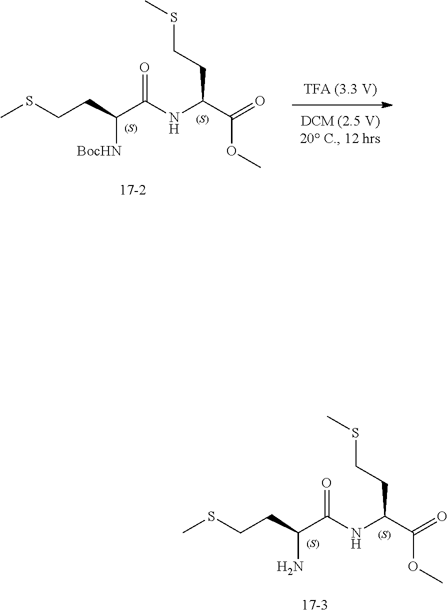

To a solution of compound 17-2 (3.2 g, 8.11 mmol, 1 eq) in DCM (8 mL) was added TFA (16.26 g, 142.63 mmol, 10.56 mL, 17.59 eq). The mixture was stirred at 20° C. for 12 hrs. TLC (Petroleum ether:Ethyl acetate=2:1, R_{f}=0.00) indicated Compound 17-2 was consumed completely and one new spot formed. The reaction mixture was concentrated under reduced pressure. Compound 17-3 (4 g, crude, TFA) was obtained as a brown oil.

### Step 3. Preparation of Compound 17-4

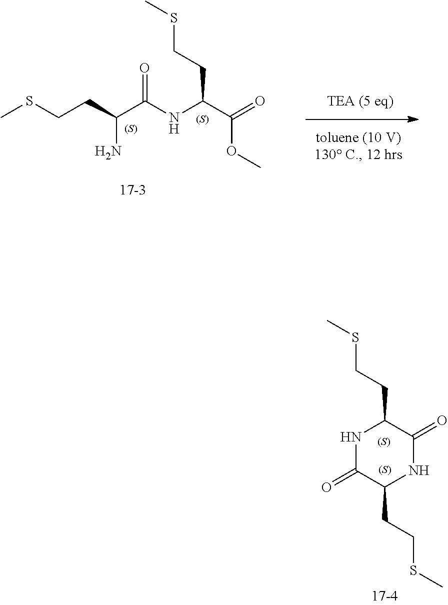

The Compound 17-3 (4 g, 13.59 mmol, 1 eq) was dissolved in toluene (40 mL) was added TEA (6.87 g, 67.93 mmol, 9.45 mL, 5 eq) and then stirred at 130° C. for 12 hrs. ¹HNMR showed the starting material was consumed completely. The reaction mixture was diluted with EtOAc (20 mL) and filtered; the filter cake was concentrated. The crude product was used into the next step without further purification. Compound 17-4 (1.7 g, 6.48 mmol, 47.69% yield) was obtained as a white solid.

¹H NMR: 400 MHz DMSO-d₆

δ 8.23 (s, 2H) 3.96 (t, J=5.50 Hz, 2H) 2.50-2.59 (m, 4H) 2.04 (s, 6H) 1.83-2.02 (m, 4H)

### Step 4. Preparation of Compound C17

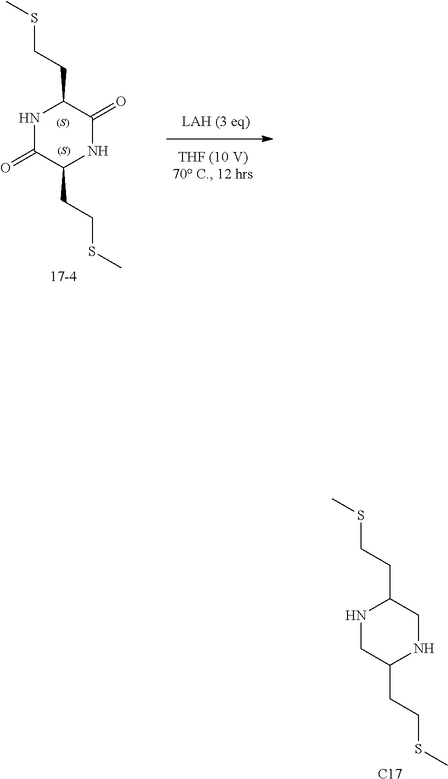

Compound 17-4 (3 g, 11.43 mmol, 1 eq) was added in portions to a solution of LiAlH₄ (1.30 g, 34.30 mmol, 3 eq) in THF (30 mL) at 20° C., the reaction mixture was stirred at 70° C. for 12 hrs. LCMS (ET54476-68-P1A1, product: RT=0.048 min) showed the starting material was consumed completely. The reaction mixture was cooled to 0° C. and quenched by dropwise added water 1.3 mL at 0° C., and then diluted with a solution of 15% NaOH 1.3 mL and water 3.9 mL, filtered and concentrated under reduced pressure to give a residue. Compound C17 (2.9 g, crude) was obtained as a yellow oil.

¹H NMR: 400 MHz CDCl₃

δ 2.78-2.88 (m, 4H) 2.68-2.75 (m, 2H) 2.52-2.60 (m, 4H) 2.09-2.13 (m, 6H) 1.80-1.91 (m, 2H) 1.68-1.77 (m, 2H) 1.48-1.64 (m, 2H)

### Step 5. Preparation of Compound 17-5

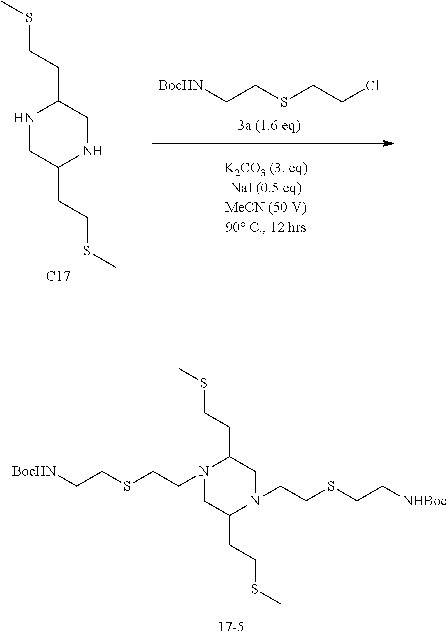

To a solution of Compound 3a (818.22 mg, 3.41 mmol, 1.6 eq) in MeCN (25 mL) was added K₂CO₃ (884.36 mg, 6.40 mmol, 3 eq) and NaI (159.85 mg, 1.07 mmol, 0.5 eq), Compound C17 (500 mg, 2.13 mmol, 1 eq). The mixture was stirred at 90° C. for 12 hrs. LCMS (ET54476-72-P1A1, product: RT=0.586 min) showed the starting material was consumed completely. TLC (Dichloromethane:Methanol=10:1, R_{f}=0.50) indicated Compound C17 was consumed completely and many new spots formed. The reaction mixture was filtered, the filter cake was washed with ACN. Combined with ET54476-71-p1 for purification. The residue was purified by column chromatography (SiO₂, Petroleum ether/Ethyl acetate=20/1 to 0/1). Compound 17-5 (0.5 g, 30.45% yield) was obtained as a yellow oil.

¹H NMR: 400 MHz CDCl₃

δ 4.97 (br s, 2H) 3.32 (br d, J=6.00 Hz, 4H) 2.74-2.84 (m, 2H) 2.52-2.72 (m, 16H) 2.38-2.50 (m, 4H) 2.12 (s, 6H) 1.82-1.97 (m, 2H) 1.69-1.80 (m, 2H) 1.45 (s, 18H)

### Step 6. Preparation of Compound 17-6

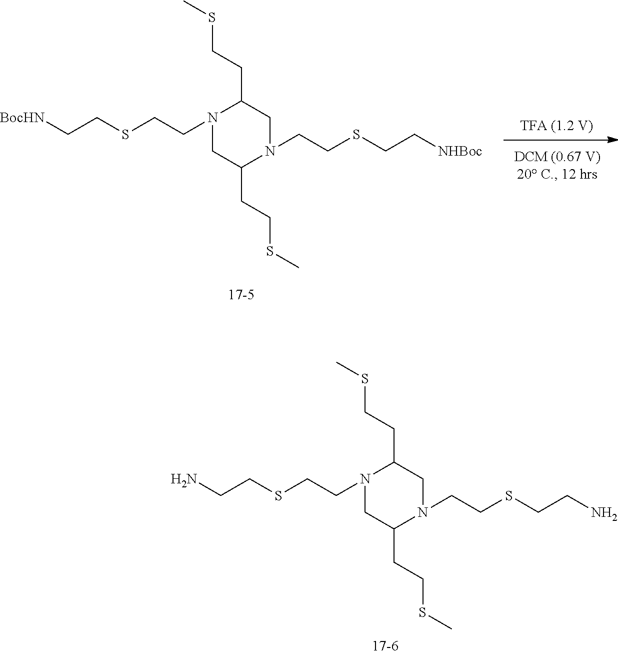 [SMILES: CSCCC1CN(CCSCCNC(=O)OC(C)(C)C)C(CCSC)CN1CCSCCNC(=O)OC(C)(C)C ; CSCCC1CN(CCSCCN)C(CCSC)CN1CCSCCN]

To a solution of Compound 17-5 (0.6 g, 936.00 μmol, 1 eq) in DCM (0.4 mL) was added TFA (1.08 g, 9.45 mmol, 0.7 mL, 10.10 eq). The mixture was stirred at 20° C. for 12 hr. TLC (Dichloromethane:Methanol=10:1, R_{f}=0.00) indicated Compound 17-5 was consumed completely and one new spot formed. The reaction mixture was concentrated to remove the TFA. The crude product was used into the next step without further purification. Compound 17-6 (0.8 g, crude, TFA) was obtained as a brown oil.

### Step 6. Preparation of Compound SIL2-17

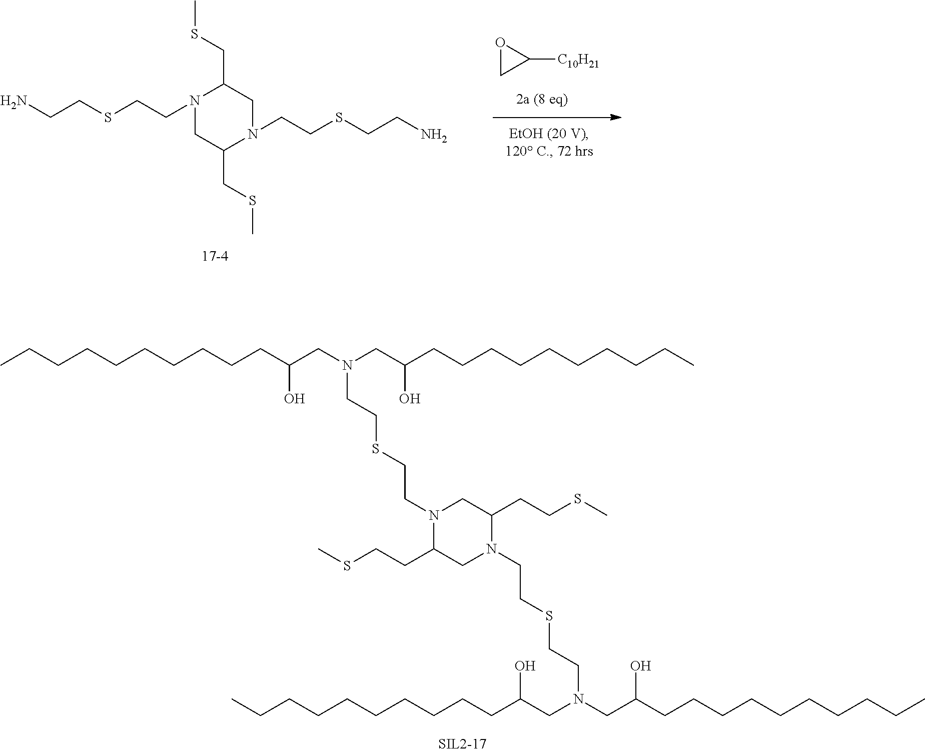

The Compound 17-6 (0.4 g, 720.96 μmol, 1 eq, TFA) was dissolved in methanol and free with alkaline resin to pH=8-9, filter and concentrated to give 180 mg residue. To a solution of the residue in EtOH (8 mL) was added Compound 2a (1.06 g, 5.77 mmol, 8 eq). The mixture was stirred at 120° C. for 72 hrs. LCMS (ET54476-88-P1A2, product: RT=0.945) showed the starting material was consumed completely. TLC (Dichloromethane:Methanol=10:1, R_{f}=0.34) indicated Compound 17-6 was consumed completely and many new spots formed. The reaction mixture was concentrated. The residue was purified by column chromatography (SiO₂, Petroleum ether/Ethyl acetate=100/1 to 0/1). Compound SIL2-17 (80 mg, 67.91 μmol, 9.42% yield) was obtained as a yellow oil.

¹H NMR: 400 MHz CDCl₃

δ 3.63 (br s, 4H) 3.00-3.40 (m, 4H) 2.53-2.91 (m, 24H) 2.27-2.51 (m, 10H) 2.12 (s, 6H) 1.87 (br d, J=6.88 Hz, 2H) 1.68-1.79 (m, 2H) 1.24-1.48 (m, 72H) 0.89 (br t, J=6.75 Hz, 12H)

Compounds SIL2-18 to SIL2-21 were prepared similarly with the methods described above. Their structures and 1H-NMR data are summarized in Table 2. In addition, Compounds SIL2-14 and SIL2-16 can be prepared similarly with the methods described above.

TABLE 2

| | ¹H NMR (δ ppm, |
|---|---|
| Compound | 400 MHz) |
| 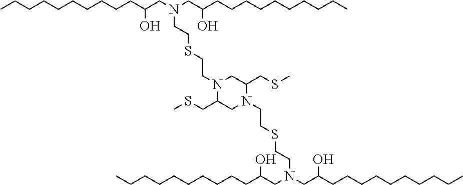 [SMILES: CCCCCCCCCCC(O)CN(CCSCCN1CC(CSC)N(CCSCCN(CC(O)CCCCCCCCCC)CC(O)CCCCCCCCCC)CC1CSC)CC(O)CCCCCCCCCC] | 3.57 − 3.80 (m, 4 H), 2.33 − 3.35 (m, 36 H), 2.06 − 2.18 (m, 5 H), 1.13 − 1.57 (m, 76 H), 0.83 − 0.96 (m, 12 H) (CDCl3) |
| SIL2-18 | |
| 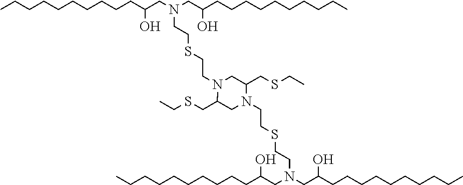 [SMILES: CCCCCCCCCCC(O)CN(CCSCCN1CC(CSCC)N(CCSCCN(CC(O)CCCCCCCCCC)CC(O)CCCCCCCCCC)CC1CSCC)CC(O)CCCCCCCCCC] | 3.65 (s, 7H), 2.55 (m, 31H), 1.35 (m, 78H), 0.90 (s, 12H) (CDCl3) |
| SIL2-19 | |
| 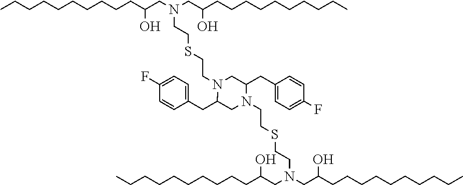 [SMILES: CCCCCCCCCCC(O)CN(CCSCCN1CC(Cc2ccc(F)cc2)N(CCSCCN(CC(O)CCCCCCCCCC)CC(O)CCCCCCCCCC)CC1Cc1ccc(F)cc1)CC(O)CCCCCCCCCC] | 7.10 − 7.20 (m, 4 H), 6.98 (t, J = 8.50 Hz, 4 H), 3.52 − 3.73 (m, 5 H), 3.07 − 3.27 (m, 3 H), 2.23 − 2.97 (m, 29 H), 1.20 − 1.51 (m, 74 H), 0.89 (t, J = 6.75 Hz, 12 H) (CDCl3) |
| SIL2-20 | |
| 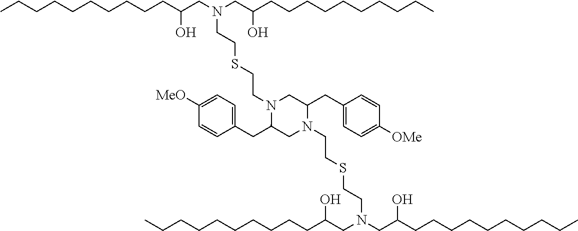 [SMILES: CCCCCCCCCCC(O)CN(CCSCCN1CC(Cc2ccc(OC)cc2)N(CCSCCN(CC(O)CCCCCCCCCC)CC(O)CCCCCCCCCC)CC1Cc1ccc(OC)cc1)CC(O)CCCCCCCCCC] | 7.10 (d, J = 8.13 Hz, 4 H), 6.84 (d, J = 8.25 Hz, 4 H), 3.81 (s, 6 H), 3.62 (s, 4 H), 3.17 (s, 4 H), 2.23 − 2.95 (m, 33 H), 1.15 − 1.57 (m, 74 H), 0.89 (t, J = 6.75 Hz, 12 H) (CDCl3) |
| SIL2-21 | |

### Example 10. Synthesis of SIL2-22

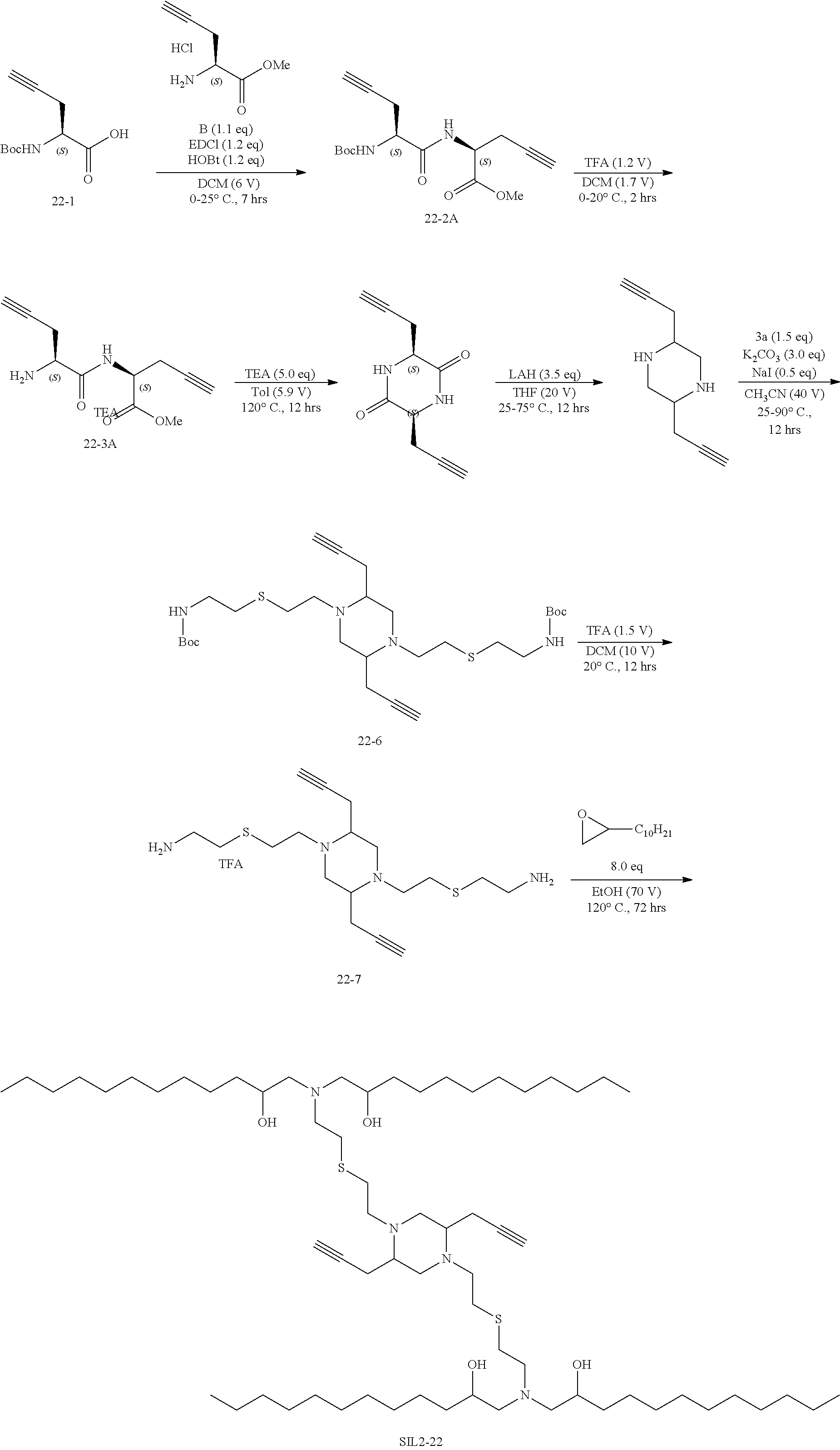

### Step 1. Preparation of Compound 22-2A

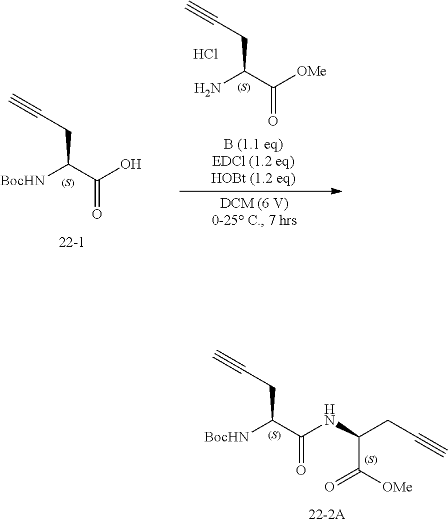

At 0° C., to a solution of Compound 22-1 (4.00 g, 18.8 mmol, 1.00 eq) in DCM (24.0 mL) were added HOBt (3.04 g, 22.5 mmol, 1.20 eq) and EDCI (4.32 g, 22.5 mmol, 1.20 eq). After 30 min, Compound B (3.38 g, 20.6 mmol, 1.10 eq, HCl) and DIEA (3.03 g, 23.5 mmol, 4.08 mL, 1.25 eq) were added. Stir reaction at 25° C. for 7 hrs. TLC (Dichloromethane:Methanol=10:1) indicated the material was consumed. The DCM solution washed with 1 N HCl, saturated NaHCO₃ and brine, dried over MgSO₄, and evaporated under vacuum. Without further purification. Compound 22-2A (6.80 g, crude) was obtained as a brown oil.

¹H NMR: (400 MHz, CDCl₃)

δ ppm 7.12 (d, J=6.00 Hz, 1H), 4.73-4.75 (m, 1H), 3.81 (s, 3H), 2.65-2.81 (m, 3H), 2.60-2.65 (m, 1H), 2.12 (d, J=2.40 Hz, 1H), 2.02-2.04 (m, 1H), 1.48 (s, 9H).

Step 2. General procedure for preparation of Compound 22-3A

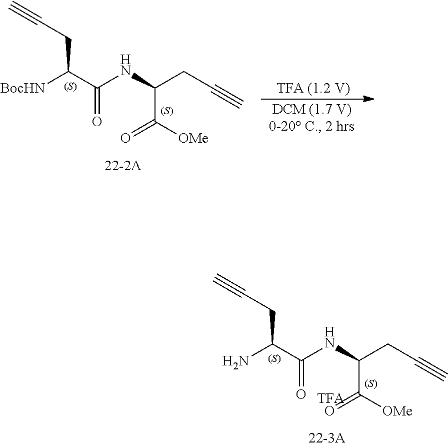

Dissolve Compound 22-2A (6.00 g, 19.3 mmol, 1.00 eq) in DCM (10.0 mL), degass and purge the mixture with N₂ for 3 times. Add TFA (11.1 g, 97.0 mmol, 7.20 mL, 5.03 eq) into the mixture at 0° C. Stir the reaction at 20° C. for 2 hrs. TLC (Dichloromethane:Methanol=10:1, the product Rr=0.10) indicated the material was consumed. Concentrate under reduced pressure to give a residue. Without further purification. Compound 22-3A (9.20 g, crude) was obtained as a brown oil.

### Step 3. Preparation of Compound 22-4A

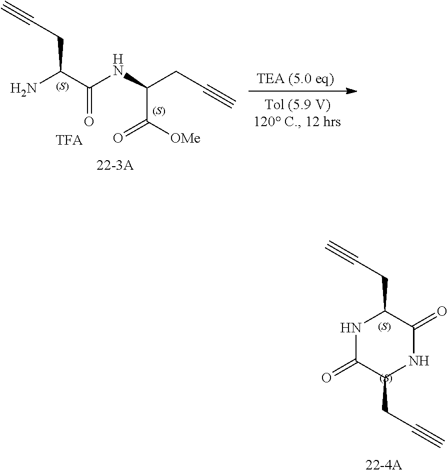

Dissolved Compound 22-3A (9.20 g, 43.8 mmol, 1.00 eq) in Tol. (54.0 mL), degassed and purged the mixture with N₂ for 3 times. Added TEA (22.2 g, 219 mmol, 30.5 mL, 5.00 eq) to the mixture. Stirred the mixture at 120° C. for 12 hrs. TLC (Dichloromethane:Methanol=5:1, the material Rr=0.31, Dichloromethane:Methanol=20:1 the product rf=0.33) indicated the material was consumed. The crude product was concentrated under reduced pressure to give a residue. The crude product was triturated with H₂O at 20° C. for 30 min. Without further purification. Compound 22-4A (3.00 g, 15.8 mmol, 36.0% yield) was obtained as a white solid.

¹H NMR: (400 MHz, DMSO-d₆)

δ ppm 8.25 (s, 2H), 4.05 (s, 2H), 2.85 (s, 2H), 2.65-2.75 (m, 2H), 2.55 (br s, 2H).

### Step 4. Preparation of Compound 22-5

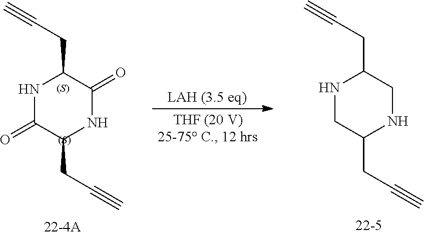

Dissolve Compound 22-4A (2.00 g, 11.0 mmol, 1.00 eq) in THE (40.0 mL). Add LAH (1.40 g, 36.8 mmol, 3.50 eq) into the reaction at 25° C. Stir the reaction at 75° C. for 12 hrs. TLC (Dichloromethane:Methanol=5:1, the material rf=0.63) indicated the material was remained. Add 1.40 mL of H₂O into the reaction at 0° C. and pour 1.40 mL of 15% NaOH aq, 4.20 mL of H₂O into the mixture. Add anhydrous sodium sulfate into the reaction. Filter and concentrated under reduced pressure to give a residue. Without further purification. Compound 22-5 (2.75 g, crude) was obtained as a brown solid.

### Step 5. Preparation of Compound 22-6

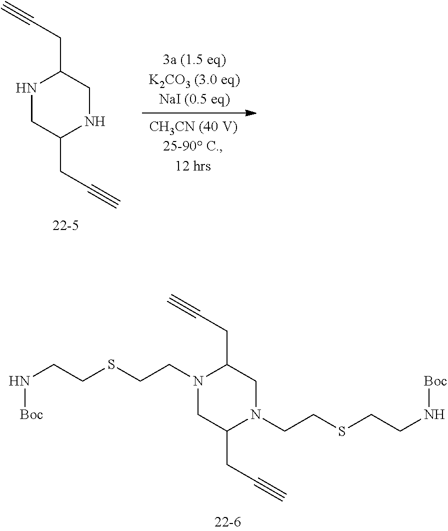 [SMILES: C#CCC1CNC(CC#C)CN1 ; C#CCC1CN(CCSCCNC(=O)OC(C)(C)C)C(CC#C)CN1CCSCCNC(=O)OC(C)(C)C]

Dissolve Compound 22-5 (1.00 g, 6.16 mmol, 1.00 eq) in MeCN (40.0 mL) and add K₂CO₃ (2.56 g, 18.5 mmol, 3.00 eq), NaI (462 mg, 3.08 mmol, 0.50 eq) into the mixture at 20° C. Add Compound 3a (2.22 g, 9.25 mmol, 1.50 eq) into the mixture at 20° C. Stir the reaction at 90° C. for 12 hrs. TLC (Petroleum ether:Ethyl acetate=0:1, the product rf=0.52) indicated the material was consumed. Filter and concentrated under reduced pressure to give a residue. The residue was purified by column chromatography (SiO₂, Petroleum ether/Ethyl acetate=1/0 to 2/1). Compound 22-6 (0.99 g, 1.74 mmol, 28.2% yield) was obtained as a brown oil.

¹H NMR: (400 MHz, CDCl₃) δ ppm 4.97 (s, 1H), 4.14-4.14 (m, 1H), 3.32 (s, 4H), 2.63-2.68 (m, 20H), 2.01-2.05 (m, 3H), 1.46 (s, 18H), 1.27 (t, J=7.20 Hz, 2H). Step 6.

### Step 6. Preparation of Compound 22-7

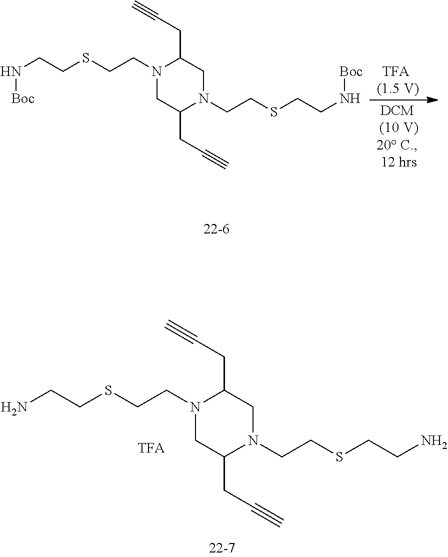 [SMILES: C#CCC1CN(CCSCCNC(=O)OC(C)(C)C)C(CC#C)CN1CCSCCNC(=O)OC(C)(C)C ; C#CCC1CN(CCSCCN)C(CC#C)CN1CCSCCN]

Dissolve Compound 22-6 (0.30 g, 527 μmol, 1.00 eq) with DCM (3.00 mL). Add TFA (693 mg, 6.08 mmol, 0.45 mL, 11.5 eq) into the mixture at 20° C. Stir the mixture for 12 hrs. TLC (Petroleum ether:Ethyl acetate=0:1, the material rf=0.42, the product rf=0.00) indicated the material was consumed. Concentrate under reduced pressure to give a residue. Add resin (5.00 g) to adjust pH=7-9. Without further purification. Compound 22-7 (0.168 g, 455 μmol, 86.4% yield) was obtained as a brown oil.

### Step 7. Preparation of Compound SIL2-22

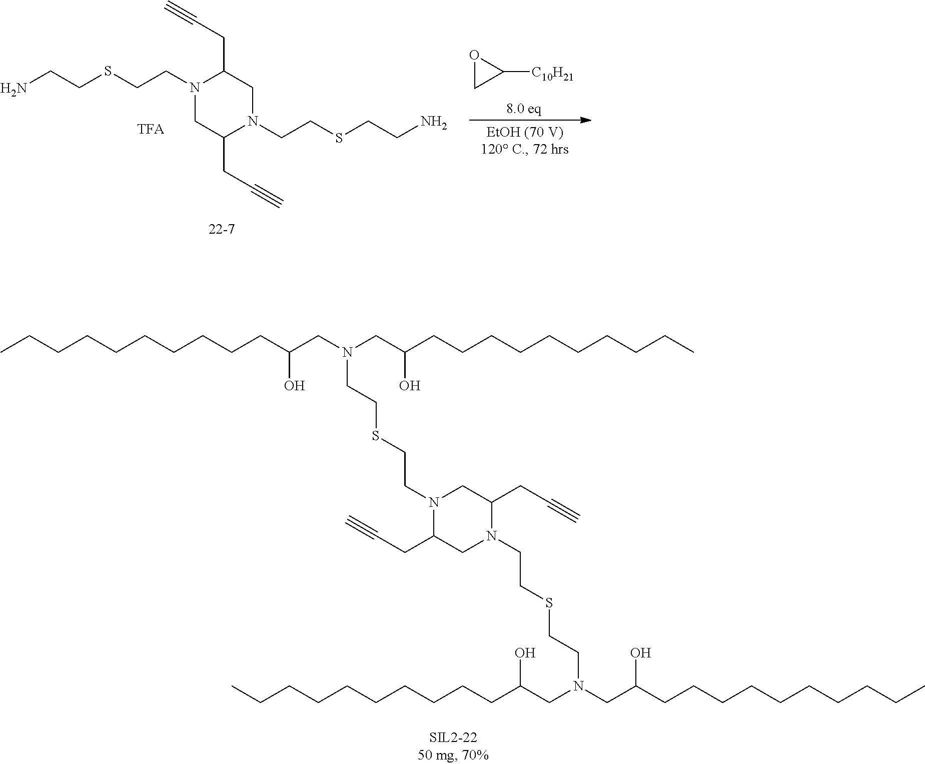

Dissolve Compound 22-7 (0.168 g, 455 μmol, 1.00 eq) with EtOH (40.0 mL). Add 2-decyloxirane (672 mg, 3.65 mmol, 8.00 eq) into the mixture. Stir the mixture at 120° C. for 72 hrs. TLC (Dichloromethane:Methanol=10:1, the product rf=0.49) indicated the material was consumed. Concentrate under reduced pressure to give a residue. The residue was purified by column chromatography (SiO₂, Petroleum ether/Ethyl acetate=10/1 to 0/1). Compound SIL2-22 (0.05 g, 28.9 μmol, 6.35% yield) was obtained as a brown oil.

¹H NMR: (400 MHz, CDCl₃)

δ ppm 6.62 (br s, 1H), 6.05 (t, J=2.80 Hz, 1H), 5.87 (s, 1H), 4.02 (t, J=7.20 Hz, 3H), 3.60-3.64 (m, 8H), 2.98-3.16 (m, 2H), 2.26 (s, 18H), 1.94-1.97 (m, 1H), 1.21-1.27 (m, 77H), 0.89 (t, J=6.80 Hz, 12H).

LCMS: (MS/2+H=553, RT=3.275 min)

### Synthesis of Compounds of Formula (II)

### Example 1. Synthesis of Compound SIL3-1

Step 1: preparation of Compound A2-1

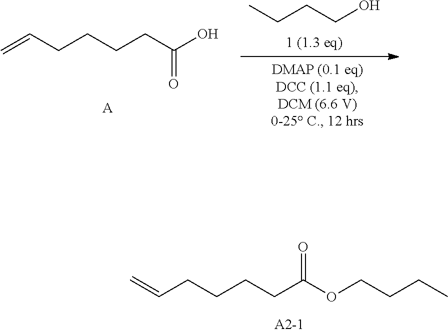

To a solution of Compound A (1.50 g, 11.7 mmol, 1.00 eq) and compound 1 (1.30 g, 17.6 mmol, 1.61 mL, 1.50 eq) in DCM (10 mL) was added DMAP (143 mg, 1.17 mmol, 0.10 eq) and DCC (2.66 g, 12.9 mmol, 2.60 mL, 1.10 eq) at 0° C. The mixture was stirred at 25° C. for 12 hrs. TLC (Petroleum ether:Ethyl acetate=10:1, R_{f}=0.43) indicated ˜5% of Compound A remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound A2-1 (2.00 g, 10.9 mmol, 92.7% yield) was obtained as colorless oil.

¹H NMR: 400 MHz CDCl₃

δ 5.75-5.84 (m, 1H), 4.92-5.09 (m, 2H), 4.08 (t, J=6.8 Hz, 2H), 2.31 (t, J=7.6 Hz, 2H), 2.01-2.14 (m, 2H), 1.57-1.69 (m, 5H), 1.31-1.50 (m, 4H), 0.94 (t, J=7.6 Hz, 3H).

Step 2: preparation of Compound A2-2

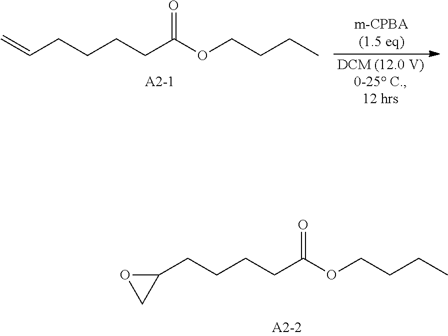

To a solution of Compound A2-1 (2.50 g, 13.6 mmol, 1.00 eq) in DCM (30 mL) was added in-CPBA (4.39 g, 20.4 mmol, 80% purity, 1.50 eq) at 0° C. The mixture was stirred at 25° C. for 12 hrs. TLC (Petroleum ether:Ethyl acetate=10:1) indicated Compound A2-1 was consumed completely. The reaction mixture was filtered. The filtrate was quenched by addition NaHSO₃ (150 mL), and then extracted with petroleum ether (100 mL). The combined organic layers were washed with Na₂CO₃ (50 mL). The combined organic layers were washed with brine (50 mL), dried over (Na₂SO4), filtered and concentrated under reduced pressure to give a residue. The crude product was dissolved by petroleum ether. The solid was precipitated at −10° C., filtered and the filtrate was concentrated under reduced pressure. Compound A2-2 (2.60 g, 13.0 mmol, 95.7% yield) was obtained as colorless oil.

¹H NMR: 400 MHz CDCl₃

δ 4.08 (t, J=6.8 Hz, 2H), 2.86-2.97 (m, 1H), 2.76 (t, J=4.4 Hz, 1H), 2.48 (dd, J=4.8, 2.8 Hz, 1H), 2.29-2.36 (m, 2H), 1.62-1.74 (m, 3H), 1.46-1.60 (m, 5H), 1.33-1.44 (m, 2H), 0.94 (t, J=7.2 Hz, 3H).

### Step 3. Preparation of Compound SIL3-1

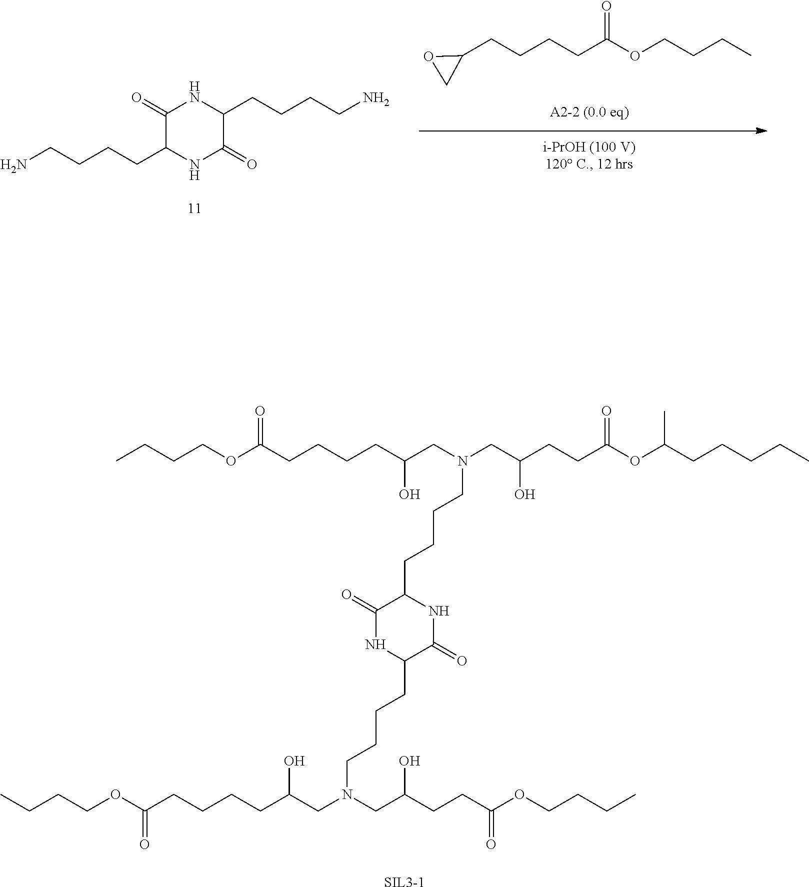 [SMILES: CCCCOC(=O)CCCCC1CO1 ; NCCCCC1NC(=O)C(CCCCN)NC1=O ; "A2-2 (0.0 eq)" ; CCCCCC(C)OC(=O)CCC(O)CN(CCCCC1NC(=O)C(CCCCN(CC(O)CCCCC(=O)OCCCC)CC(O)CCC(=O)OCCCC)NC1=O)CC(O)CCCCC(=O)OCCCC ; "SIL3-1"]

To a solution of Compound 11 (150 mg, 585 μmol, 1.00 eq) in i-PrOH (15 mL) was added Compound A2-2 (703 mg, 3.51 mmol, 6.00 eq). The mixture was stirred at 120° C. for 12 hrs. TLC (Dichloromethane:Methanol=8:1, product R_{f}=0.31). The mixture was concentrated under reduced pressure to give a residue. The residue was purified by column chromatography (SiO₂, Dichloromethane:Methanol=100:1 to 40:1) to obtain Compound SIL3-1 (70.0 mg, 66.2 μmol, 11.3% yield, 100% purity) as yellow oil.

¹H NMR: 400 MHz CDCl₃

δ 3.08-4.08 (m, 13H), 2.86-3.07 (m, 12H), 2.24 (t, J=7.3 Hz, 9H), 1.59-1.91 (m, 4H), 1.26-1.41 (m, 53H), 1.26 (br s, 2H), 0.93 (t, J=7.3 Hz, 12H)

### Example 2. Synthesis of Compound SIL3-2

### Step 1. Preparation of Compound A4-1

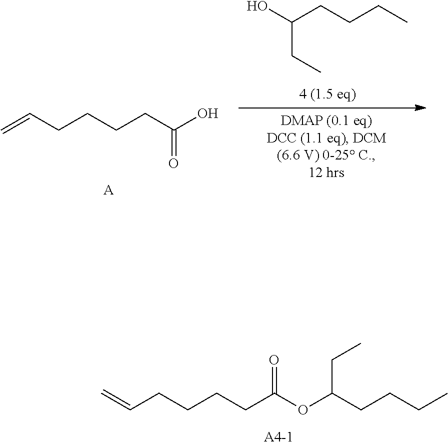

To a solution of Compound A (1.50 g, 11.7 mmol, 1.00 eq) and Compound 4 (2.04 g, 17.6 mmol, 3.21 mL, 1.50 eq) in DCM (10 mL) was added DMAP (1423 mg, 1.17 mmol, 0.10 eq) and DCC (2.66 g, 12.9 mmol, 2.60 mL, 1.10 eq) at 0° C. The mixture was stirred at 25° C. for 12 hrs. TLC (Petroleum ether:Ethyl acetate=10:1, R_{f}=0.43) indicated ˜5% of Compound A remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound A4-1 (2.30 g, 10.2 mmol, 86.8% yield) was obtained as a colorless oil.

¹H NMR: 400 MHz CDCl₃

δ 5.75-5.86 (m, 1H), 4.94-5.04 (m, 2H), 4.80-4.84 (m, 1H), 2.31 (t, J=7.6 Hz, 2H), 2.03-2.10 (m, 2H), 1.37-1.70 (m, 8H), 1.20-1.35 (m, 4H), 0.80-0.95 (m, 6H).

### Step 2. Preparation of Compound A4-2

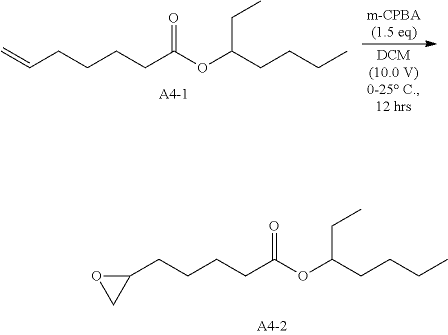 [SMILES: C=CCCCCC(=O)OC(CC)CCCC ; CCCCC(CC)OC(=O)CCCCC1CO1]

To a solution of Compound A4-1 (3.00 g, 13.3 mmol, 1.00 eq) in DCM (30 mL) was added m-CPBA (4.29 g, 19.9 mmol, 80% purity, 1.50 eq) at 0° C. The mixture was stirred at 25° C. for 12 hrs. TLC (Petroleum ether:Ethyl acetate=10:1) indicated Compound A4-1 was consumed completely. The reaction mixture was filtered. The filtrate was quenched by addition NaHSO₃ (150 mL), and then extracted with petroleum ether (100 mL). The combined organic layers were washed with Na₂CO₃ (50 mL). The combined organic layers were washed with brine (50 mL), dried over (Na₂SO4), filtered and concentrated under reduced pressure to give a residue. The crude product was dissolved by petroleum ether. The solid was precipitated at −10° C., filtered and the filtrate was concentrated under reduced pressure. Compound A4-2 (3.00 g, 12.4 mmol, 93.4% yield) was obtained as colorless oil.

¹H NMR: 400 MHz CDCl₃

δ 4.82-4.88 (m, 1H), 2.87-3.02 (m, 1H), 2.78 (t, J=4.8 Hz, 1H), 2.50 (dd, J=5.2, 2.8 Hz, 1H), 2.35 (t, J=7.2 Hz, 1H), 1.67-1.80 (m, 2H), 1.48-1.66 (m, 8H), 1.22-1.41 (m, 4H), 0.84-0.96 (m, 6H).

### Step 3. Preparation of Compound SIL3-2

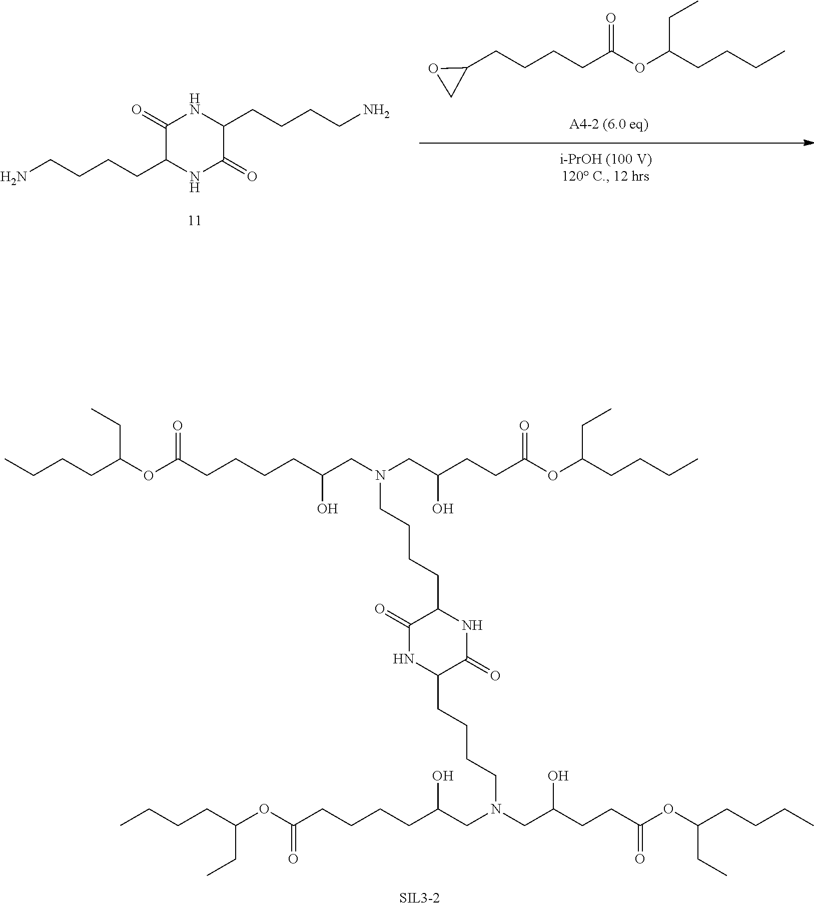 [SMILES: CCCCC(CC)OC(=O)CCCCC1CO1 ; NCCCCC1NC(=O)C(CCCCN)NC1=O ; "A4-2 (6.0 eq)" ; CCCCC(CC)OC(=O)CCCCC(O)CN(CCCCC1NC(=O)C(CCCCN(CC(O)CCCCC(=O)OC(CC)CCCC)CC(O)CCC(=O)OC(CC)CCCC)NC1=O)CC(O)CCC(=O)OC(CC)CCCC ; "SIL3-2"]

To a solution of compound 11 (100 mg, 390 μmol, 1.00 eq) in i-PrOH (10 mL) was compound A4-2 (567 mg, 2.34 mmol, 6.00 eq). The mixture was stirred at 120° C. for 12 hrs. TLC (methanol:dichloromethane=1:8) showed one main spot (R_{f}=0.15) was formed. The two mixtures were concentrated in vacuum to give the product. The residue was purified by column chromatography (SiO₂, dichloromethane:methanol=10/1 to 2/1). Compound SIL3-2 (50 mg, 40.8 μmol, 5.23% yield) was obtained as yellow oil.

¹H NMR: 400 MHz CDCl₃

δ 4.73-4.81 (m, 4H), 3.95-4.03 (m, 2H), 3.72-3.82 (m, 4H), 2.69-2.95 (m, 8H), 2.28-2.34 (m, 2H), 1.89-1.92 (m, 5H), 1.20-1.67 (m, 70H), 0.82-0.91 (m, 24H).

### Example 3. Synthesis of Compound SIL3-3

### Step 1. Preparation of Compound A5-1

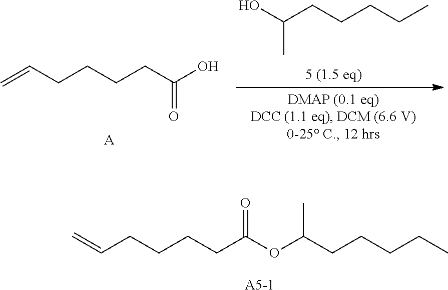

To a solution of Compound 5 (2.04 g, 17.6 mmol, 3.21 mL, 1.50 eq) and Compound A (1.50 g, 11.7 mmol, 1.00 eq) in DCM (10 mL) was added DMAP (143 mg, 1.17 mmol, 0.10 eq) and DCC (2.66 g, 12.9 mmol, 2.60 mL, 1.10 eq) at 0° C. The mixture was stirred at 25° C. for 12 hrs. TLC (Petroleum ether:Ethyl acetate=10:1, R_{f}=0.43) indicated ˜5% of Compound A remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound A5-1 (2.20 g, 9.72 mmol, 83.1% yield) was obtained as colorless oil.

¹H NMR: 400 MHz CDCl₃

δ 5.76-5.84 (m, 1H), 4.89-5.04 (m, 1H), 2.28 (t, J=7.6 Hz, 2H), 2.01-2.06 (m, 2H), 1.53-1.70 (m, 3H), 1.38-1.52 (m, 1H), 1.25-1.37 (m, 6H), 1.20 (d, J=6.0 Hz, 3H), 0.86-0.92 (m, 3H).

### Step 2. Preparation of Compound A5-2

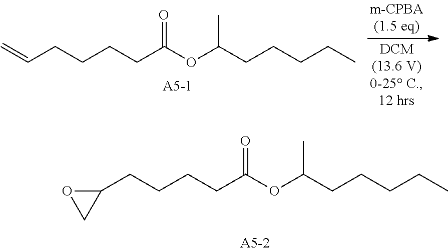

To a solution of Compound A5-1 (2.20 g, 9.72 mmol, 1.00 eq) in DCM (30 mL) was added m-CPBA (3.14 g, 14.58 mmol, 80% purity, 1.50 eq) at 0° C. The mixture was stirred at 25° C. for 12 hrs. TLC (Petroleum ether:Ethyl acetate=10:1) indicated Compound A5-1 was consumed completely. The reaction mixture was filtered. The filtrate was quenched by addition NaHSO₃ (150 mL), and then extracted with petroleum ether (100 mL). The combined organic layers were washed with Na₂CO₃ (50 mL). The combined organic layers were washed with brine (50 mL), dried over Na₂SO₄, filtered and concentrated under reduced pressure to give a residue. The crude product was dissolved by petroleum ether. The solid was precipitated at −10° C., filtered and the filtrate was concentrated under reduced pressure. Compound A5-2 (2.00 g, 8.25 mmol, 84.9% yield) was obtained as colorless oil (HNMR: ET40436-15-P1A1).

¹H NMR: 400 MHz CDCl₃

δ 4.86-4.95 (m, 1H), 2.86-2.97 (m, 1H), 2.76 (t, J=5.2 Hz, 1H), 2.47 (dd, J=4.8, 2.4 Hz, 1H), 2.30 (t, J=7.2 Hz, 2H), 1.64-1.75 (m, 2H), 1.41-1.60 (m, 6H), 1.24-1.36 (m, 6H), 1.20 (d, J=6.0 Hz, 3H), 0.81-0.95 (m, 3H).

### Step 3. Preparation of Compound SIL3-3

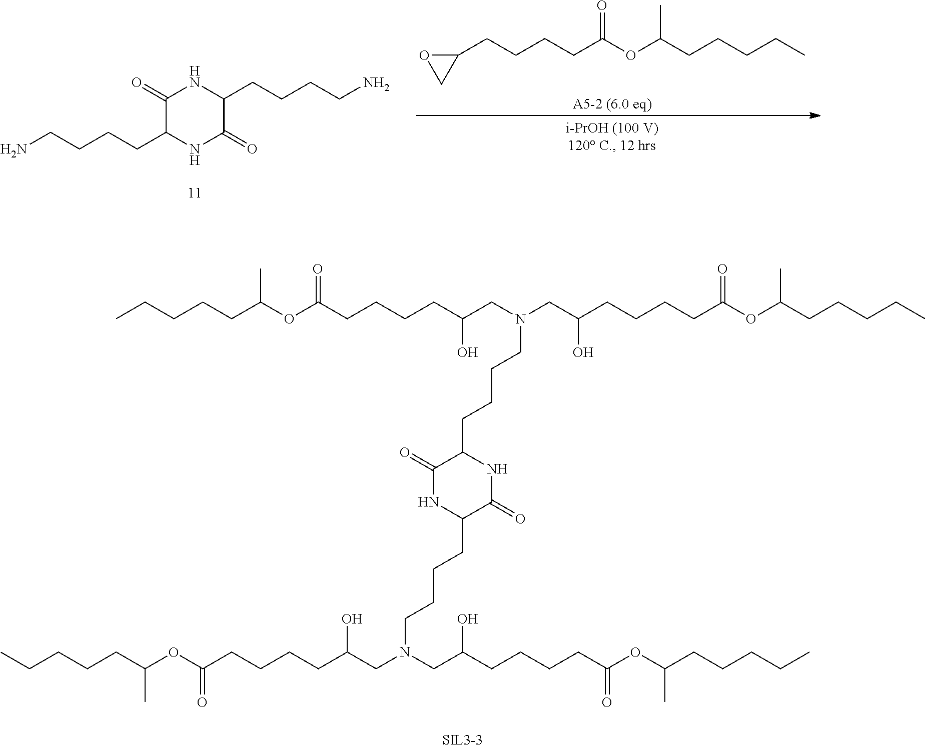 [SMILES: NCCCCC1NC(=O)C(CCCCN)NC1=O ; CCCCCC(C)OC(=O)CCCCC1CO1 ; "A5-2 (6.0 eq)" ; CCCCCC(C)OC(=O)CCCCC(O)CN(CCCCC1NC(=O)C(CCCCN(CC(O)CCCCC(=O)OC(C)CCCCC)CC(O)CCCCC(=O)OC(C)CCCCC)NC1=O)CC(O)CCCCC(=O)OC(C)CCCCC ; "SIL3-3"]

To a solution of Compound 11 (150 mg, 585 μmol, 1.00 eq) in i-PrOH (15 mL) was added Compound A5-2 (851 mg, 3.51 mmol, 6.00 eq). The mixture was stirred at 120° C. for 12 hrs. TLC (Dichloromethane:Methanol=10:1 product R_{f}=0.33). LCMS (ET42086-10-P1A1), product Rt=0.697 min. The mixture was evaporated to dryness. The residue was purified by column chromatography (SiO₂, Dichloromethane:Methanol=100:1 to 40:1). Compound SIL3-3 (60 mg, 46.45 μmol, 3.97% yield, 94.9%, purity) was obtained as yellow oil.

¹H NMR: 400 MHz CDCl₃

δ 4.85-4.92 (m, 4H), 4.04-4.07 (m, 2H), 3.62-3.70 (m, 4H), 2.27-2.70 (m, 19H), 1.65-2.00 (m, 4H), 1.25-1.65 (m, 63H), 1.20-1.28 (m, 12H), 0.87-0.90 (m, 12H).

### Example 4. Synthesis of Compound SIL3-4

### Step 1. Preparation of Compound A6-1

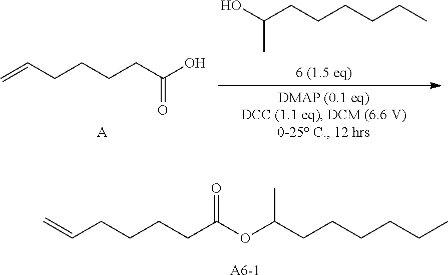

To a solution of Compound A (1.50 g, 11.7 mmol, 1.00 eq) and Compound 6 (2.29 g, 17.6 mmol, 3.21 mL, 1.50 eq) in DCM (10 mL) was added DMAP (143 mg, 1.17 mmol, 0.10 eq) and DCC (2.66 g, 12.9 mmol, 2.60 mL, 1.10 eq) at 0° C. The mixture was stirred at 25° C. for 12 hrs. TLC (Petroleum ether:Ethyl acetate=10:1, R_{f}=0.43) indicated ˜5% of Compound A remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound A6-1 (1.20 g, 4.99 mmol, 42.7% yield) was obtained as colorless oil.

¹H NMR: 400 MHz CDCl₃

δ 5.76-5.85 (m, 1H), 4.82-5.07 (m, 3H), 2.29 (t, J=7.6 Hz, 2H), 2.06-2.12 (m, 2H), 1.61-1.69 (m, 2H), 1.38-1.52 (m, 3H), 1.24-1.34 (m, 9H), 1.20 (d, J=6.4 Hz, 3H), 0.84-0.93 (m, 2H), 0.84-0.93 (m, 1H).

### Step 2. Preparation of Compound A6-2

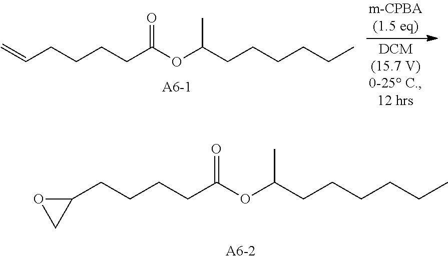

To a solution of Compound A6-1 (1.90 g, 7.90 mmol, 1.00 eq) in DCM (30 mL) was added m-CPBA (2.56 g, 11.86 mmol, 80% purity, 1.50 eq) at 0° C. The mixture was stirred at 25° C. for 12 hrs. TLC (Petroleum ether:Ethyl acetate=10:1) indicated Compound A6-1 was consumed completely. The reaction mixture was filtered. The filtrate was quenched by addition NaHSO₃ (150 mL), and then extracted with petroleum ether (100 mL). The combined organic layers were washed with Na₂CO₃ (50 mL). The combined organic layers were washed with brine (50 mL), dried over (Na₂SO4), filtered and concentrated under reduced pressure to give a residue. The crude product was dissolved by petroleum ether. The solid was precipitated at −10° C., filtered and the filtrate was concentrated under reduced pressure. Compound A6-2 (2.00 g, 7.80 mmol, 98.7% yield) was obtained as colorless oil. (¹H NMR: ET40436-16-P1A1).

¹H NMR: 400 MHz CDCl₃

δ 4.86-4.95 (m, 1H), 2.88-2.95 (m, 1H), 2.76 (t, J=4.8 Hz, 1H), 2.47 (dd, J=5.2, 2.8 Hz, 1H), 2.26-2.35 (m, 2H), 1.41-1.75 (m, 8H), 1.28 (br s, 7H), 1.20 (d, J=6.0 Hz, 3H), 0.88 (br t, J=6.4 Hz, 3H).

### Step 3. Preparation of Compound SIL3-4

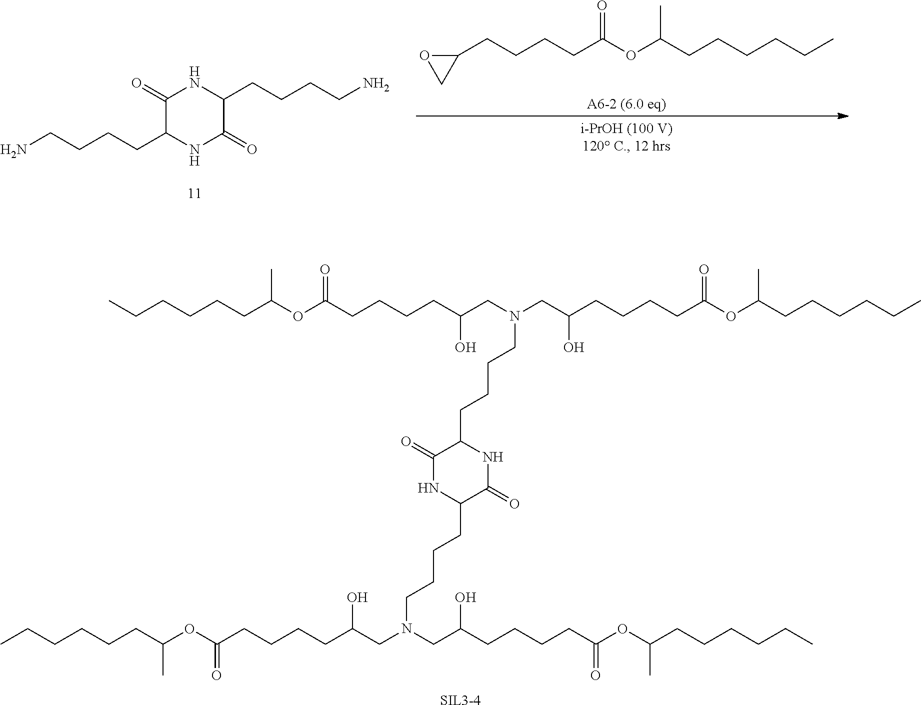 [SMILES: NCCCCC1NC(=O)C(CCCCN)NC1=O ; CCCCCCC(C)OC(=O)CCCCC1CO1 ; "A6-2 (6.0 eq)" ; CCCCCCC(C)OC(=O)CCCCC(O)CN(CCCCC1NC(=O)C(CCCCN(CC(O)CCCCC(=O)OC(C)CCCCCC)CC(O)CCCCC(=O)OC(C)CCCCCC)NC1=O)CC(O)CCCCC(=O)OC(C)CCCCCC ; "SIL3-4"]

To a solution of Compound 11 (150 mg, 585 μmol, 1.00 eq) in i-PrOH (15 mL) was added Compound A6-2 (900 mg, 3.51 mmol, 6.00 eq). The mixture was stirred at 120° C. for 12 hrs. LCMS (ET42086-5-P1A1, product: Rₜ=0.748 min). TLC (Dichloromethane:Methanol=8:1, product R_{f}=0.31). The mixture was concentrated in vacuo. The residue was purified by column chromatography (SiO₂, Dichloromethane:Methanol=100:1 to 8:1). Compound SIL3-4 (70.0 mg, 52.8 μmol, 9.02% yield, 96.7%, purity) was obtained as yellow oil.

¹H NMR: 400 MHz CDCl₃

δ 7.27-7.73 (m, 2H), 4.86-4.93 (m, 4H), 4.05-4.08 (m, 2H), 3.72-3.80 (m, 4H), 2.26-2.73 (m, 19H), 2.27-2.30 (m, 8H), 1.65-2.00 (m, 4H), 1.28-1.63 (m, 73H), 1.20-1.28 (m, 12H), 1.19-1.20 (m, 2H), 0.87-0.90 (m, 12H)

### Example 5. Synthesis of Compound SIL3-5

### Step 1. Preparation of Compound A7-1

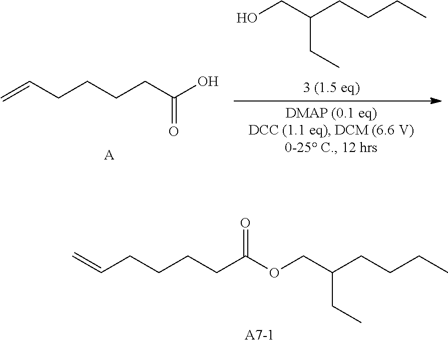

To a solution of Compound A (1.50 g, 11.7 mmol, 1.00 eq) and Compound 3 (2.29 g, 17.6 mmol, 3.21 mL, 1.50 eq) in DCM (10 mL) was added DMAP (143 mg, 1.17 mmol, 0.10 eq) and DCC (2.66 g, 12.9 mmol, 2.60 mL, 1.10 eq) at 0° C. The mixture was stirred at 25° C. for 12 hrs. TLC (Petroleum ether:Ethyl acetate=10:1, R_{f}=0.43) indicated ˜5% of Compound A remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound A7-1 (2.40 g, 9.98 mmol, 85.3% yield) was obtained as colorless oil.

¹H NMR: 400 MHz CDCl₃

δ 5.75-5.84 (m, 1H), 4.91-5.10 (m, 2H), 3.95-4.03 (m, 2H), 2.32 (t, J=7.6 Hz, 2H), 2.03-2.13 (m, 2H), 1.60-1.72 (m, 2H), 1.52-1.57 (m, 1H), 1.22-1.49 (m, 10H), 0.82-0.96 (m, 6H).

### Step 2. Preparation of Compound A7-2

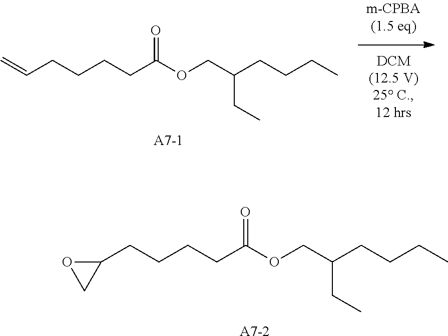 [SMILES: C=CCCCCC(=O)OCC(CC)CCCC ; "A7-1" ; CCCCC(CC)COC(=O)CCCCC1CO1 ; "A7-2"]

To a solution of Compound A7-1 (2.40 g, 9.98 mmol, 1.00 eq) in DCM (30 mL) was added m-CPBA (3.23 g, 14.9 mmol, 80% purity, 1.50 eq) at 0° C. The mixture was stirred at 25° C. for 12 hrs. TLC (Petroleum ether:Ethyl acetate=10:1) indicated Compound A7-1 was consumed completely. The reaction mixture was filtered. The filtrate was quenched by addition NaHSO₃ (150 mL), and then extracted with petroleum ether (100 mL). The combined organic layers were washed with Na₂CO₃ (50 mL). The combined organic layers were washed with brine (50 mL), dried over (Na₂SO4), filtered and concentrated under reduced pressure to give a residue. The crude product was dissolved by petroleum ether. The solid was precipitated at −10° C., filtered and the filtrate was concentrated under reduced pressure. Compound A7-2 (2.50 g, 9.75 mmol, 97.7% yield) was obtained as colorless oil.

¹H NMR: 400 MHz CDCl₃

δ 3.89-4.06 (m, 2H), 2.88-2.97 (m, 1H), 2.76 (t, J=4.4 Hz, 1H), 2.48 (dd, J=5.2, 2.8 Hz, 1H), 2.30-2.39 (m, 2H), 1.66-1.73 (m, 2H), 1.47-1.63 (m, 6H), 1.23-1.40 (m, 8H), 0.81-0.96 (m, 6H).

### Step 3. Preparation of Compound SIL3-5

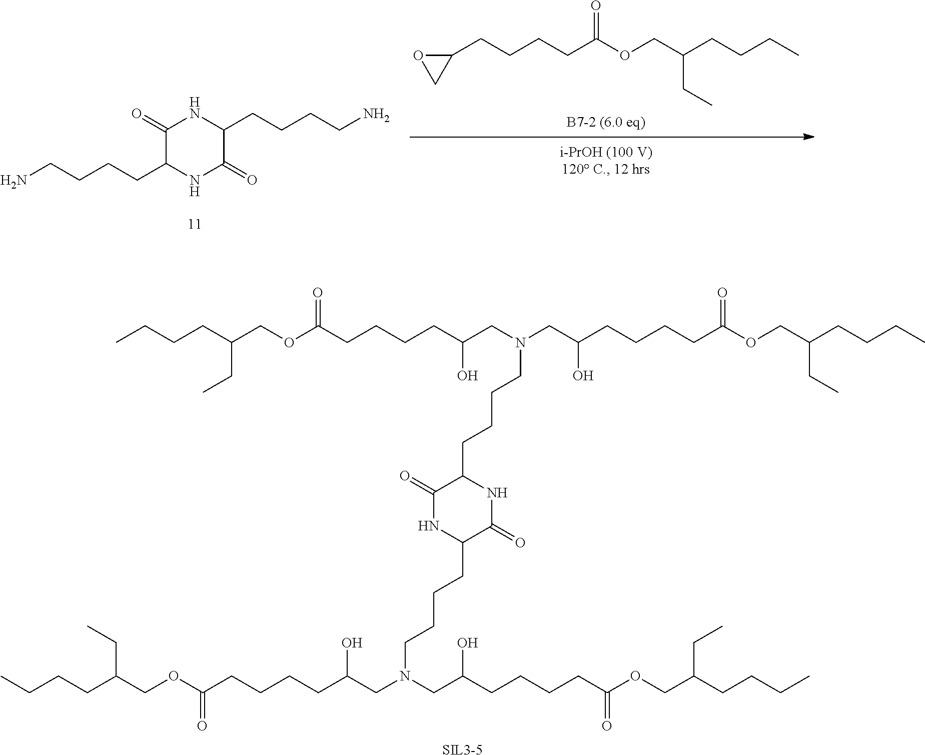 [SMILES: CCCCC(CC)COC(=O)CCCCC1CO1 ; NCCCCC1NC(=O)C(CCCCN)NC1=O ; "B7-2 (6.0 eq)" ; CCCCC(CC)COC(=O)CCCCC(O)CN(CCCCC1NC(=O)C(CCCCN(CC(O)CCCCC(=O)OCC(CC)CCCC)CC(O)CCCCC(=O)OCC(CC)CCCC)NC1=O)CC(O)CCCCC(=O)OCC(CC)CCCC ; "SIL3-5"]

A mixture of Compound 11 (100 mg, 390 μmol, 1.00 eq), Compound A7-2 (600 mg, 2.34 mmol, 6.00 eq) in i-PrOH (10 mL) was degassed and purged with N₂ for 3 times, and then the mixture was stirred at 120° C. for 12 hrs. TLC (methanol:dichloromethane=1:8) showed Compound 11 (R_{f}=0.02) was consumed and no one main spot (R_{f}=0.25) was formed. The two reactions were combined and concentrated in vacuum to give the product. The residue was purified by column chromatography (SiO₂, dichloromethane:methanol=1/0 to 10/1) Compound SIL3-5 (70.0 mg, 117 μmol, 15.0% yield) was obtained as yellow oil.

¹H NMR: 400 MHz CDCl₃

δ 4.03-4.13 (m, 2H), 3.90-4.01 (m, 7H), 3.69-3.89 (m, 3H), 2.50-2.94 (m, 8H), 2.26-2.37 (m, 8H), 1.76-2.00 (m, 3H), 1.16-1.74 (m, 66H), 0.82-0.96 (m, 24H)

### Example 6. Synthesis of Compound SIL3-12

### Step 1. Preparation of Compound B3-1

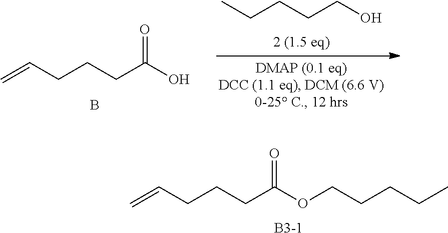

To a solution of Compound B (3.00 g, 26.3 mmol, 3.12 mL, 1.00 eq) and Compound 2 (3.47 g, 39.4 mmol, 4.28 mL, 1.50 eq) in DCM (20 mL) was added DMAP (321 mg, 2.63 mmol, 0.10 eq) and DCC (5.97 g, 28.9 mmol, 5.85 mL, 1.10 eq) at 0° C. The mixture was stirred at 25° C. for 12 hrs. TLC (Petroleum ether:Ethyl acetate=10:1, R_{f}=0.43) indicated ˜5% of Compound B remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound B3-1 (4.50 g, 24.4 mmol, 92.9% yield) was obtained as colorless oil.

### Step 2. Preparation of Compound B3-2

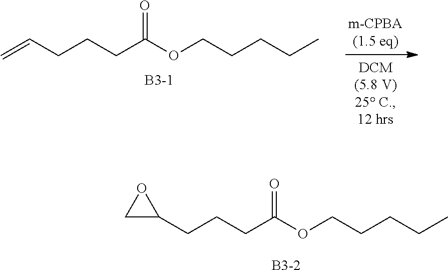

To a solution of Compound B3-1 (6.00 g, 32.6 mmol, 1.00 eq) in DCM (35 mL) was added m-CPBA (10.6 g, 48.8 mmol, 80% purity, 1.50 eq) at 0° C. The mixture was stirred at 25° C. for 12 hrs. TLC (Petroleum ether:Ethyl acetate=10:1) indicated Compound B3-1 was consumed completely. The reaction mixture was filtered. The filtrate was quenched by addition NaHSO₃ (300 mL), and then extracted with petroleum ether (200 mL). The combined organic layers were washed with Na₂CO₃ (100 mL). The combined organic layers were washed with brine (50 mL), dried over Na₂SO₄, filtered and concentrated under reduced pressure to give a residue. The crude product was dissolved by petroleum ether. The solid was precipitated at −10° C., filtered and the filtrate was concentrated under reduced pressure. Compound B3-2 (4.00 g, 20.0 mmol, 61.3% yield) was obtained as colorless oil.

### Step 3. Preparation of Compound SIL3-12

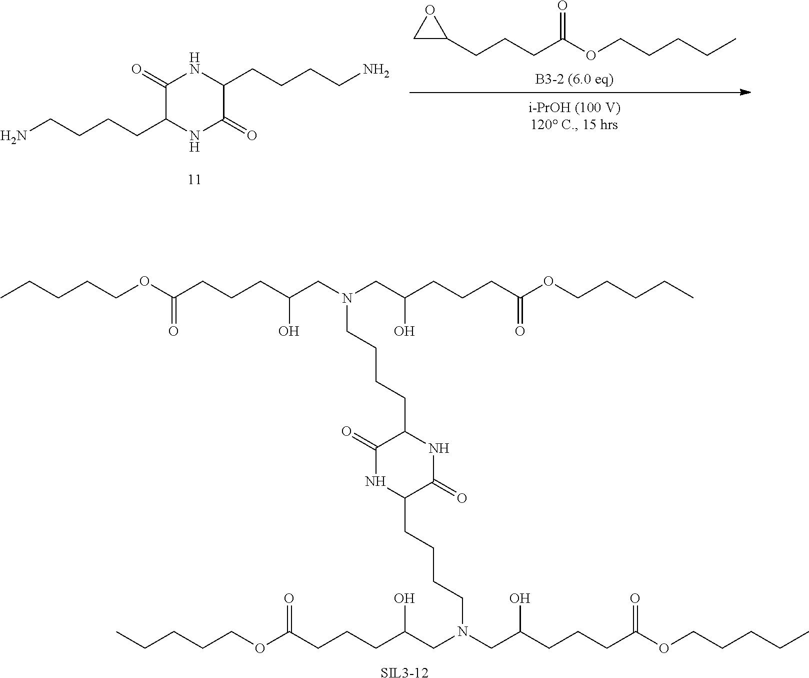

A mixture of Compound 11 (0.30 g, 1.17 mmol, 1.00 eq), Compound B3-2 (1.41 g, 7.02 mmol, 6.00 eq) in IPA (30 mL) was degassed and purged with N₂ for 3 times, and then the mixture was stirred at 120° C. for 15 hrs under N₂ atmosphere. TLC (dichloromethane:methanol=10:1, R_{f}=0.17) showed desired compound was detected. The reaction mixture was concentrated under reduced pressure to give a residue. The residue was purified by column chromatography (SiO₂, DCM:MeOH=30/1 to 10/1). The residue was purified by prep-HPLC (column: Phenomenex Luna C 18 100*30 mm*5 μm; mobile phase: [water (0.1% TFA)-ACN]; B %: 40%-70%, 10 min). The pH value was adjusted to 8 by aq. NaHCO₃, and extracted with DCM (10 mL). The combined organic layers were washed with brine (5 mL), filtered and concentrated under reduced pressure to give a residue. Compound SIL3-12 (0.04 g, 37.83 μmol, 3.23% yield) was obtained as yellow oil.

¹H NMR: 400 MHz CDCl₃

δ 6.47-6.71 (m, 2H), 4.06 (t, J=6.8 Hz, 9H), 4.00-4.17 (m, 1H), 3.68 (br s, 4H), 2.24-2.72 (m, 19H), 1.30-1.95 (m, 56H), 0.91 (br t, J=6.4 Hz, 12H).

### Example 7. Synthesis of Compound SIL3-6

### Step 1. Preparation of Compound B4-1

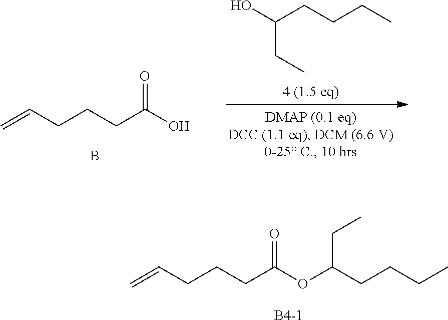

To a mixture of Compound B (2.50 g, 21.9 mmol, 2.60 mL, 1.00 eq), Compound 4 (3.82 g, 32.9 mmol, 1.50 eq) in DCM (25 mL) was added DMAP (268 mg, 2.19 mmol, 0.10 eq), then DCC (4.97 g, 24.1 mmol, 4.87 mL, 1.10 eq) was added at 0° C. After addition, the cooling bath was removed, the mixture was stirred at 25° C. for 10 hrs. TLC (Petroleum ether:Ethyl acetate=10:1, R_{f}=0.48) indicated Compound B was consumed completely and two new spots formed. The reaction mixture was filtered and the filtrate was concentrated under reduced pressure to give a residue. The residue was purified by column chromatography (SiO₂, Petroleum ether/Ethyl acetate=50/1). Compound B4-1 (3.50 g, 16.5 mmol, 75.3% yield) was obtained as colorless oil.

¹H NMR: 400 MHz CDCl₃

δ 5.76-5.84 (m, 1H), 4.98-5.06 (m, 2H), 4.80-4.87 (m, 1H), 2.32 (t, J=7.6 Hz, 2H), 2.05-2.20 (m, 2H), 1.70-1.85 (m, 2H), 1.48-1.64 (m, 11H), 1.20-1.38 (m, 4H), 0.82-0.95 (m, 6H).

### Step 2. Preparation of Compound B4-2

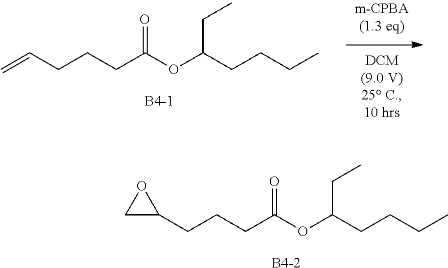

To a solution of Compound B4-1 (4.00 g, 18.8 mmol, 1.00 eq) in DCM (36 mL) was added m-CPBA (5.28 g, 24.5 mmol, 80% purity, 1.3 eq). The mixture was stirred at 25° C. for 10 hrs. TLC (Petroleum ether:Ethyl acetate=10:1) indicated Compound B4-1 was consumed completely and many new spots one new spot formed. ¹H NMR showed desired compound was detected. The mixture was filtered and filtrate was diluted with 10% w/v aqueous sodium thiosulfate solution (200 mL) and stirred vigorously for five minutes. The mixture was diluted with saturated aqueous sodium bicarbonate (300 mL), the organic layer separated and the aqueous layer extracted with chloroform (300 mL, 100 mL). The pooled organic extracts were washed with saturated aqueous sodium bicarbonate (100 mL), brine (50 mL), dried over sodium sulfate and concentrated in vacuo. The crude product was purified by re-crystallization from Petroleum ether (50 mL) at −10° C. The mixture was mixture was filtered and filtrate was concentrated in vacuo. Compound B4-2 (3.20 g, 14.0 mmol, 74.4% yield) was obtained as colorless oil.

¹H NMR: 400 MHz CDCl₃

δ 4.81-4.88 (m, 1H), 2.94-2.96 (m, 1H), 2.78 (t, J=4.8 Hz, 1H), 2.50 (dd, J=7.6, 2.8 Hz, 1H), 2.33-2.43 (m, 2H), 1.75-1.93 (m, 2H), 1.47-1.69 (m, 6H), 1.21-1.41 (m, 4H), 0.80-0.98 (m, 6H).

### Step 3. Preparation of Compound SIL3-6

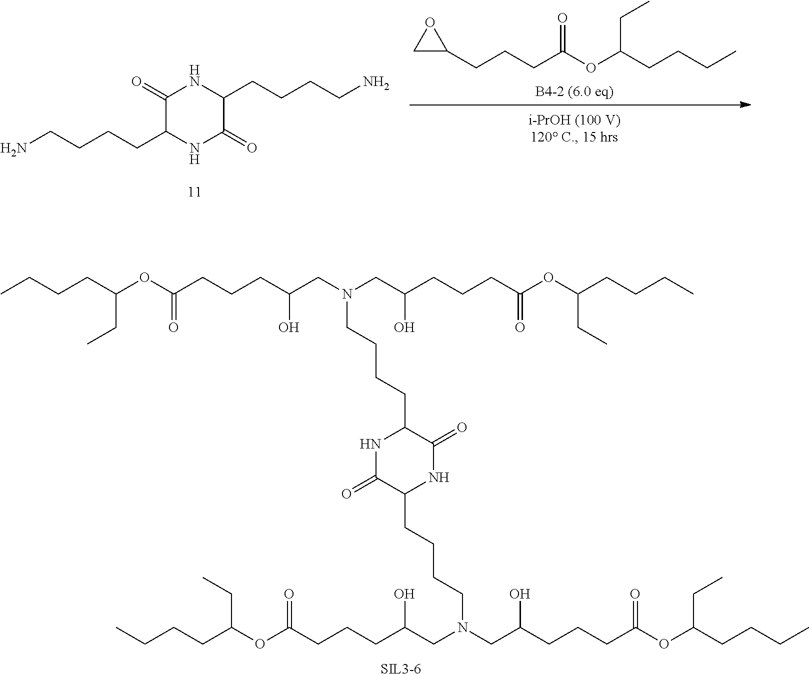

A mixture of Compound 11 (0.20 g, 780 μmol, 1.00 eq), Compound B4-2 (1.07 g, 4.68 mmol, 6.00 eq) in IPA (20 mL) was degassed and purged with N₂ for 3 times, and then the mixture was stirred at 120° C. for 15 hrs under N₂ atmosphere. LC-MS showed desired compound was detected. The reaction mixture concentrated under reduced pressure to give a residue. The residue was purified by column chromatography (SiO₂, DCM:MeOH=40:1 to 15:1, R_{f}=0.17). SIL3-6 (0.04 g, 34.20 μmol, 4.38% yield) was obtained as yellow oil.

¹H NMR: 400 MHz CDCl₃

δ 4.78-4.82 (m, 4H), 3.70-4.20 (m, 6H), 2.25-2.52 (m, 10H), 1.06-2.15 (m, 77H), 0.80-0.95 (m, 24H).

### Example 8. Synthesis of Compound SIL3-7

### Step 1. Preparation of Compound B5-1

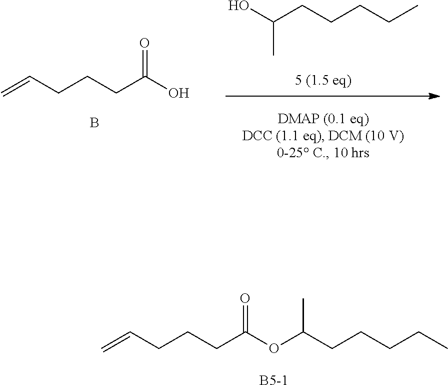

To a mixture of Compound B (2.50 g, 21.9 mmol, 2.60 mL, 1.00 eq), Compound 5 (3.82 g, 32.8 mmol, 1.50 eq) in DCM (25 mL) was added DMAP (268 mg, 2.19 mmol, 0.10 eq), then DCC (4.97 g, 24.1 mmol, 4.87 mL, 1.10 eq) was added at 0° C. After addition, the cooling bath was removed and the mixture was stirred at 25° C. for 10 hrs. TLC (Petroleum ether:Ethyl acetate=10:1, R_{f}=0.48) indicated Compound B was consumed completely and two new spots formed. The reaction mixture was filtered and the filtrate was concentrated under reduced pressure to give a residue. The residue was purified by column chromatography (SiO₂, Petroleum ether/Ethyl acetate=50/1). Compound B5-1 (4.50 g, 21.2 mmol, 96.8% yield) was obtained as colorless oil.

¹H NMR: 400 MHz CDCl₃

δ 5.71-5.93 (m, 1H), 4.97-5.11 (m, 2H), 4.83-4.96 (m, 1H), 2.30 (t, J=7.2 Hz, 2H), 2.10 (q, J=7.2 Hz, 2H), 1.68-1.84 (m, 2H), 1.53-1.62 (m, 1H), 1.41-1.52 (m, 1H), 1.26-1.39 (m, 1H), 1.26-1.39 (m, 6H), 1.21 (d, J=6.4 Hz, 3H), 0.89 (br t, J=6.4 Hz, 3H).

### Step 2. Preparation of Compound B5-2

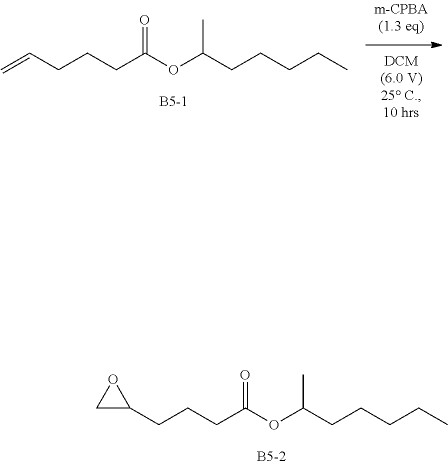

To a solution of Compound B5-1 (5.00 g, 23.6 mmol, 1.00 eq) in DCM (30 mL) was added m-CPBA (6.60 g, 30.6 mmol, 80% purity, 1.30 eq). The mixture was stirred at 25° C. for 10 hrs. TLC (Petroleum ether:Ethyl acetate=10:1) indicated Compound B5-1 was consumed completely and one new spot formed. ¹H NMR showed desired compound was detected. The mixture was filtered and filtrate was diluted with 10% w/v aqueous sodium thiosulfate solution (200 mL) and stirred vigorously for five minutes. The mixture was diluted with saturated aqueous sodium bicarbonate (300 mL), the organic layer separated and the aqueous layer extracted with chloroform (300 mL, 100 mL). The pooled organic extracts were washed with saturated aqueous sodium bicarbonate (100 mL), brine (50 mL), dried over sodium sulfate and concentrated in vacuo. The crude product was purified by re-crystallization from Petroleum ether (70 mL) at −10° C. The mixture was mixture was filtered and filtrate was concentrated in vacuo. Compound B5-2 (5.10 g, 22.3 mmol, 94.8% yield) was obtained as colorless oil.

¹H NMR: ET40296-15-P1A1 400 MHz CDCl₃

δ 4.85-5.00 (m, 1H), 2.90-2.99 (m, 1H), 2.78 (t, J=4.4 Hz, 1H), 2.50 (dd, J=5.2, 2.8 Hz, 1H), 2.33-2.41 (m, 2H), 1.73-1.94 (m, 2H), 1.55-1.71 (m, 3H), 1.42-1.54 (m, 1H), 1.26-1.40 (m, 6H), 1.22 (d, J=6.4 Hz, 3H), 0.88-0.93 (m, 3H).

### Step 3. General Procedure for Preparation of Compound SIL3-7

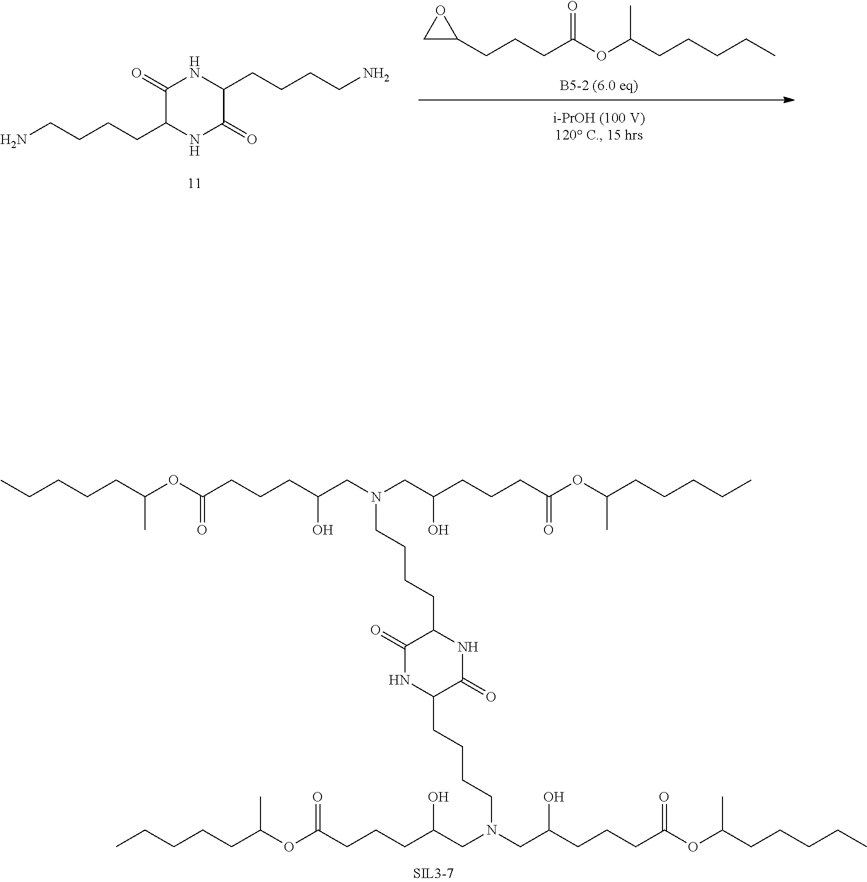

A mixture of Compound 11 (0.20 g, 780 μmol, 1.00 eq), Compound B5-2 (1.07 g, 4.68 mmol, 6.00 eq) in IPA (20 mL) was degassed and purged with N₂ for 3 times, and then the mixture was stirred at 120° C. for 15 hrs under N₂ atmosphere. LC-MS showed desired compound was detected. The reaction mixture concentrated under reduced pressure to give a residue. The residue was purified by column chromatography (SiO₂, DCM:MeOH=40:1 to 15:1, R_{f}=0.17). Compound SIL3-7 (0.05 g, 42.75 μmol, 5.48% yield) was obtained as yellow oil.

¹H NMR: 400 MHz CDCl₃

δ 4.77-5.03 (m, 4H), 4.07 (br s, 2H), 3.68-3.87 (m, 4H), 2.24-2.83 (m, 18H), 1.37-2.03 (m, 38H), 1.11-1.37 (m, 38H), 0.86-0.92 (m, 12H).

### Example 9. Synthesis of Compound SIL3-8

### Step 1. Preparation of Compound B6-1

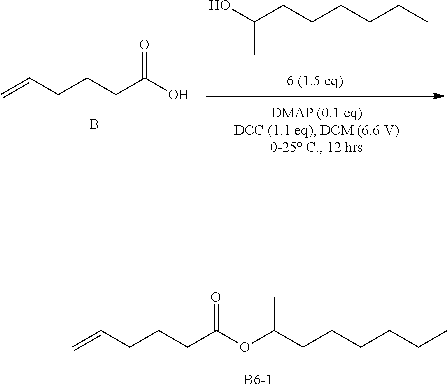

To a solution of Compound B (3.00 g, 26.3 mmol, 3.12 mL, 1.00 eq) and Compound 6 (5.13 g, 39.4 mmol, 4.28 mL, 1.50 eq) in DCM (20 mL) was added DMAP (321 mg, 2.63 mmol, 0.10 eq) and DCC (5.97 g, 28.9 mmol, 5.85 mL, 1.10 eq) at 0° C. The mixture was stirred at 25° C. for 12 hrs. TLC (Petroleum ether:Ethyl acetate=10:1, R_{f}=0.43) indicated ˜5% of Compound B remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound B6-1 (5.50 g, 24.3 mmol, 92.5% yield) was obtained as colorless oil.

### Step 2. Preparation of Compound B6-2

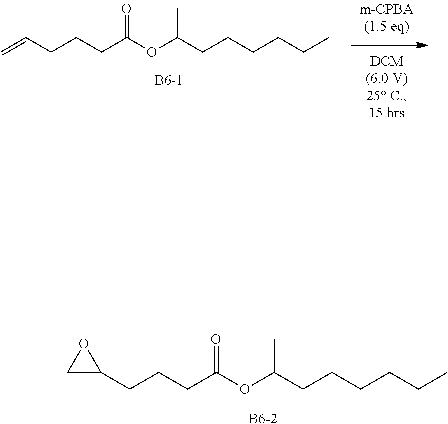

To a solution of Compound B6-1 (10.0 g, 44.2 mmol, 1.00 eq) in DCM (60 mL) was added m-CPBA (14.3 g, 66.3 mmol, 80% purity, 1.50 eq) at 0° C. The mixture was stirred at 25° C. for 15 hrs. TLC (Petroleum ether:Ethyl acetate=10:1) indicated Compound B6-1 was consumed completely and two new spots formed. The reaction mixture was filtered. The filtrate was quenched by addition NaHSO₃ (500 mL), and then extracted with petroleum ether (200 mL). The combined organic layers were washed with Na₂CO₃ (200 mL). The combined organic layers were washed with brine (50 mL), dried over (Na₂SO₄), filtered and concentrated under reduced pressure to give a residue. The crude product was dissolved by petroleum ether. The solid was precipitated at −10° C., filtered and the filtrate was concentrated under reduced pressure. Compound B6-2 (3.00 g, 12.4 mmol, 28.0% yield) was obtained as a colorless oil.

¹H NMR: 400 MHz CDCl₃

δ 4.87-4.95 (m, 1H), 2.87-2.98 (m, 1H), 2.76 (t, J=4.8 Hz, 1H), 2.48 (dd, J=4.8, 2.4 Hz, 1H), 2.26-2.42 (m, 2H), 1.71-1.90 (m, 2H), 1.55-1.63 (m, 2H), 1.42-1.52 (m, 2H), 1.17-1.37 (m, 11H), 0.89 (t, J=6.4 Hz, 3H).

### Step 3. Preparation of Compound 6

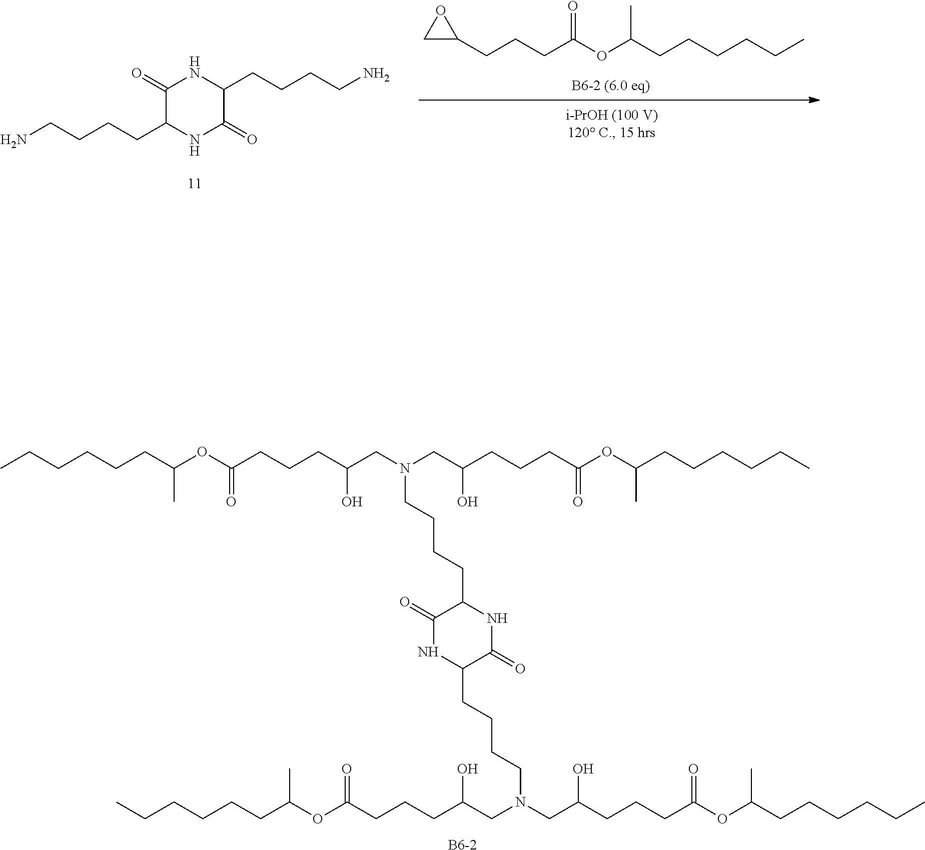

A mixture of Compound 11 (0.20 g, 780 μmol, 1.00 eq), Compound B6-2 (1.13 g, 4.68 mmol, 6.00 eq) in IPA (20 mL) was degassed and purged with N₂ for 3 times, and then the mixture was stirred at 120° C. for 15 hrs under N₂ atmosphere. LC-MS showed 46.6% of desired compound was detected. The reaction mixture was concentrated under reduced pressure to give a residue. The residue was purified by column chromatography (SiO₂, DCM:MeOH=30/1 to 10/1). The residue was purified by prep-HPLC (column: Phenomenex Luna C18 100*30 mm*5 um; mobile phase: [water (0.1% TFA)-ACN]; B %: 55%-85%, 10 min). The pH value was adjusted to 8 by aq. NaHCO₃, and extracted with DCM (10 mL). The combined organic layers were washed with brine (5 mL), filtered and concentrated under reduced pressure to give a residue. Compound SIL3-8 (0.045 g, 36.7 μmol, 15.00% yield) was obtained as yellow oil.

¹H NMR: 400 MHz CDCl₃

δ 6.56 (br s, 1H), 4.84-4.95 (m, 4H), 4.08 (br s, 2H), 3.74 (br s, 4H), 2.39-2.79 (m, 8H), 2.32 (br t, J=6.8 Hz, 8H), 1.38-2.05 (m, 34H), 1.16-1.35 (m, 50H), 0.86-0.91 (m, 12H).

### Example 10. Synthesis of Compound SIL3-9

### Step 1. Preparation of Compound B7-1

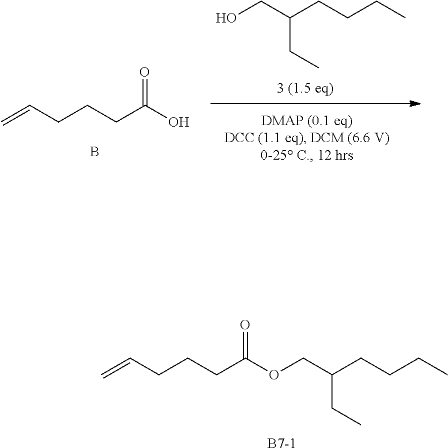

To a solution of Compound B (3.00 g, 26.3 mmol, 3.12 mL, 1.00 eq) and Compound 3 (5.13 g, 39.4 mmol, 1.50 eq) in DCM (20 mL) was added DMAP (321 mg, 2.63 mmol, 0.10 eq) and DCC (5.97 g, 28.9 mmol, 5.85 mL, 1.10 eq) at 0° C. The mixture was stirred at 25° C. for 12 hrs. TLC (Petroleum ether:Ethyl acetate=10:1, R_{f}=0.43) indicated ˜5% of Compound B remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound B7-1 (5.60 g, 24.7 mmol, 94.1% yield) was obtained as colorless oil.

### Step 2. Preparation of Compound B7-2

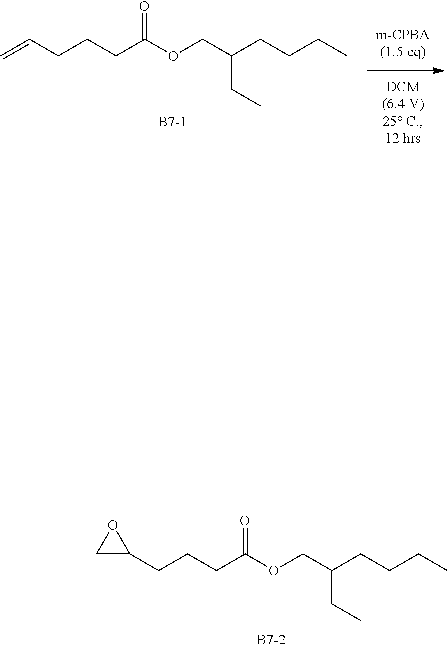

To a solution of Compound B7-1 (7.00 g, 30.9 mmol, 1.00 eq) in DCM (45 mL) was added m-CPBA (10.0 g, 46.4 mmol, 80% purity, 1.50 eq) at 0° C. The mixture was stirred at 25° C. for 12 hrs. TLC (Petroleum ether:Ethyl acetate=10:1) indicated Compound B7-1 was consumed completely. The reaction mixture was filtered. The filtrate was quenched by addition NaHSO₃ (300 mL), and then extracted with petroleum ether (200 mL). The combined organic layers were washed with Na₂CO₃ 100 mL. The combined organic layers were washed with brine 50 mL, dried over (Na₂SO₄), filtered and concentrated under reduced pressure to give a residue. The crude product was dissolved by petroleum ether. The solid was precipitated at −10° C., filtered and the filtrate was concentrated under reduced pressure. Compound B7-2 (4.00 g, 16.5 mmol, 53.4% yield) was obtained as colorless oil.

### Step 3. Preparation of Compound SIL3-9

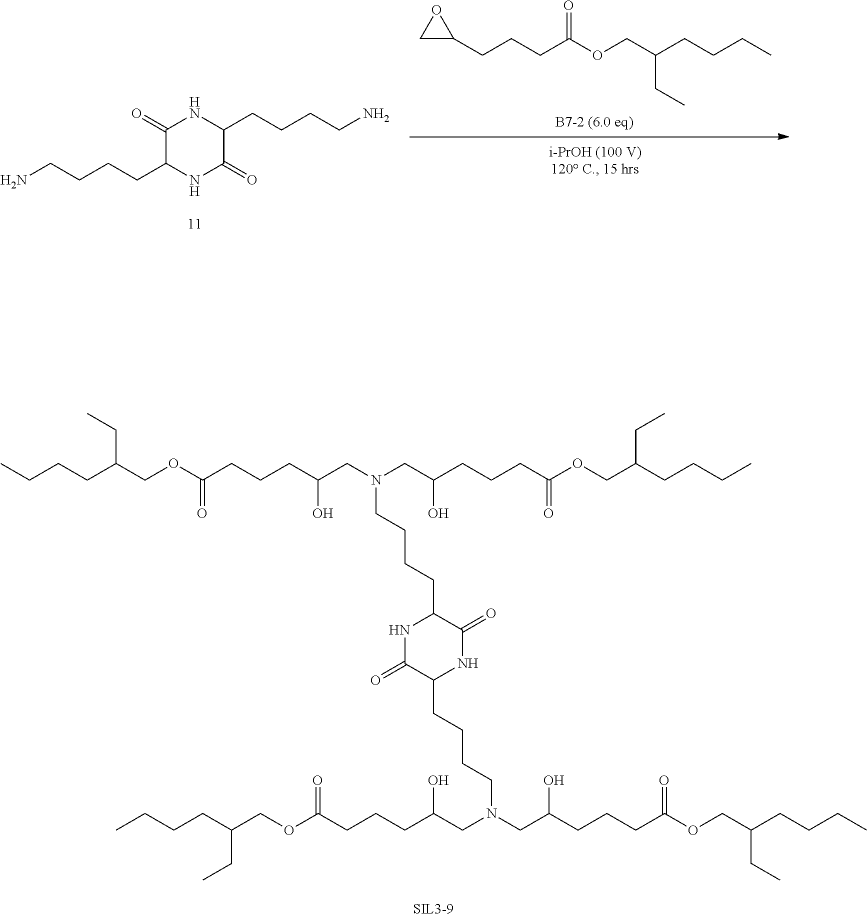 [SMILES: CCCCC(CC)COC(=O)CCCC1CO1 ; NCCCCC1NC(=O)C(CCCCN)NC1=O ; "B7-2 (6.0 eq)" ; CCCCC(CC)COC(=O)CCCC(O)CN(CCCCC1NC(=O)C(CCCCN(CC(O)CCCC(=O)OCC(CC)CCCC)CC(O)CCCC(=O)OCC(CC)CCCC)NC1=O)CC(O)CCCC(=O)OCC(CC)CCCC ; "SIL3-9"]

A mixture of Compound B (0.30 g, 1.17 mmol, 1.00 eq), Compound B7-2 (1.70 g, 7.02 mmol, 6.00 eq) in IPA (30 mL) was degassed and purged with N₂ for 3 times, and then the mixture was stirred at 120° C. for 15 hrs under N₂ atmosphere. TLC (Dichloromethane:Methanol=10:1, R_{f}=0.17) showed desired compound was detected. The reaction mixture was concentrated under reduced pressure to give a residue. The residue was purified by column chromatography (SiO₂, DCM:MeOH=30/1 to 10/1). The residue was purified by prep-HPLC (column: Phenomenex Gemini-NX C18 75*30 mm*3 μm; mobile phase: [water (0.1% TFA)-ACN]; B %: 50%-80%, 12 min). The pH value was adjusted to 8 by aq. NaHCO₃, and extracted with DCM (10 mL). The combined organic layers were washed with brine (5 mL), filtered and concentrated under reduced pressure to give a residue. Compound SIL3-9 (62.0 mg, 50.58 μmol, 2.16% yield) was obtained as yellow oil.

¹H NMR: 400 MHz CDCl₃

δ 6.62 (br s, 1H), 3.63-4.18 (m, 16H), 2.46-2.83 (m, 5H), 2.28-2.46 (m, 9H), 1.24-2.05 (m, 73H), 0.85-0.95 (m, 24H).

### Example 11. Synthesis of Compound SIL3-10

### Step 1. Preparation of Compound C4-1

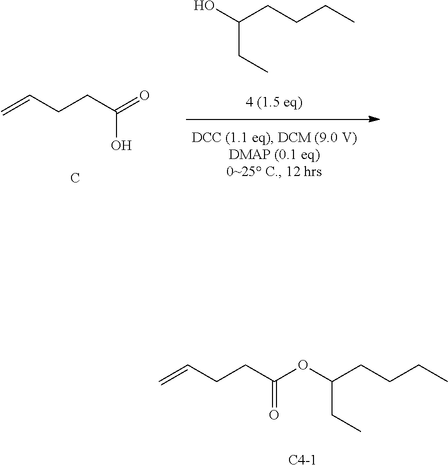

To a solution of Compound C (10.0 g, 100 mmol, 10.2 mL, 1.00 eq) and Compound 4 (17.4 g, 150 mmol, 535 μL, 1.50 eq) in DCM (90 mL) was added DMAP (1.22 g, 9.99 mmol, 0.10 eq) and DCC (22.7 g, 110 mmol, 22.2 mL, 1.10 eq) at 0° C. The mixture was stirred at 25° C. for 12 hrs. TLC (Petroleum ether:Ethyl acetate=10:1, R_{f}=0.43) indicated ˜5% of Compound C remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound C4-1 (15.0 g, 75.6 mmol, 75.7% yield) was obtained as colorless oil.

¹H NMR: 400 MHz CDCl₃

δ 5.70-5.95 (m, 1H), 4.95-5.17 (m, 2H), 4.80-4.90 (m, 1H), 2.33-2.45 (m, 4H), 1.51-1.60 (m, 4H), 1.26-1.32 (m, 5H), 0.86-0.91 (m, 7H).

### Step 2. Preparation of Compound C4-2

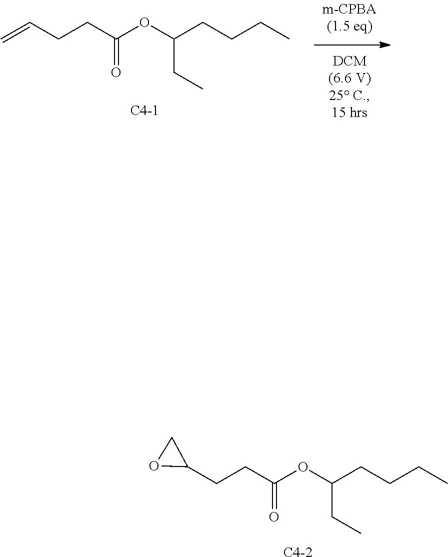

To a solution of Compound C4-1 (15.0 g, 75.6 mmol, 1.00 eq) in DCM (100 mL) was added m-CPBA (24.5 g, 113 mmol, 80% purity, 1.50 eq) at 0° C. The mixture was stirred at 15° C. for 15 hrs. TLC (Petroleum ether:Ethyl acetate=10:1) indicated Compound C4-1 was consumed completely and two new spots formed. The reaction mixture was filtered. The filtrate was quenched by addition NaHSO₃ (1000 mL), and then extracted with petroleum ether (500 mL). The combined organic layers were washed with Na₂CO₃ (300 mL). The combined organic layers were washed with brine (100 mL), dried over (Na₂SO₄), filtered and concentrated under reduced pressure to give a residue. The crude product was dissolved by petroleum ether. The solid was precipitated at −10° C., filtered and the filtrate was concentrated under reduced pressure. The residue was purified by column chromatography (SiO₂, Petroleum ether/Ethyl acetate=1/0 to 30/1). Compound C4-2 (5.00 g, 23.3 mmol, 30.8% yield) was obtained as colorless oil.

¹H NMR: 400 MHz CDCl₃

δ 4.80-4.87 (m, 1H), 2.93-3.04 (m, 1H), 2.77 (t, J=4.8 Hz, 1H), 2.52 (dd, J=4.8, 2.8 Hz, 1H), 2.43-2.49 (m, 2H), 1.92-2.02 (m, 1H), 1.74-1.86 (m, 1H), 1.49-1.63 (m, 4H), 1.24-1.36 (m, 4H), 0.86-0.91 (m, 6H).

### Step 3. Preparation of Compound SIL3-10

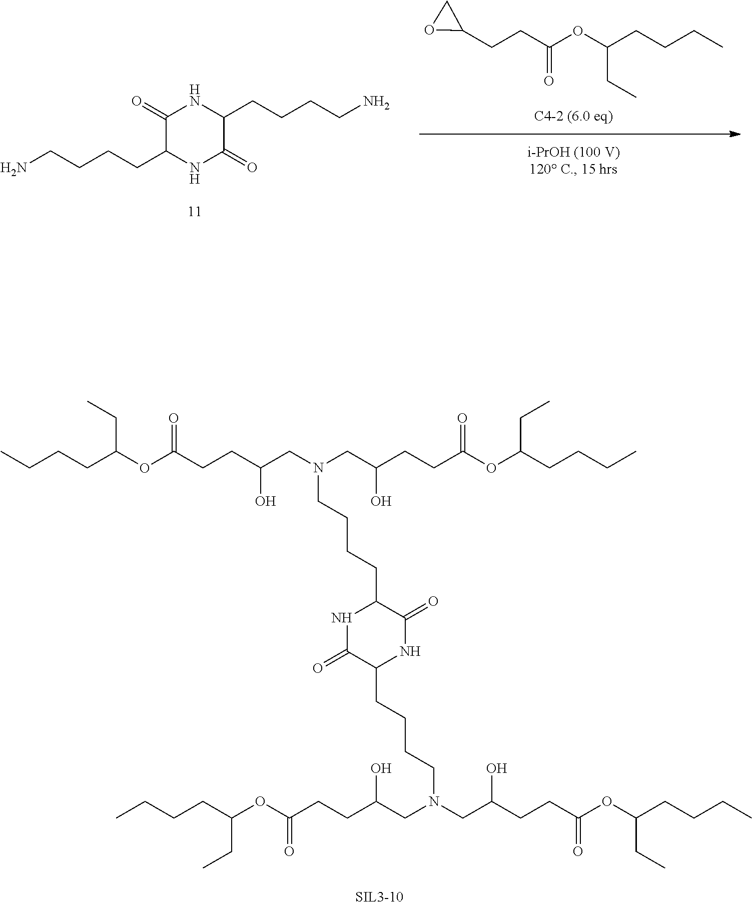

A mixture of Compound 11 (0.2 g, 780.20 μmol, 1 eq), Compound C4-2 (1.00 g, 4.68 mmol, 6.00 eq) in IPA (20 mL) was degassed and purged with N₂ for 3 times, and then the mixture was stirred at 120° C. for 15 hrs under N₂ atmosphere. TLC (Dichloromethane:Methanol=10:1, R_{f}=0.17) indicated many new spots formed. The reaction mixture was concentrated under reduced pressure to give a residue. The residue was purified by column chromatography (SiO₂, DCM:MeOH=30/1 to 10/1). The residue was purified by prep-HPLC (column: Phenomenex Gemini-NX C18 75*30 mm*3 um; mobile phase: [water (0.1% TFA)-ACN]; B %: 40%-70%, 10 min). The Ph value was adjusted to 8 by aq. NaHCO₃, and extracted with DCM (10 mL). The combined organic layers were washed with brine 5 mL, filtered and concentrated under reduced pressure to give a residue. Compound SIL3-10 (114 mg, 102 μmol, 38.00% yield) was obtained as yellow oil.

¹H NMR: 400 MHz CDCl₃

δ 6.48 (br s, 1H), 4.79-4.85 (m, 4H), 4.08 (br s, 2H), 3.78 (br s, 4H), 2.29-2.89 (m, 20H), 1.42-2.11 (m, 38H), 1.18-1.40 (m, 18H), 0.85-0.92 (m, 24H).

### Example 11. Synthesis of Compound SIL3-11

### Step 1. Preparation of Compound C5-1

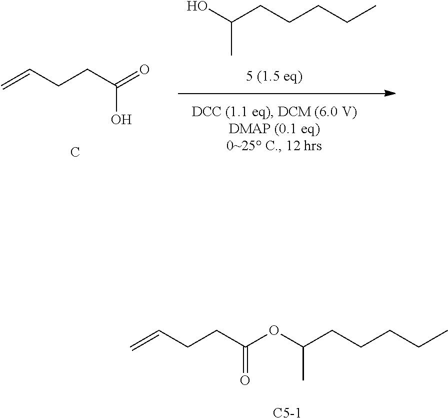

To a solution of Compound C (15.0 g, 150 mmol, 15.3 mL, 1.00 eq) and Compound 5 (26.1 g, 225 mmol, 1.50 eq) in DCM (90 mL) was added DMAP (1.83 g, 15.0 mmol, 0.10 eq) and DCC (34.0 g, 165 mmol, 33.3 mL, 1.10 eq) at 0° C. The mixture was stirred at 25° C. for 12 hrs. TLC (Petroleum ether:Ethyl acetate=10:1, R_{f}=0.43) indicated ˜5% of Compound C remained, and three major new spots were detected. The reaction mixture was filtered and the filtrate was concentrated under reduced pressure to give a residue. The residue was purified by column chromatography (SiO₂, Petroleum ether/Ethyl acetate=1/0 to 30/1). Compound C5-1 (25.0 g, 126 mmol, 84.1% yield) was obtained as colorless oil.

¹H NMR: 400 MHz CDCl₃

δ 5.70-5.94 (m, 1H), 4.83-5.14 (m, 3H), 2.32-2.44 (m, 4H), 1.44-1.64 (m, 2H), 1.23-1.37 (m, 6H), 1.20 (d, 0.1=6.4 Hz, 3H), 0.85-0.92 (m, 3H).

### Step 2. Preparation of Compound C5-2

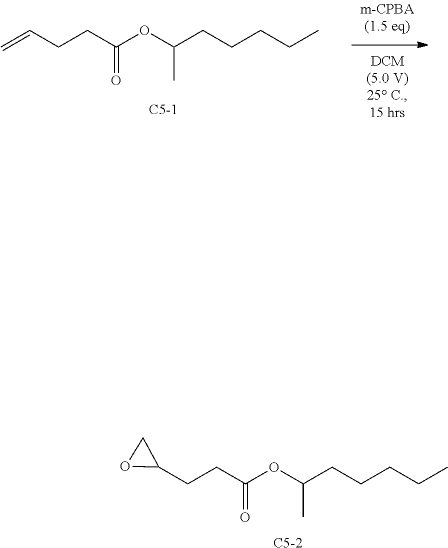

To a solution of Compound C5-1 (10.0 g, 50.4 mmol, 1.00 eq) in DCM (50 mL) was added m-CPBA (16.3 g, 75.6 mmol, 80% purity, 1.50 eq) at 0° C. The mixture was stirred at 15° C. for 15 hrs. TLC (Petroleum ether:Ethyl acetate=10:1) indicated Compound C5-1 was consumed completely and two new spots formed. The reaction mixture was filtered. The filtrate was quenched by addition NaHSO₃ (500 mL), and then extracted with petroleum ether (500 mL). The combined organic layers were washed with Na₂CO₃ (200 mL). The combined organic layers were washed with brine (50 mL), dried over (Na₂SO₄), filtered and concentrated under reduced pressure to give a residue. The crude product was dissolved by petroleum ether. The solid was precipitated at −10° C., filtered and the filtrate was concentrated under reduced pressure. Compound C5-2 (3.00 g, 14.0 mmol, 27.8% yield) was obtained as colorless oil.

### Step 3. Preparation of Compound C5

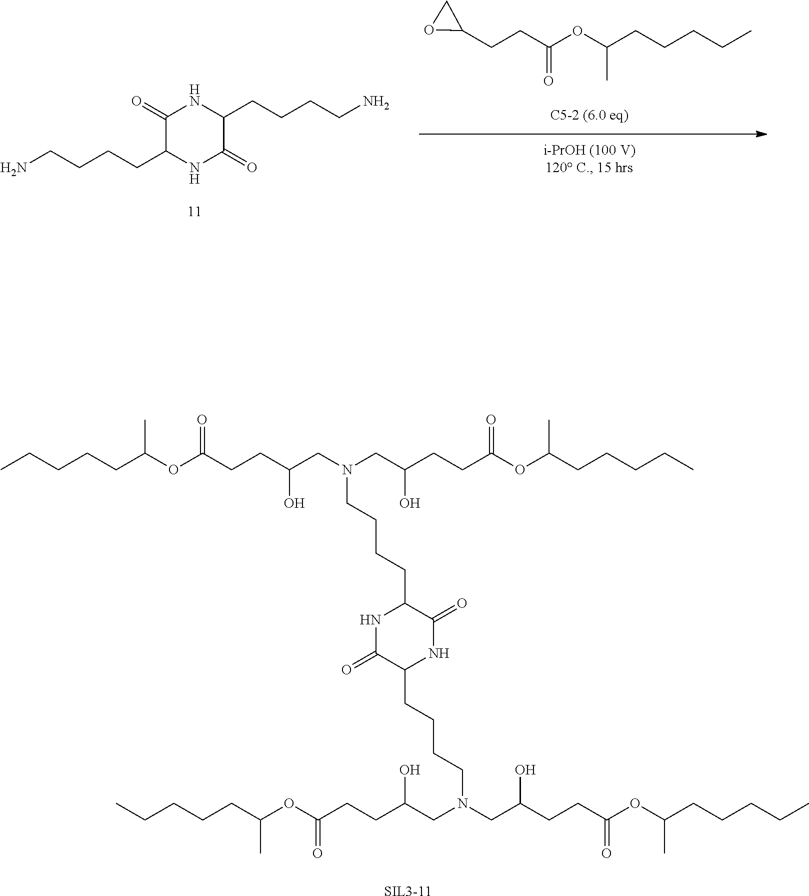

A mixture of Compound 11 (0.20 g, 780 μmol, 1.00 eq), Compound C5-2 (1.00 g, 4.68 mmol, 6.00 eq) in IPA (20 mL) was degassed and purged with N₂ for 3 times, and then the mixture was stirred at 120° C. for 15 hrs under N₂ atmosphere. LC-MS showed desired compound was detected. The reaction mixture was concentrated under reduced pressure to give a residue. The residue was purified by column chromatography (SiO₂, DCM:MeOH=30/1 to 10/1). The residue was purified by prep-HPLC (column: Phenomenex Gemini-NX C18 75*30 mm*3 um; mobile phase: [water (0.1% TFA)-ACN]; B %: 40%-70%, 10 min). The Ph value was adjusted to 8 by aq. NaHCO₃, and extracted with DCM (10 mL). The combined organic layers were washed with brine (5 mL), filtered and concentrated under reduced pressure to give a residue. Compound SIL3-11 (71.0 g, 63.7 μmol, 7.89% yield) was obtained as yellow oil.

¹H NMR: 400 MHz CDCl₃

δ 6.28-6.65 (m, 1H), 4.82-4.99 (m, 4H), 4.07 (br s, 2H), 3.75 (br s, 3H), 2.22-2.81 (m, 18H), 0.95-2.07 (m, 72H), 0.86-0.91 (m, 12H).

### Example 12. Preparation of Lipid Nanoparticle Formulations Using Microfluidics Device

Lipid nanoparticle (LNP) formulations comprising the inventive lipids described herein were prepared as follows.

Briefly, the DNA or mRNA solution was prepared in 10-100 mM citrate buffer solution at pH 3-5, while the lipid mixture was dissolved in ethanol. The DNA or mRNA aqueous solution and the lipid ethanolic solution were mixed on NanoAssemblr at 5:1 to 3:1 flow rate ratio with total flow rate of 12 mL/min. Subsequently, the LNP solution was transferred to float-lyzer, and dialyzed against 1×DPBS buffer for 2 hrs, then another 2 hrs against newly made 1×DPBS at room temperature. Finally, they were dialyzed against another newly made 1×DPBS solution overnight at 0° C.

Next, a concentration step was performed. The intermediate LNP was concentrated with Amicon Ultra-15 (10 kD MWCO) tube at 2000 g, 23° C. till it was concentrated 5-15×. Then the LNP was filtered through a 0.2 μm pore sterile filter. The particle size of LNP was determined by quasielastic light scattering using a Malvern Zetasizer Nano ZS (Malvern, UK) and the DNA or mRNA encapsulation as well as quantification was measured by Quant-iT RiboGreen mRNA Assay Kit (Thermo Fisher Scientific). Based on the DNA or mRNA quantification results, the LNP solutions were further diluted with 1×DPBS buffer as well as 60% w/w sucrose solution, and stored at −80° C.

### Example 13. Lipid Nanoparticle Formulation Characterizations

Upon preparation of the lipid nanoparticle formulations as described in Example 12, lipid nanoparticle size and zeta potential, and encapsulation efficiency of nucleic acid into the lipid nanoparticles were analyzed. Particle size was determined by dynamic light scattering and zeta potential was measured by electrophoretic light scattering (Zetasizer Nano ZS, Malvern Instruments).

Encapsulation efficiency (ee %) of DNA or mRNA in lipid particles as well as the quantification was determined by RiboGreen® (Thermo Scientific) kit. Ribogreen® is an ultra-sensitive fluorescent nucleic acid stain for quantitating mRNA but can also be used to quantify dsDNA. The ee % was determined by performing a membrane impermeable fluorescent dye exclusion assay. Briefly, each LNP sample was divided into two groups, one treated with TE buffer, and the other treated with Triton-X100, a nonionic surfactant known to break the LNPs. In addition, a series of known concentrations of the DNA or mRNA solution were also prepared as the standard solutions for quantification. Subsequently, all the wells were treated with Ribogreen®, and after 5-10 mins the fluorescence readings were taken. The ee % is calculations as ee %=(ITriton-ITE)/ITriton, where ITriton is the fluorescence of the Triton-X₁₀₀ treated LNP samples, while the ITE was the fluorescence of the TE buffer treated LNP group. For the quantification of the DNA or mRNA, a standard linear curve was constructed based on the fluorescence intensity vs. known concentrations of the DNA or mRNA standard solutions. To obtain the DNA or mRNA total quantification of the LNP samples, the ITriton fluorescence value was plugged into the standard linear curve to calculate the DNA or mRNA concentration of the LNP in question.

### Example 14. Cell Culture Methods

### THP1-Dual Cell Culture:

Culture flask (Corning) was removed from the incubator (37 C, 5% CO₂, 80% RH). Cell suspension was removed from the culture flask and centrifuged at 1000 rpm for 5 minutes. Supernatant was aspirated and cell pellet was reconstituted in growth medium (RPMI (Gibco) with 10% FBS (Gibco), 1% Pen-Strep (Gibco), 100 ug/mL Normocin (Invivogen)). An aliquot of the cell suspension was diluted in 0.4% Trypan blue (Gibco) for cell counting. Cells were passed into a culture flask at 5×10⁵ cells/ml in growth media and flask was placed in the incubator. Every other passage, add 10 μg/ml of blasticidin (Invivogen) and 100 μg/ml of Zeocin (Invivogen) to growth media.

When seeding cell plates for assay, transferred 200 uL/well of cell suspension at 3×10⁵ cells/mL to a 96-well U-bottom tissue culture plate (Costar). Cell seeding was performed the day before the assay.

### HepG2 Cell Culture:

Culture flask (Corning) was removed from the incubator (37C, 5% CO₂, 80% RH). Supernatant was removed from the culture flask and cell layer was rinsed with 1×DPBS. Trypsin-EDTA (Gibco) was added to the cell layer and flask was placed in the incubator for cell detachment. Growth medium (DMEM (Gibco) with 10% FBS (Gibco), and 1% Pen-Strep (Gibco)) was added to the flask. An aliquot of the cell suspension was diluted in 0.4% Trypan blue (Gibco) for cell counting. Cells were passed into a gelatin-coated culture flask with growth media and flask was placed in the incubator.

When seeding cell plates for assay, 200 μL of cell suspension at 1.5×10⁵ cells/mL was transferred to a gelatin-coated, 96-well flat-bottom tissue culture plate (Costar). Cell seeding was performed the day before the assay.

### Example 15. Cell-Based Assays

Gaussia-LNP or hFIX-LNP was diluted in culture media to 4 μg/mL and 25 μL were added to each well in a 96-well plate containing 200 μL THP-1 dual or HepG2 cells. ApoE4 (Peprotech) was diluted in culture media to 10 μg/mL and 25 μL were added to 96-well plates containing either cell line. The final amount of LNP added and final ApoE4 concentration in 250 μL media was 100 ng per well and 1 μg/mL, respectively.

The THP-1 dual cells were used to detect NFkB and IRF activation 24 hr post LNP-stimulation. To detect NFkB activation, supernatant was collected after 24 hours and diluted 1:10 in Quanti-Blue Solution (InvivoGen). OD values were read on a microplate reader at 620 nm absorbance. To detect IRF activation, 24 hour supernatant was diluted 1:3.5 in QUANTI-Luc (InvivoGen). Luminescence units were determined using a luminometer.

Gaussia expression by hepg2 cells was determined by collecting supernatant 24 hours after LNP stimulation and diluting supernatant 1:3.5 in QUANIT-Luc. Luminescence units were determined using a luminometer.

hFIX expression by hepg2 cells was determined by collecting supernatant 24 hours after LNP stimulation and diluting supernatant 1:1 in assay media. hFIX concentration was determined using the FIX ELISA protocol outlined below.

### Example 16. FIX ELISA Assay Protocol

96-well ELISA plates (Costar) were coated with capture antibody (Affinity Biologicals) then sealed and incubated at RT. Plates were washed 3 times with PBS-Tween wash buffer and blotted to remove excess liquid. Standards were prepared by serial-diluting human pooled normal plasma (George King Biomedical) in mouse control plasma (BioIVT). Prepared standards and samples were diluted in sample diluent. Transferred standard and sample dilutions to plates and incubated at RT. Plates were washed 3 times with wash buffer and blotted to remove excess liquid. Detection antibody (Affinity Biologicals) was added to each well of the plates and incubated at RT. Plates were washed 3 times with PBS-Tween wash buffer and blotted to remove excess liquid. TMB (Thermo Scientific) was added to each well of the plates and incubated in the dark at RT. Sulfuric acid was added to each well and plates were read for absorbance at 450 nm on a plate reader (Biotek). A 4-parameter curve was generated for the standards. Samples results were interpolated on the standard curve.

### Example 17. LNP-dsDNA Formulations Using Lipids from SIL1 Series Lipids

In one study, LNPs were formulated using method described in Example 12. These LNPs contain selected the inventive lipids of SIL1 series, DSPC, cholesterol and DMG-PEG2000, as well as erythropoietin (EPO) mRNA. The weight ratio of the ionizable lipid to the FIX plasmid is between 5-15. Table 3 shows exemplary LNPs prepared in this study, as well as their characterizations.

TABLE 3
Exemplary LNPs prepared using SIL1 lipids in this study.

| | | | | | 14:0 | |
|---|---|---|---|---|---|---|
| | | Ionizable | | | PEG2000 | |
| | Ionizable | lipid | Cholesterol | DSPC | PE | |
| LNP # | lipid | mol % | mol % | mol % | mol % | Payload |
| 1 | SIL1-1 | 50 | 46.5 | 16 | 2.5 | EPO mRNA |
| 2 | SIL1-3 | 50 | 46.5 | 16 | 2.5 | EPO mRNA |
| 3 | SIL1-4 | 50 | 46.5 | 16 | 2.5 | EPO mRNA |
| 4 | SIL1-5 | 50 | 46.5 | 16 | 2.5 | EPO mRNA |
| 5 | SIL1-9 | 50 | 46.5 | 16 | 2.5 | EPO mRNA |
| 6 | SIL1-10 | 50 | 46.5 | 16 | 2.5 | EPO mRNA |
| 7 | SIL1-13 | 50 | 46.5 | 16 | 2.5 | EPO mRNA |
| 8 | SIL1-14 | 50 | 46.5 | 16 | 2.5 | EPO mRNA |
| 9 | SIL1-15 | 50 | 46.5 | 16 | 2.5 | EPO mRNA |
| 10 | MC3 | 50 | 46.5 | 16 | 2.5 | EPO mRNA |
| 11 | MC9 | 50 | 46.5 | 16 | 2.5 | EPO mRNA |

### Example 18. In Vivo Evaluation of LNPs 1-11

A 11-arm study was conducted in female Balb/C mice for a total duration of two days. 11 groups of mice weighing approximately 25 grams (6-10 weeks old) (n=4 per time point) were administered an intravenous (IV) dose as described in Table 4.

The first plasma sample was collected via serial sampling from Group 1 one day prior to IV dosing. In all Groups, samples were collected at 4 hours and 24 hours post-dose. A target total of 80 μL of whole blood was drawn via orbital eye bleed. The blood was transferred to lithium heparin tubes and centrifuged at 9,800×g for 10 minutes at 2-5° C. Plasma were harvested and frozen at ≤−70° C. until shipment to the Sponsor. A total of 40 μL plasma was the target volume; however, if less plasma is available, tubes were noted as such. After collection, samples were frozen and stored at ≤−70° C. for further analysis.

TABLE 4
Study Design for Administration & Sampling

| | | | | Day 0 | | |
|---|---|---|---|---|---|---|
| Group | Mouse Strain | n | Day −1 | Dose at T = 0 | +4 hours | +24 hours |
| 1 | Balb/C | 4 | Bleed | LNP 1 | Bleed | Bleed |
| 2 | Balb/C | 4 | NA | LNP 2 | Bleed | Bleed |
| 3 | Balb/C | 4 | NA | LNP 3 | Bleed | Bleed |
| 4 | Balb/C | 4 | NA | LNP 4 | Bleed | Bleed |
| 5 | Balb/C | 4 | NA | LNP 5 | Bleed | Bleed |
| 6 | Balb/C | 4 | NA | LNP 6 | Bleed | Bleed |
| 7 | Balb/C | 4 | NA | LNP 7 | Bleed | Bleed |
| 8 | Balb/C | 4 | NA | LNP 8 | Bleed | Bleed |
| 9 | Balb/C | 4 | NA | LNP 9 | Bleed | Bleed |
| 10 | Balb/C | 4 | NA | LNP 10 | Bleed | Bleed |
| 11 | Balb/C | 4 | NA | LNP 11 | Bleed | Bleed |

### Example 19. In Vivo Study Results

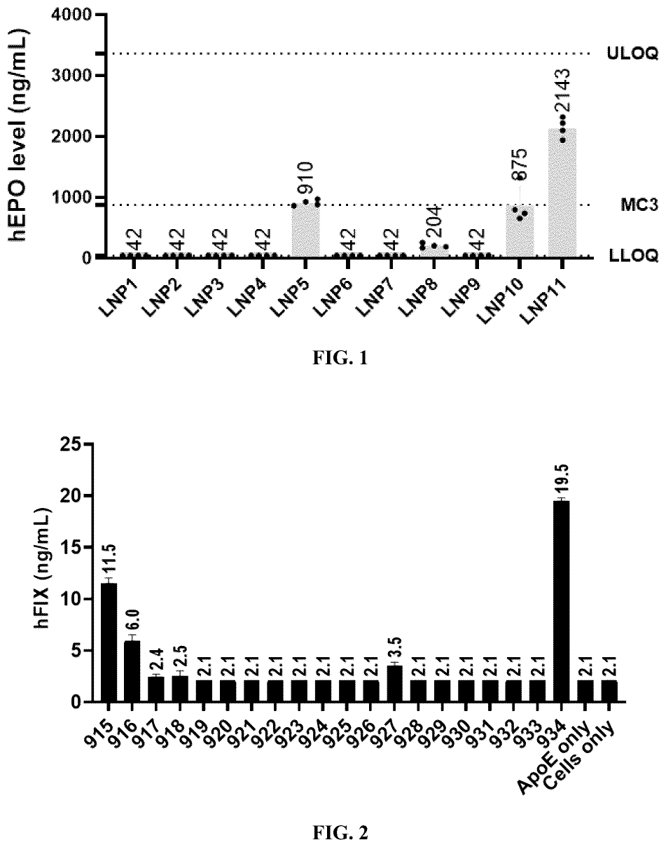

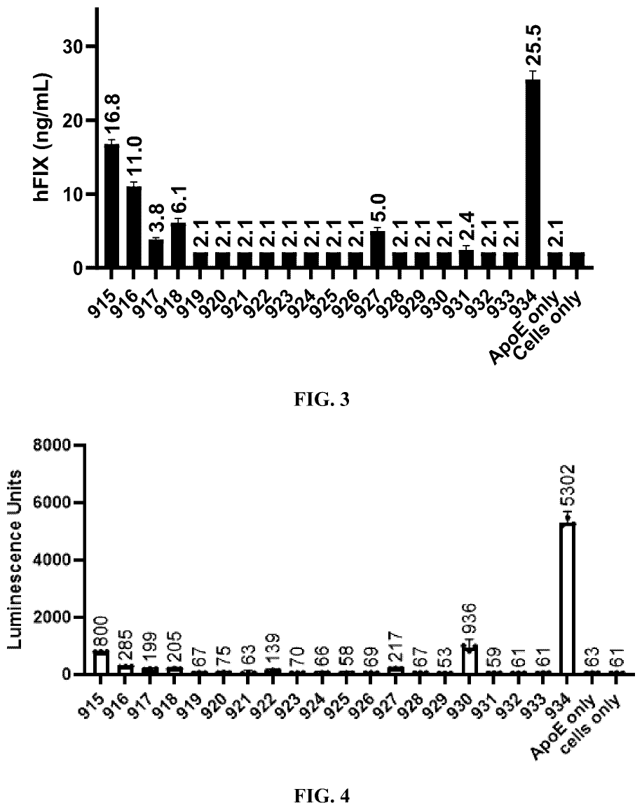

hEPO levels in the plasma were evaluated at 4 hrs using EPO-ELISA assay. The results were shown in FIG. 1.

### Example 20. LNP-dsDNA Formulations Using SIL2 Series Lipids

In one study, LNPs were formulated using method described in Example 12. These LNPs contain exemplary SIL2 series lipids (SIL2-1 to SIL2-22, as well as a reference lipid bckk-E12), DOPE, cholesterol and DMG-PEG2000, and C18-PEG2000-TriGalnac ligand (mol ratio of 35:16 46.5:2.4:0.1), as well as FIX plasmid. The weight ratio of the ionizable lipid to the FIX plasmid is about 10, giving a NP ratio of ca. 6.5. Table 5 shows exemplary LNPs prepared in this study, as well as their characterizations.

TABLE 5
Exemplary LNPs prepared using SIL2 lipids in this study.

| | | | | | 14:0 | | | | | |
|---|---|---|---|---|---|---|---|---|---|---|
| | | Ionizable | | | PEG2000 | C18 PEG | | | | |
| LNP | Ionizable | lipid | Cholesterol | DOPE | PE | GalNAc | | Size | | |
| # | lipid | mol % | mol % | mol % | mol % | mol % | DNA | (nm) | PDI | ee % |
| 915 | SIL2-1 | 35 | 46.5 | 16 | 2.4 | 0.1 | FIX | 64.72 | 0.089 | 93 |
| | | | | | | | 40 | | | |
| 916 | SIL2-2 | 35 | 46.5 | 16 | 2.4 | 0.1 | FIX | 66.49 | 0.086 | 95 |
| | | | | | | | 40 | | | |
| 917 | SIL2-3 | 35 | 46.5 | 16 | 2.4 | 0.1 | FIX | 64.08 | 0.076 | 93 |
| | | | | | | | 40 | | | |
| 918 | SIL2-4 | 35 | 46.5 | 16 | 2.4 | 0.1 | FIX | 65.48 | 0.07 | 95 |
| | | | | | | | 40 | | | |
| 919 | SIL2-5 | 35 | 46.5 | 16 | 2.4 | 0.1 | FIX | 73.29 | 0.024 | 87 |
| | | | | | | | 40 | | | |
| 920 | SIL2-6 | 35 | 46.5 | 16 | 2.4 | 0.1 | FIX | 65.31 | 0.078 | 96 |
| | | | | | | | 40 | | | |
| 921 | SIL2-7 | 35 | 46.5 | 16 | 2.4 | 0.1 | FIX | 73.23 | 0.223 | 92 |
| | | | | | | | 40 | | | |
| 922 | SIL2-8 | 35 | 46.5 | 16 | 2.4 | 0.1 | FIX | 65.77 | 0.099 | 91 |
| | | | | | | | 40 | | | |
| 923 | SIL2-10 | 35 | 46.5 | 16 | 2.4 | 0.1 | FIX | 65.66 | 0.122 | 91 |
| | | | | | | | 40 | | | |
| 924 | SIL2-11 | 35 | 46.5 | 16 | 2.4 | 0.1 | FIX | 75.89 | 0.118 | 90 |
| | | | | | | | 40 | | | |
| 925 | SIL2-12 | 35 | 46.5 | 16 | 2.4 | 0.1 | FIX | 64.36 | 0.09 | 89 |
| | | | | | | | 40 | | | |
| 926 | SIL2-13 | 35 | 46.5 | 16 | 2.4 | 0.1 | FIX | 105.7 | 0.085 | 81 |
| | | | | | | | 40 | | | |
| 927 | SIL2-15 | 35 | 46.5 | 16 | 2.4 | 0.1 | FIX | 67.71 | 0.111 | 89 |
| | | | | | | | 40 | | | |
| 928 | SIL2-17 | 35 | 46.5 | 16 | 2.4 | 0.1 | FIX | 78.52 | 0.097 | 90 |
| | | | | | | | 40 | | | |
| 929 | SIL2-18 | 35 | 46.5 | 16 | 2.4 | 0.1 | FIX | 64.29 | 0.08 | 92 |
| | | | | | | | 40 | | | |
| 930 | SIL2-19 | 35 | 46.5 | 16 | 2.4 | 0.1 | FIX | 71.26 | 0.132 | 97 |
| | | | | | | | 40 | | | |
| 931 | SIL2-20 | 35 | 46.5 | 16 | 2.4 | 0.1 | FIX | 67.66 | 0.086 | 93 |
| | | | | | | | 40 | | | |
| 932 | SIL2-21 | 35 | 46.5 | 16 | 2.4 | 0.1 | FIX | 67.61 | 0.104 | 92 |
| | | | | | | | 40 | | | |
| 933 | SIL2-22 | 35 | 46.5 | 16 | 2.4 | 0.1 | FIX | 67.77 | 0.095 | 89 |
| | | | | | | | 40 | | | |
| 934 | bCKK- | 35 | 46.5 | 16 | 2.4 | 0.1 | FIX | 69.91 | 0.069 | 99 |
| | E12 | | | | | | 40 | | | |

Note:
the structure of the reference lipid bckk-E12 is

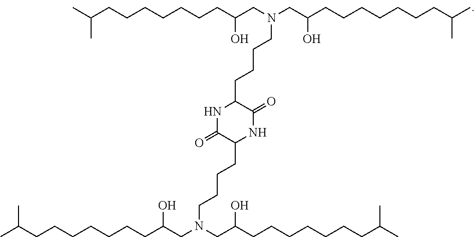 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

In conclusion, all LNPs using SIL-2 novel ionizable lipids were formulated successfully, and all have excellent ee % that is over 85%. The sizes of the LNPs are between 60-75 nm with a relatively narrow size distribution (PDI<0.2). As a results, these LNPs were evaluated next in vitro for efficacy and immunogenicity.

### Example 21. In Vitro Evaluation of LNPs 915-934

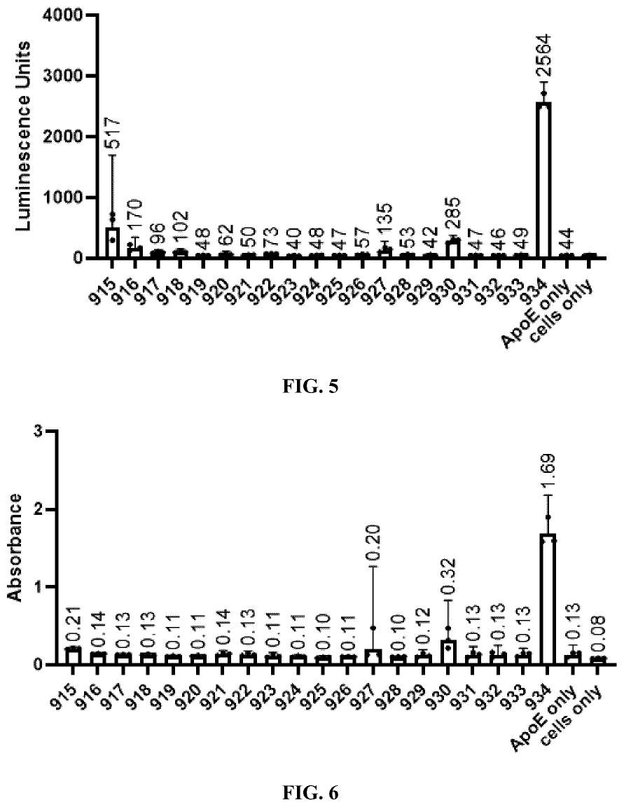

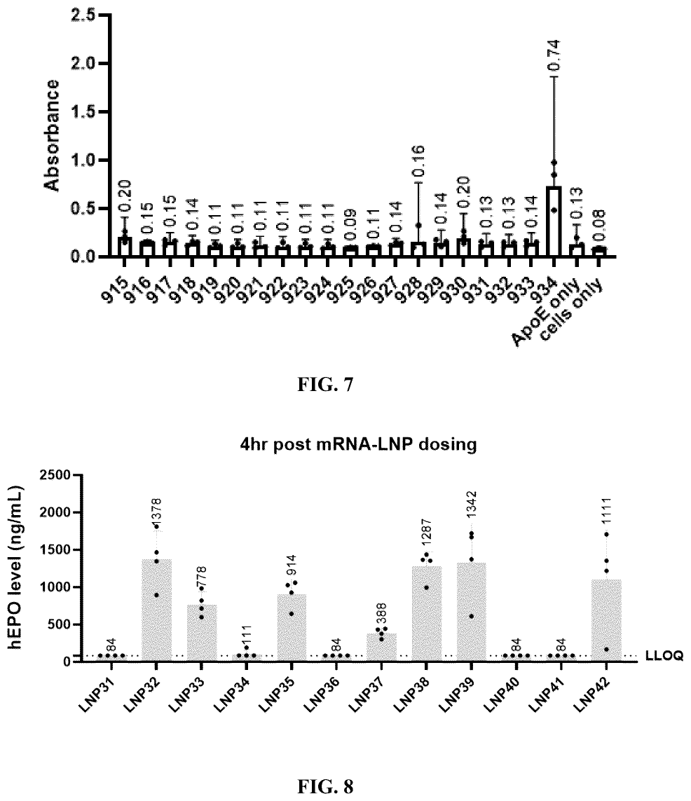

LNPs 915-934 were tested in vitro with THP-1 cells to evaluate their immunogenicity, and in HepG2 cells for their efficacies. The cell-based assays were carried out according to protocols outlined in Example 15 and 16. Briefly, LNPs 915-934 were incubated with either HepG2 cells or THP-1 cells at either 75 ng or 150 ng of DNA per well concentrations. hFIX expression by HepG2 cells was determined by collecting supernatant 24 hours after LNP stimulation and diluting supernatant 1:1 in assay media (FIGS. 2 & 3). hFIX concentration was determined using the FIX ELISA protocol outlined in Example 16. The THP-1 dual cells were used to detect NFkB and IRF activation 24 hr post LNP-stimulation and results are summarized in FIGS. 4-7.

### Example 22. LNP-mRNA Formulations Using Lipids from SIL3 Series Lipids

In one study, LNPs were formulated using method described in Example 12. These LNPs contain selected novel ionizable lipids of SIL3 series, DOPE, cholesterol and DMG-PEG2000, as well as erythropoietin (EPO) mRNA. The weight ratio of the ionizable lipid to the FIX plasmid is between 5-15. Table 6 shows exemplary LNPs prepared in this study, as well as their characterizations.

TABLE 6
Exemplary LNP-mRNA compositions prepared using SIL3 lipids in this study

| | | | | | 14:0 | |
|---|---|---|---|---|---|---|
| | | Ionizable | | | PEG2000 | |
| | Ionizable | lipid | Cholesterol | DOPE | PE | |
| LNP # | lipid | mol % | mol % | mol % | mol % | Payload |
| 31 | SIL3-1 | 35 | 46.5 | 16 | 2.5 | EPO mRNA |
| 32 | SIL3-2 | 35 | 46.5 | 16 | 2.5 | EPO mRNA |
| 33 | SIL3-3 | 35 | 46.5 | 16 | 2.5 | EPO mRNA |
| 34 | SIL3-4 | 35 | 46.5 | 16 | 2.5 | EPO mRNA |
| 35 | SIL3-5 | 35 | 46.5 | 16 | 2.5 | EPO mRNA |
| 36 | SIL3-6 | 35 | 46.5 | 16 | 2.5 | EPO mRNA |
| 37 | SIL3-7 | 35 | 46.5 | 16 | 2.5 | EPO mRNA |
| 38 | SIL3-8 | 35 | 46.5 | 16 | 2.5 | EPO mRNA |
| 39 | SIL3-9 | 35 | 46.5 | 16 | 2.5 | EPO mRNA |
| 40 | SIL3-10 | 35 | 46.5 | 16 | 2.5 | EPO mRNA |
| 41 | SIL3-11 | 35 | 46.5 | 16 | 2.5 | EPO mRNA |
| 42 | bcKK- | 35 | 46.5 | 16 | 2.5 | EPO mRNA |
| | E12 | | | | | |

### Example 23. In Vivo Evaluation of LNPs 31-42

A 12-arm study was conducted in female Balb/C mice for a total duration of two days. 12 groups of mice weighing approximately 25 grams (6-10 weeks old) (n=4 per time point) were administered an intravenous (IV) dose as described in Table 7.

The first plasma sample was collected via serial sampling from Group 1 one day prior to IV dosing. In all Groups, samples were collected at 4 hours and 24 hours post-dose. A target total of 80 μL of whole blood was drawn via orbital eye bleed. The blood was transferred to lithium heparin tubes and centrifuged at 9,800×g for 10 minutes at 2-5° C. Plasma were harvested and frozen at ≤−70° C. until shipment to the Sponsor.

A total of 40 μL plasma will be the target volume; however, if less plasma is available, tubes were noted as such. After collection, samples were frozen and stored at −70° C. for further analysis.

TABLE 7
Study Design for Administration & Sampling

| | | | | Day 0 | | |
|---|---|---|---|---|---|---|
| Group | Mouse Strain | n | Day −1 | Dose at T = 0 | +4 hours | +24 hours |
| 1 | Balb/C | 4 | Bleed | LNP 31 | Bleed | Bleed |
| 2 | Balb/C | 4 | NA | LNP 32 | Bleed | Bleed |
| 3 | Balb/C | 4 | NA | LNP 33 | Bleed | Bleed |
| 4 | Balb/C | 4 | NA | LNP 34 | Bleed | Bleed |
| 5 | Balb/C | 4 | NA | LNP 35 | Bleed | Bleed |
| 6 | Balb/C | 4 | NA | LNP 36 | Bleed | Bleed |
| 7 | Balb/C | 4 | NA | LNP 37 | Bleed | Bleed |
| 8 | Balb/C | 4 | NA | LNP 38 | Bleed | Bleed |
| 9 | Balb/C | 4 | NA | LNP 39 | Bleed | Bleed |
| 10 | Balb/C | 4 | NA | LNP 40 | Bleed | Bleed |
| 11 | Balb/C | 4 | NA | LNP 41 | Bleed | Bleed |
| 12 | Balb/C | 4 | NA | LNP 42 | Bleed | Bleed |

### Example 24. In Vivo Study Results

hEPO levels in the plasma were evaluated at 4 hrs using EPO-ELISA assay, and the results are shown in FIG. 8.

### Example 25. LNP-DNA Formulations Using Lipids from SIL3 Series Lipids

In one study, LNPs were formulated using method described in Example 12. These LNPs contain selected novel ionizable lipids of SIL3 series, DOPE, cholesterol and DMG-PEG2000, as well as erythropoietin (EPO) mRNA. The weight ratio of the ionizable lipid to the FIX plasmid is between 5-15. Table 8 shows exemplary LNPs prepared in this study, as well as their characterizations.

TABLE 8
Exemplary LNP-DNA compositions using SIL-3 lipids in this study

| | | Ionizable | | | 14:0 | |
|---|---|---|---|---|---|---|
| | Ionizable | lipid | Cholesterol | DOPE | PEG2000 | |
| LNP # | lipid | mol % | mol % | mol % | mol % | PEPayload |
| 43 | SIL3-1 | 35 | 46.5 | 16 | 2.5 | EPO nanoplasid |
| 44 | SIL3-2 | 35 | 46.5 | 16 | 2.5 | EPO nanoplasmid |
| 45 | SIL3-3 | 35 | 46.5 | 16 | 2.5 | EPO nanoplasmid |
| 46 | SIL3-4 | 35 | 46.5 | 16 | 2.5 | EPO nanoplasmid |
| 47 | SIL3-5 | 35 | 46.5 | 16 | 2.5 | EPO nanoplasmid |
| 48 | SIL3-6 | 35 | 46.5 | 16 | 2.5 | EPO nanoplasmid |
| 49 | SIL3-7 | 35 | 46.5 | 16 | 2.5 | EPO nanoplasmid |
| 50 | SIL3-8 | 35 | 46.5 | 16 | 2.5 | EPO nanoplasmid |
| 51 | SIL3-9 | 35 | 46.5 | 16 | 2.5 | EPO nanoplasmid |
| 52 | SIL3-10 | 35 | 46.5 | 16 | 2.5 | EPO nanoplasmid |
| 53 | SIL3-11 | 35 | 46.5 | 16 | 2.5 | EPO nanoplasmid |
| 54 | bcKK- | 35 | 46.5 | 16 | 2.5 | EPO nanoplasmid |
| | E12 | | | | | |

### Example 26. In Vivo Evaluation of LNPs 43-54

A 12-arm study was conducted in female Balb/C mice for a total duration of two days. 12 groups of mice weighing approximately 25 grams (6-10 weeks old) (n=4 per time point) were administered an intravenous (IV) dose as described in Table 9.

The first blood samples were collected via serial sampling from Group 1 one day prior to IV dosing. In all Groups, a sample will be collected at 4 and 16 hours on the day of dosing, and additional samples were collected at week 1 and week 2 post-dose. A target total of 80 μL of whole blood was drawn via orbital eye bleed.

For the samples collected at pre-dose, 4 and 16 hours, the blood was allowed to clot before being centrifuged at 9,800×g for 10 minutes at 2-5° C. to prepare serum. Serum was frozen at ≤−70° C. until shipment to the Sponsor.

For the samples collected at 1 and 2 weeks, the blood was transferred to lithium heparin tubes and centrifuged at 9,800×g for 10 minutes at 2-5° C. to prepare plasma. Plasma was harvested and frozen at ≤−70° C. until shipment to the Sponsor. A total of 40 μL plasma was the target volume; however, if less plasma is available, tubes were noted as such.

Animal weights were recorded during the study on day −1, day 2, day 4, week 1, and week 2. After collection, samples were frozen and stored at ≤−70° C.

TABLE 9
Animal Study Design

| | | | | Day 0 | | | |
|---|---|---|---|---|---|---|---|
| Group | Mouse Strain | n | Day −1 | Dose at T = 0 | +4 hours | Week 1 | Week 2 |
| 1 | Balb/C | 4 | Bleed | LNP 43 | Bleed | Bleed | Bleed |
| 2 | Balb/C | 4 | NA | LNP 44 | Bleed | Bleed | Bleed |
| 3 | Balb/C | 4 | NA | LNP 45 | Bleed | Bleed | Bleed |
| 4 | Balb/C | 4 | NA | LNP 46 | Bleed | Bleed | Bleed |
| 5 | Balb/C | 4 | NA | LNP 47 | Bleed | Bleed | Bleed |
| 6 | Balb/C | 4 | NA | LNP 48 | Bleed | Bleed | Bleed |
| 7 | Balb/C | 4 | NA | LNP 49 | Bleed | Bleed | Bleed |
| 8 | Balb/C | 4 | NA | LNP 50 | Bleed | Bleed | Bleed |
| 9 | Balb/C | 4 | NA | LNP 51 | Bleed | Bleed | Bleed |
| 10 | Balb/C | 4 | NA | LNP 52 | Bleed | Bleed | Bleed |
| 11 | Balb/C | 4 | NA | LNP 53 | Bleed | Bleed | Bleed |
| 12 | Balb/C | 4 | NA | LNP 54 | Bleed | Bleed | Bleed |

### Example 27. In Vivo Study Results

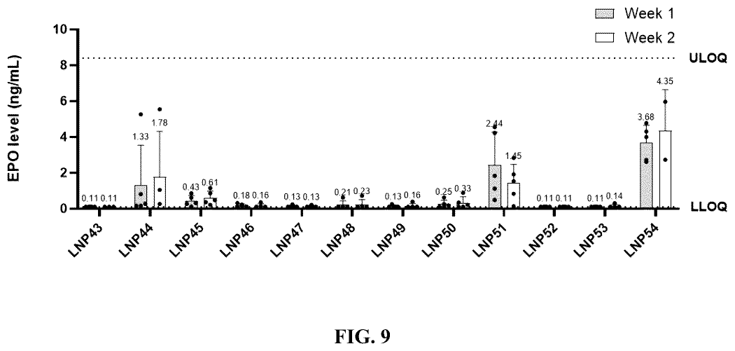

hEPO levels in the plasma were evaluated at week 1 and week 2 using EPO-ELISA assay and are shown in FIG. 9.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description. All documents cited herein are incorporated by reference.

1. A compound of formula (I): 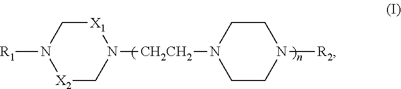
or a stereoisomer, tautomer, pharmaceutically acceptable salt or solvate thereof,
wherein,
n is 0 or 1;
X₁ and X₂ are different or identical, and are each independently CHR₃;
R₁ and R₂ are different or identical, and each independently selected from the group consisting of C₁₋₄ alkyl and —C₁₋₄ alkyl Y, wherein Y is —OR₄, —SR₄, or N(R₄)₂, and the C₁₋₄ alkyl is optionally substituted with one or more selected from the group consisting of halogen and C₁₋₄ alkyl;
R₃ is, independently at each occurrence, selected from the group consisting of hydrogen, alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, cycloalkyl, and heterocycle and said aryl or cycloalkyl or heterocycle is optionally substituted with one or more selected from the group consisting of halogen, hydroxyl, alkoxy, and amino;
R₄ is, independently at each occurrence, selected from the group consisting of alkyl and —C₁₋₄ alkyl Z, wherein Z is —OR₅, —SR₅, or N(R₅)², and the alkyl is optionally substituted with one or more hydroxyl groups;
R₅ is, independently at each occurrence, selected from the group consisting of hydroxyl-substituted alkyl and (—C₁₋₄ alkyl O)ₘ—C₁₋₄-alkyl-N(R₆)₂, wherein m is 0 or 1, R₆ is hydroxyl-substituted alkyl; and
with a proviso that at least one of R₁ and R₂ is not C₁₋₄ alkyl.

2. The compound of claim 1, wherein X₁ and X₂ are identical.

3. (canceled)

4. The compound of claim 1, being a compound of formula (I-A):

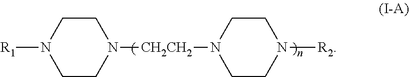

5. The compound of claim 1, wherein n is 0.

6. The compound of claim 1, wherein R₁ is —C₁₋₄ alkyl-Y.

7. The compound of claim 1, wherein R₂ is —C₁₋₄ alkyl-Y.

8. The compound of claim 1, wherein Y is —OR₄, —SR₄, or N(R₄)₂.

9. The compound of claim 1, wherein R₄ is —C₁₋₄ alkyl-Z.

10. The compound of claim 1, wherein Z is N(R₅)₂.

11. The compound of claim 1, wherein R₅ is hydroxyl-substituted alkyl.

12. (canceled)

13. (canceled)

14. The compound of claim 1, selected from the group consisting of:

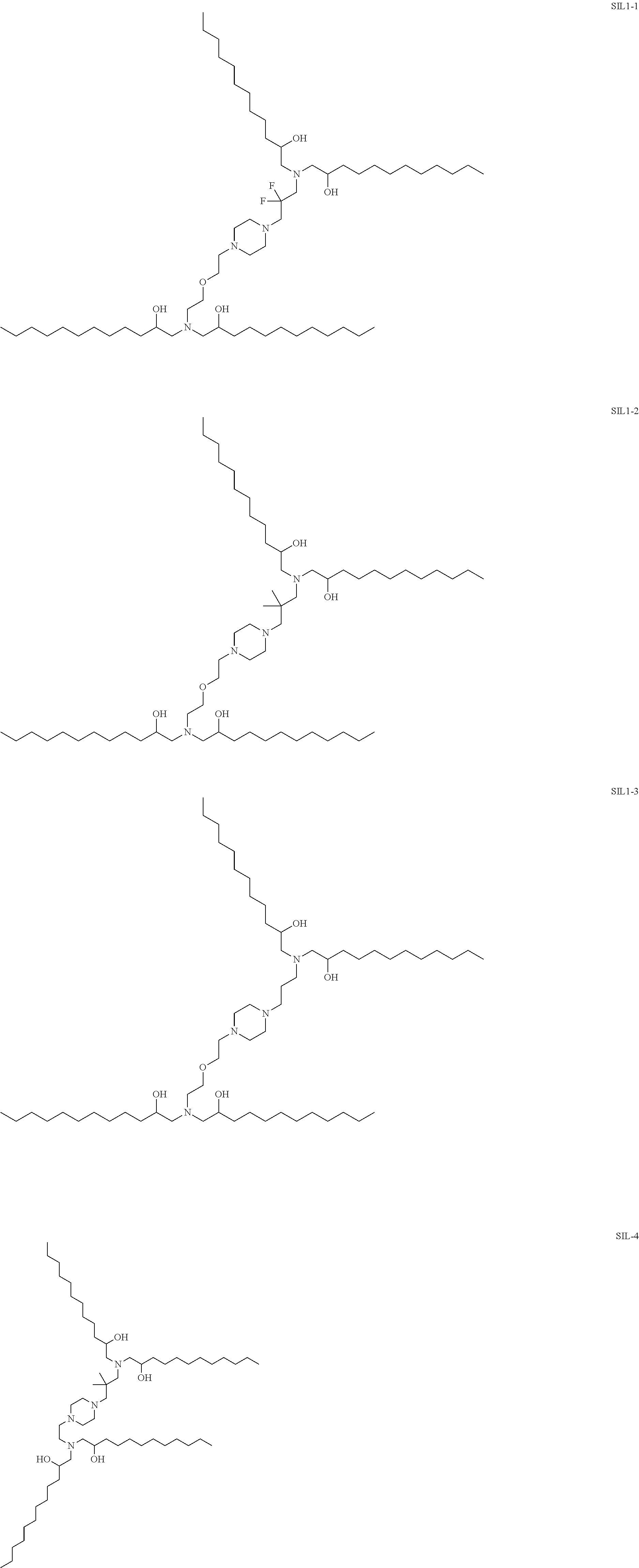

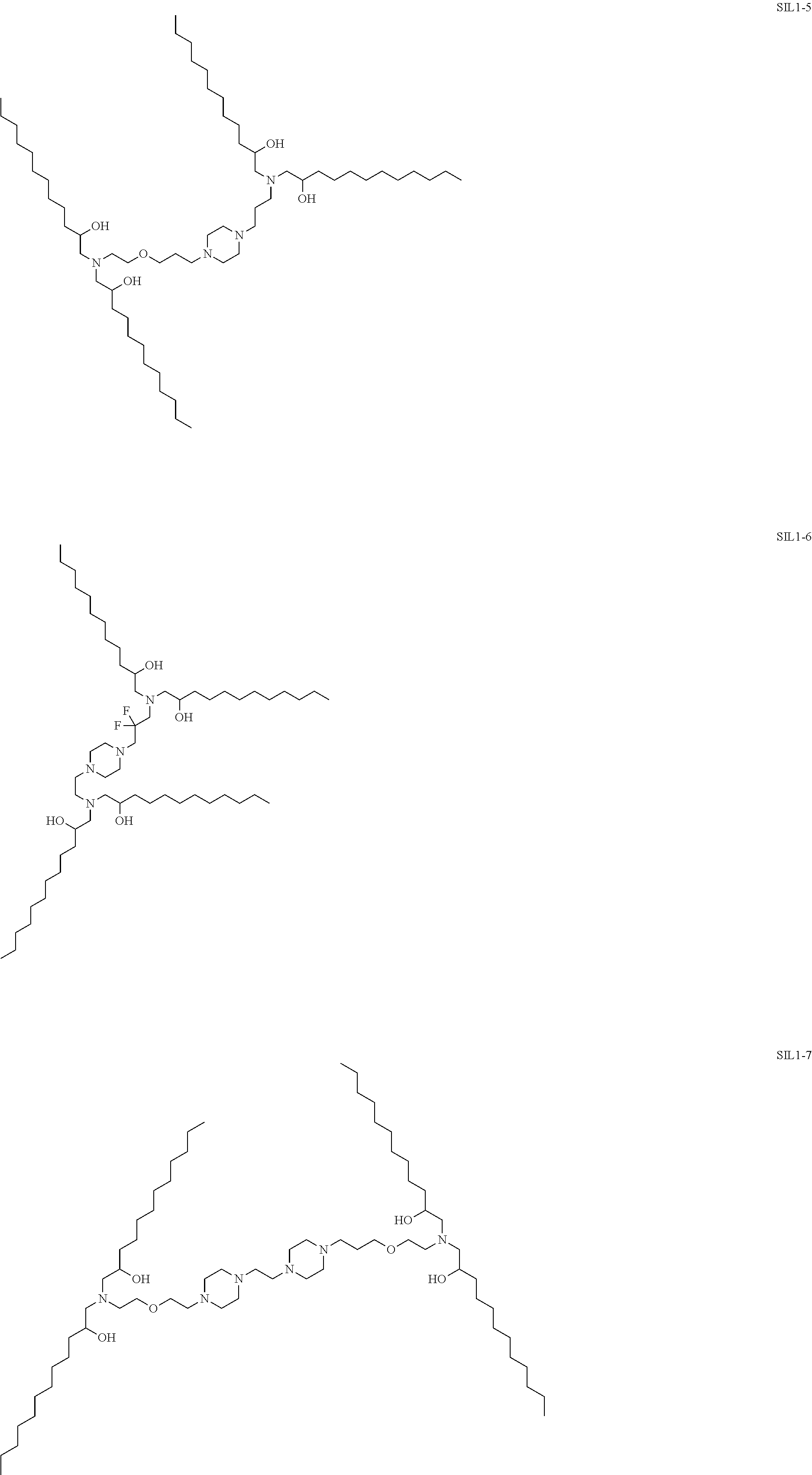

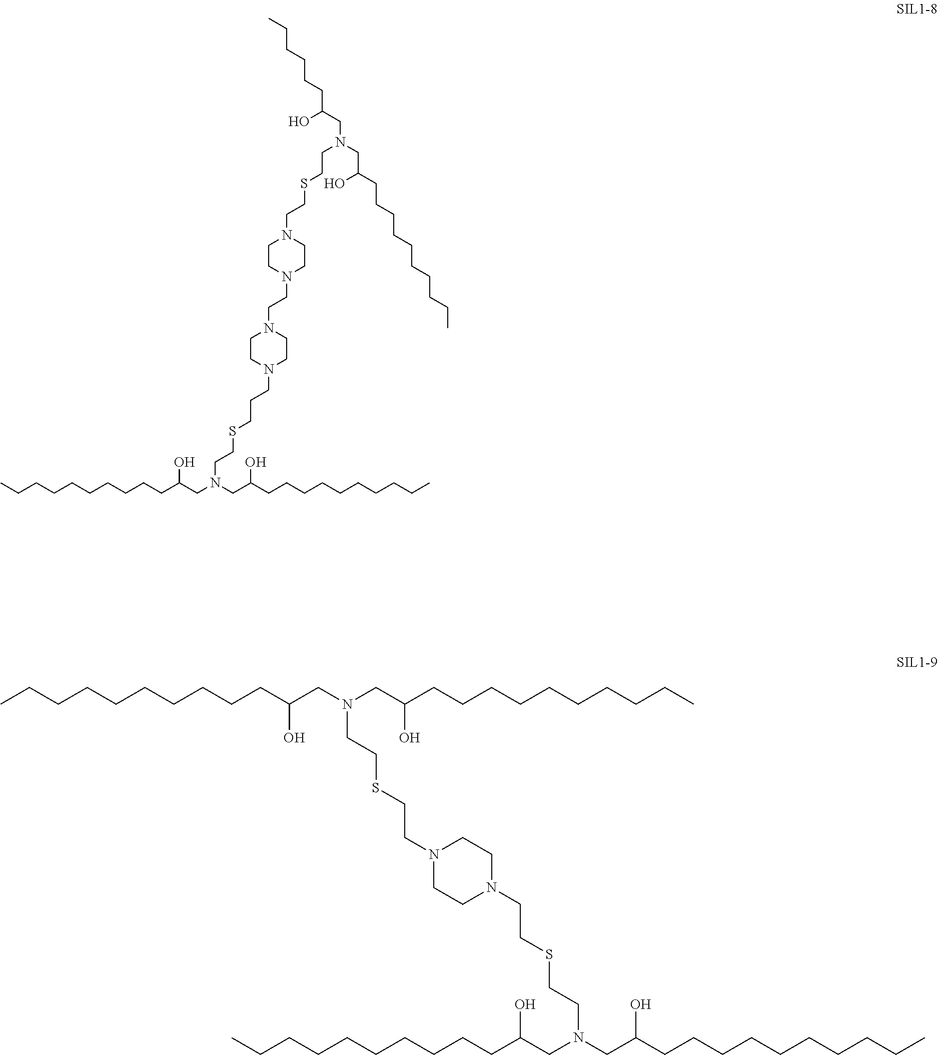 [SMILES: CCCCCCCCCCC(O)CN(CCSCCCN1CCN(CCN2CCN(CCSCCN(CC(O)CCCCCC)CC(O)CCCCCCCCCC)CC2)CC1)CC(O)CCCCCCCCCC ; CCCCCCCCCCC(O)CN(CCSCCN1CCN(CCSCCN(CC(O)CCCCCCCCCC)CC(O)CCCCCCCCCC)CC1)CC(O)CCCCCCCCCC]

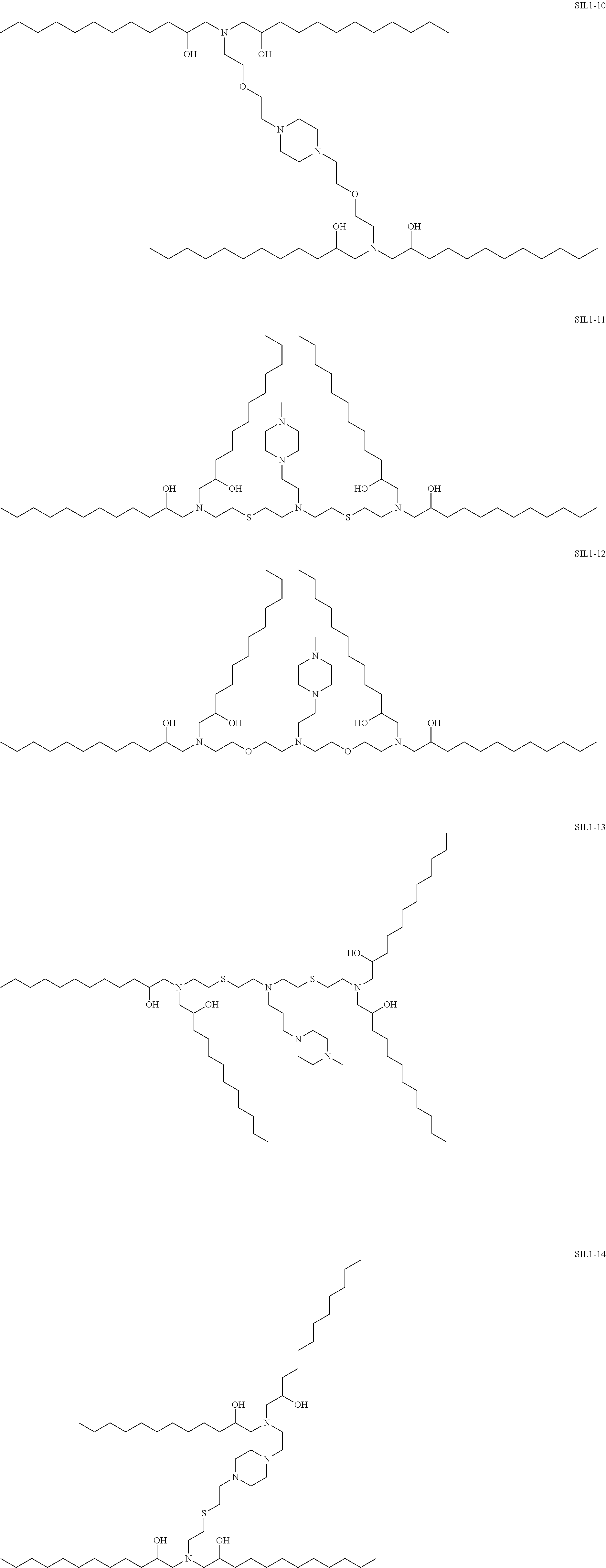

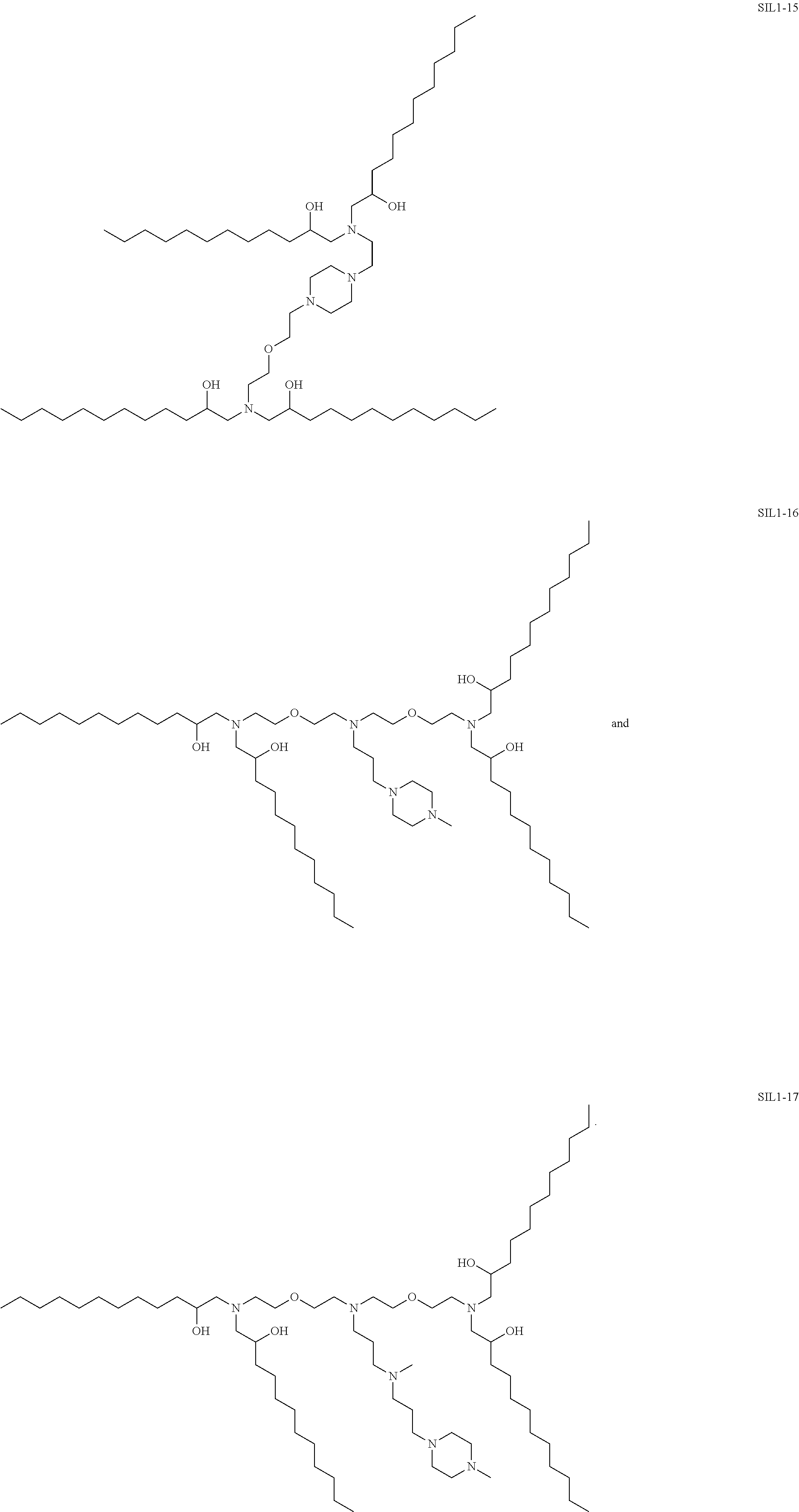

15. The compound of claim 1, wherein R₃ is, independently at each occurrence, selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, and said aryl or cycloalkyl is optionally substituted with one or more selected from the group consisting of halogen, hydroxyl, and alkoxy.

16.-24. (canceled)

25. The compound of claim 15, being a compound of formula (I-B1): 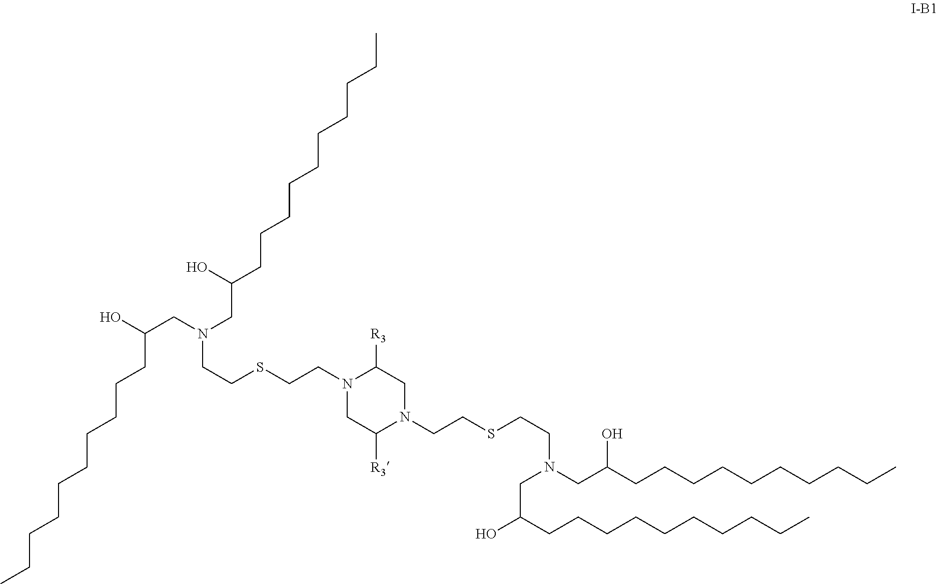
wherein R₃ and R_{3′} are different or identical, and each independently selected from the group consisting of alkyl, alkenyl, alkynyl, and cycloalkyl, wherein the alkyl, alkenyl, alkynyl, or cycloalkyl is optionally substituted with one or more selected from the group consisting of hydroxyl, —SH, alkoxyl, alkylthio, aryl, and cycloalkyl, wherein the aryl or cycloalkyl is optionally substituted with one or more selected from the group consisting of halogen, hydroxyl, and alkoxy.

26. The compound of claim 25, wherein R₃ and R_{3′} are identical.

27. The compound of claim 26, being a compound of formula (I-B2):

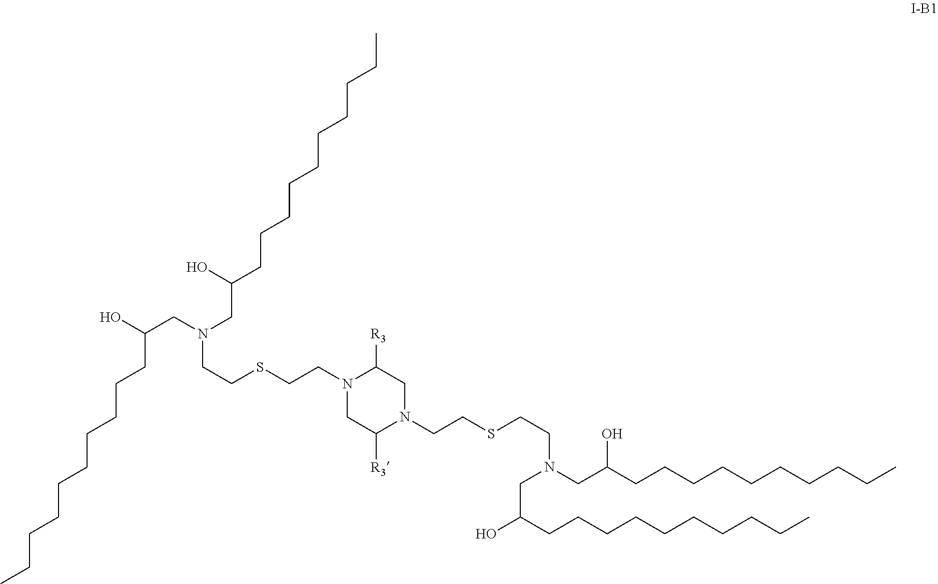

28. The compound of claim 25, selected from the group consisting of:

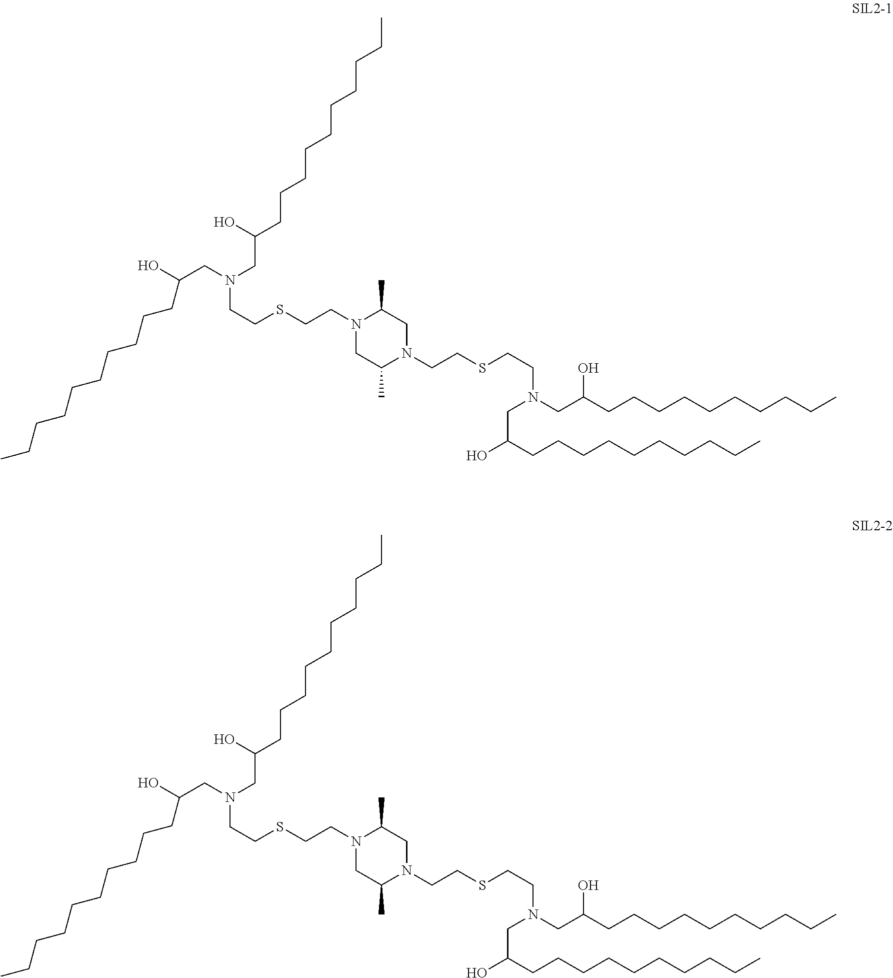

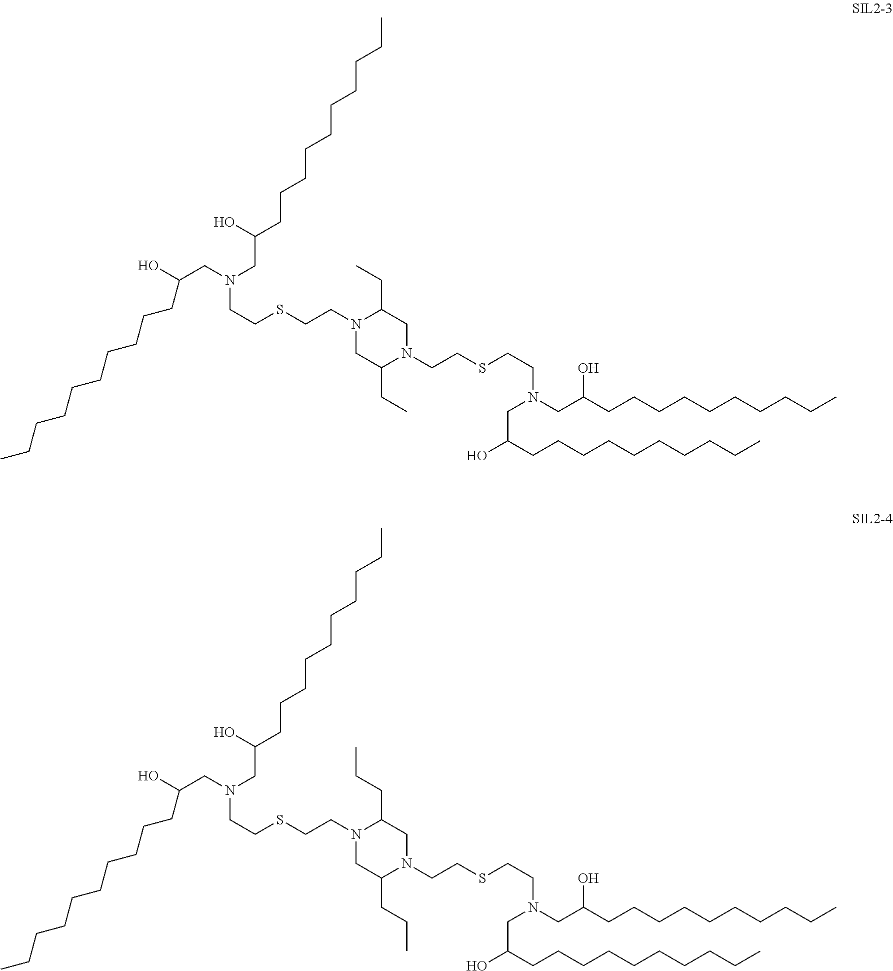

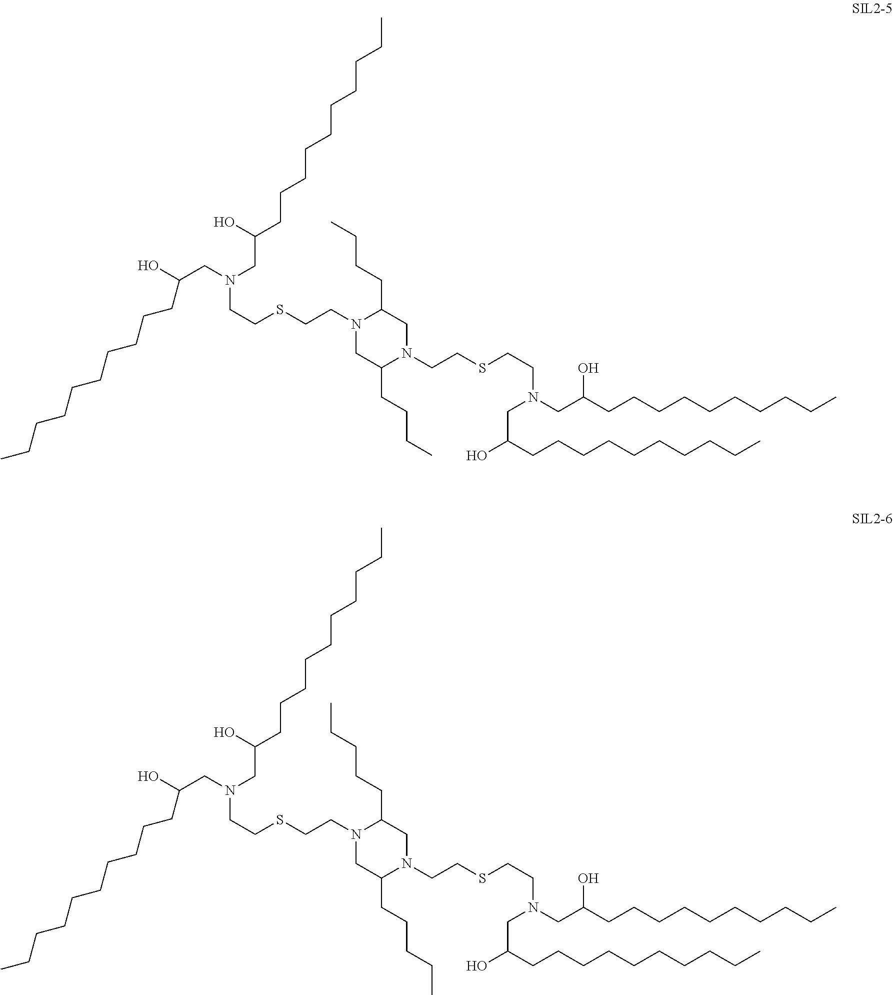

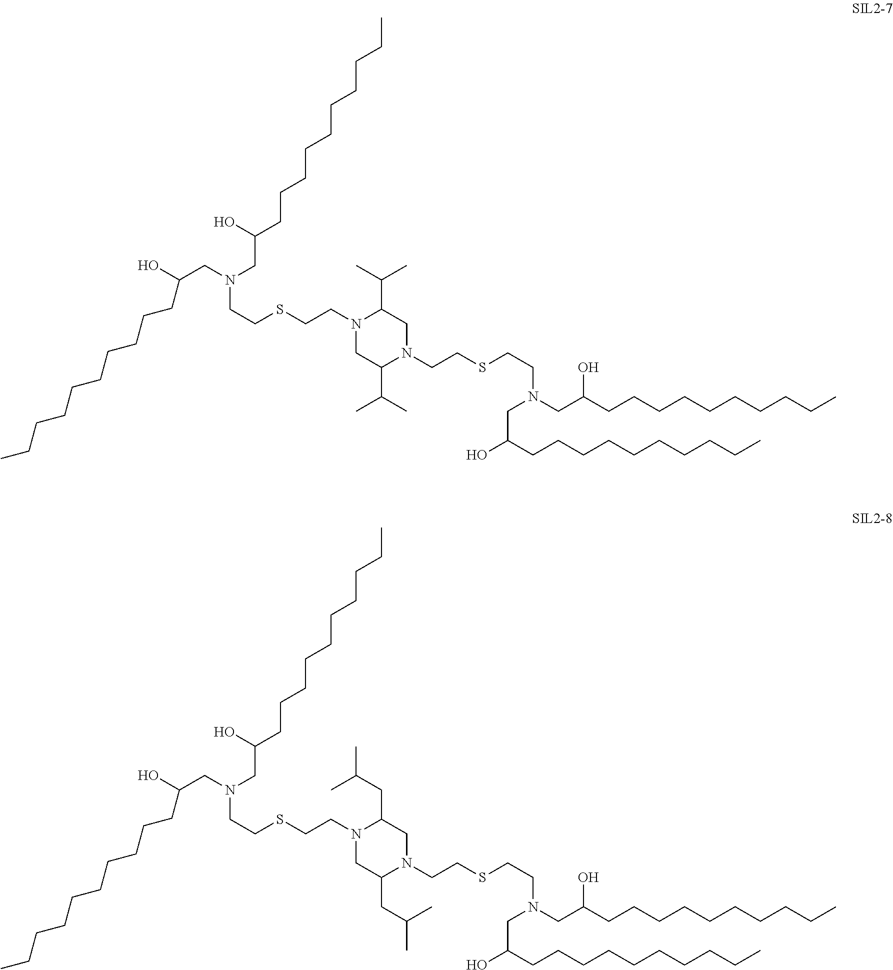

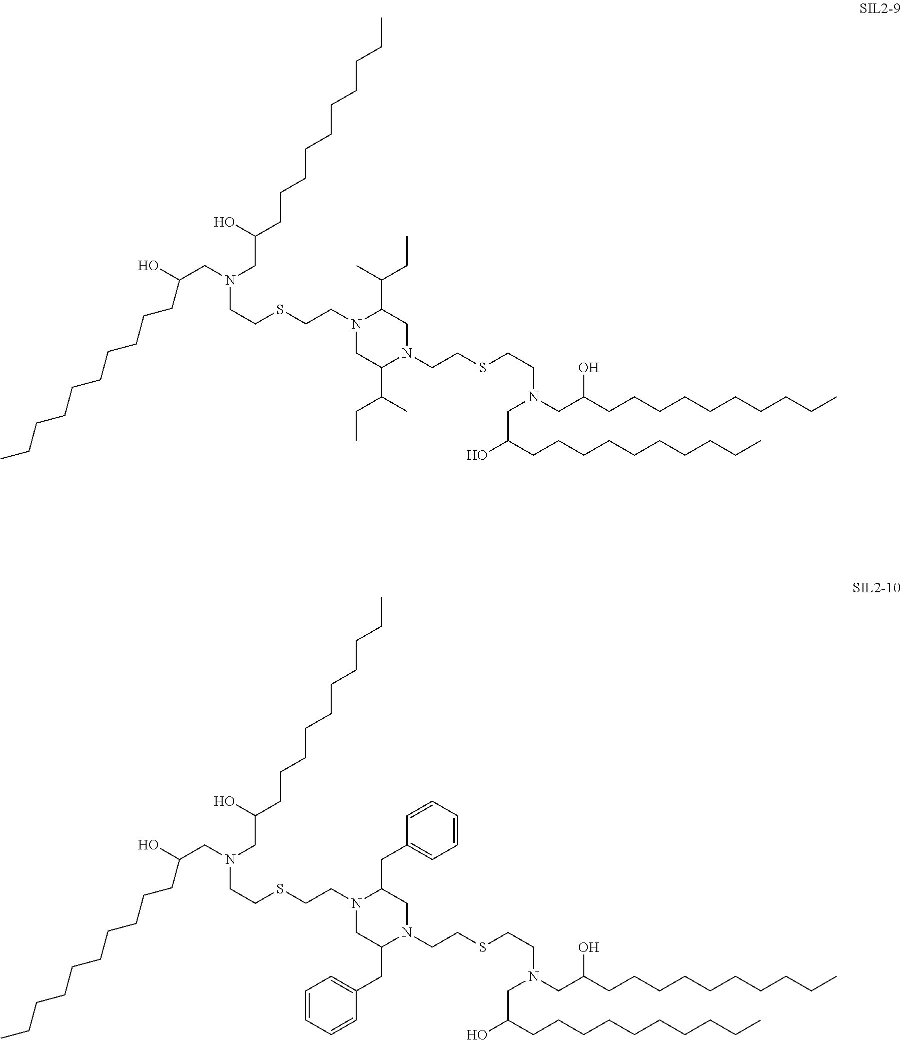

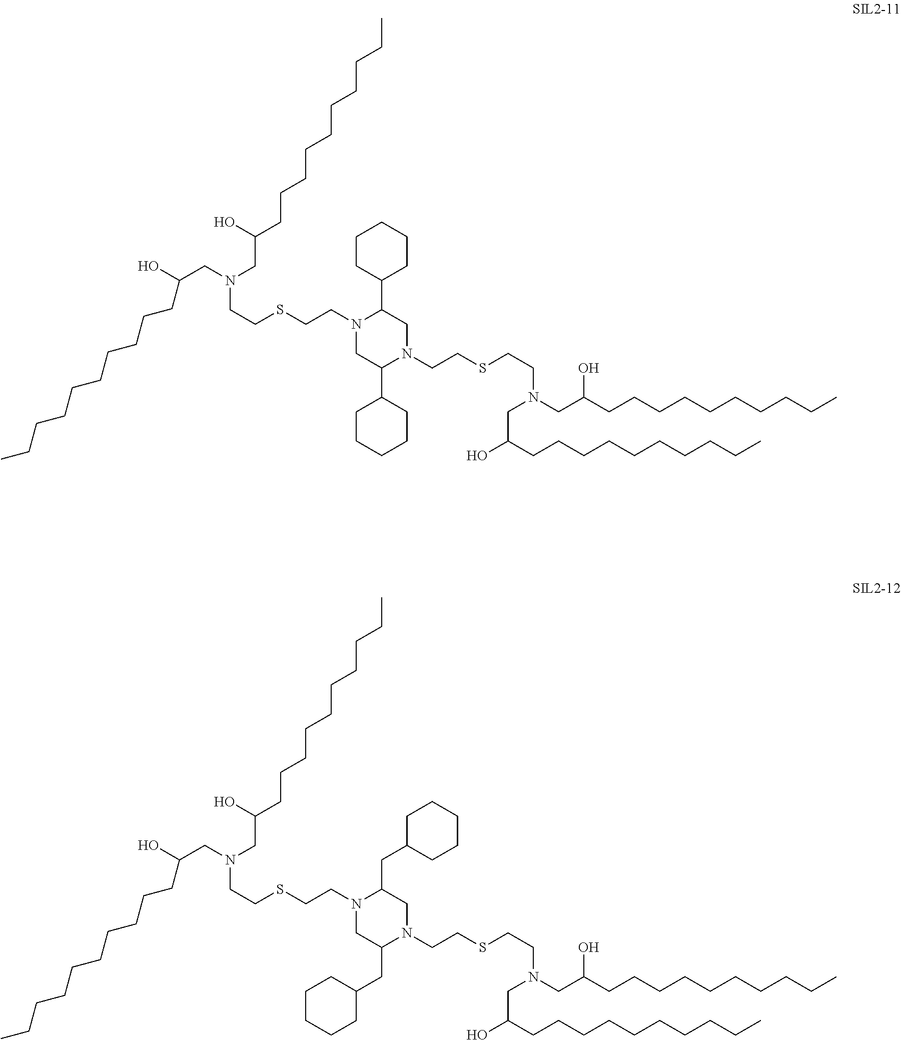

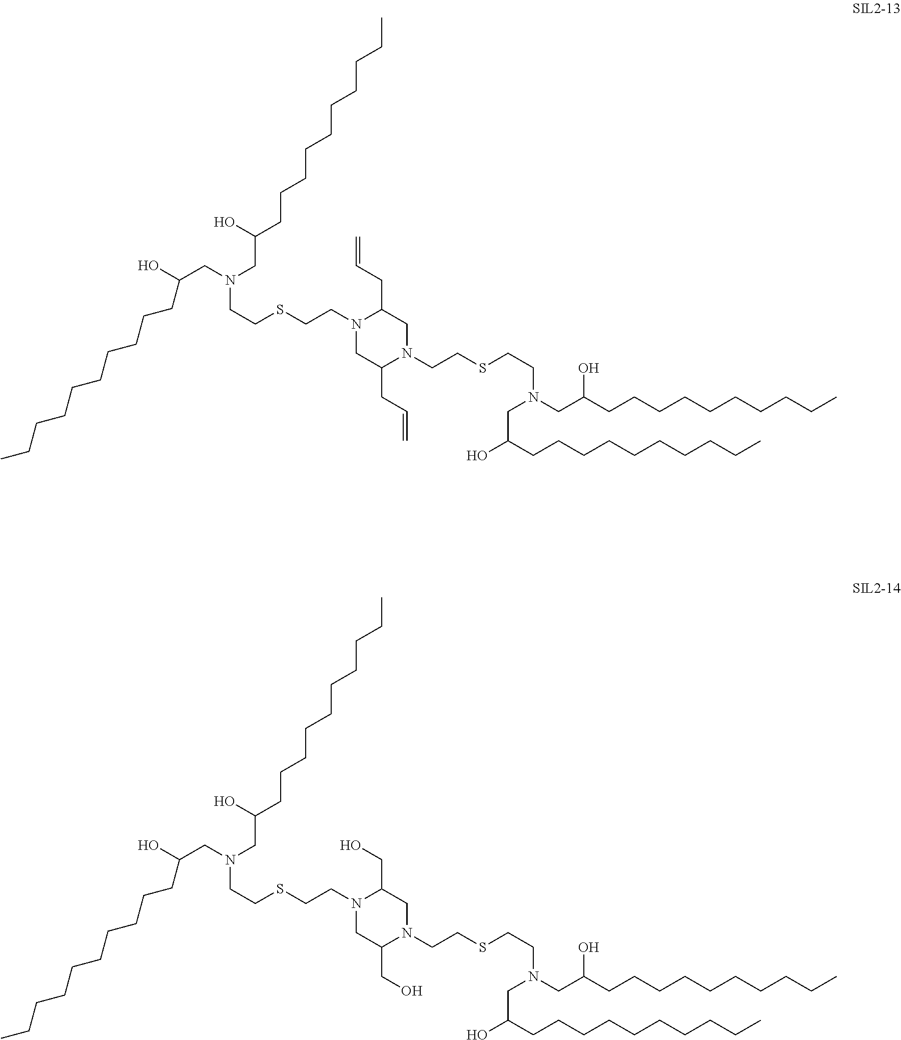

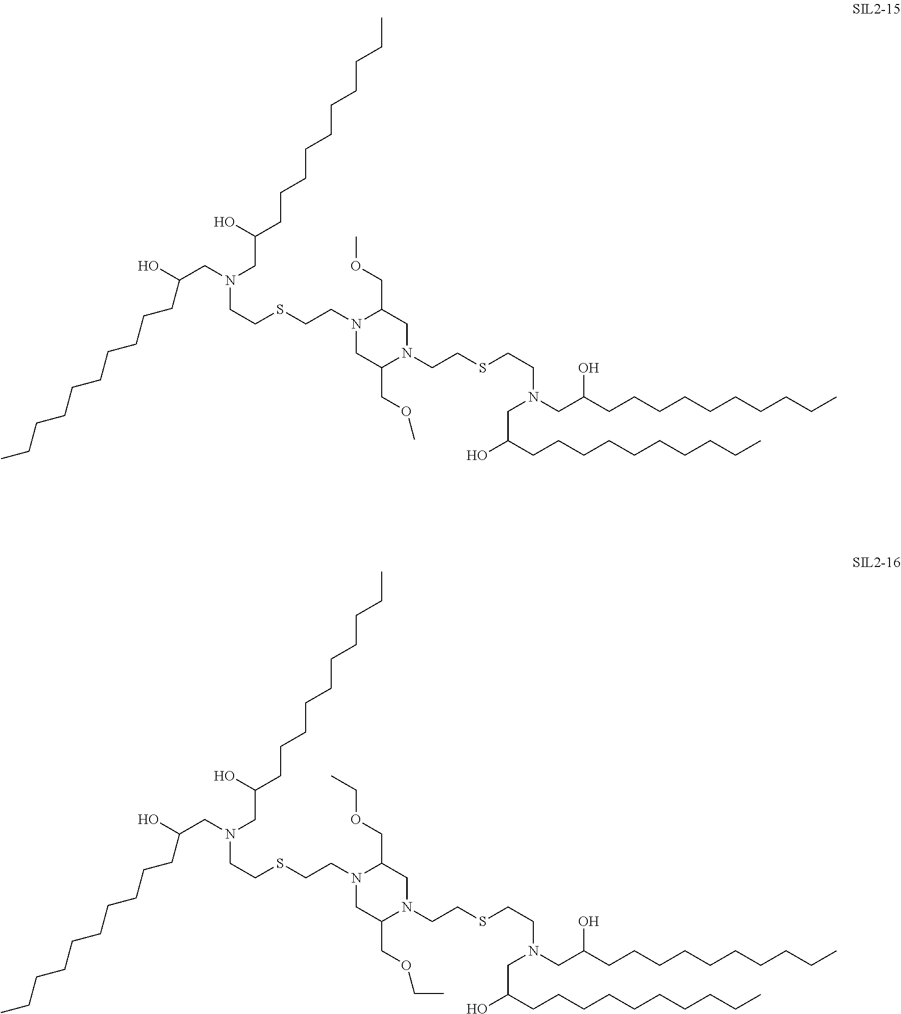

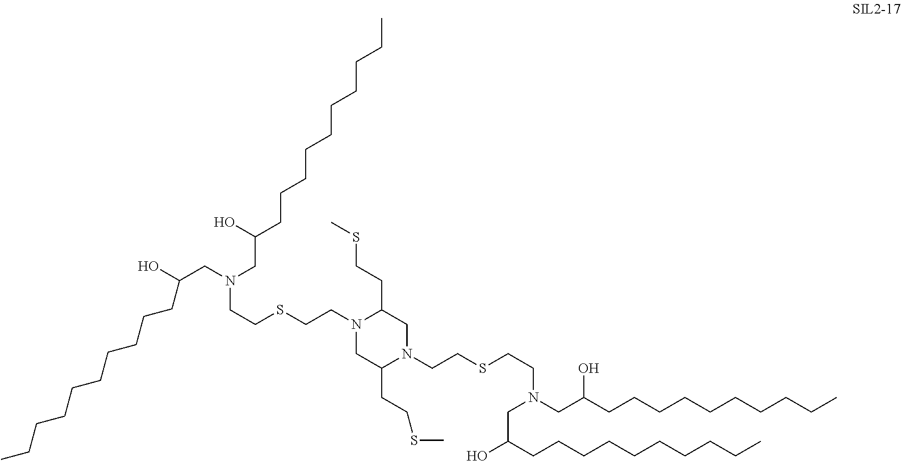 [SMILES: CCCCCCCCCCC(O)CN(CCSCCN1CC(CCSC)N(CCSCCN(CC(O)CCCCCCCCCC)CC(O)CCCCCCCCCC)CC1CCSC)CC(O)CCCCCCCCCC]

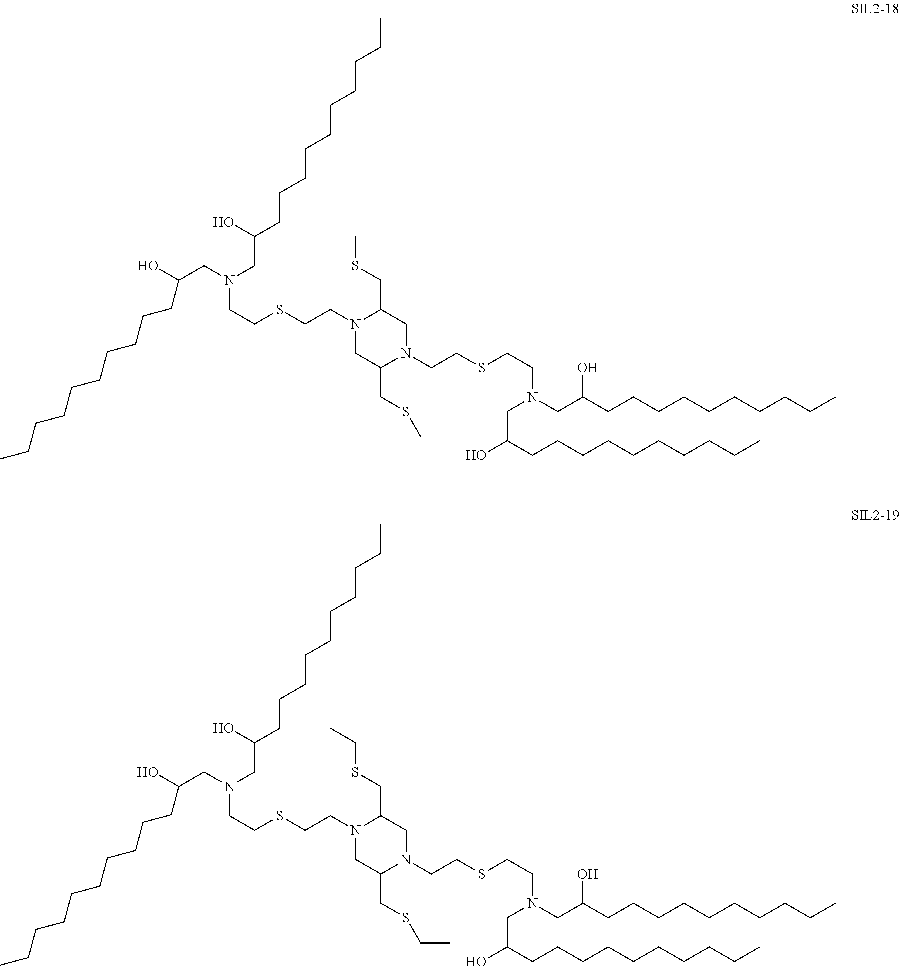

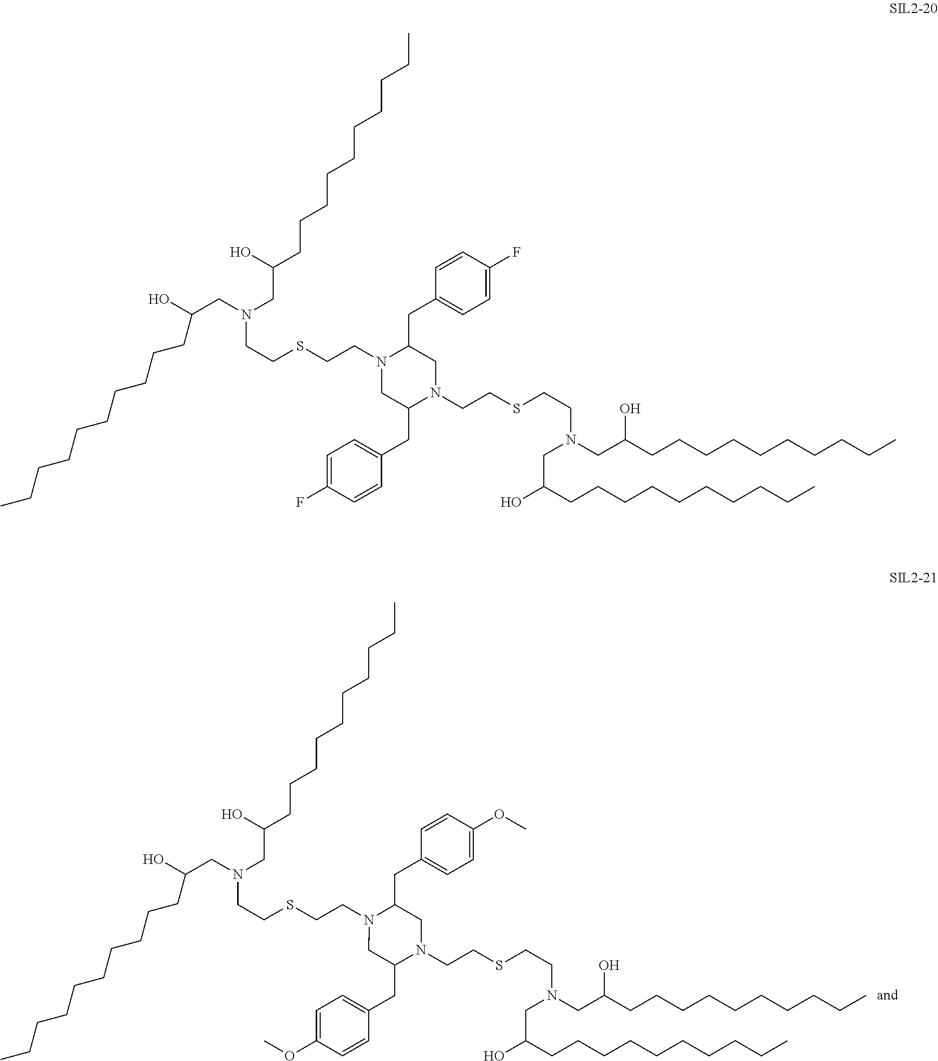

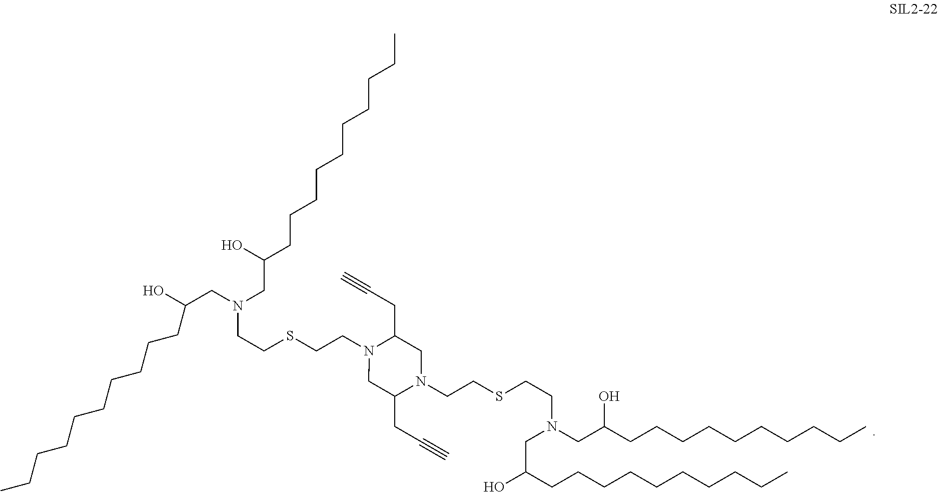 [SMILES: C#CCC1CN(CCSCCN(CC(O)CCCCCCCCCC)CC(O)CCCCCCCCCC)C(CC#C)CN1CCSCCN(CC(O)CCCCCCCCCC)CC(O)CCCCCCCCCC]

29. A compound of formula (II): 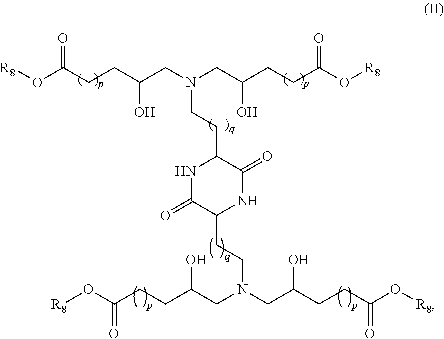
or a stereoisomer, tautomer, pharmaceutically acceptable salt or solvate thereof, wherein,
p is an integer from 1 to 10;
q is an integer from 3 to 8; and
R₈ is unbranched or branched alkyl.

30.-33. (canceled)

34. The compound of claim 29, selected from the group consisting of:

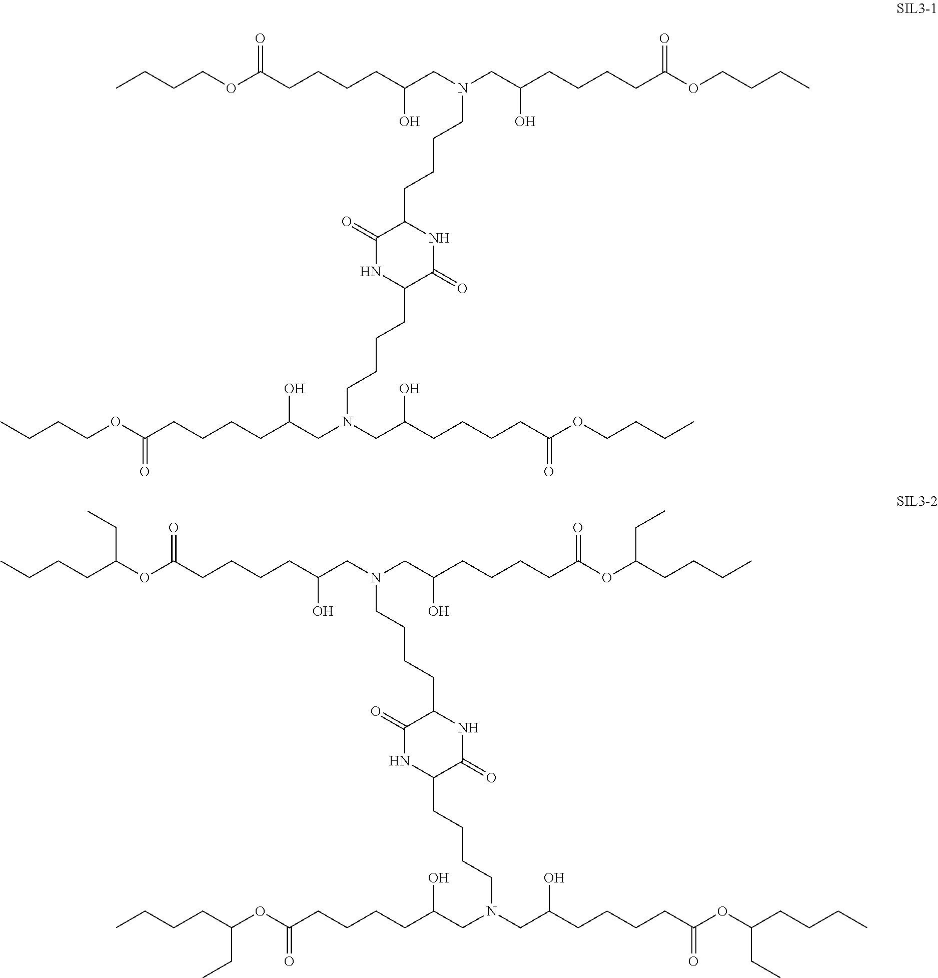

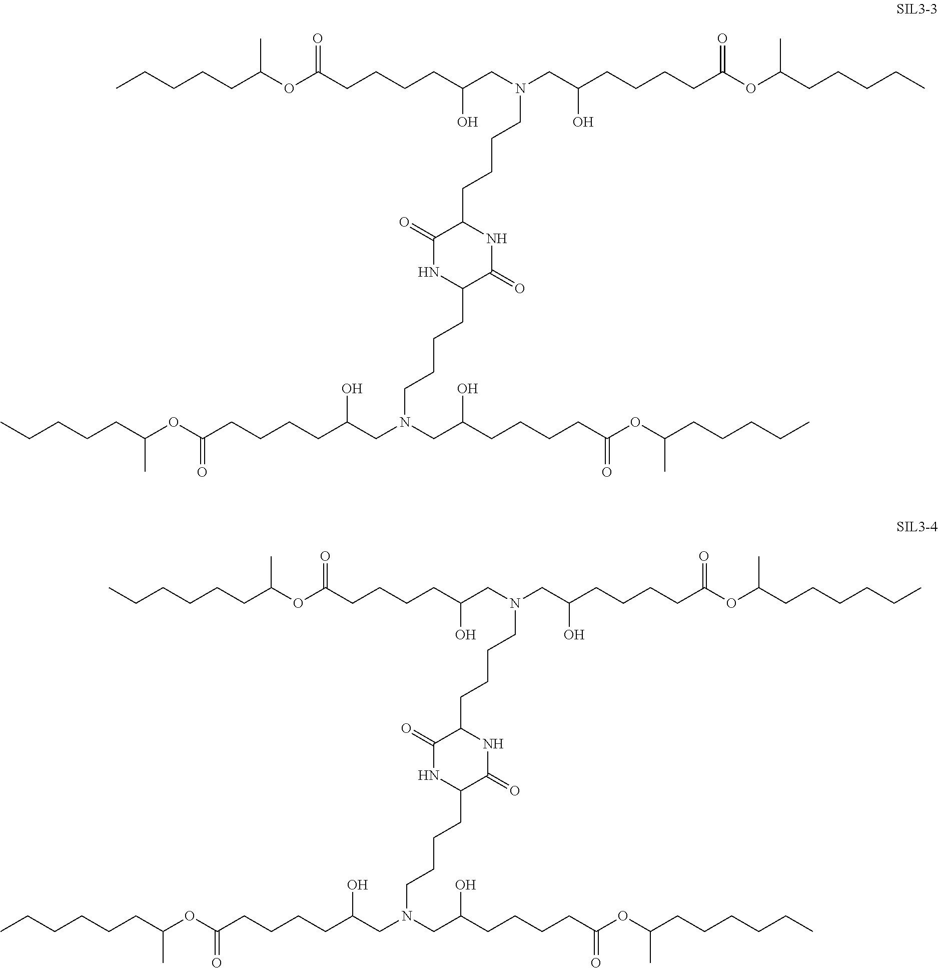

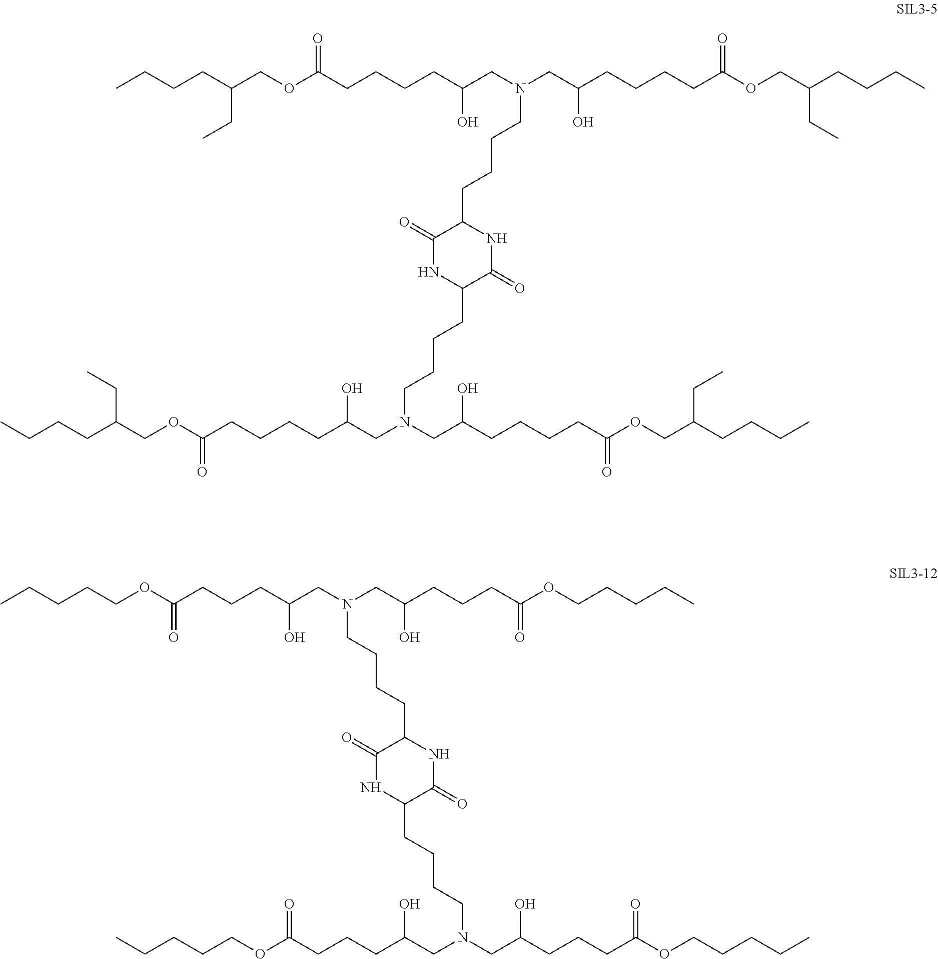

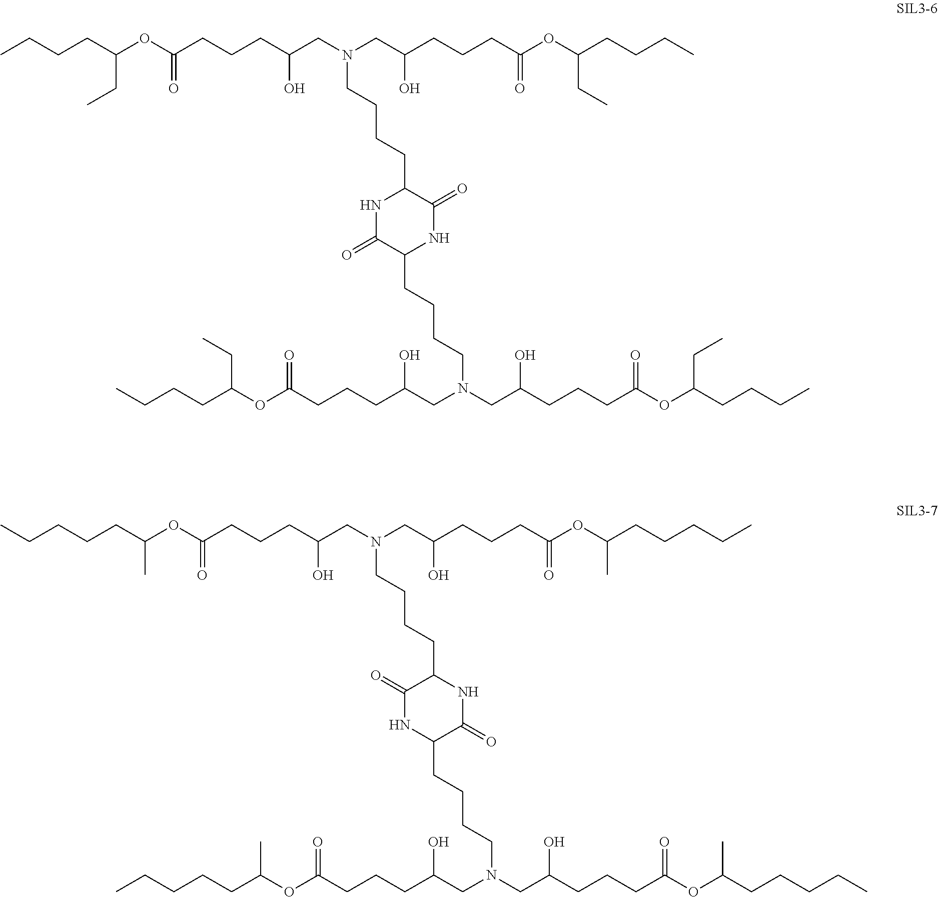

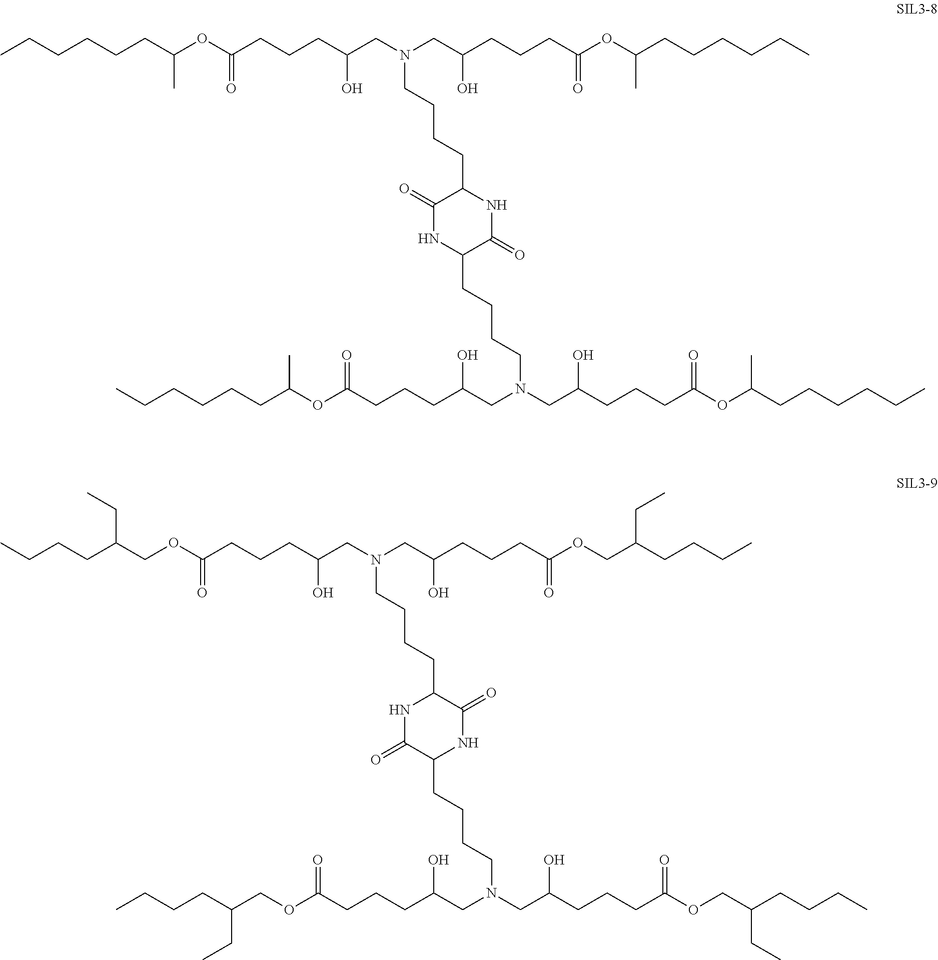

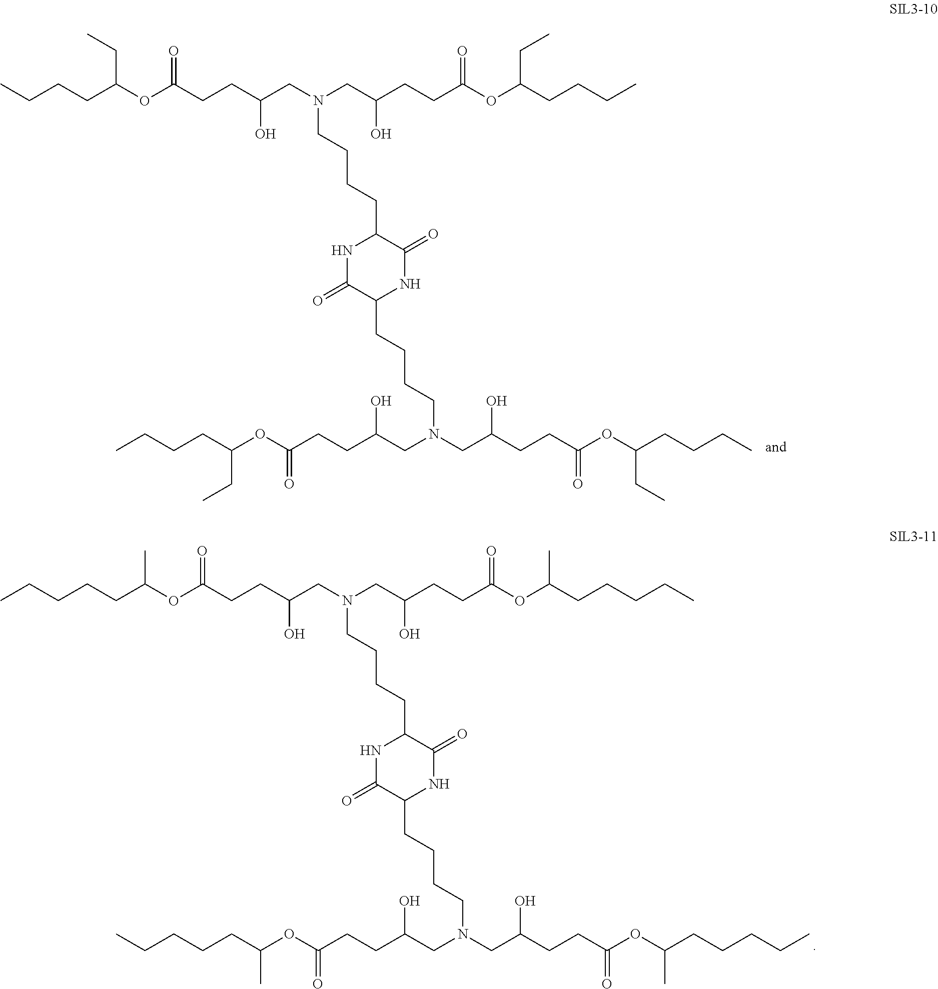

35. A method for drug delivery or cancer immunotherapy in a subject in need thereof, the method comprising administering to the subject an effective amount of the compound of claim 1.

36. (canceled)

37. A method for drug delivery or cancer immunotherapy in a subject in need thereof, the method comprising administering to the subject an effective amount of the compound of claim 29.
